(19) United States
(12) Patent Application Publication
(10) Pub. No.: US 2025/0349383 A1
(43) Pub. Date: Nov. 13, 2025

(54) SYSTEMS, DEVICES AND METHODS FOR PERSONALIZED MEDICINE IN PHARMACOGENOMICS

(71) Applicant: MyEngene Inc., Wilmington, DE (US)

(72) Inventors: Trent Marx, Calgary (CA); Ioannis Mikros, Patras (GR); Georgios Patrinos, Athens (GR); Alexandros Kanterankis, Heraklion (GR); Christoforos Nikitas Kasimatis, Piraeus (GR)

(21) Appl. No.: 18/657,515

(22) Filed: May 7, 2024

(51) Int. Cl. G16B 20/00; G16B 40/20; G16H 70/40

(52) U.S. Cl. CPC G16B 20/00; G16B 40/20; G16H 70/40

(57) ABSTRACT

Described herein are computer-implemented systems, methods, and devices for pharmacogenomic determination. The system includes a data processor configured to receive pharmacogenomic data representing at least one pharmacogenomic annotation in association with at least one gene; a database configuration engine configured to receive at least one genomic variation of the at least one gene and to search the pharmacogenomic data for at least one association with each genomic variation to return the associated data, the associated data being a haplotype or diplotype and a phenotype; a report generator configured to generate at least one report comprising the associated data with the genomic variation associated.

### FIELD

The present specification relates to tables pharmacogenomic platforms, and specifically to detecting genomic variants and reporting pharmacogenomic data.

### BACKGROUND

Pharmacogenomics relates to the use of information about a person's genome to choose the drugs and doses that are likely to work best for that patient. This scientific field combines the science of how drugs work, called pharmacology, with the science of the human genome, called genomics.

### SUMMARY

In some embodiments a pharmacogenomics determination system includes a data processor configured to receive pharmacogenomic data representing at least one pharmacogenomic annotation in association with at least one gene; a database configuration engine configured to receive at least one genomic variation of the at least one gene and to search the pharmacogenomic data for at least one association with each genomic variation to return the associated data, the associated data being a haplotype or diplotype and a phenotype; a report generator configured to generate at least one report comprising the associated data with the genomic variation associated; and a display generator configured to generate a display based on the at least one report, the display further comprising at least one interface element representing the associated data with the genomic variation associated.

In some embodiments, the phenotype includes adverse drug reactions, metabolizing status, efficacy indications, dosing data, alternative drug data, pharmacogenomic indication, or prescribing data.

In some embodiments the report generator is configured to receive at least one text-based file representing at least one genetic sequence and generate at least one binary file representing at least one genetic sequence, at least one index file for the at least one binary file, and at least one format text file for the at least one binary file.

In some embodiments a machine learning engine is configured to predict at least one genomic variant, wherein at least one of the at least one genomic variation is determined as the at least one genomic variant.

In some embodiments the machine learning engine is configured to detect genomic variants leading to altered protein function, the machine learning engine including a non-transitory memory storing one or more features from an annotated variant dataset of at least one variant; a variant validator configured to determine one or more validated variants of the annotated variant dataset, each validated variant matching one or more known variants of a known variant dataset, each known variant leading to altered protein function; a machine learning model configured to assign a classification to one or more predicted variants of variants of the annotated variant dataset not selected as validated variants, each predicted variant leading to altered protein function, the assigning by the machine learning model based on at least one of the one or more features stored in the memory; and a loss-of-function detector configured to determine one or more sequence ontology variants of the variants of the annotated variant dataset not selected as validated variants and not classified as predicted variants, each sequence ontology variant being a loss-of-function variant, the determining by the loss-of-function detector based on at least one of the features stored in the memory.

In some embodiments, the annotated variant dataset is generated using a Variant Effect Predictor (VEP).

In some embodiments, each sequence ontology variant is determined by filtering based on sequence ontology data.

In some embodiments, the loss-of-function variant is a splice acceptor variant, a splice donor variant, a stop gained variant, a frameshift variant, a stop loss variant, or a start loss variant.

In some embodiments the machine learning model is trained using a training dataset of annotated variants, the training dataset of annotated variants generated based on protein functional domain data, sequence ontology data, at least one prediction score, a LoF indicator feature representing a loss-of-function variant and generated using the sequence ontology data, and an Interpro indicator feature representing an effect on an Interpro domain and generated using the Interpro domain data; wherein the protein functional domain data is Interpro domain data; and wherein the sequence ontology data represents a splice acceptor variant, a splice donor variant, a stop gained variant, a frameshift variant, a stop lost variant, a start lost variant, or a combination thereof.

In some embodiments, the interface generator configured to generate one or more user interface objects on a graphical interface of a display, the one or more user interface objects representing: variant data, the variant data generated based on each validated variant, each predicted variant, and each sequence ontology variant; wherein the one or more user interface objects is generated based on gene location, functional effect, evidence tag, novelty, or pharmacogenomic data; and wherein each evidence tag is assigned to each validated variant by the variant validator, each predicted variant by the machine learning model, or each sequence ontology variant by the loss-of-function detector.

In some embodiments, the interface generator is configured to: receive additional data; determine an association, if any, between the additional data and each validated variant, each predicted variant, and each sequence ontology variant; and generate the one or more user interface objects to represent the additional data, if any, associated with each validated variant, each predicted variant, and each sequence ontology variant.

In some embodiments, the classification represents altered protein function corresponding to predicted variants in CYP2B6, CYP2C19, CYP2C9, CYP2D6, DPYD, NUDT15, RYR1, SLCO1B1, TPMT, UGT1A1, BRCA1, BRCA2, or combination thereof.

In some embodiments, the machine learning engine is configured: using the one or more validated variants, the one or more predicted variants, and the one or more sequence ontology variants to determine a clinical intervention.

In some embodiments, the machine learning engine is configured, using the one or more validated variants, the one or more predicted variants, and the one or more sequence ontology variants to determine responsiveness for a treatment of psychiatric disease;

In some embodiments, the machine learning engine is configured, using the one or more validated variants, the one or more predicted variants, and the one or more sequence ontology variants for multiomics.

In some embodiments the machine learning engine includes at least one processor; and at least one non-transitory memory storing computer-executable instructions which, when executed, cause the at least one processor to perform a method, the method including: generating at least one annotated variant training dataset, the generating including: receiving at least one annotated variant dataset, annotated based on protein functional domain data, sequence ontology data, and at least one prediction score; and applying k-nearest neighbour (kNN) imputation to the at least one annotated variant dataset to generate one or more values for missing data; and training the machine learning model using the at least one annotated variant training dataset; wherein the at least one annotated variant dataset is annotated using a Variant Effect Predictor (VEP).

In some embodiments the machine learning engine is configured wherein each prediction score is generated using LoFtool, DEOGEN2, MPC, BayesDel_addAF, FATHMM, integrated_fitCons, or LIST.S2; wherein the protein functional domain data is Interpro domain data; wherein the sequence ontology data represents a splice acceptor variant, a splice donor variant, a stop gained variant, a frameshift variant, a stop lost variant, a start lost variant, or a combination thereof; wherein generating at least one annotated variant training dataset further comprises: generating a LoF indicator feature using the sequence ontology data, the LoF indicator feature representing a loss-of-function variant; and wherein generating at least one annotated variant training dataset further comprises: generating an Interpro indicator feature using the Interpro domain data, the Interpro indicator feature representing an effect on an Interpro domain.

In some embodiments, the machine learning model is a random forest classifier having decision trees, the machine learning model configured to assign a classification based on bootstrap aggregation using the decision trees; wherein the kNN imputation is kNN imputation with weighted mean; wherein generating at least one annotated variant training dataset further includes: removing data from the at least one annotated variant dataset, wherein the data corresponds to a variant having a percentage greater than or equal to 40%, collectively, of missing values for the annotations, the removing performed before kNN imputation is applied to the at least one annotated variant dataset; and removing data from the at least one annotated variant dataset, wherein the data corresponds to a feature having a percentage greater than or equal to 40%, collectively, of missing values for variants represented in the at least one annotated variant dataset, the removing performed before kNN imputation is applied to the at least one annotated variant dataset.

In some embodiments, generating at least one annotated variant training dataset further includes: performing variant deduplication on the at least one annotated variant dataset to generate at least one new annotated variant dataset; extracting features from the at least one annotated variant dataset, the features comprising protein functional domain data, sequence ontology data, at least one prediction score, at least one variant identifier, and at least one sequence identifier; generating a LoF indicator feature using the sequence ontology data, the LoF indicator feature representing a loss-of-function variant; and generating an Interpro indicator feature using the Interpro domain data, the Interpro indicator feature representing an effect on an Interpro domain.

In some embodiments a method for pharmacogenomic determination includes receiving pharmacogenomic data representing at least one pharmacogenomic annotation in association with at least one gene; receiving at least one genomic variation of the at least one gene, searching the pharmacogenomic data for at least one association with each genomic variation, and returning the associated data, the associated data being a haplotype or diplotype and a phenotype; generating at least one report comprising the associated data with the genomic variation associated; and generating a display based on the at least one report, the display further comprising at least one interface element representing the associated data with the genomic variation associated.

In some embodiments the machine learning engine further includes predicting at least one genomic variant, wherein at least one of the at least one genomic variation is determined as the at least one genomic variant.

In some embodiments, the at least one text-based file is a FASTQ file; wherein the at least one binary file is at least one BAM file, the at least one index file is at least one bai file, and the at least one format file is at least one VCF file.

In some embodiments a method for pharmacogenomic determination includes: receiving pharmacogenomic data representing at least one pharmacogenomic annotation in association with at least one gene; receiving at least one genomic variation of the at least one gene and searching the pharmacogenomic data for at least one association with each genomic variation to return the associated data; the associated data being a haplotype or diplotype and a phenotype; generating at least one report comprising the associated data with the genomic variation associated; and generate a display based on the at least one report, the display further comprising at least one interface element representing the associated data with the genomic variation associated.

In some embodiments, a system for pharmacogenomic determination, includes a data processor configured to receive pharmacogenomic data representing at least one pharmacogenomic annotation in association with at least one gene; a database configuration engine configured to determine at least one genomic variation and to search the pharmacogenomic data for at least one association with each genomic variation to return the associated data; and a report generator configured to generate at least one report comprising the associated data with the genomic variation associated.

In some embodiments, the report generator is configured to receive at least one text-based file representing at least one genetic sequence and generate at least one binary file representing at least one genetic sequence, at least one index file for the at least one binary file, and at least one format file for the at least one binary file.

In some embodiments, the system, further includes a machine learning engine configured to predict at least one genomic variant, wherein the database configuration engine is configured to determine the at least one of the at least one genomic variation as the at least one genomic variant.

In some embodiments, the at least one text-based file is a FASTQ file.

In some embodiments, the at least one binary file is at least one BAM file, the at least one index file is at least one bai file, and the at least one format file is at least one VCF file.

In some embodiments, the database configuration engine is configured to determine at least one diplotype for genes of interest and to determine a phenotype corresponding to the at least one diplotype.

In some embodiments, the system further includes a display generator configured to generate a display based on the at least one report, the display further comprising at least one interface element representing the associated data with the genomic variation associated.

In some embodiments, a method for pharmacogenomic determination includes:
receiving pharmacogenomic data representing at least one pharmacogenomic annotation in association with at least one gene; determining at least one genomic variation and searching the pharmacogenomic data for at least one association with each genomic variation to return the associated data; and generating at least one report comprising the associated data with the genomic variation associated.

In some embodiments the method, further includes receiving at least one text-based file representing at least one genetic sequence and generating at least one binary file representing at least one genetic sequence, at least one index file for the at least one binary file, and at least one format file for the at least one binary file.

In some embodiments the method, further includes predicting at least one genomic variant, wherein at least one of the at least one genomic variation is determined as the at least one genomic variant.

In some embodiments, the at least one text-based file is a FASTQ file.

In some embodiments, the at least one binary file is at least one BAM file, the at least one index file is at least one bai file, and the at least one format file is at least one VCF file.

In some embodiments, the method, further comprising: determining at least one diplotype for genes of interest and determining a phenotype corresponding to the at least one diplotype; and wherein the at least one report comprises the phenotype.

In some embodiments the method, further includes generating a display based on the at least one report, the display further comprising at least one interface element representing the associated data with the genomic variation associated.

In some embodiments, there is provided a non-transitory computer readable medium storing a set of machine-interpretable instructions, which, when executed, cause a processor to perform a method for pharmacogenomic determination, the method includes: receiving pharmacogenomic data representing at least one pharmacogenomic annotation in association with at least one gene;

determining at least one genomic variation and searching the pharmacogenomic data for at least one association with each genomic variation to return the associated data; and generating at least one report comprising the associated data with the genomic variation associated.

In some embodiments a system for detecting genomic variants leading to altered protein function includes: a non-transitory memory storing one or more features from an annotated variant dataset of at least one variant; a variant validator configured to determine one or more validated variants of the annotated variant dataset, each validated variant matching one or more known variants of a known variant dataset, each known variant leading to altered protein function; a machine learning model configured to assign a classification to one or more predicted variants of variants of the annotated variant dataset not selected as validated variants, each predicted variant leading to altered protein function, the assigning by the machine learning model based on at least one of the one or more features stored in the memory; and a loss-of-function detector configured to determine one or more sequence ontology variants of the variants of the annotated variant dataset not selected as validated variants and not classified as predicted variants, each sequence ontology variant being a loss-of-function variant, the determining by the loss-of-function detector based on at least one of the features stored in the memory.

In some embodiments, the annotated variant dataset is generated using a Variant Effect Predictor (VEP).

In some embodiments, each sequence ontology variant is determined by filtering based on sequence ontology data.

In some embodiments, the machine learning model is trained using a training dataset of annotated variants, the training dataset of annotated variants generated based on protein functional domain data, sequence ontology data, at least one prediction score, a LoF indicator feature representing a loss-of-function variant and generated using the sequence ontology data, and an Interpro indicator feature representing an effect on an Interpro domain and generated using the Interpro domain data; wherein the protein functional domain data is Interpro domain data; and wherein the sequence ontology data represents a splice acceptor variant, a splice donor variant, a stop gained variant, a frameshift variant, a stop lost variant, a start lost variant, or a combination thereof.

In some embodiments the loss-of-function variant is a splice acceptor variant, a splice donor variant, a stop gained variant, a frameshift variant, a stop loss variant, or a start loss variant.

In some embodiments the system, further includes: an interface generator configured to generate one or more user interface objects on a graphical interface of a display, the one or more user interface objects representing: variant data, the variant data generated based on each validated variant, each predicted variant, and each sequence ontology variant; wherein the one or more user interface objects is generated based on gene location, functional effect, evidence tag, novelty, or pharmacogenomic data; and wherein each evidence tag is assigned to each validated variant by the variant validator, each predicted variant by the machine learning model, or each sequence ontology variant by the loss-of-function detector.

In some embodiments the system, wherein the interface generator is configured to: receive additional data; determine an association, if any, between the additional data and each validated variant, each predicted variant, and each sequence ontology variant; and generate the one or more user interface objects to represent the additional data, if any, associated with each validated variant, each predicted variant, and each sequence ontology variant.

In some embodiments the classification represents altered protein function corresponding to predicted variants in CYP2B6, CYP2C19, CYP2C9, CYP2D6, DPYD, NUDT15, RYR1, SLCO1B1, TPMT, UGT1A1, BRCA1, BRCA2, or combination thereof.

In some embodiments the system, further includes using the one or more validated variants, the one or more predicted variants, and the one or more sequence ontology variants to determine a clinical intervention.

In some embodiments the system, further includes using the one or more validated variants, the one or more predicted variants, and the one or more sequence ontology variants to determine responsiveness for a treatment of psychiatric disease.

In some embodiments the system, further includes using the one or more validated variants, the one or more predicted variants, and the one or more sequence ontology variants for multiomics.

In some embodiments, a system for training a machine learning model for detecting genomic variants includes: at least one processor; and at least one non-transitory memory storing computer-executable instructions which, when executed, cause the at least one processor to perform a method, the method comprising: generating at least one annotated variant training dataset, the generating comprising: receiving at least one annotated variant dataset, annotated based on protein functional domain data, sequence ontology data, and at least one prediction score; and applying k-nearest neighbour (kNN) imputation to the at least one annotated variant dataset to generate one or more values for missing data; and training a machine learning model using the at least one annotated variant training dataset.

In some embodiments the at least one annotated variant dataset is annotated using a Variant Effect Predictor (VEP).

In some embodiments each prediction score is generated using LoFtool, DEOGEN2, MPC, BayesDel_addAF, FATHMM, integrated_fitCons, or LIST.S2.

In some embodiments the protein functional domain data is Interpro domain data.

In some embodiments, the sequence ontology data represents a splice acceptor variant, a splice donor variant, a stop gained variant, a frameshift variant, a stop lost variant, a start lost variant, or a combination thereof.

In some embodiments, generating at least one annotated variant training dataset further includes: generating a LoF indicator feature using the sequence ontology data, the LoF indicator feature representing a loss-of-function variant.

In some embodiments, the system for training a machine learning model, wherein generating at least one annotated variant training dataset further comprises: generating an Interpro indicator feature using the Interpro domain data, the Interpro indicator feature representing an effect on an Interpro domain.

In some embodiments, the machine learning model is a random forest classifier having decision trees, the machine learning model configured to assign a classification based on bootstrap aggregation using the decision trees.

In some embodiments, the kNN imputation is kNN imputation with weighted mean.

In some embodiments, generating at least one annotated variant training dataset further includes: removing data from the at least one annotated variant dataset, wherein the data corresponds to a variant having a percentage greater than or equal to 40%, collectively, of missing values for the annotations, the removing performed before kNN imputation is applied to the at least one annotated variant dataset; and removing data from the at least one annotated variant dataset, wherein the data corresponds to a feature having a percentage greater than or equal to 40%, collectively, of missing values for variants represented in the at least one annotated variant dataset, the removing performed before kNN imputation is applied to the at least one annotated variant dataset.

In some embodiments, generating at least one annotated variant training dataset further includes: performing variant deduplication on the at least one annotated variant dataset to generate at least one new annotated variant dataset; extracting features from the at least one annotated variant dataset, the features comprising protein functional domain data, sequence ontology data, at least one prediction score, at least one variant identifier, and at least one sequence identifier; generating a LoF indicator feature using the sequence ontology data, the LoF indicator feature representing a loss-of-function variant; and generating an Interpro indicator feature using the Interpro domain data, the Interpro indicator feature representing an effect on an Interpro domain.

A non-transitory computer readable medium storing a set of machine-interpretable instructions, which, when executed, cause a processor to perform a method for detecting genomic variants leading to altered protein function, the method includes: storing, in a non-transitory memory, one or more features from an annotated variant dataset of at least one variant; determining one or more validated variants of the annotated variant dataset, each validated variant matching one or more known variants of a known variant dataset, each known variant leading to altered protein function; assigning a classification to one or more predicted variants of variants of the annotated variant dataset not selected as validated variants, each predicted variant leading to altered protein function, the assigning by the machine learning model based on at least one of the features stored in the memory; and determining one or more sequence ontology variants of the variants of the annotated variant dataset not selected as validated variants and not classified as predicted variants, each sequence ontology variant being a loss-of-function variant, the determining by the loss-of-function detector based on at least one of the features stored in the memory.

Other aspects and features according to the present application will become apparent to those ordinarily skilled in the art upon review of the following description of embodiments of the invention in conjunction with the accompanying figures.

### BRIEF DESCRIPTION

The principles of the invention may better be understood with reference to the accompanying figures provided by way of illustration of an exemplary embodiment, or embodiments, incorporating principles and aspects of the present invention, and in which:

FIG. 1 is code implemented by a pharmacogenomic platform, according to some embodiments;

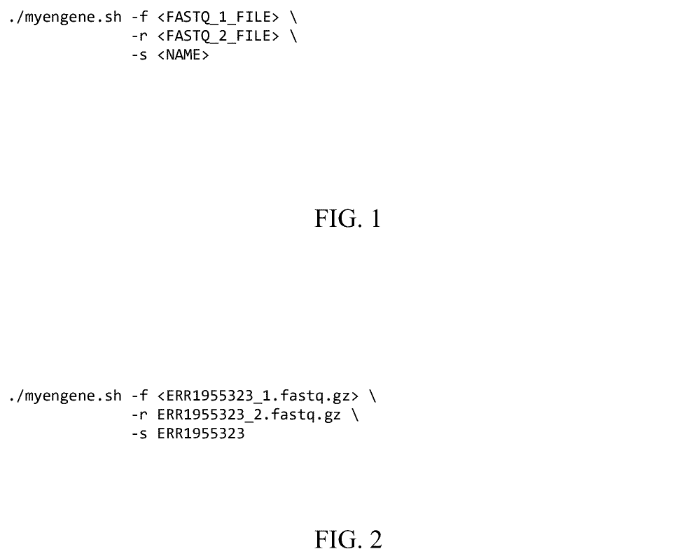

FIG. 2 is code implemented by a pharmacogenomic platform, according to some embodiments;

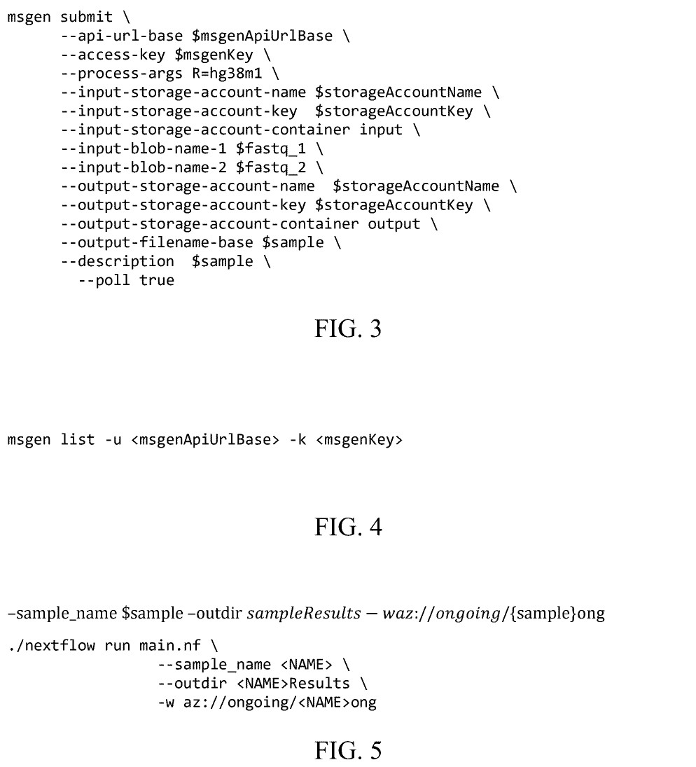

FIG. 3 is code implemented by a pharmacogenomic platform, according to some embodiments;

FIG. 4 is code implemented by a pharmacogenomic platform, according to some embodiments;

FIG. 5 is code implemented by a pharmacogenomic platform, according to some embodiments;

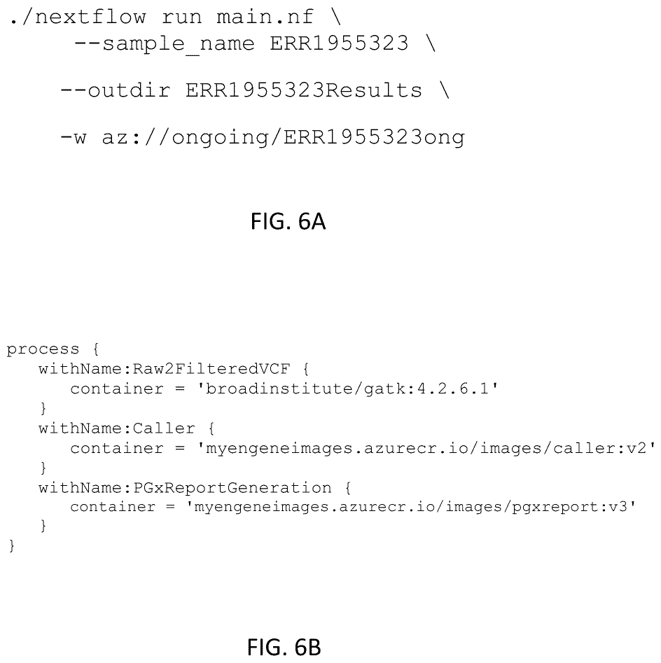

FIG. 6A and FIG. 6B is code implemented by a pharmacogenomic platform, according to some embodiments;

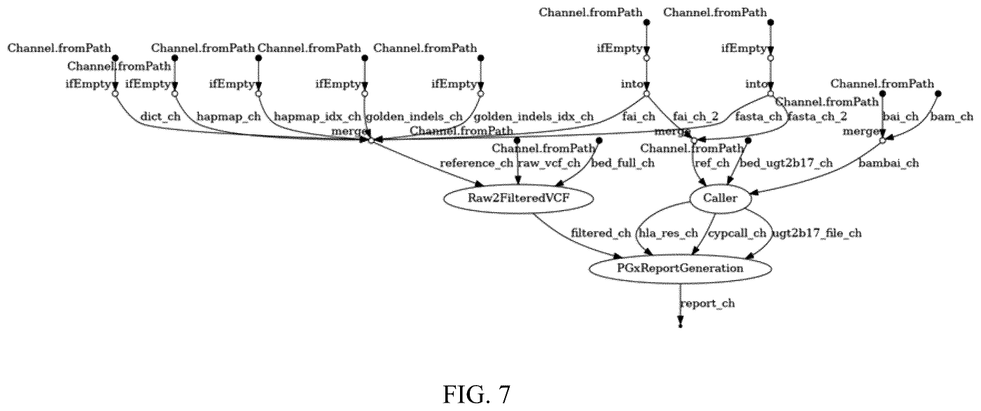

FIG. 7 is a method implemented by a pharmacogenomic platform, according to some embodiments;

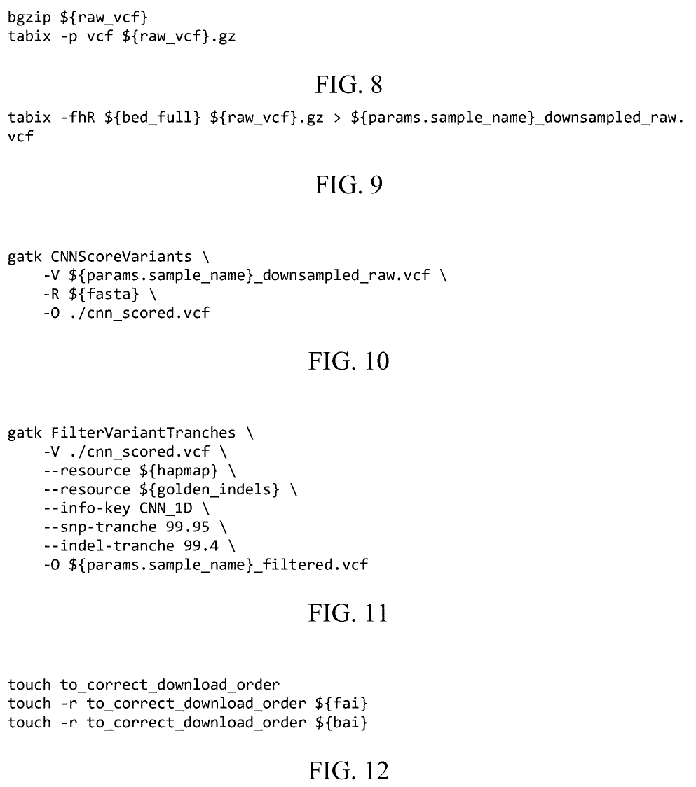

FIG. 8 is code implemented by a pharmacogenomic platform, according to some embodiments;

FIG. 9 is code implemented by a pharmacogenomic platform, according to some embodiments;

FIG. 10 is code implemented by a pharmacogenomic platform, according to some embodiments;

FIG. 11 is code implemented by a pharmacogenomic platform, according to some embodiments;

FIG. 12 is code implemented by a pharmacogenomic platform, according to some embodiments;

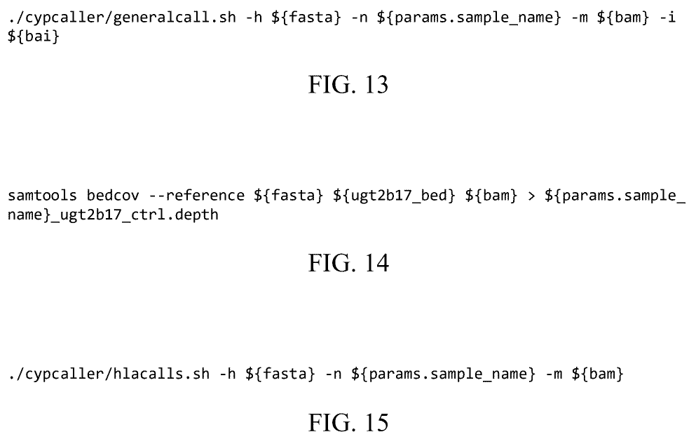

FIG. 13 is code implemented by a pharmacogenomic platform, according to some

FIG. 14 is code implemented by a pharmacogenomic platform, according to some embodiments;

FIG. 15 is code implemented by a pharmacogenomic platform, according to some embodiments;

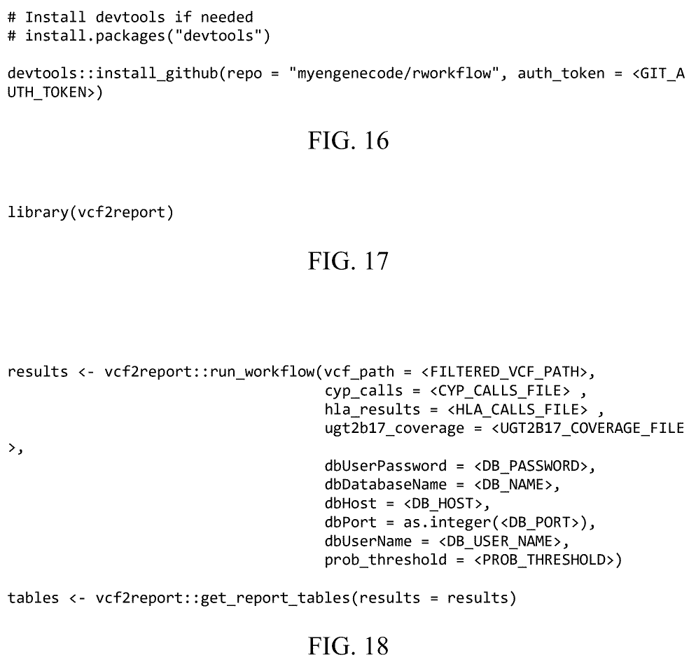

FIG. 16 is code implemented by a pharmacogenomic platform, according to some embodiments;

FIG. 17 is code implemented by a pharmacogenomic platform, according to some embodiments;

FIG. 18 is code implemented by a pharmacogenomic platform, according to some embodiments;

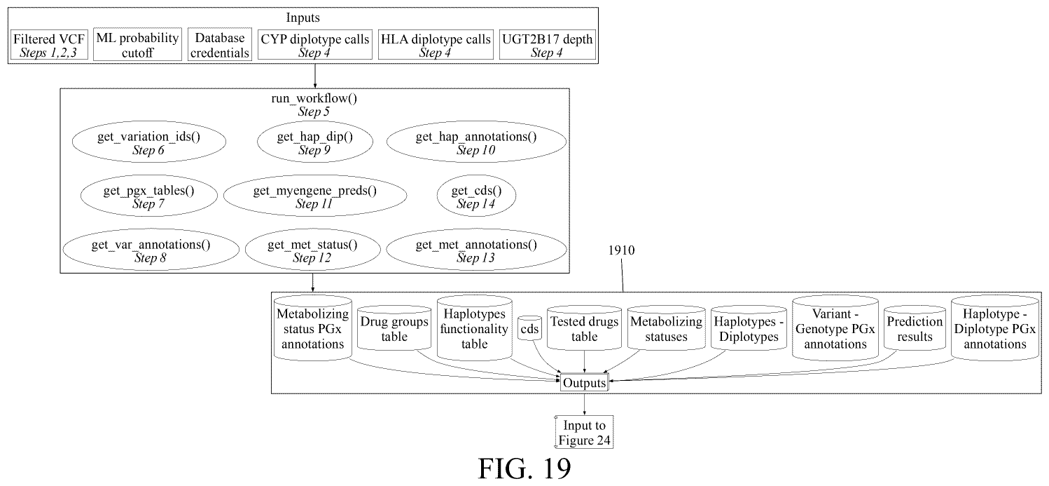

FIG. 19 is a method implemented by a pharmacogenomic platform, according to some embodiments;

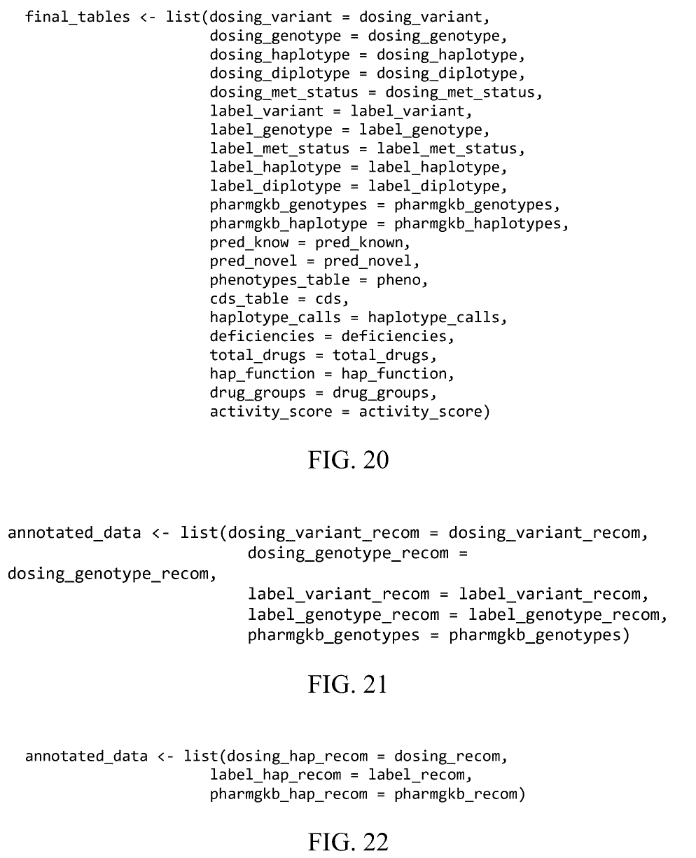

FIG. 20 is code implemented by a pharmacogenomic platform, according to some embodiments;

FIG. 21 is code implemented by a pharmacogenomic platform, according to some embodiments;

FIG. 22 is code implemented by a pharmacogenomic platform, according to some embodiments;

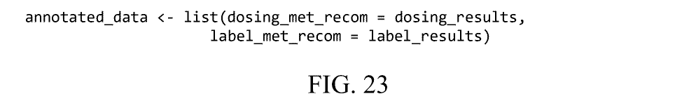

FIG. 23 is code implemented by a pharmacogenomic platform, according to some embodiments;

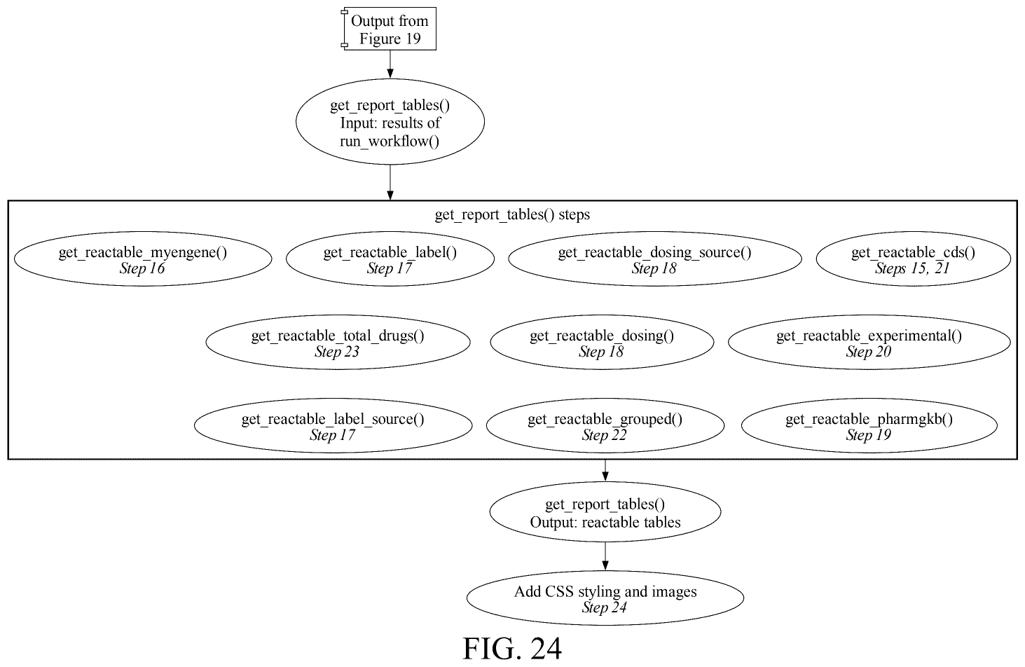

FIG. 24 is a method implemented by a pharmacogenomic platform, according to some embodiments;

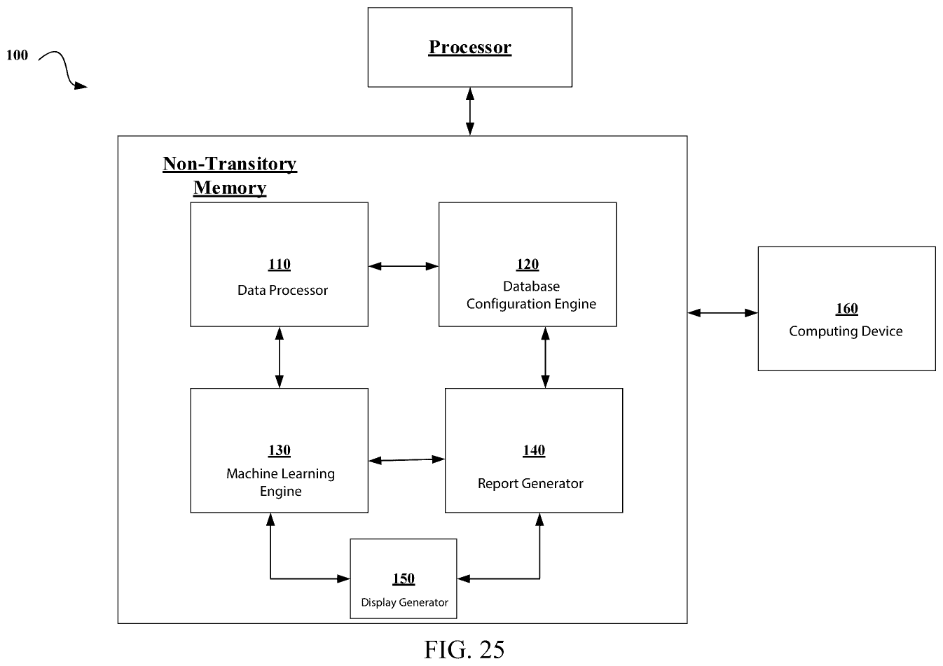

FIG. 25 is a schematic view of a pharmacogenomic platform, according to some embodiments;

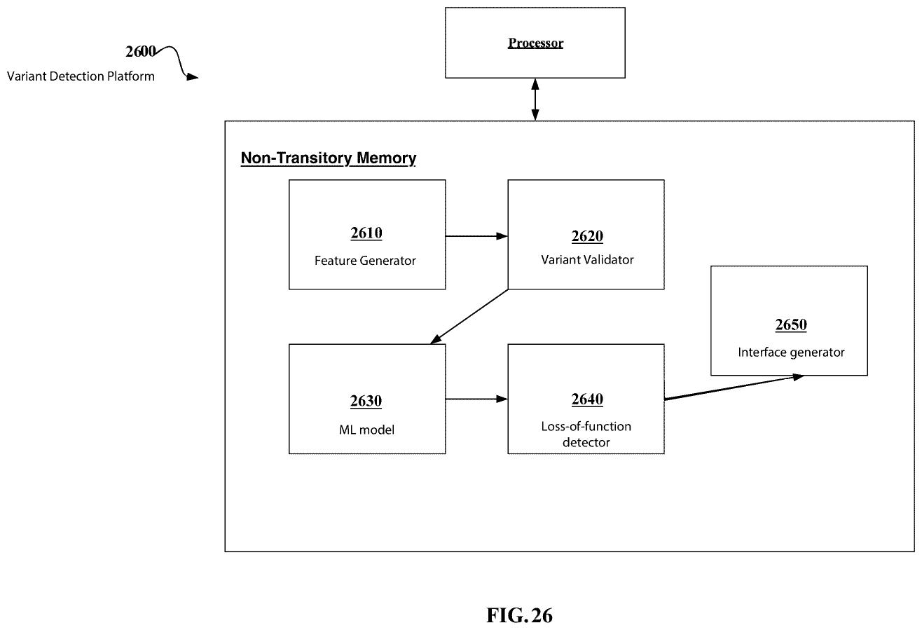

FIG. 26 is a schematic diagram of a variant detection platform, according to some embodiments;

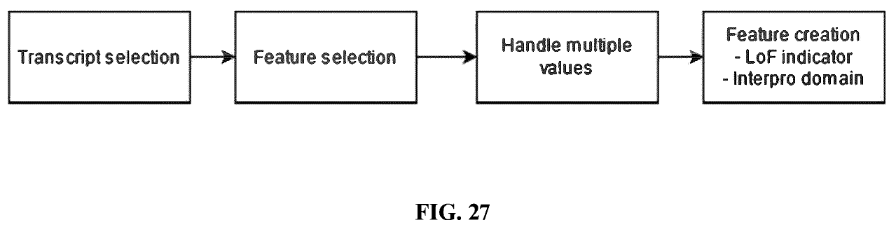

FIG. 27 is a flow diagram of a process for generating one or more features from an annotated variant dataset, according to some embodiments;

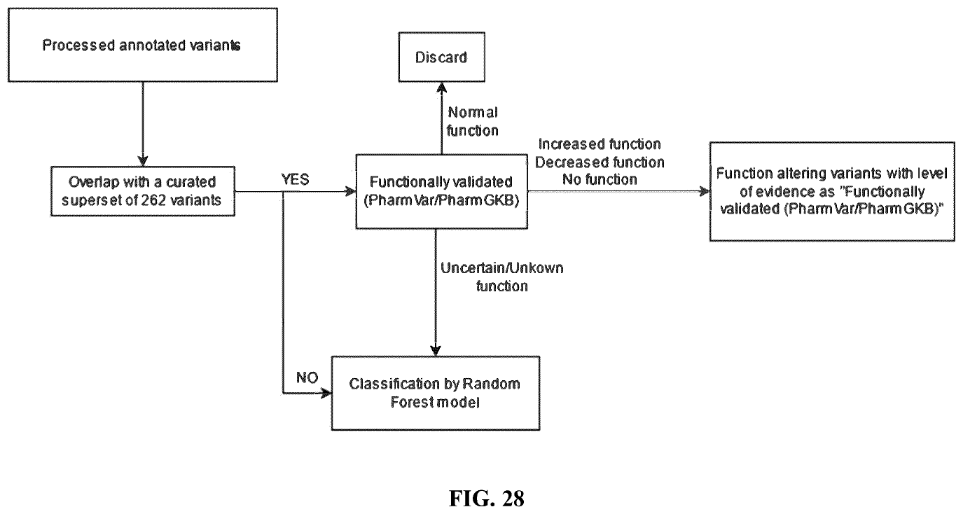

FIG. 28 is a flow diagram of a process for variant validation, according to some embodiments;

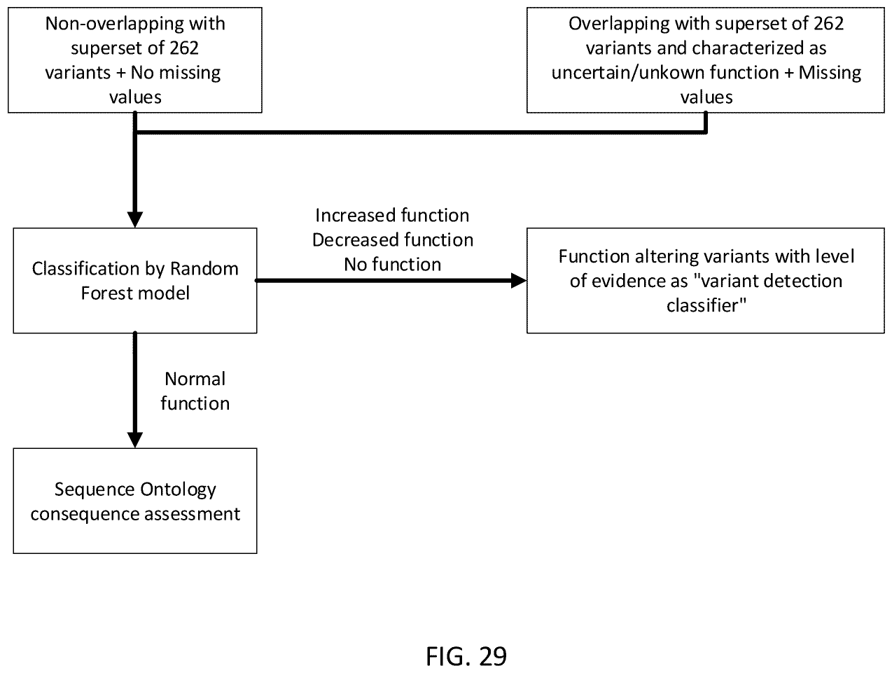

FIG. 29 is a flow diagram of a process for machine learning model, according to some embodiments;

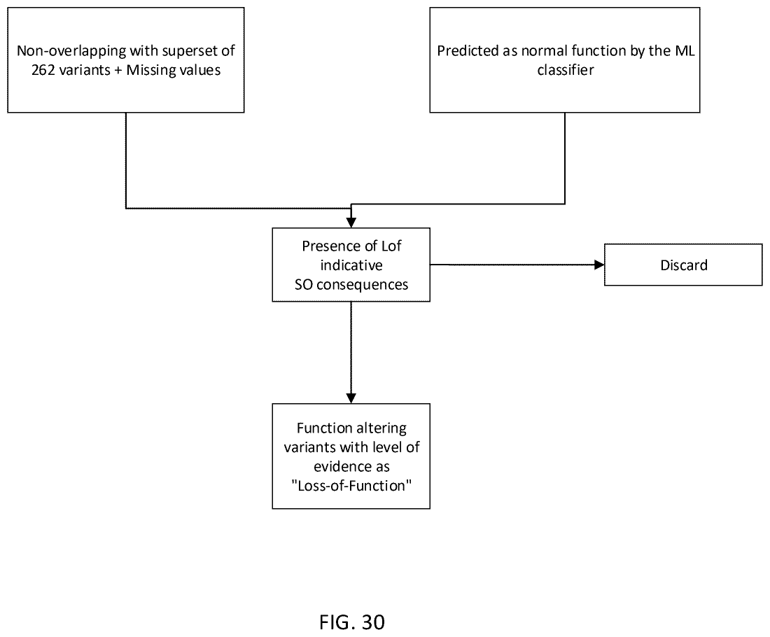

FIG. 30 is a flow diagram of a process for detecting loss-of-function, according to some embodiments;

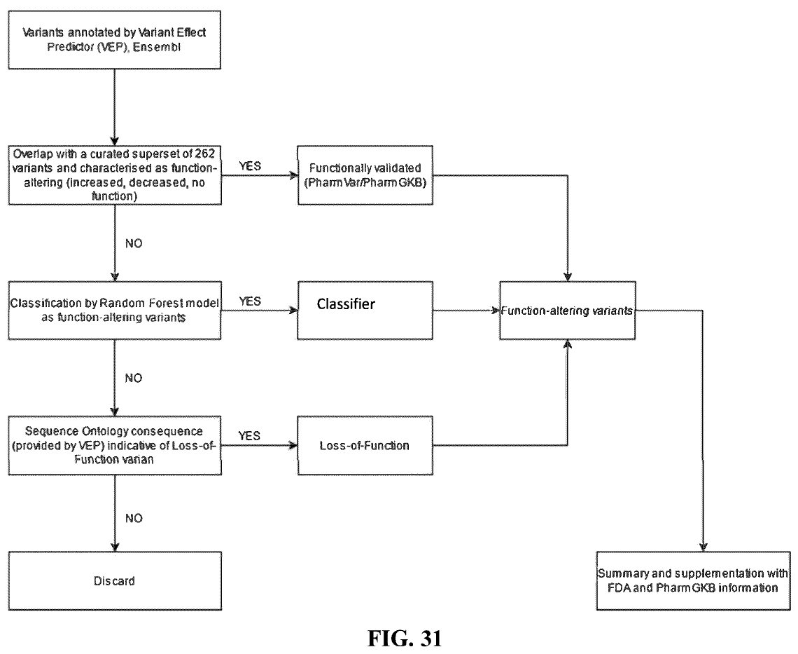

FIG. 31 is a flow diagram of a process for variant detection platform, according to some embodiments;

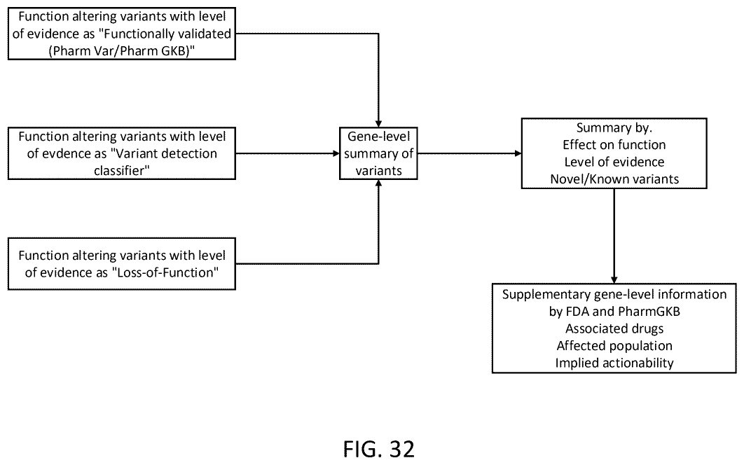

FIG. 32 is a flow diagram of a process generating an interface, according to some embodiments;

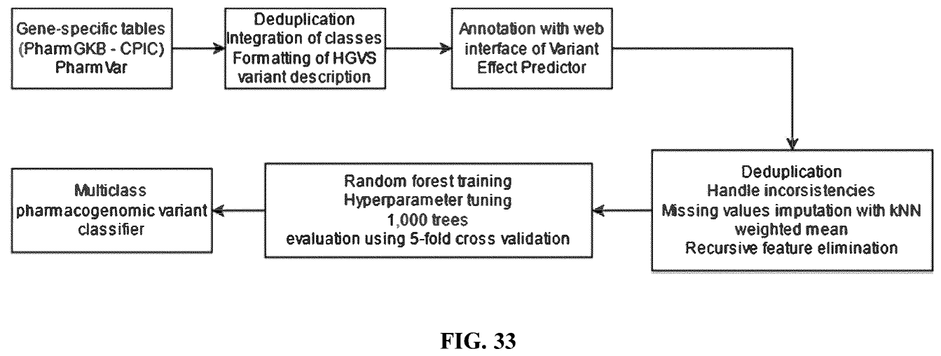

FIG. 33 is a flow diagram of a process for training a machine learning model, according to some embodiments;

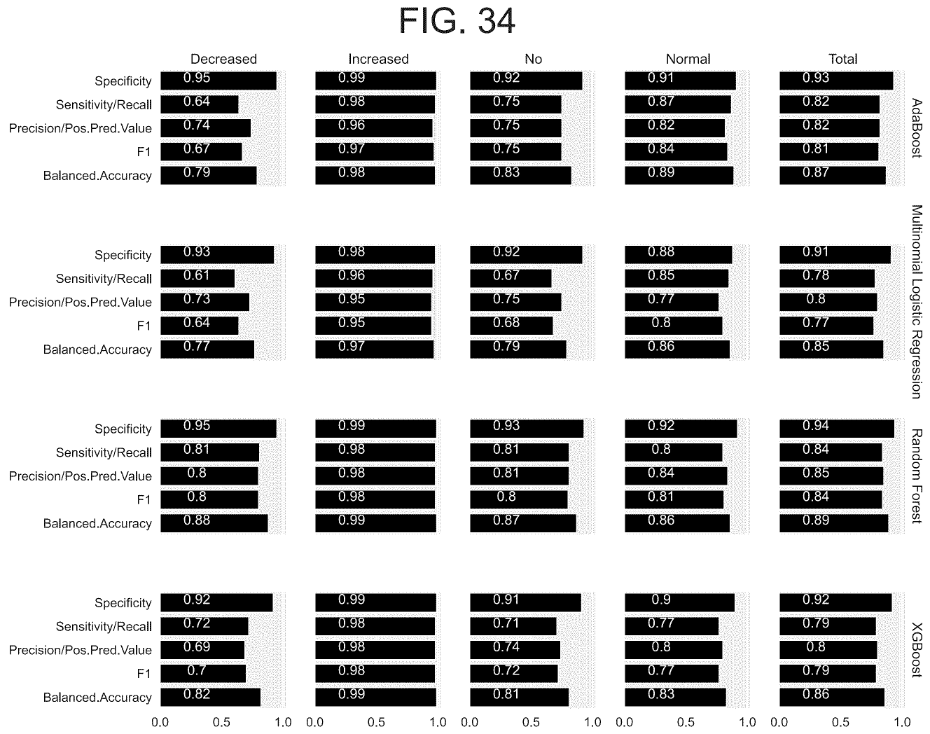

FIG. 34 are charts illustrating performance of different classifiers, according to some embodiments;

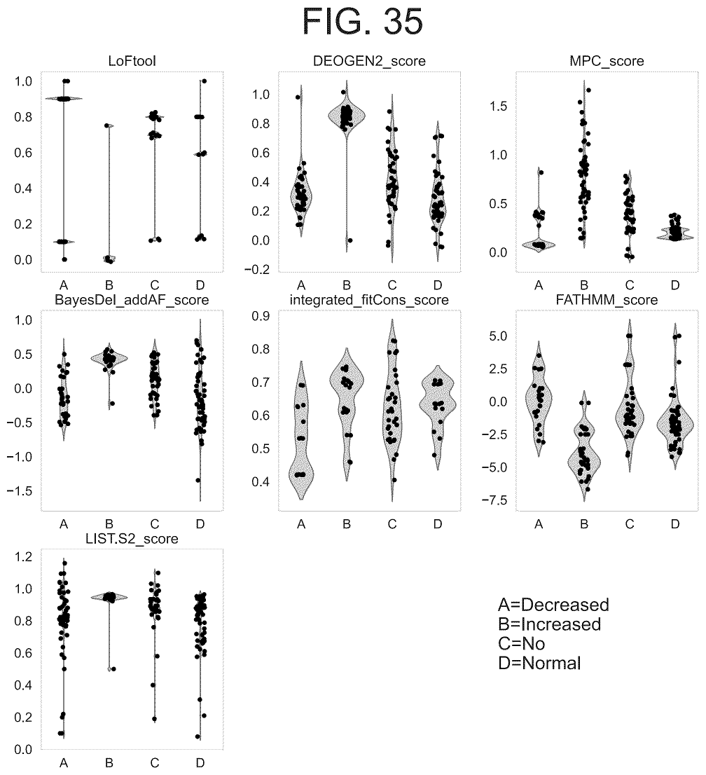

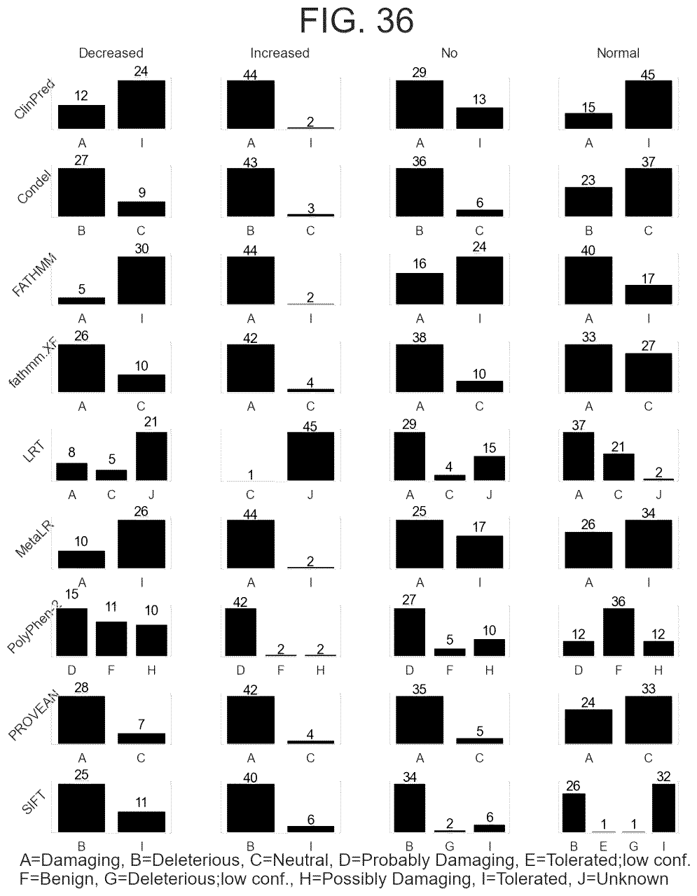

FIG. 35 are violin plots depicting the distribution of each functionality class for certain in silico predictions tools, according to some embodiments;

FIG. 36 are charts illustrating classification of variants, according to some embodiments;

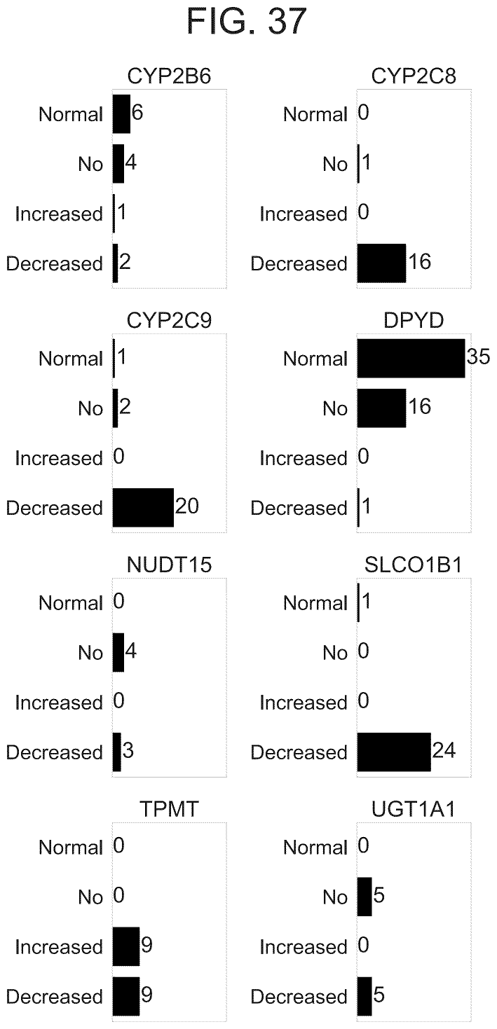

FIG. 37 are charts illustrating classification of variants, according to some embodiments;

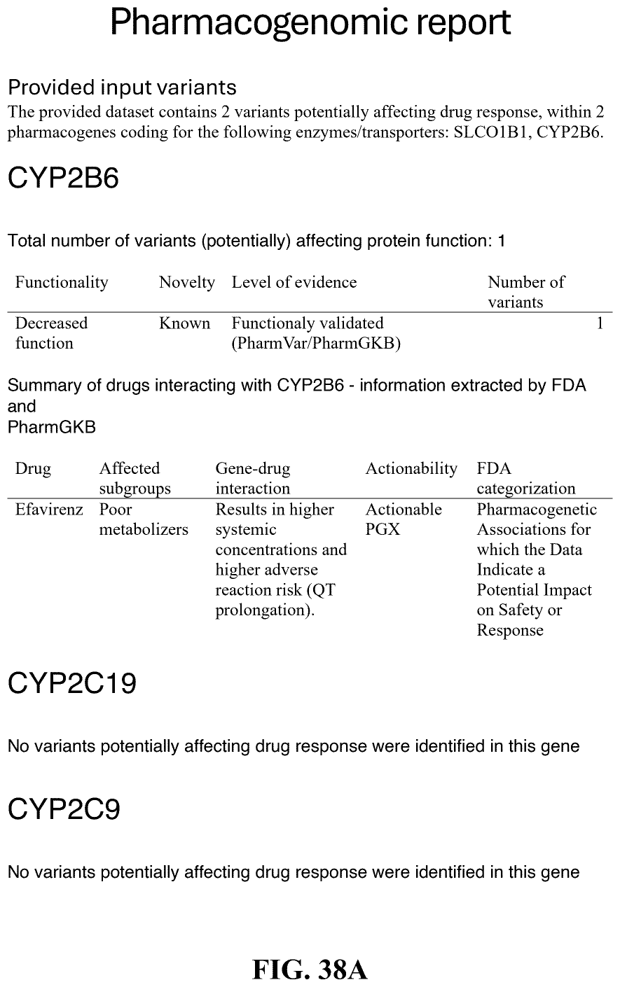

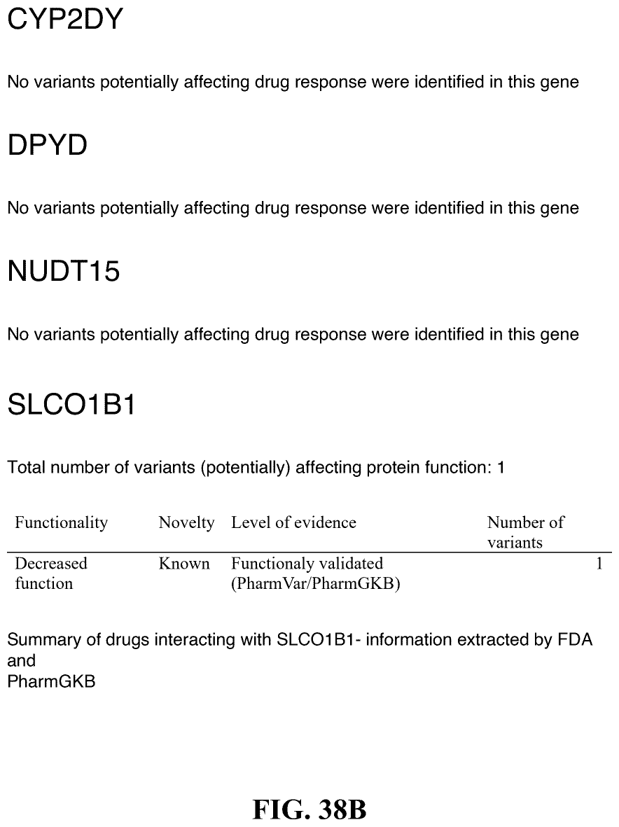

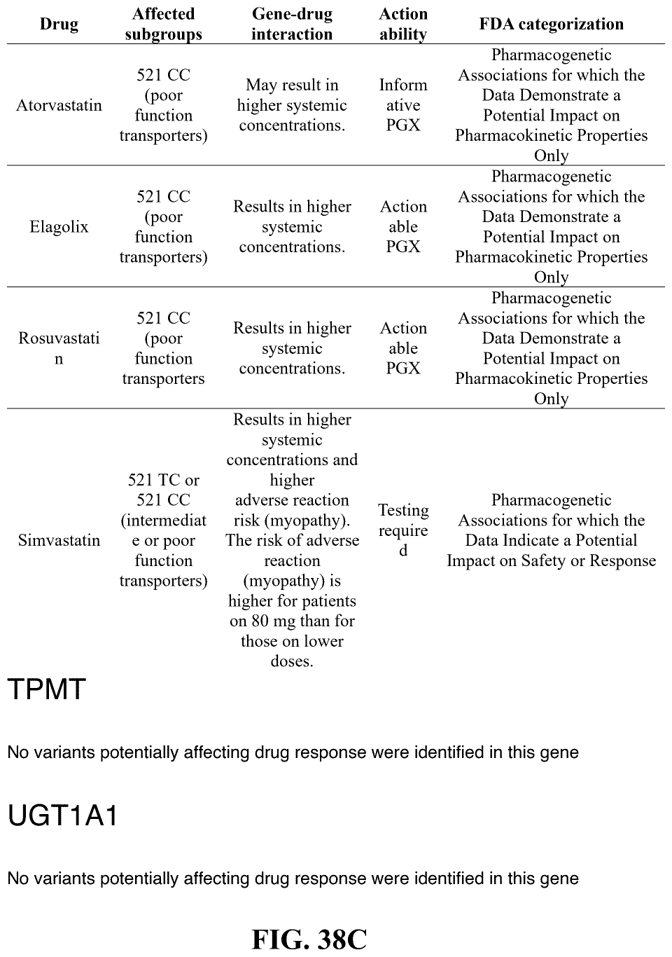

FIG. 38A, FIG. 38B and FIG. 38C show interfaces generated by variant detection platform, according to some embodiments;

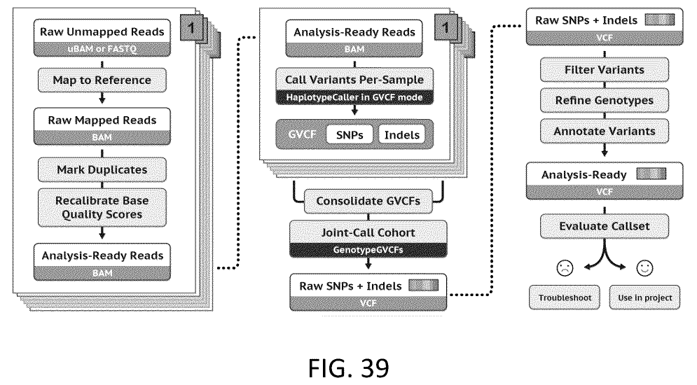

FIG. 39 is a flow diagram of a process for generation of an annotated variant dataset, according to some embodiments;

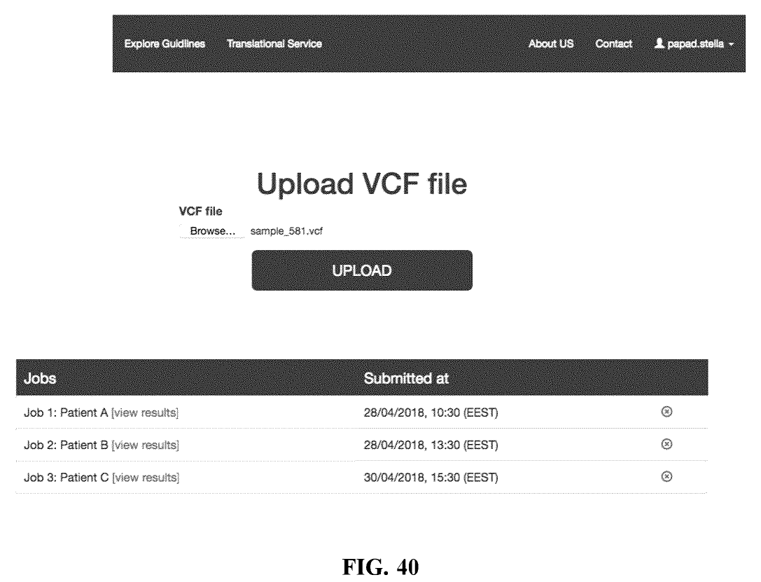

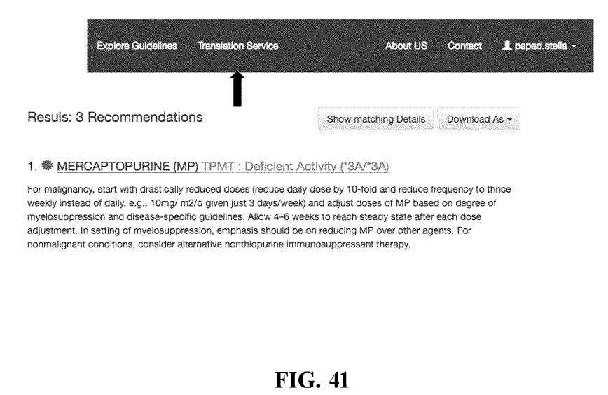

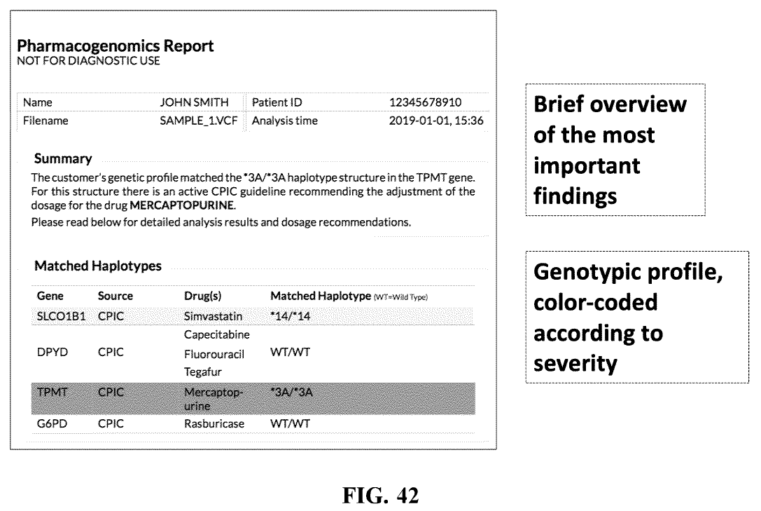

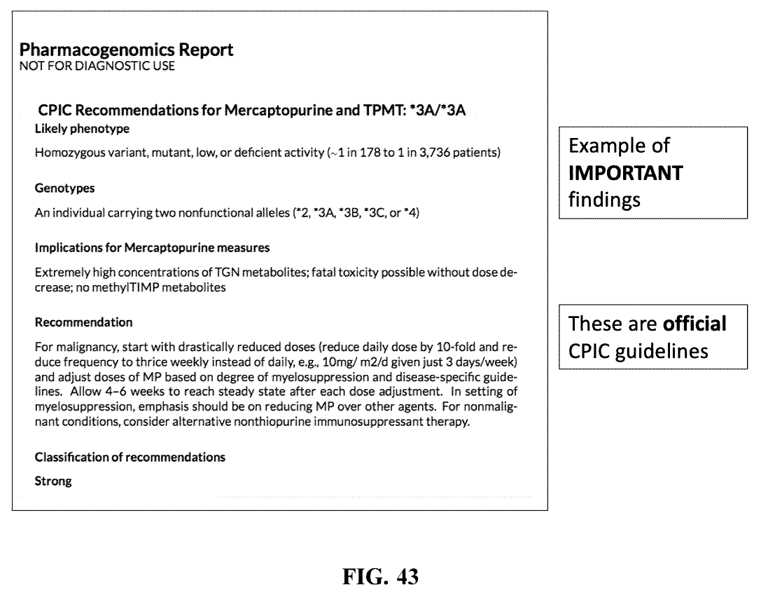

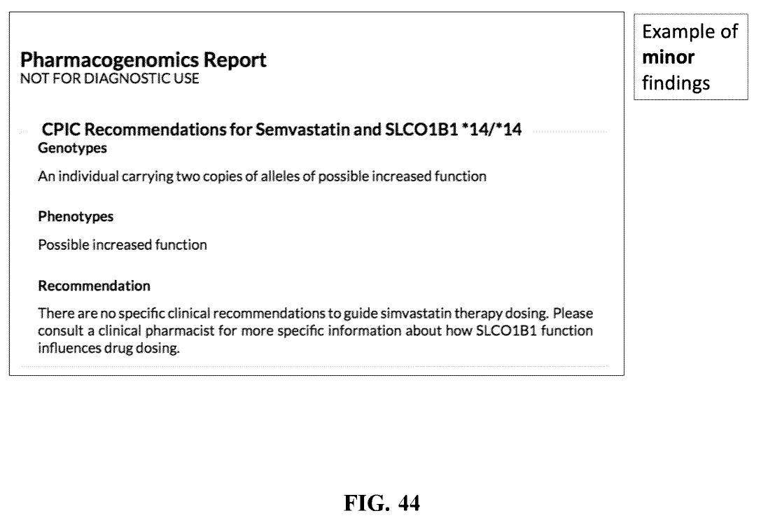

FIG. 40, FIG. 41, FIG. 42, FIG. 43, and FIG. 44 show interfaces generated, according to some embodiments;

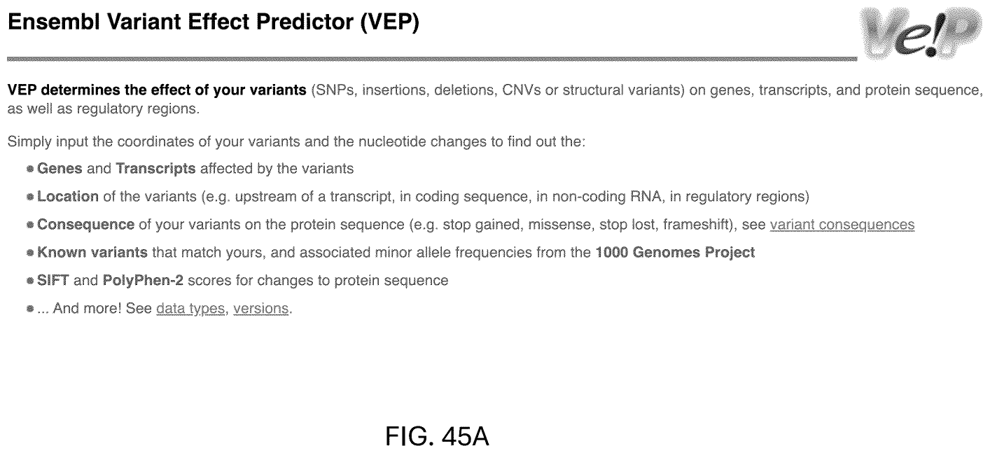

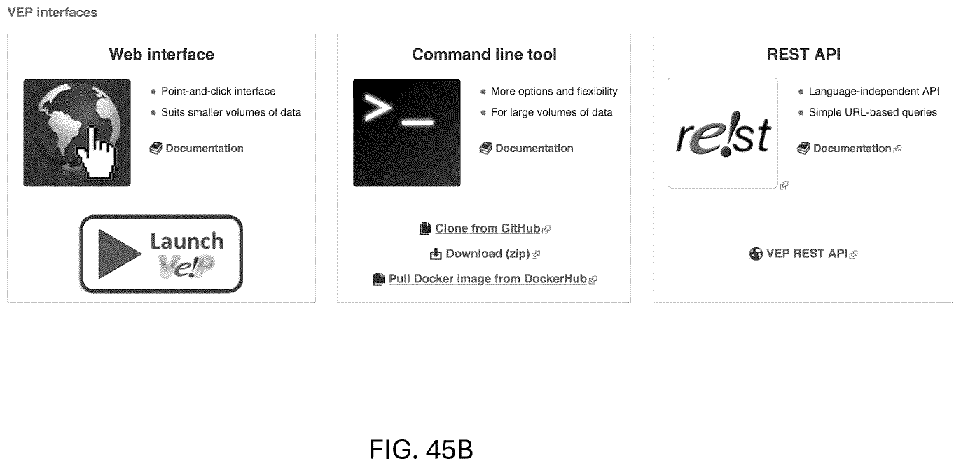

FIG. 45A and FIG. 45B show an interface for a VEP, according to some embodiments;

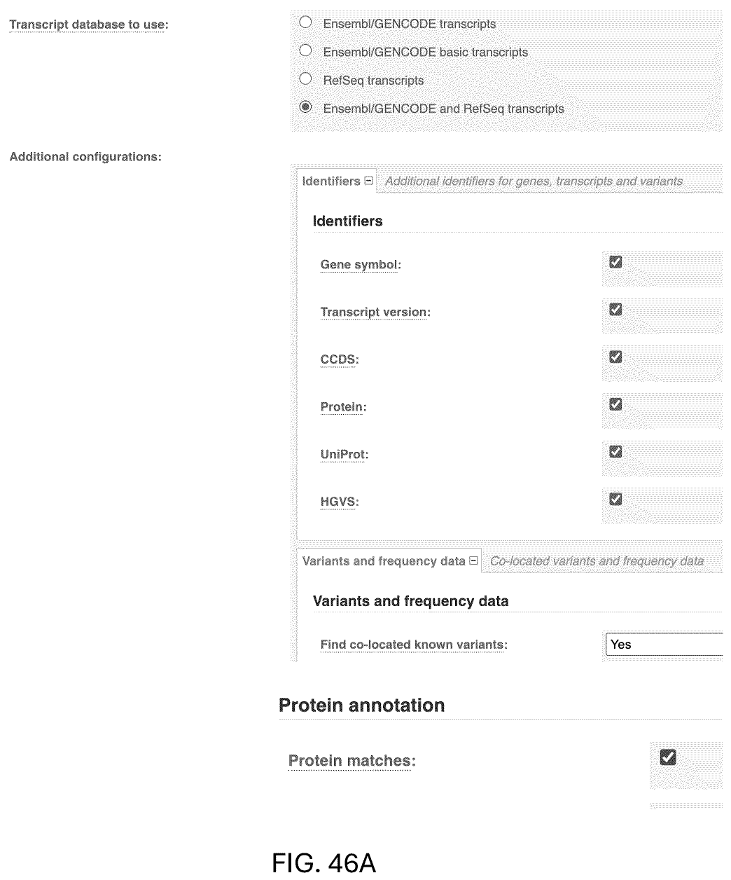

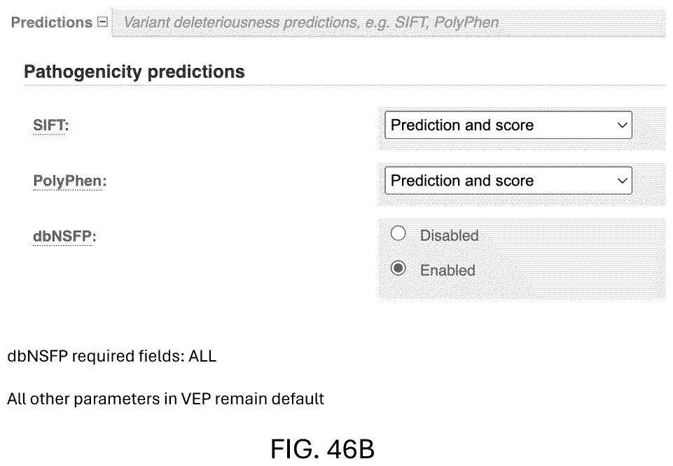

FIG. 46A and FIG. 46B show VEP parameters on an example interface, according to some embodiments;

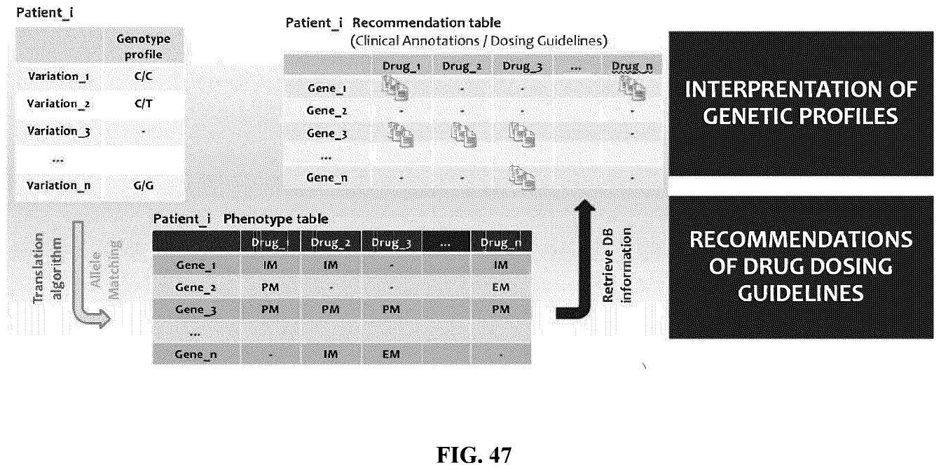

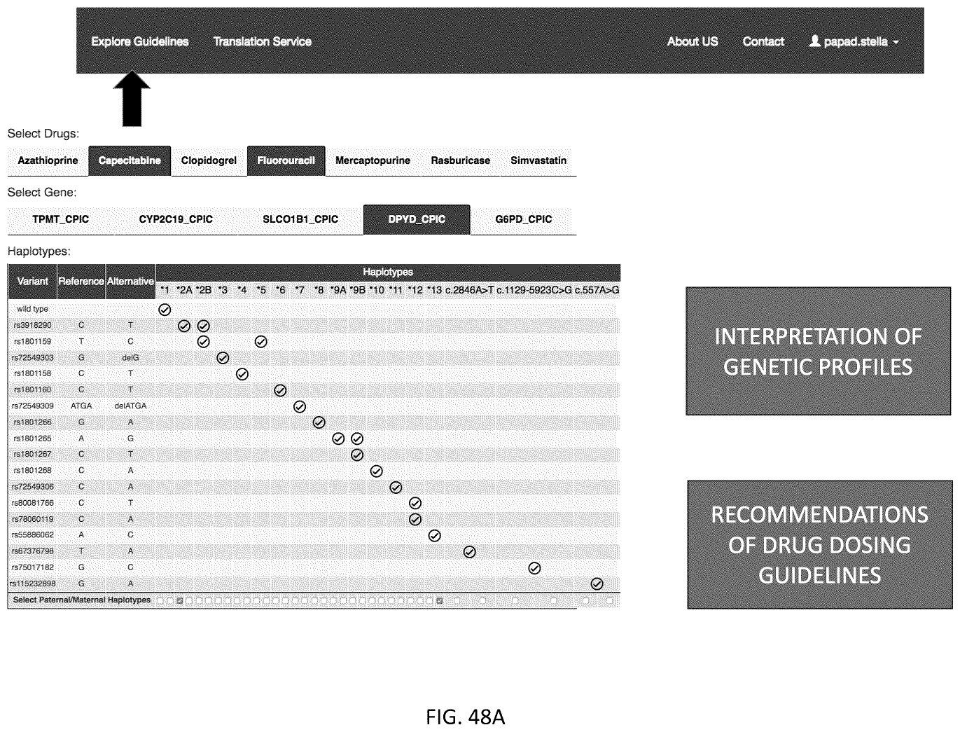

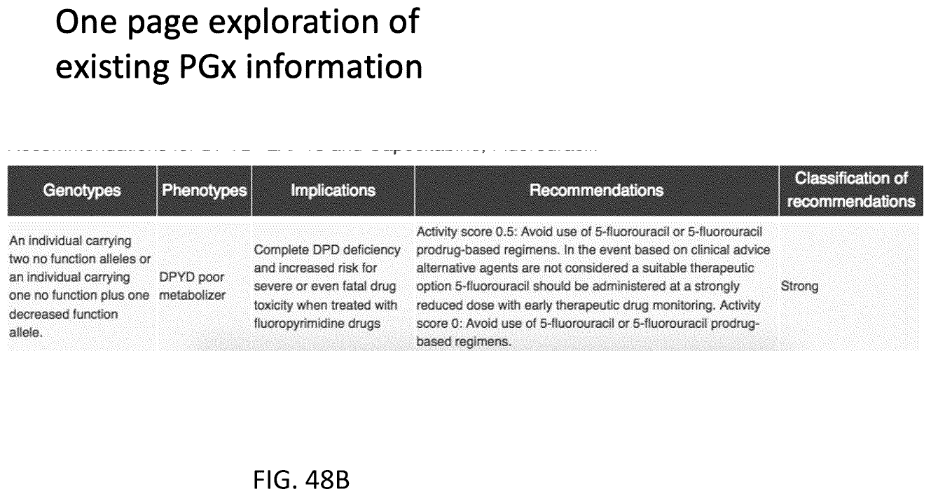

FIG. 47, FIG. 48A and FIG. 48B show example interfaces generated, according to some embodiments;

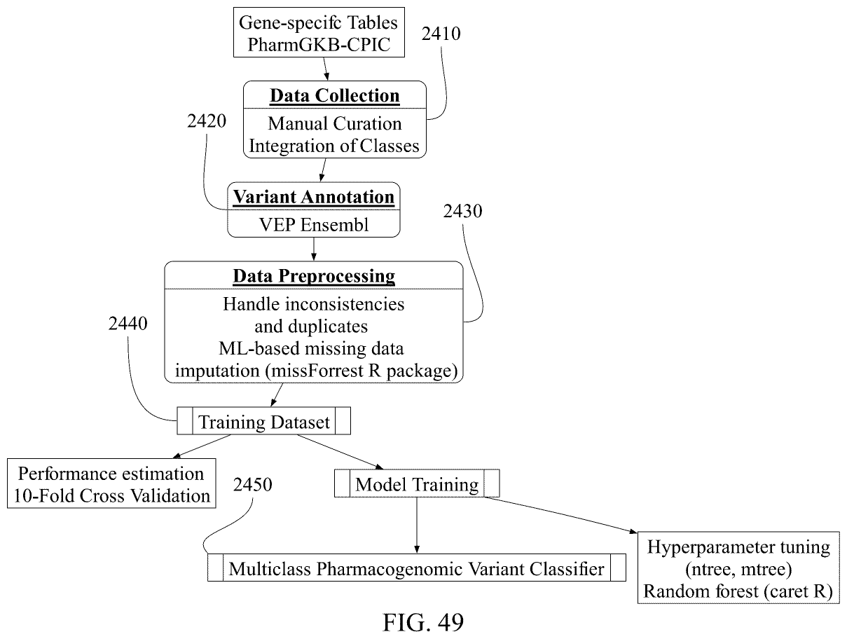

FIG. 49 is a flow diagram illustrating a method of performing classification of the effect of a variant, according to some embodiments;

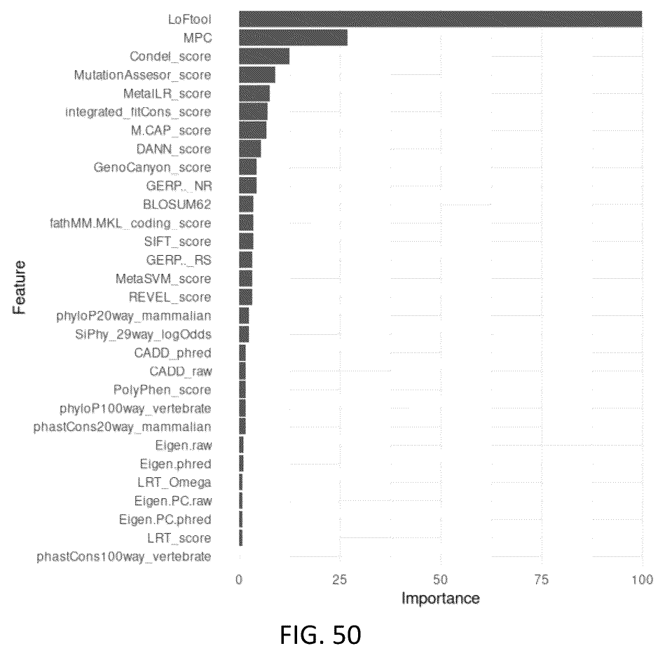

FIG. 50 is a plot illustrating variable importance, extracted from a model trained with 156 SNVs, categorized into 5 classes, where LoFtool is proposed as a valuable predictive feature, according to some embodiments;

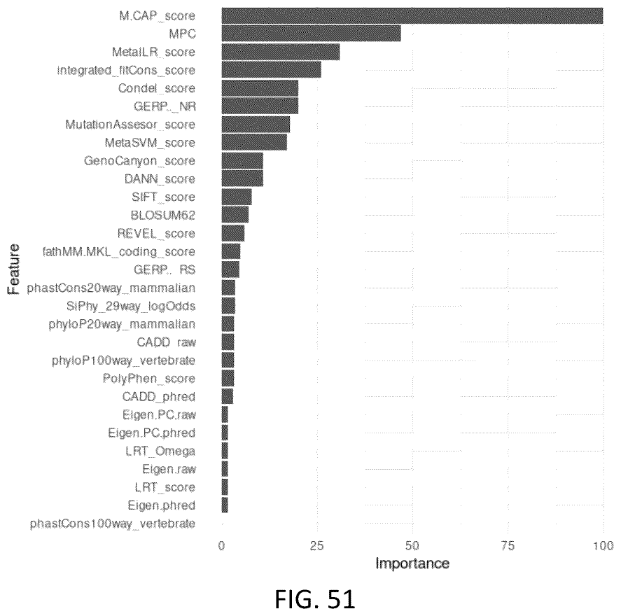

FIG. 51 is a plot illustrating variable importance for the model trained with 156 SNVs (in 5 classes), after removing LoFtool from the training variables, according to some embodiments;

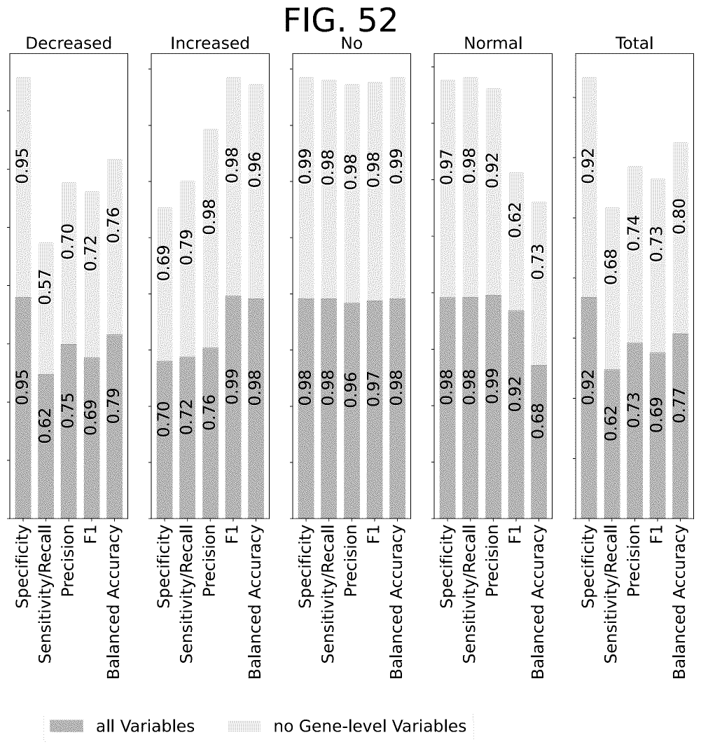

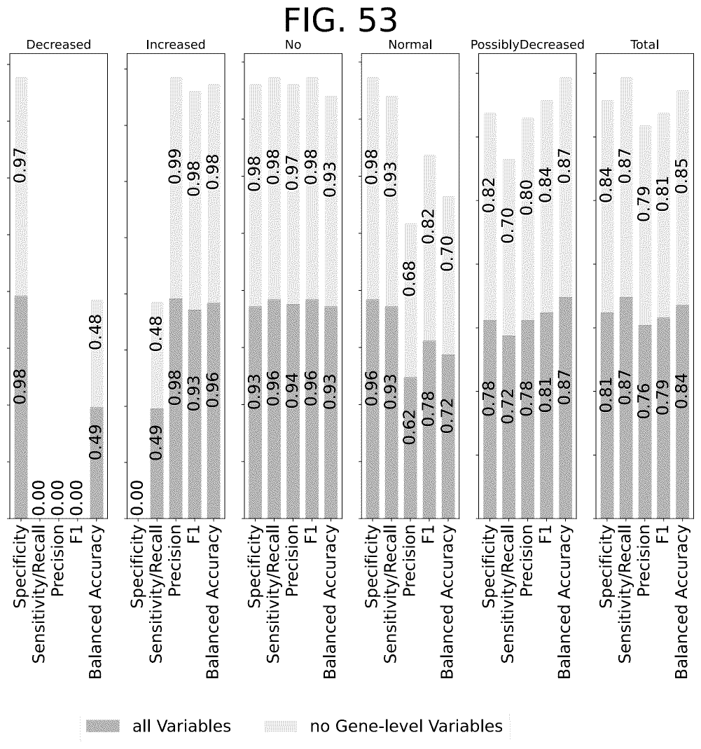

FIG. 52 illustrates performance metrics computed for a 5-class containing training set, with (all Variables) and without (no gene-level variables) LoFtool as a training feature, according to some embodiments;

FIG. 53 illustrates performance metrics computed for the 4-class containing training set, with (all Variables) and without (no gene-level variables) LoFtool as a training feature, according to some embodiments;

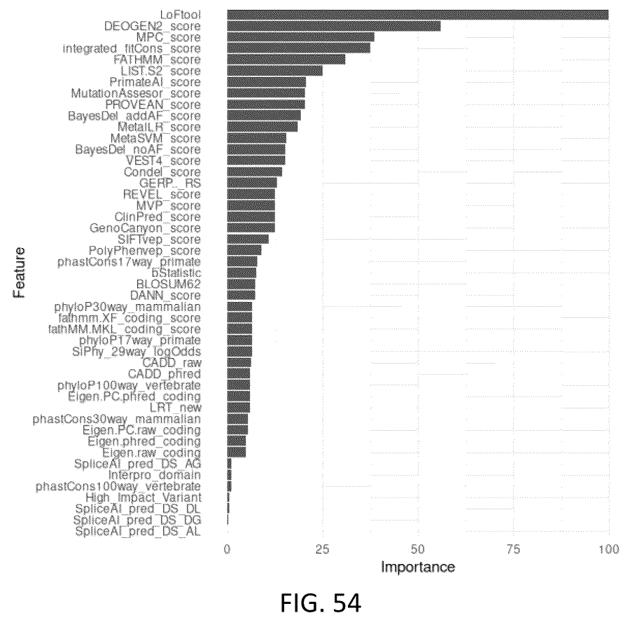

FIG. 54 is a graph illustrating annotation features examined as training variables in a machine learning model, according to some embodiments;

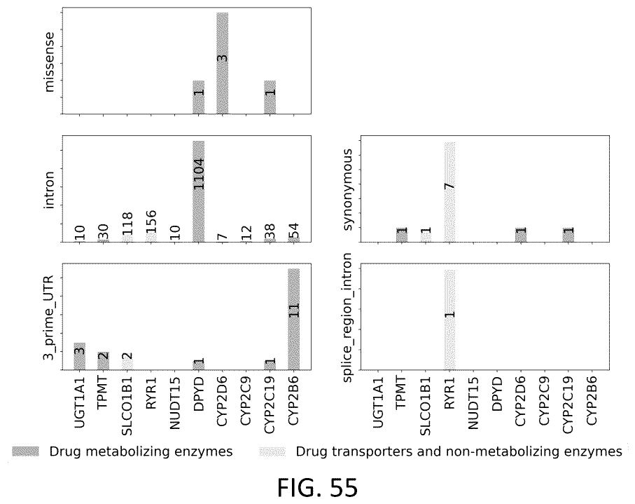

FIG. 55 is a chart illustrating a distribution of pharmacogenomic variants identified, according to some embodiments;

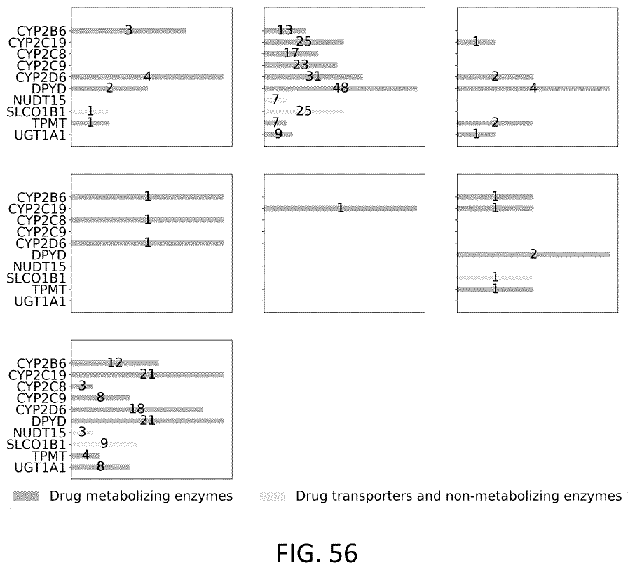

FIG. 56 is a chart illustrating sequence ontology consequences for pharmacogenomic variants, according to some embodiments;

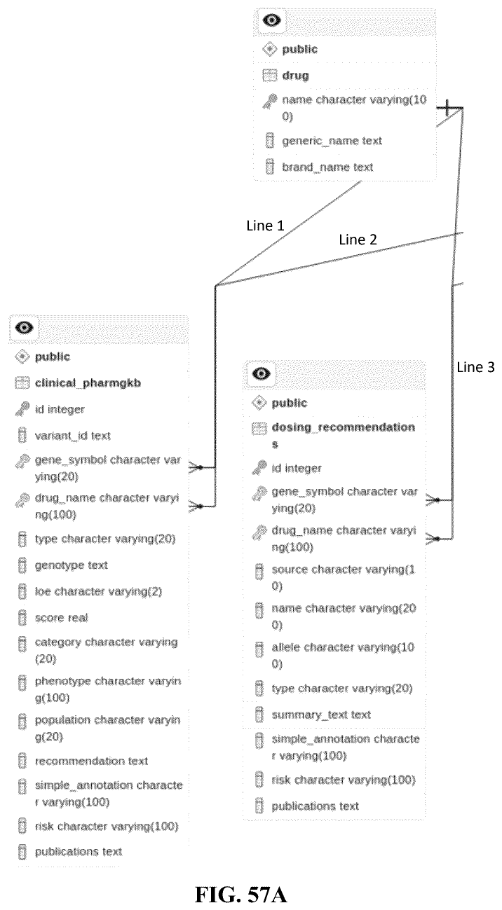

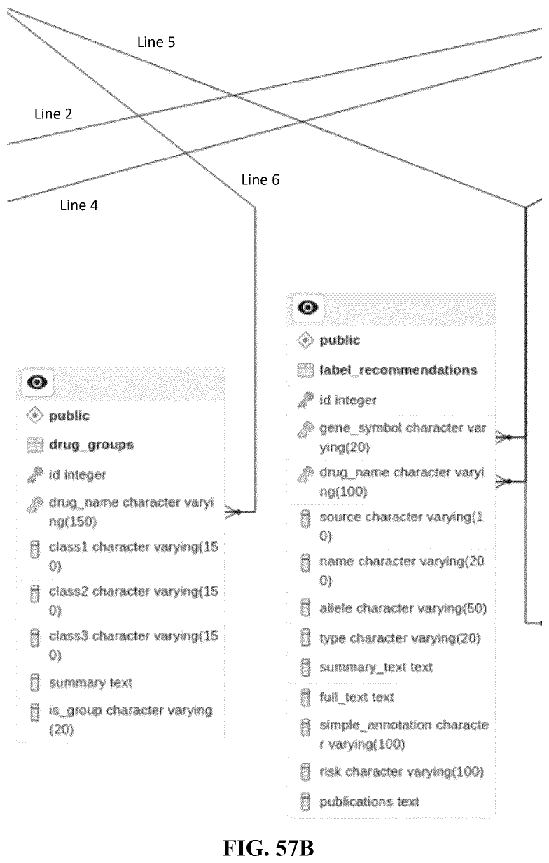

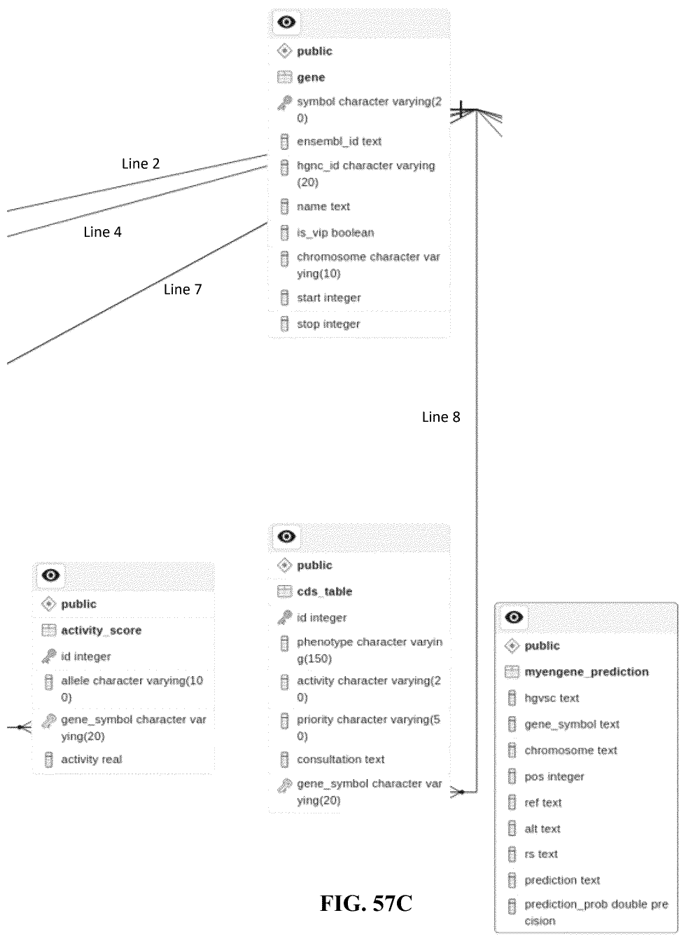

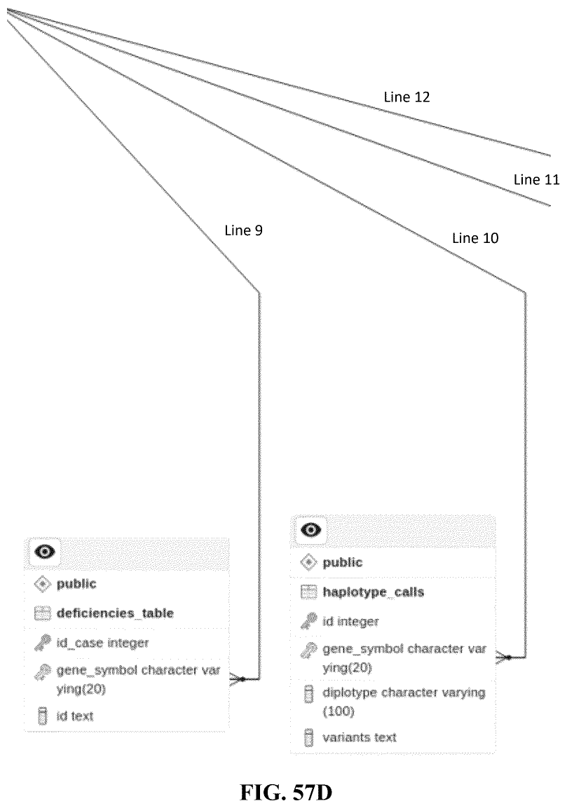

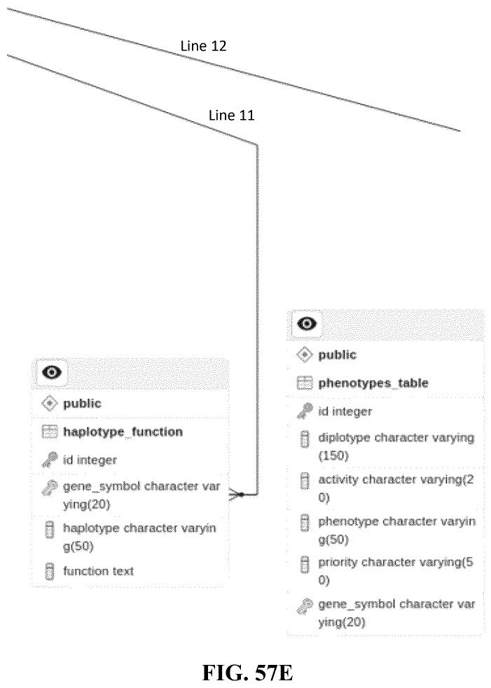

FIG. 57A, 57B, 57C, 57D and 57E are a database schema, according to some embodiments;

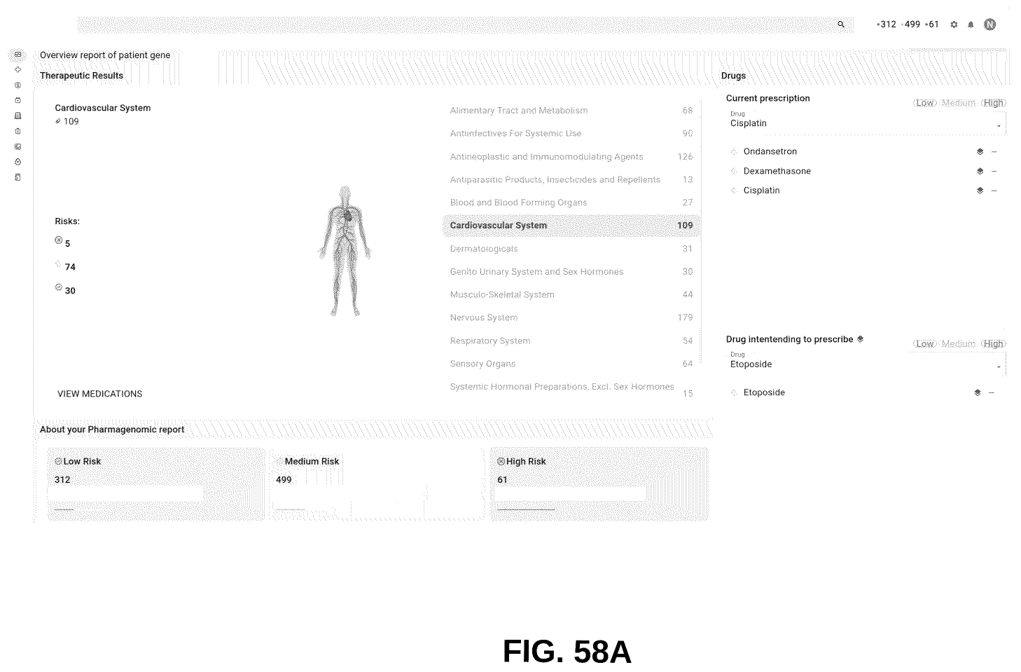

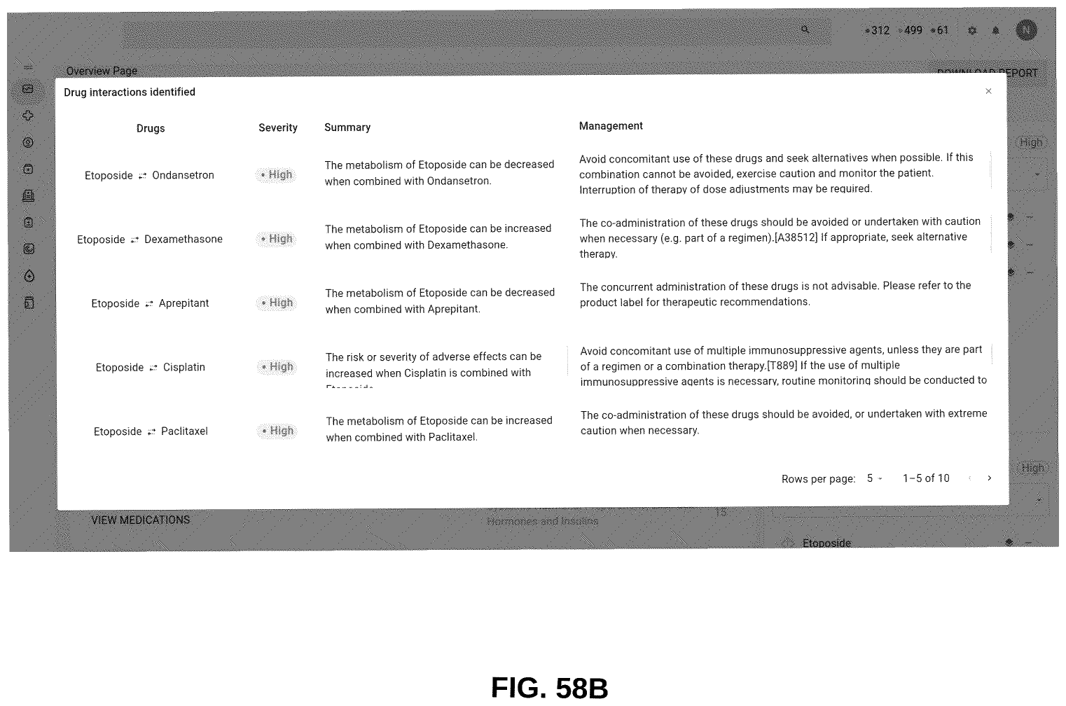

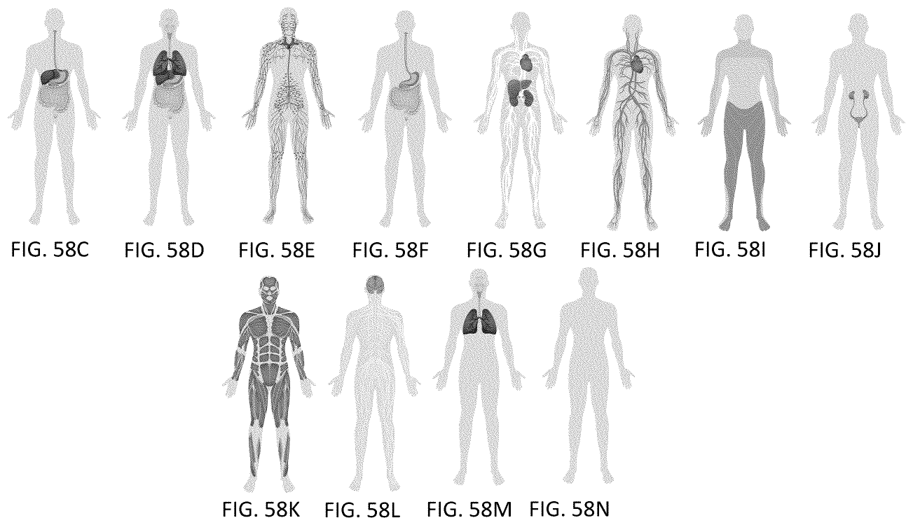

FIG. 58A is a user interface showing an overview webapp page, according to some embodiments;

FIG. 58B is the drug interaction display page, according to some embodiments;

FIG. 58C-58N contain human svg images representing each of the therapeutic areas, according to some embodiments;

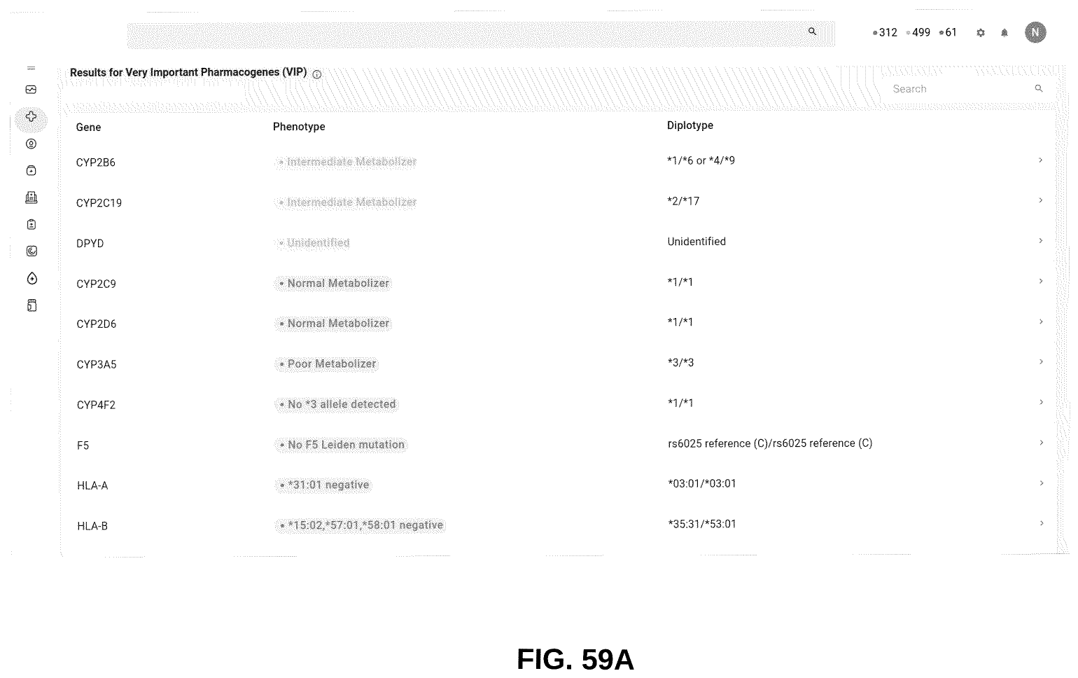

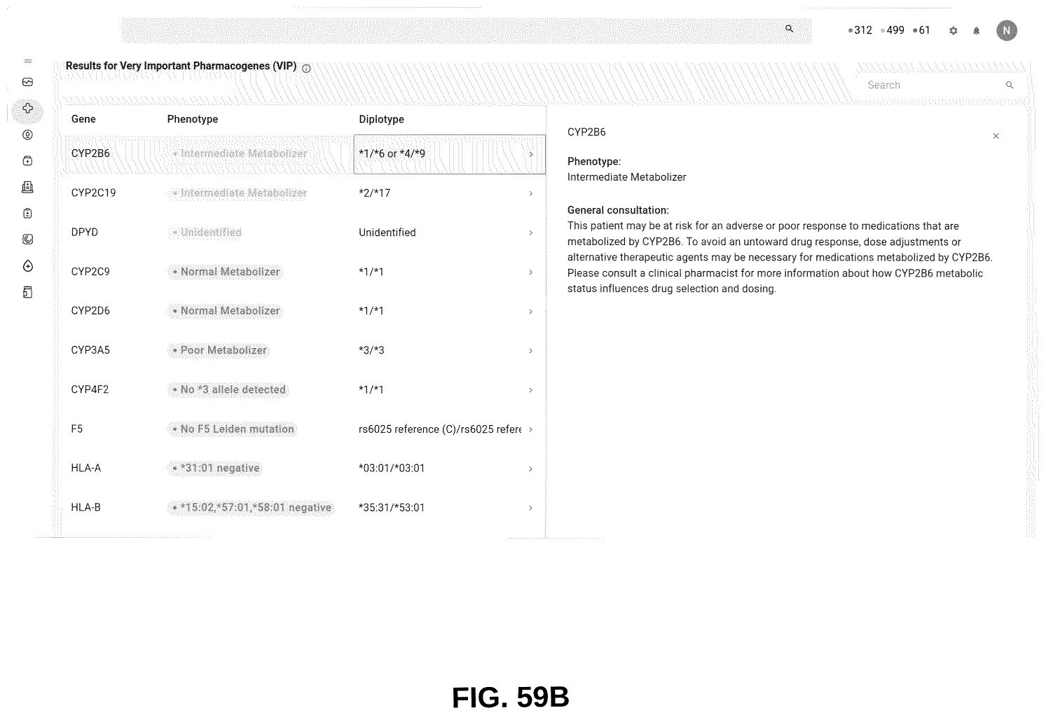

FIG. 59A and FIG. 59B are the Results for Very Important Pharmacogenes (VIP) webapp page, according to some embodiments;

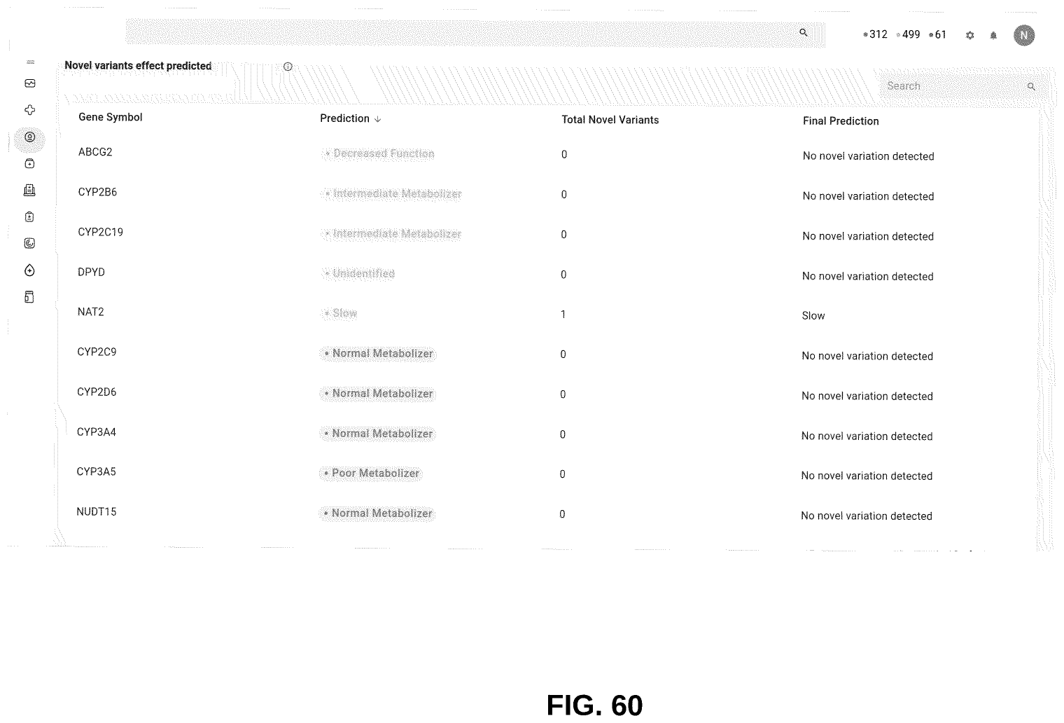

FIG. 60 Novel Variant Effect Predicted on webapp page, according to some embodiments;

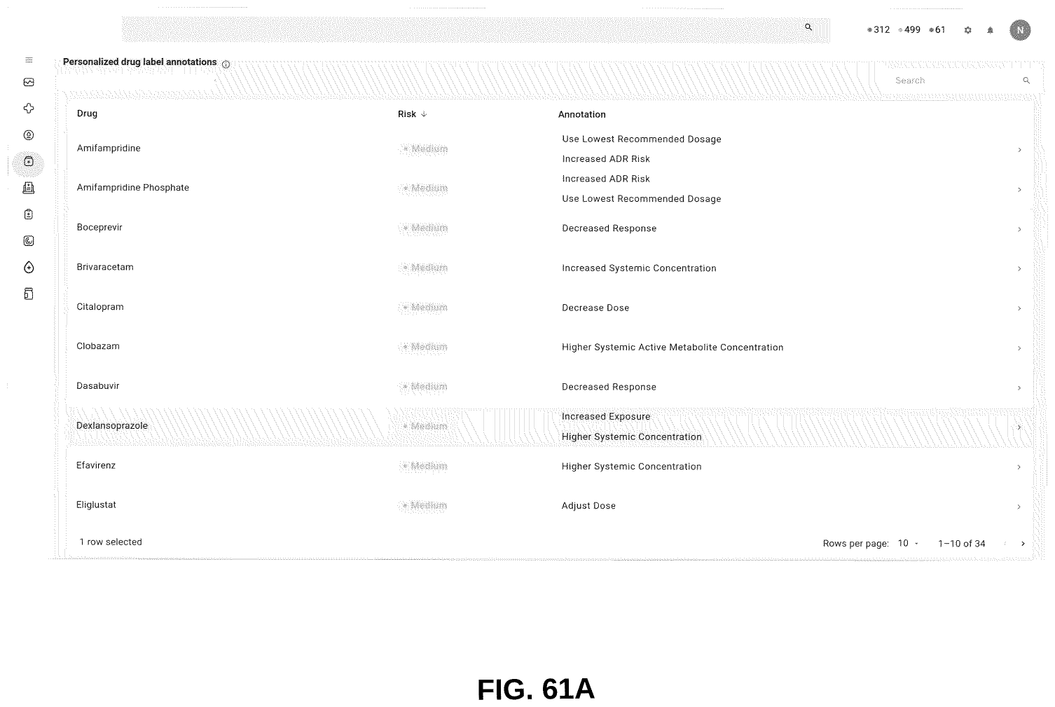

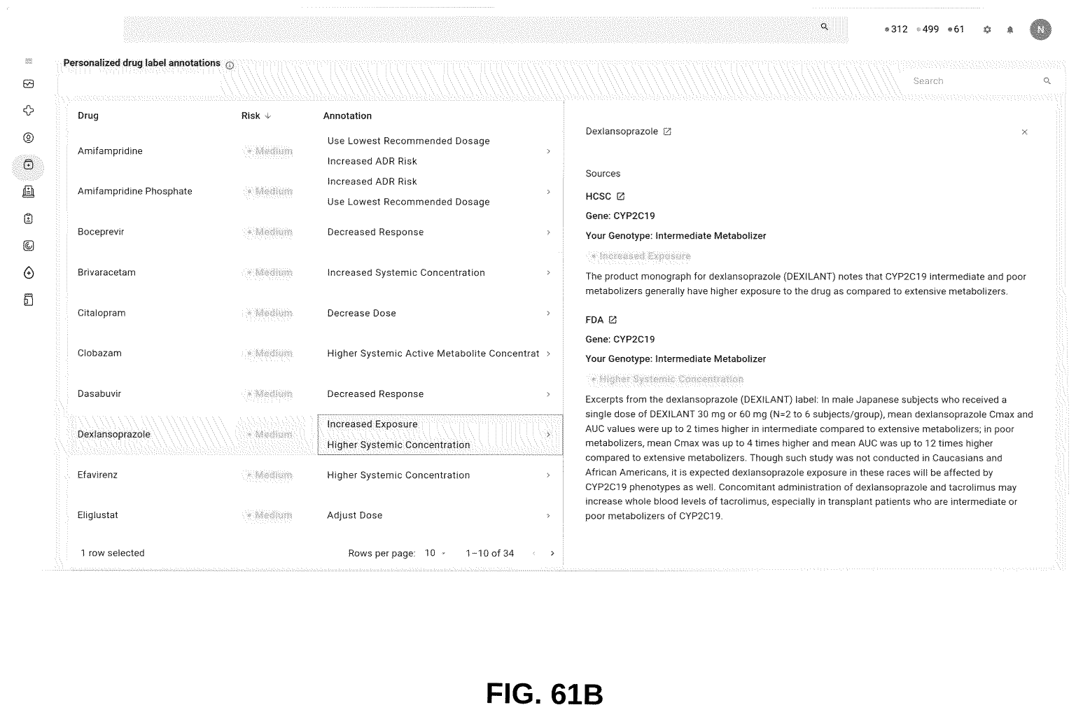

FIG. 61A and FIG. 61B are the Personalized Drug Label Annotations webapp page, according to some embodiments;

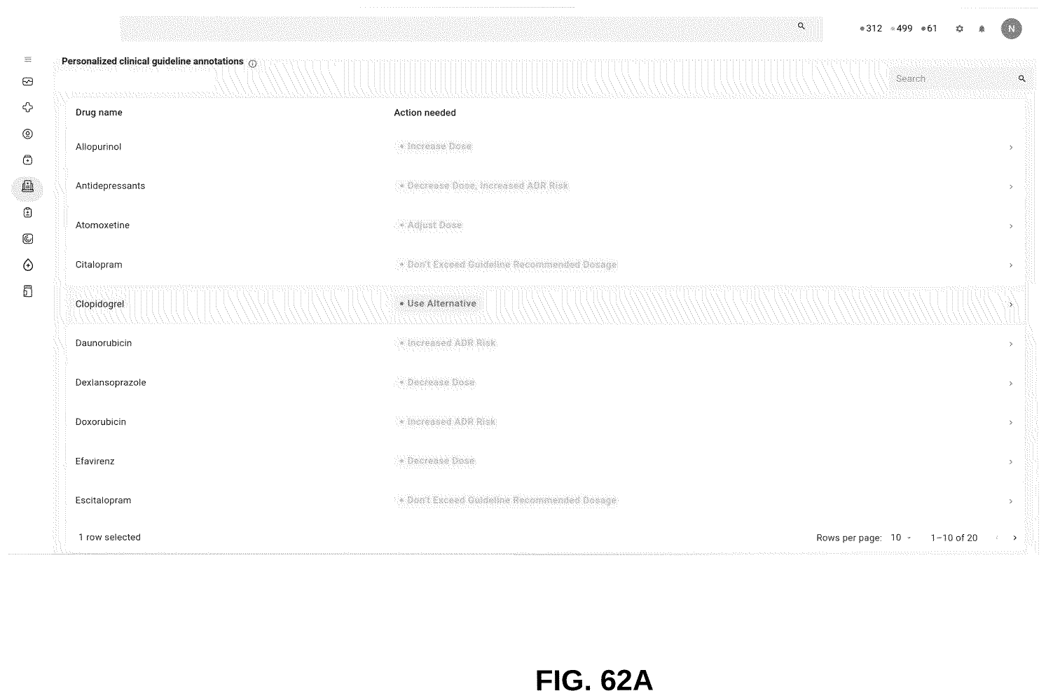

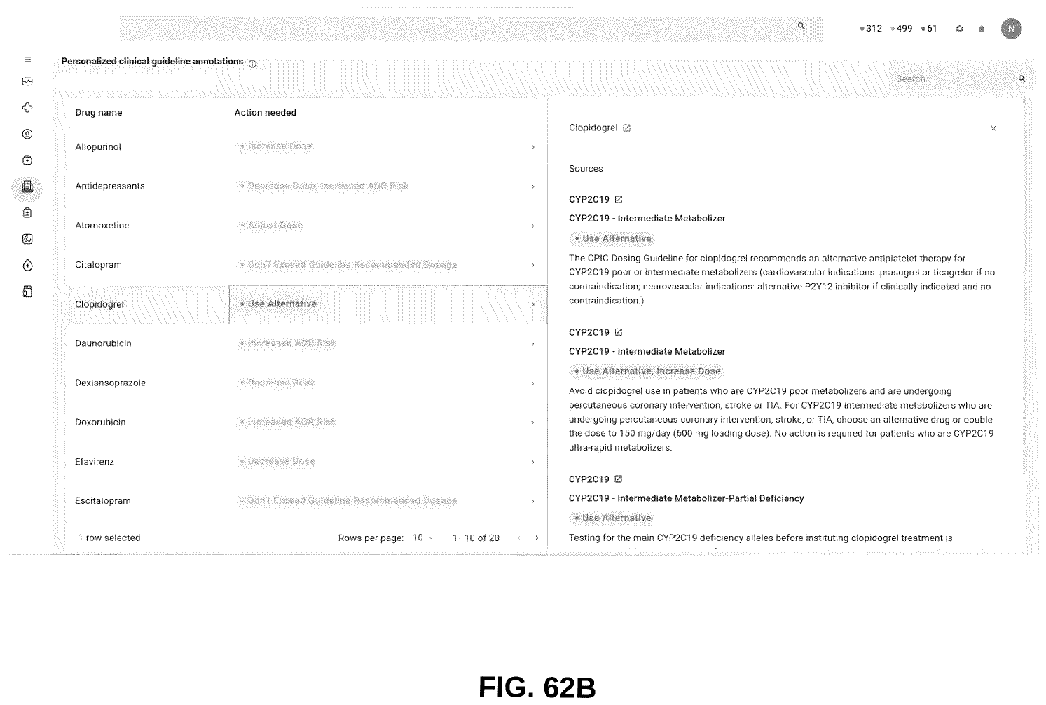

FIG. 62A and FIG. 62B are the Personalized Clinical Guideline Annotation webapp page, according to some embodiments;

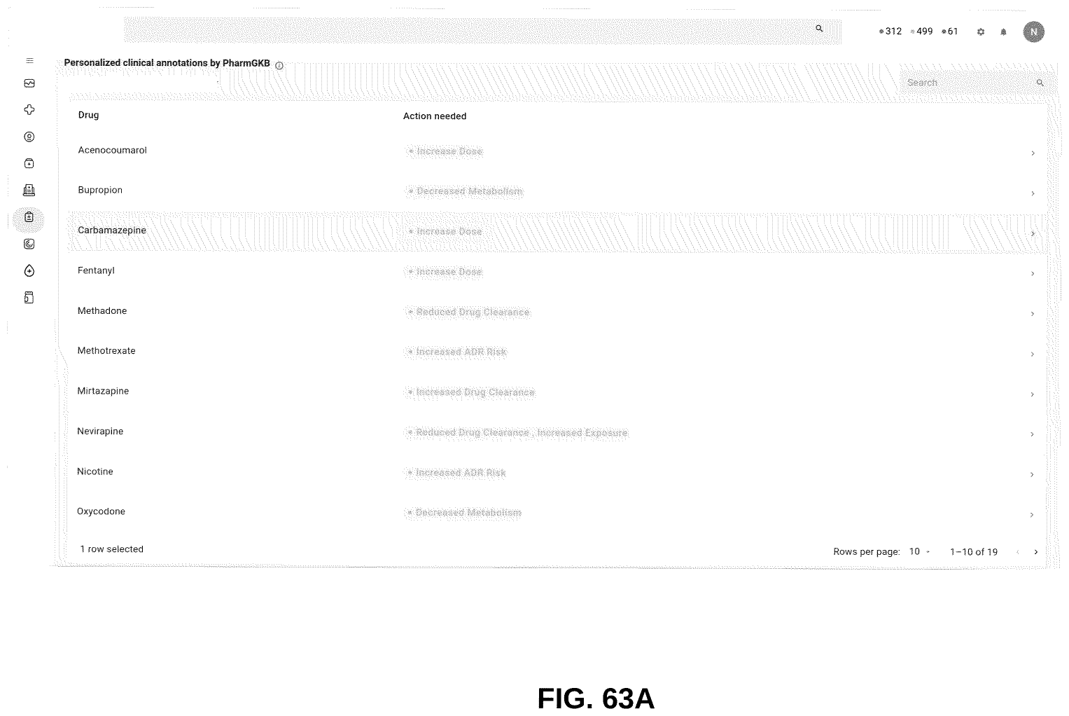

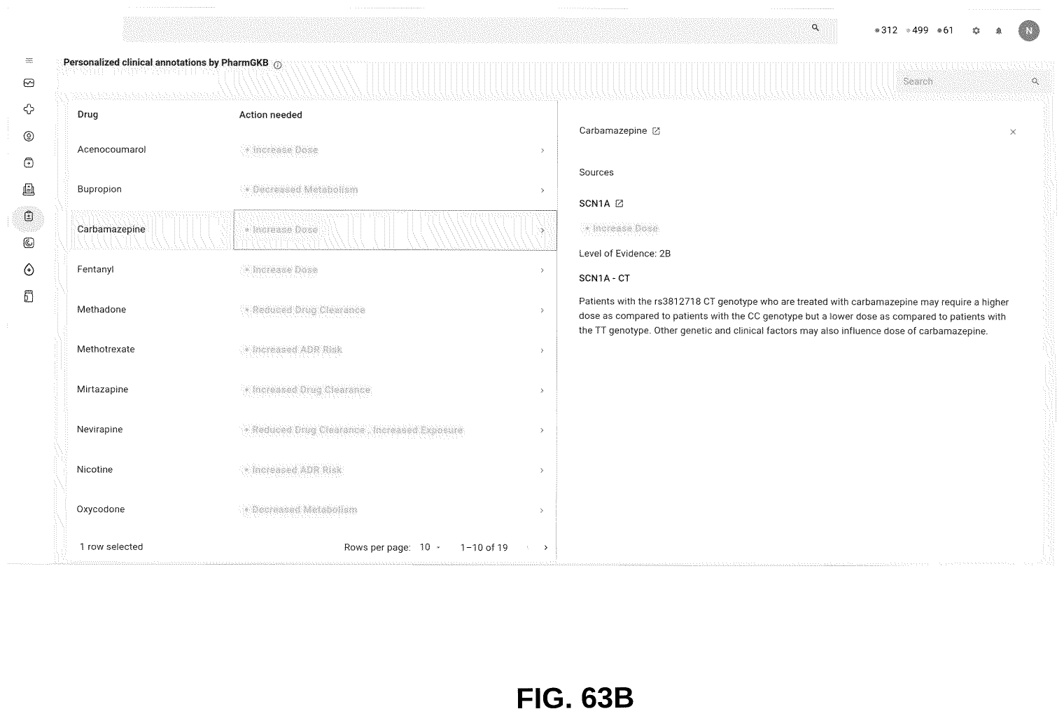

FIG. 63A and FIG. 63B are the Total Results by Therapeutic Area webapp page, according to some embodiments;

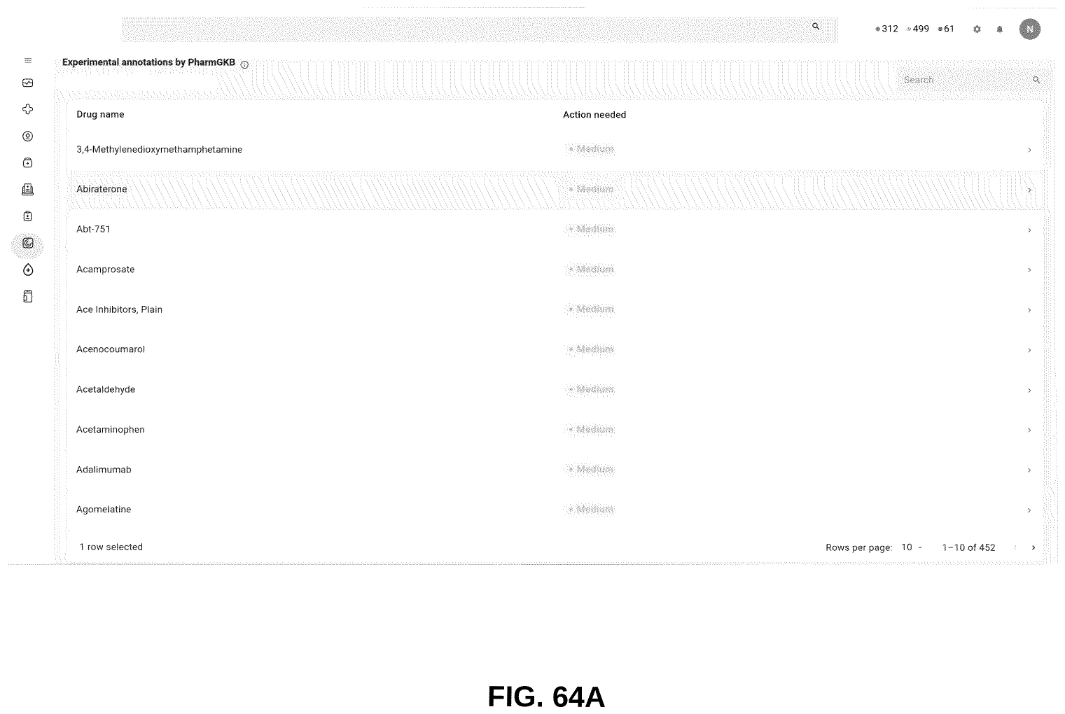

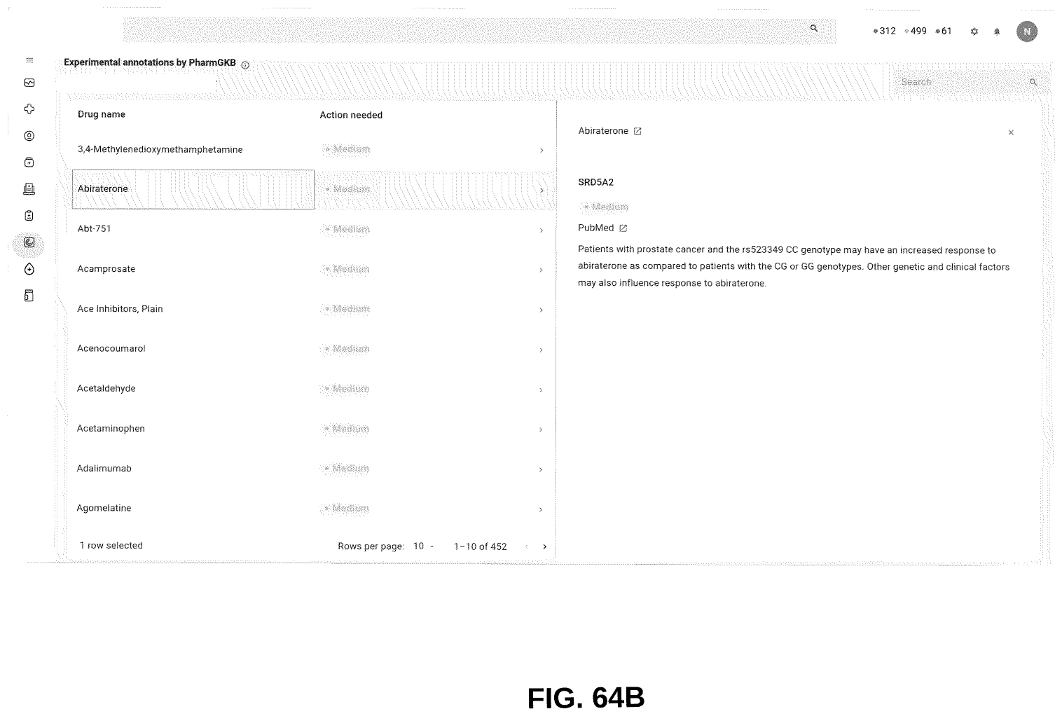

FIG. 64A and FIG. 64B are the Experimental Annotations by PharmGKB webapp page, according to some embodiments;

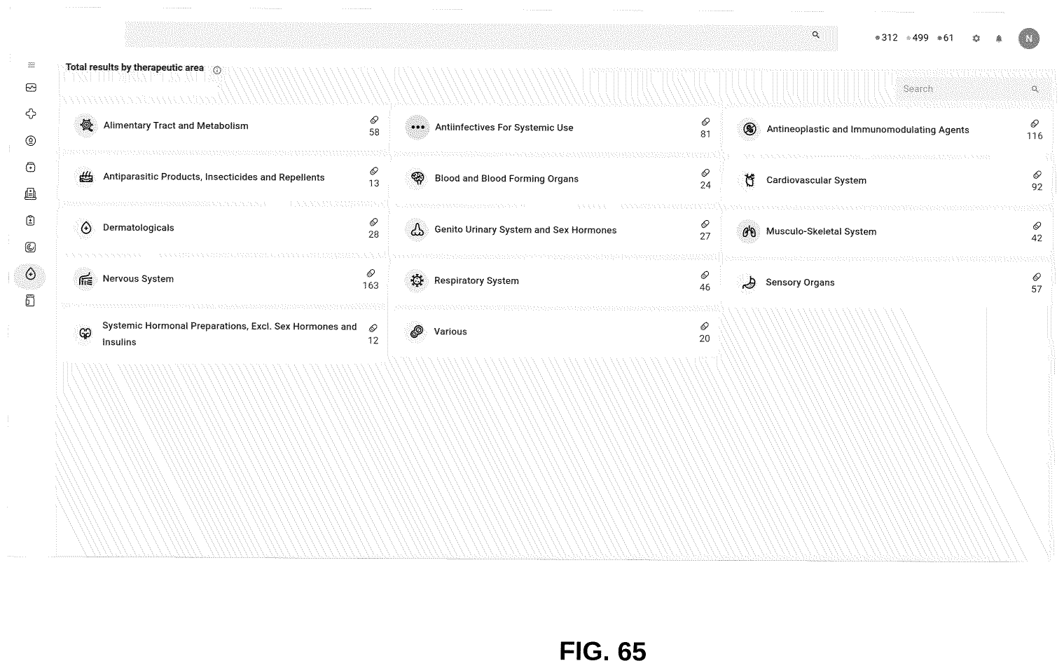

FIG. 65 is the Total Results by Therapeutic Area table webapp page, according to some embodiments;

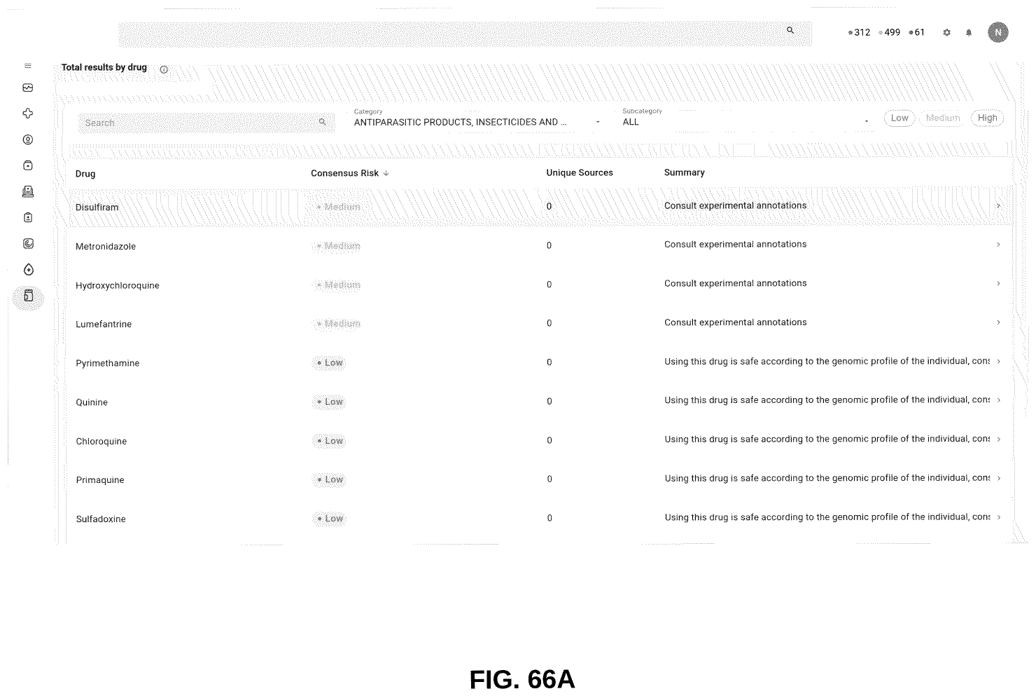

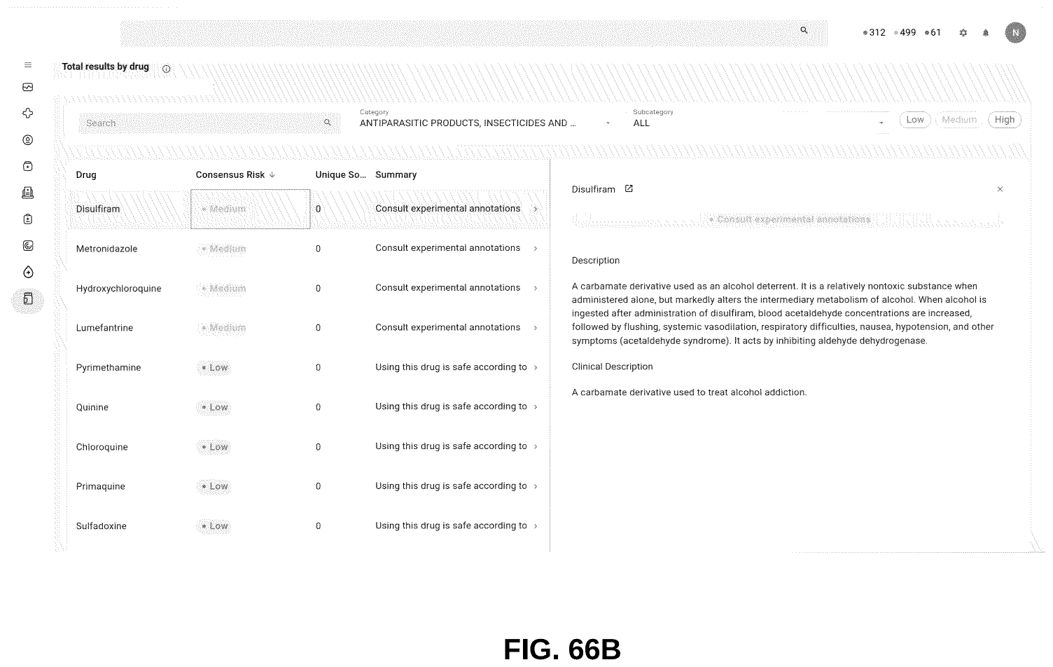

FIG. 66A and FIG. 66B are the Total Results by Drug webapp page, according to some embodiments; and

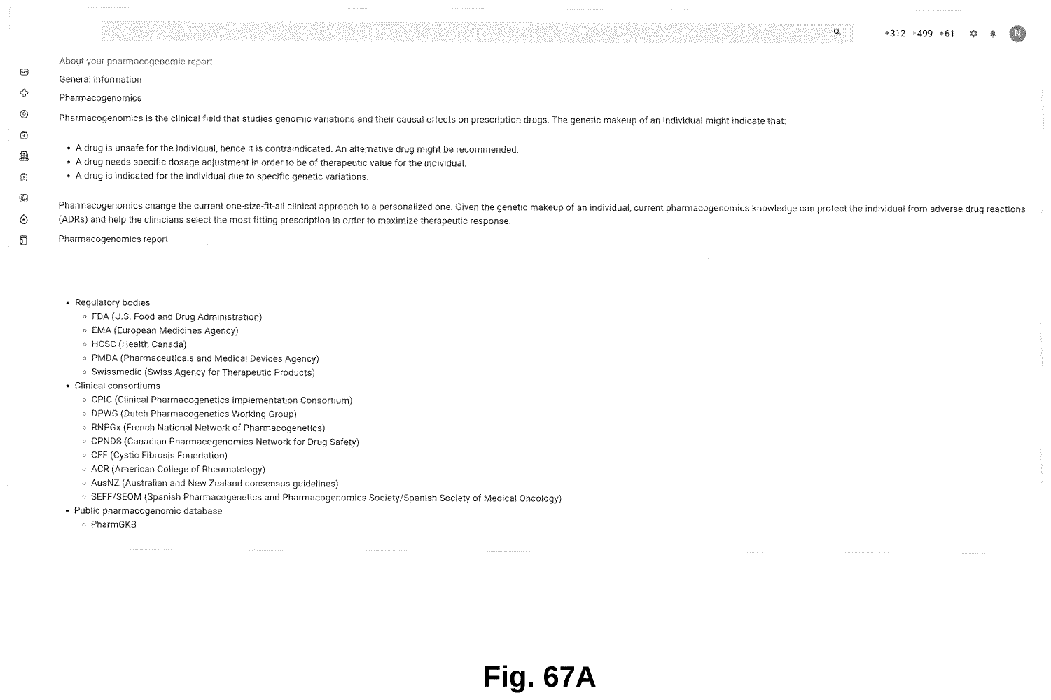

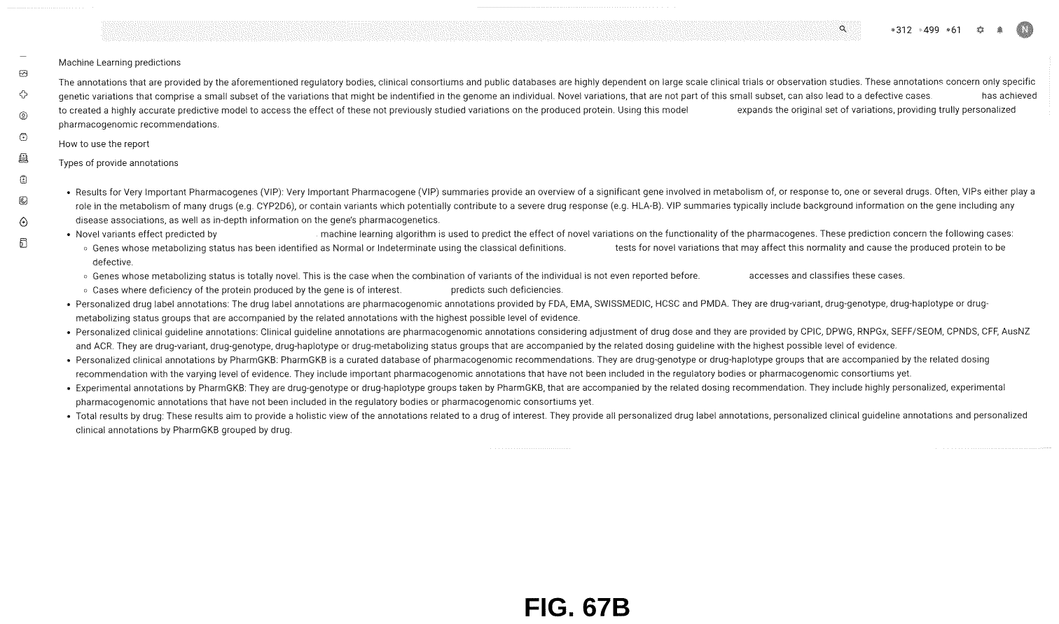

FIG. 67A and FIG. 67B is the How to Use the Report and Disclaimer webapp page, according to some embodiments.

### DETAILED DESCRIPTION OF EMBODIMENTS

The description that follows, and the embodiments described therein, are provided by way of illustration of an example, or examples, of embodiments of principles. These examples are provided for explanation, not limitation, of those principles. In the description, like parts are marked throughout the specification and the drawings with the same respective reference numerals. The drawings are not necessarily to scale, and, in some instances, proportions may have been exaggerated in order more clearly to depict certain features.

FIG. 25 is a schematic view of an example pharmacogenomic platform 100 according to some embodiments. In some embodiments, pharmacogenomic platform 100 includes one or more processing devices and one or more storage devices. The processing device is configured to execute instructions in memory to configure data processor 110, database configuration engine 120, machine learning engine 130, report generator 140, and display generator 150. A computing device 160, such as a mobile device running a mobile application or a remote server, is configured to connect with pharmacogenomic platform 100 and allow for user engagement. Computing device 160 is configured to present a display generated by display generator 150, according to some embodiments.

Pharmacogenomic platform 100 is configured to generate a personalized pharmacogenomic report derived from paired-end, shortread FASTQ files provided by a user, in some embodiments. In some embodiments, long-read FASTQ files are used. Using these FASTQ files, all DNA variations of interest are determined and searched for association with known pharmacogenomic annotations. These annotations are then processed and arranged appropriately and provided as an HTML, webapp, CSV, PDF, or JSON report in another format to the user. The report is arranged by report generator 140 and, for example, can be accessed by a contents panel on the left side of the page. By engaging with a display generated by display generator 150 at the content of interest, the results are delivered to the user as a sub-page, according to some embodiments. The contents of the report are described herein according to various embodiments.

### Sections of the Pharmacogenomic Report

Report generator 140 is configured to generate the pharmacogenomic report. In some embodiments, report generator 140 configures the pharmacogenomic report with interface elements arranged to depict the following sections (or analogous sections): About your Pharmacogenomic Report; Results for Very Important Pharmacogenes (VIP); Novel Variants Effect Predicted by pharmacogenomic platform 100; Personalized Drug Label Annotations; Personalized Clinical Guideline Annotations; Personalized Clinical Annotations by an external data source (e.g., PharmGKB); Experimental Annotations by an external data source (e.g., PharmGKB); Total Results by Therapeutic Area; and Total Results by Drug. In some embodiments, one or more of these sections are omitted and/or other sections are included.

According to some embodiments, report generator 140 is configured to generate the pharmacogenomic report as follows, arranging user interface elements depicting a unique arrangement of data that improves the accessibility and discovery of pertinent data. In other embodiments, report generator 140 is configured to generate a pharmacogenomic report that displays the features in a different manner.

In some embodiments, the About your Pharmacogenomic Report subsection is the only sub-page that is static, meaning that it always provides the same text for all the samples. It is meant to provide information about the report and how to use it. The information is grouped into three sections: General Information; How to Use the Report; and Disclaimer.

The “General Information” section contains the following data, including definitions, that is, general definitions about the terms nucleotide, DNA sequence, genome, gene, variant, novel variant, genotype, haplotype, diplotype and metabolizing/functionality status; a general definition of pharmacogenomics; an explanation on the goal of the pharmacogenomics report generated by pharmacogenomic platform 100 along with the sources that the pharmacogenomic annotations were mined from; and an explanation of how pharmacogenomic platform 100 is configured to use machine learning to detect pharmacogenomic related annotations for variants that have not been studied or uncovered before, as shown in FIG. 67A and FIG. 67B.

The “How to Use the Report” section contains the following data, including the types of provided annotations, which explains what results every other sub-page of the report is meant to provide; and the level of evidence for provided annotations, which explains that the most important sources for pharmacogenomic evidence are the regulatory bodies and clinical consortiums. Then it provides a clear explanation about level of evidence 1A, 1B, 2A, 2B, and 3-3 for the pharmacogenomic annotations provided by PharmGKB (See https://www.pharmgkb.org/page/clinAnnLevels). Finally, it informs clinicians or other users that they should consider annotations of those levels of evidence to act about prescribing a drug, as shown in FIG. 67A and FIG. 67B.

The “Disclaimer” section contains text that follows the disclaimers indicated by FDA (US Food and Drug Administration), other regulator guidance and research consortia guidelines, and any referenced clinical consortia or other data sources which will be indicated in FIG. 67.

The Overview page, as shown in FIG. 58A, contains an overview of all the patient's genomic profile. The user can toggle between all the therapeutic areas, using their keyboard, touch screen, or mouse to click through each image, represented as a human figure as shown in FIG. 58C-58N, respectively. Each therapeutic area provides the patient with a breakdown of all the “low risk”, “medium risk” or “high risk” drugs. On the right-hand side panel, the patient or doctor can add that individual's drug history by clicking on “Add drug” and choosing from a list of options made available from our current database.

The two right-hand side panels shown in FIG. 58A are broken down into Current Prescription and Drugs intending to prescribe. Once the user enters their current prescription in the top panel, any subsequent drugs added in the drugs intending to prescribe section, are checked against our drug interaction database. If there are any interactions present, the user will be notified as is shown in FIG. 58B. The report or user interface as described or as shown in the figures is different in different embodiments.

The Results for Very Important Pharmacogenes (VIP) subsection contain general pharmacogenomic consultation about the genes that were selected as Very Important Pharmacogenes (VIP) by pharmacogenomic platform 100, as shown in FIG. 59A and FIG. 59B. According to an example embodiment, these genes are CYP2C19, CYP2C9, CYP2D6, CYP2B6, CYP3A5, CYP4F2, DPYD, F5, HLA-A, HLA-B, SLCO1B1, UGT1A1, NUDT15, VKORC1 and TPMT. In other embodiments, other genes are selected. The page starts with a definition of the VIP genes as provided by an external data source, such as, in some embodiments, PharmGKB (https://www.pharmgkb.org/vips). Then, under the result section, a table is provided containing the results detected for a user. The table's fields include the identified phenotype field, which contains information about the identified phenotype and diplotype for each VIP gene. A red label is applied to diplotypes requiring adjustments for drugs according to pharmacogenomic annotations as needed; the identified diplotype field, lists the diplotypes that was identified for the user which led to the called phenotype; and general consultation field, contains text about whether drug dose adjustments are needed, without referring to specific drugs. This is a general consultation text. For example, the text is populated by report generator 140 from an external data source, such as PharmGKB. For cases that no such text exists custom texts are received by pharmacogenomic platform 100 and used to populate this section.

According to some embodiments, if the pharmacogenomic platform 100 is unable to detect the diplotype (or haplotype)/phenotype then report generator 140 is configured to display diplotype and phenotype fields in the report marked as “Unidentified.” As used herein, references to a diplotype is intended to be a reference to a haplotype in other embodiments. For greater clarity, data associated with a genomic variation of a gene can be a haplotype, diplotype, and/or phenotype. In some embodiments, a phenotype includes Adverse Drug Reactions, Metabolizing Status, Efficacy indications, Dosing data, Alternative drug data, Pharmacogenomic indication (a drug is indicated for a particular genotype), and/or Prescribing data.

Adverse Drug Reactions are undesirable and unintended responses to medications, which can range from mild side effects to severe allergic reactions or even life-threatening conditions. The following provides general examples of these terms, according to some embodiments. Metabolizing Status refers to an individual's ability to metabolize drugs, which can vary based on genetic factors, liver function, and interactions with other medications. Efficacy Indications indicate how effective a drug is in treating a specific condition or disease, based on clinical trials, studies, and real-world evidence. Dosing Data provides information about the recommended dosage of a drug, which can vary based on factors such as age, weight, renal function, and the severity of the condition being treated. Alternative Drug Data refers to alternative medications or treatment options that can be considered if the primary drug is ineffective, contraindicated, or not well-tolerated by the patient. Pharmacogenomic Indication indicates whether a drug is specifically recommended or contraindicated based on an individual's genetic makeup or genotype, helping to personalize treatment and optimize therapeutic outcomes. Prescribing Data includes information about the prescribing practices for a particular drug, such as prescribing patterns, guidelines, restrictions, and off-label uses, which can influence clinical decision-making and patient outcomes.

According to some embodiments, if the pharmacogenomic platform 100 (at database configuration engine 120) is configured to detect more than one possible diplotype/phenotype, since short reads can sometimes not discriminate the specific diplotype/phenotype of the user, then the report generator 140 is configured to generate a list of identified diplotypes separated by “or” for example. If all the diplotypes lead to the exact same phenotype, then the results for that phenotype are added to the report in FIG. 59A. Else, the phenotypes and general consultation texts for all cases are provided and separated by the “or” string.

The subsection of the report displaying outputs relating to Novel Variants Effect predicted by pharmacogenomic platform 100, can be seen in FIG. 60. These result from pharmacogenomic platform 100 which is configured by report generator 140 to include a description of what pharmacogenomic platform 100's machine learning model 2630 is configured to achieve. Then, the effect of possible novel variations for genes selected by pharmacogenomic platform 100 are assessed. The genes currently tested are the following: CYP2C19, CYP2C9, CYP2D6, CYP2B6, CYP3A4, CYP3A5, NAT2, SLCO1B1, UGT1A1, ABCG2, NUDT15, TPMT, G6PD and DPYD. A table is provided containing the results detected for the patient. The fields of the table include a field containing a gene symbol denoting the gene of interest; and the final functionality prediction field. If no novel variations are found within the gene, then the final functionality is suffixed by “with no novel variations”. If novel variations (which lead to a non-Normal case) are detected, then the phenotype derived from the variations is added to the table and suffixed with “due to detected novel variations.”

The Personalized Drug Label Annotations subsection is presented as a table, as can be seen in FIG. 61A. The table contains the pharmacogenomic annotations that apply to the genomic profile of the user and are derived from FDA (US Food and Drug Administration), EMA (European Medicines Agency), SWISSMEDIC, HCSC (Health Canada Santé Canada) and PMDA (Pharmaceuticals and Medical Devices Agency). The page starts with a description of the purpose of this sub-page. Then, the user can select whether they want to sort the result by drug name, by the source of evidence, or search the drug by typing it in search box.

The resulting display table includes the following information fields, namely, drug name; identified case, that is, the identified variant that relates to the annotation. Action needed column on FIG. 61 summarizes the actions that should be taken, for example whether to increase or decrease the dose, or whether ADRs (Adverse Drug Reactions) are likely.

The data may initially only display drug or action needed, such that, upon clicking the drug or source information, further fields of information will be revealed within the right-side panel of the webapp as per FIG. 61B.

Drugs listed in this table have an associated pharmacogenomic annotation denoting that some action should be taken.

The Personalized Clinical Guideline Annotations subsection is presented as a table, as can be seen in FIG. 62A. This table contains the pharmacogenomic annotations that apply to the genomic profile of the user. The data input may contain information from an external provider, consortium or regulatory body. In some embodiments this information is derived from CPIC (Clinical Pharmacogenetics Implementation Consortium), DPWG (Dutch Pharmacogenetics Working Group), RNPGx (The French National Network of Pharmacogenetics), SEFF/SEOM (Spanish Pharmacogenetics and Pharmacogenomics Society and the Spanish Society of Medical Oncology), CPNDS (Canadian Pharmacogenomics Network for Drug Safety), CFF (Cystic Fibrosis Foundation), AusNZ (Australian and New Zealand consensus guidelines) and ACR (American College of Rheumatology). The page starts with a description of the purpose of this sub-page. Then, the user can select whether they want to sort the result by drug name or by the action needed. After the selection, the table can be searched using the search box. The table includes the following information fields, namely, drug name; action needed, and when the user clicks on the drug, more information is revealed on the right-side panel as is shown in FIG. 62B. There, the patient can find the source along with a detailed description of the annotation based on each available source as well as the identified variant that relates to this annotation. Action needed summarizes the actions that the patient may take based on the available information, for example, whether the patient should increase or decrease their dose of that particular drug. The data may initially only display drug or action needed, such that, upon clicking the drug or action needed, further fields of information will be revealed on the right-hand side panel as per FIG. 62B.

Any drug listed in this table represents that there is an associated pharmacogenomic annotation and that some action should be taken by the patient.

The Personalized Clinical Annotations subsection is presented in the form of a table as is shown in FIG. 63A. The table contains the pharmacogenomic annotations that are considered important (level of evidence “1A”, “1B”, “2A”, “2B”). The page starts with a description of the purpose of this sub-page. The fields of the personalized table include the drug name; action needed, and the identified variant that relates to the annotation when the user clicks on a particular drug. Each source of evidence, contains URLs to the pages supporting the annotation; action needed summarizes the actions that should be taken which are accompanied by their relevant pharmacogenomic annotations and sources of evidence.

The data may initially only display drug information, such that, upon clicking the drug information, further fields of information will be revealed on the right-hand side panel as is shown in FIG. 63B, according to some embodiments.

Each drug listed in this table has an associated pharmacogenomic annotation and that some action should be taken.

The Experimental Annotations by PharmGKB subsection are presented in the form of a table as is shown in FIG. 64A, according to some embodiments. The table contains the pharmacogenomic annotations that are considered experimental (level of evidence “3”) and have not been accepted by a regulatory body/clinical consortium yet. The page starts with a description of the purpose of this sub-page. The fields of the personalized table include the drug name, the identified case, the source of evidence, and the pharmacogenomic annotation. The field identified case contains the respective genomic variant for this annotation. This field includes the gene, the located variant and if found the respective list of haplotypes and the list of haplotype functions; identified case, that is, the identified variant that relates to the annotation. The field source of evidence contains URLs to the pages supporting the annotation and the field phrmacogenomic annotation contains a textual representation of the respective pharmacogenomic annotation.

The data may initially only display the drug name upon clicking on the drug name, further information will be revealed as per FIG. 64B.

Having a drug in this table means that there is an associated pharmacogenomic annotation and the action that should be taken at the sole discretion of the clinician.

The Total Results by Therapeutic Area subsection is presented in the form of a table as can be seen in FIG. 65. The table contains all the pharmacogenomic annotations from all sources, that apply to the genomic profile of the user. The page starts with a description of the purpose of this sub-page. Then, the user can select the therapeutic area they want to investigate by clicking on the relevant category listed, whether they want to see the result for All drugs, only for Drugs with PGx annotations, only for Drugs with experimental annotations or drugs that are safe to use (Default is All Drugs). Each drug in the therapeutic area is broken down into subcategories as defined by the World Health Organization (WHO) Anatomical Therapeutic Chemical (ATC) classification system (https://www.who.int/tools/atc-ddd-toolkit/atc-classification). Safe drugs are considered those that do not have elevated level of evidence or experimental pharmacogenomic annotations. All the tables have the same fields, but the rows that are provided are different. This searchable table provides drugs grouped by therapeutic and sub-therapeutic areas. After selecting the type of drugs (All drugs, Drugs with PGx annotations, or Drugs with experimental annotations) the table can be searched by drug name.

The fields of the produced table include category, that is, the category that the drug belongs to; subcategory, that is, the sub-category that the drug belongs to; drug name, that is, the drug name followed by a red label implies that there is high level of evidence, a yellow label if only experimental exist or a green label if no annotations exist. Some cases relate to other annotations of drug groups and these are suffixed as “(Other PGx annotation) “; description of the drug's usage; and pharmacogenomic annotations, that is, text containing the fields identified case, action needed and the pharmacogenomic text per case. In case of drugs with high LOE (Level Of Evidence) these annotations are replaced by the appropriate texts which includes the identified case, the action needed and the respective pharmacogenomic annotation. This is exactly the same information that is show in the “Personalized clinical guideline annotations” part of the report. In the case of drugs that have only experimental annotations (given that the doctor might not want to follow those) the following text is added “Identified_case: Only experimental level annotations detected Action needed: Decide prescription after consulting the experimental annotation Pharmacogenomic annotation: Using the drug is potentially safe.” Finally, for safe drugs the following text is used “Identified_case: None of the related genes are affected by variations Action needed: None Pharmacogenomic annotation: Using the drug is safe according to the genomic profile of the individual.”

The Total Results by Drug subsection is presented in the form of a table, as can be seen in FIG. 66A. The table contains all the pharmacogenomic annotations from all sources, that apply to the genomic profile of the user. The page starts with a description of the purpose of this sub-page. The important thing about this table is that it provides the total results by drug name and can be easily searched. The field of the produced table includes drug name; and category can be filtered using a drop-down field. In the case of drugs with pharmacogenomic annotation the source of annotation is kept in a consistent format which adheres to the following pattern: (SOURCE) [POSSIBLY A URL ABOUT THE SOURCE] GENE: (GENE NAME) CASE: (THE RELATED VARIANT) [POSSIBLY ALSO Combination of (LIST OF HAPLOTYPES (FUNCTIONS OF HAPLOTYPES))]. An example is: SOURCE: DPWG URL OF SOURCE: https://pubmed.ncbi.nlm.nih.gov/21412232/, Gene: CYP2C19, CASE: Intermediate Metabolizer

In case of drugs that only have experimental annotations, the field is assigned the text “Only experimental level annotations detected”. For the safe case the text is “None of the related genes are affected by variations”; action needed: In the case of drugs with pharmacogenomic annotation it is a summary text about what the pharmacogenomic annotation states. In case of drugs that only have experimental annotations, the field is assigned the text “Decide prescription after consulting the experimental annotation”. For the safe case the text is “None”; and pharmacogenomic annotations, that is, in the case of drugs with pharmacogenomic annotation the pharmacogenomic annotation text. In case of drugs that only have experimental annotations, the field is assigned the text “Using the drug is potentially safe”. For the safe case the text is “Using the drug is safe according to the genomic profile of the individual”.

After clicking on a specific drug, a right-hand side panel opens as per FIG. 66B. There is a general description of the drug with sources in superscript, a clinical description, which is then followed by all the sources with appropriate links to the URL, which are all provided by DrugBank. If available, based on the individual's genomic profile, a recommendation along with their sources are provided for each drug.

Report generator 140 includes report generation scripts stored, for example, in a database such a cloud repository.

The report generator 140 may contain any subset of pharmacogenomic variants of interest that exists in the database engine 120. This allows for the use of any of the following technologies for genomic profiling; Next Generation Sequencing (NGS), Third-generation Sequencing or Single Nucleotide Polymorphism (SNP) arrays. In the case NGS is used then the complete workflow presented is applied that contains the steps of variant alignment and variant calling. In the case of the third-generation sequencing, the steps of read alignment is adjusted to accommodate for long reads. In the case a SNP array is used then the steps variant alignment and variant calling can be omitted and the genotypes that are captured in any Microarray or multiplex PCR screening device can be used.

### The Pharmacogenomic Database

In some embodiments, report generator 140 is configured to implement a process that generates the pharmacogenomic report. This report processes the genetic make-up of the individual and is based on the latest known information available in our database. This database is updated weekly for any new evidence as suggested by the regulatory bodies listed in this document. For example, the FDA, EMA, SwissMedic and PharmGKB are scraped for any new information to be added to the database. With this information each patient can make informed decisions on their health, as each drug comes with the latest recommendation, which is specifically tailored to the patient. Pharmacogenomic platform 100 is configured to use our computational workflow tool and a Cloud computing service to generate the results by running the code to implement a process workflow. The code is executed on the cloud computing service.

In some embodiments, the cloud provider infrastructure is used to assist the generation of the pharmacogenomic report. Resources include storage accounts, for the FASTQ files, intermediate files as well as the results. This data storage is configured to include at least four containers: input, output, ongoing and human reference. The input file contains the FASTQ files, the output file is where the results are stored, the ongoing file is needed to run the workflow and the humanreference is configured to store files that are used for the analysis such as the human reference genome GRCh38. The cloud provider is used to create pools of VMs that run the workflow; and their registry's resource is used to store the repository container images needed to run each step of the workflow. Our images are further described herein; the Cloud providers Database (or external database) stores the pharmacogenomic database; and the Cloud services providers, for example Azure Genomics Account, a resource is used to detect variants from the raw FASTQ files. In some embodiments, Azure is not used, but these resources are implemented without Azure. In some embodiments, the workflow is run on a cluster using Kubernetes or similar cluster management systems.

Using the resources listed above pharmacogenomic platform 100 is configured to implement a method to receive and use raw FASTQ files. This section will give a general overview of the steps taken by this method, according to some embodiments. The step numbers that correspond to function names referenced here, are also shown in FIG. 19 and FIG. 24. The method is a combination of a genomics tool whose results are then parsed into another workflow. At step 1, the Genomics tool used to convert raw FASTQ files into VCF (Variant Call Format), BAM (Binary Alignment Map), BAI (Binary Alignment Index) and log files. At step 2, supplementary files are used to increase the prediction accuracy and calls, namely, golden standard data inputs from the HapMap project and insertion/deletion tables from the 1000G project are used. To make a prediction on the UGT2B17 gene, a file containing its region is supplemented. The workflow is comprised of 3 processes: filtering the VCF using the supplementary data mentioned, making appropriate calls based on the genotype and creating output files. The output files are trimmed by selecting only regions of interest. These workflows are processed in parallel. In step 3, these processes annotate the raw VCF with a FILTER field which indicates whether a variant is of high probability to be called correctly or not. The VCF file is initially indexed using selected regions of ibgzip and the bed file is used to filter the VCF file to contain variants only for the regions of interest. Then, GATK's (Genome Analysis ToolKit) CNNScore Variants and FilterVariantTranches are applied to the raw VCF and generate the annotate the VCF at step 4, the BAM, BAI files and the reference files are now used in the Caller process. This process is part of the whole code of pharmacogenomic platform 100, and its purpose is to bring together tools to call the diplotypes for the families of CYP (Cytochrome P450) and HLA (Human Leukocyte Antigens) genes as well as to call the depth of the UGT2B17 gene. To achieve this a script uses StellarPGx, graphtyper and bedtools. Initially the process uses some code to correct the download order of the BAI file. Then, the generalcall.sh (based on StellarPGx) script is used to call the diplotypes of the CYP family of genes. Then, the bedtools tool is used to identify the depth of the UGT2B17 gene. Then, another script calls the diplotypes of the HLA family of genes. The step generates three files, one for the CYP family diplotypes, one for the HLA family diplotypes and one for the depth of the UGT2B17 gene. At step 5, the files generated by steps 1,2,3, and 4 are used as input to the report generation process which is initiated by the run_workflow( ) function. The run_workflow( ) function, combines the personalized genomic variation (contained in the files generated from Steps 1,2,3,4) with the pharmacogenomic information contained in the database. This function creates a set of tables containing all the information relevant to the personalized pharmacogenomic report. These tables are shown in rectangle 1910 in FIG. 19. The function run_workflow( ) implements steps 6,7,8,9,10,11,12,13 and 14. These steps are explained later. After the function is completed, the function get_report_tables( ) is executed next. The function get_report_tables( ) takes as input the tables with the personalized pharmacogenomic information (generated from function run_workflow( ) and converts them into tables containing user readable text and information. The function get_report_tables( ) implements steps 16,17,18,19,20,21,22 and 23 as shown in FIG. 24. These steps are explained later. After the execution of get_report_table( ) an extra step (Step 24) takes place in which the appropriate CSS styling and images are added. The final personalized pharmacogenomics report is the results of Step 24. Here we describe steps 6-24 in more detail. At step 6, the get_variation_ids( ) a function is used and to translate to select the variations that passed the filtering step and to create different id representation for each variant. The VCF file is cross-referenced against our platform'sThe exact of these ids are provided in the section get_variation_ids( ) step; at step 7, the get_pgx_tables( ) function is then used to pull all the tables of interest from platform's 100 pharmacogenomic database; at step 8, given a specific representation of the variants produced by the get_variation_ids( ) step and the tables of get_pgx_tables( ) that contain pharmacogenomic annotations of type genotype or variant from related sources, the get_var_annotations( ) function is used to detect all these pharmacogenomic annotations of the database; at step 9, given a specific representation of the variants produced by the get_variation_ids( ) step, the tables of get_pgx_tables( ) that contain Information about the relationship between a combination of variants and the associated diplotype, the CYP calls, the HLA calls and the UGT2B17 genes. the get_hap_dip( ) function is The script formats the CYP and HLA diplotype, detects the diplotype of UGT2B17 and detects the diplotypes for the other genes of interest. The algorithms to call UGT2B17 and other genes are further explained at get_var_annotations( ) step section. This step generates a table containing all the diplotypes and haplotypes for the genes of interest; at step 10, as in step 8 the haplotypes and diplotypes are now used to detect the associated annotations from a database of pharmacogenomic platform 100. The VCF file genomic locations are converted into a unique representation to quickly match them with the The function that achieves this assignment is get_hap_annotations( ) at step 11, the appropriate variant id representations from get_variation_ids( ) and the table containing the predictions of machine learning engine 130 of pharmacogenomic platform 100 are used to detect whether there are other variants whose effect is predicted by machine learning engine 130. If such variations are detected for the genes of interest the predictions “No”, “Decreased” or “Normal” made by the ML model for the variant are translated to phenotype according to a gene related mapping. The steps used in creating the ML model are described in a later section, further described at the get_myengene_pred( ) step section; at step 12, the get_met_status( ) function is then used to detect, in summary, the metabolizing statuses from the called diplotypes (generated by the step get_hap_dip( )), the predictions made by machine learning engine 130 (generated by the step get_myengene_pred( )) and the tables of the database that assign a metabolizing status to a known diplotype; at step 13, as in step 10 the metabolizing statuses are used to detect the associated annotations from the database of pharmacogenomic platform 100. The function that achieves this assignment is get_met_annotations( ) at step 14, the metabolizing statuses are used to get the general consultations about the VIP genes selected by pharmacogenomic platform 100 through the get_cds( ) function. Steps 6-14 are executed by a single function call run_workflow( ) The outputs of this function are used by the following functions; at step 15, the custom get_reactable_cds( ) function is used to generate the table of the “Results for Very Important Pharmacogenes (VIP)” page of the report; at step 16, the custom get_reactable_myengene( ) function is used to generate the table of the “Novel variants effect predicted by pharmacogenomic platform” page of the report; at step 17, the custom get_reactable_label( ) and get_reactable_label_source( ) functions are used to generate the two tables of the “Personalized drug label annotations” page of the report; at step 18, the custom get_reactable_dosing( ) and get_reactable_dosing_source( ) functions are used to generate the tables of the “Personalized clinical guideline annotations” page of the report; at step 19, the custom get_reactable_pharmgkb( ) function is used to generate the table of the “Personalized clinical annotations by PharmGKB” page of the report; at step 20, the custom get_reactable_experimental( ) function is used to generate the table of the “Experimental annotations by PharmGKB” page of the report; at step 21, the custom get_reactable_cds( ) function is used to generate the table of the “Experimental annotations by PharmGKB” page of the report; step 22, the custom get_reactable_grouped( ) function is used to generate the tables of the “Total results by therapeutic area” page of the report; at step 23, the custom get_reactable_total_drugs( ) function is used to generate the table of the “Total results by drug” page of the report. Steps 16-23 are executed at by the get_report_tables( ) function; step 24, using the personalized-pgx-report.Rmd and the associated css and images the final report is generated using the results of the get_report_tables( ) function.

Machine learning engine 130 is configured to train and generate at least one machine learning model. The machine learning model is generated according to a method described as follows. Given a variation whose effect on the functionality of the produced protein is known and using several variables related to these variations, a predictive model is generated by machine learning engine 130. The variations that were used to train the model are those described in the foregoing reference, which are derived by Pharm Var. In some embodiments, this dataset is expanded using variants from LOVD (https://databases.lovd.nl/shared/variants) that are known to affect or likely to affect the functionality of the protein. Additional data can be used, according to some embodiments. Manual curation can be received as data input and used by machine learning engine 130 to associate labels to these variants. The dataset was further expanded by adding variants from Clin Var that are related to pharmacogenomics and were not added before. This can be received as data input. Given the variant . . . , the reference sequence, alternative sequence and the prediction, new variables related to the variants were determined by machine learning engine 130 by getting the commercial version of dbNSFP and selecting all fields. In some embodiments, database configuration engine 120 is configured to generate a pharmacogenomic database for pharmacogenomic platform 100. Database configuration engine 120 is configured to generate at least one database defined by the entity relationship diagram and the respective database tables shown in the FIG. 57A, 57B, 57C, 57D, 57E, according to some embodiments. Report generator 140 is configured to receive data from the database and generate at least one interface element for presenting the data, such as according to the table design in the database. An example embodiment will now be described. Database configuration engine 120 is configured to generate each of the tables, according to some embodiments.

In some embodiments, there are several tables configured by database configuration engine 120 and generated at a display by report generator 140 in the report that contains data relating to genes, drugs, label recommendations, dosing recommendations, clinical pharmacogenomic; pharmacogenomic platform 100 predictions, drug groups, haplotype calls, activity scores, phenotypes table, CDS (Clinical Decision Support) table, haplotypes function, and deficiencies table. FIG. 57A, 57B, 57C, 57D and 57E contain the database schema with all tables and all fields.

The table containing data relating to genes include the following fields, namely, gene symbol; ensembl_id: The ensembl id of the gene; hgnc_id: The hgnc id of the gene; the name of the gene; is_vip, that is, identifier about whether the gene is considered VIP by PharmGKB or not; the chromosome of the gene; the start location on the chromosome; and the stop location on the chromosome. This table contains general information about the genes of interest. The table contains a super set of the genes of interest. For example, the table can be generated using the genes table provided by PharmGKB (https://api.pharmgkb.org/v1/download/file/data/genes.zip).

The table containing data relating to drugs includes the following fields, namely, the id drug name; the generic name of the drug, and the brand name of the drug. This table contains general information about the drugs of interest. The table contains a super set of the drugs of interest. For example, the table can be generated using the drugs table provided by PharmGKB.

The table containing data relating to label recommendations include the following fields, namely, an auto increment id of type integer; the gene symbol of the gene that the pharmacogenomic annotation relates to; the drug name of the drug that the pharmacogenomic annotation relates to; the source from which the annotation is provided. The current sources are the regulatory bodies Swissmedic, HCSC, EMA, FDA, PMDA; the name of the annotation on the PharmGKB database; the specific variation that is related to the pharmacogenomic annotation. While it is referred as an allele it can be of any type listed on the type of field. The different types are Variant (when interested only for the existence of a variant even in heterozygous case), Genotype (when both inherited variants must be known), Haplotype, Diplotype and Metabolizing_status; the summary text of the pharmacogenomic annotation mined from PharmGKB or other sources; the full text of the pharmacogenomic annotation mined from PharmGKB or other sources. For the case of FDA this is the excerpt from the drug label; the simple_annotation, that is, the text warning for dosage adjustments that might be needed; risk, that is, the text warning for potential ADRs (Adverse Drug Reactions) risks that might come up; and publications, that is, URL to the source of the original annotation. This table contains all the gene-drug-variant-pharmacogenomic annotation relationships as indicated by drug labels.

This table is generated by database configuration engine 120 by receiving data from data processor 110, which is configured to retrieve or receive data from external sources, such as by web scrapping PharmGKB in combination with the tables provided by FDA in the following link https://www.fda.gov/medical-devices/precision-medicine/table-pharmacogenetic-associations, which is hereby incorporated by reference in its entirety. The specific variation provided on the allele field and the type of field were generated by data processor 110 by text-mining the summary and full text per annotation. Manual curation can be received as data input to identify any wrong assignments. The FDA texts can be transformed into the format of the exact drug excerpt. The simple_annotation and risk fields are generated by going through the summary_text and full_text. Finally, the publications field is generated through the URLs identified by PharmGKB per annotation.

The table containing data relating to dosing recommendations includes an auto increment id of type integer; the gene symbol of the gene that the pharmacogenomic annotation relates to; the drug name of the drug that the pharmacogenomic annotation relates to; the source from which the annotation is provided. The current sources are the clinical consortiums ACR, SEFF/SEOM, RNPGx, DPWG, CPIC, AusNZ, CFF and CPNDS; the name of the annotation on the PharmGKB database; allele, that is, the specific variation that is related to the pharmacogenomic annotation. While it is referred to as an allele it can be of any type listed on the type of field; the type of the allele. The different types are Variant (when interested only for the existence of a variant even in heterozygous case), Genotype (when both inherited variants must be known), Haplotype, Diplotype and Metabolizing_status; the summary text of the pharmacogenomic annotation mined from PharmGKB or other sources; simple_annotation, that is, an text warning for dosage adjustments that might be needed; risk, that is, the text warning for potential ADR (Adverse Drug Reactions) risks that might come up; and publications, that is, the URL to the source of the original annotation. This table contains gene-drug-variant-pharmacogenomic annotation relationships as indicated by clinical consortiums.

This table was generated by database configuration engine 120 by receiving data from data processor 110, which is configured to text mine the Clinical Guideline annotations JSON file provided by PharmGKB. The specific variation provided on the allele field and the type of field are generated similarly by text-mining the summary_txt per annotation. Manual curation can be used to identify any wrong assignments. The simple_annotation and risk fields can be generated by going through the summary_text. Finally, the publications field can be generated by going through the URLs identified by the JSON per annotation.

The table containing data relating to clinical pharmacogenomic data include an auto increment id of type integer; variant_id, that is, the general id of the format (rs_id): (GENOTYPE) for variants and (GENE SYMBOL) (HAPLOTYPE) for haplotypes; the gene symbol of the gene that the pharmacogenomic annotation relates to; the drug name of the drug that the pharmacogenomic annotation relates to; the type of the variation. The diverse types are Genotype and Haplotype; genotype, that is, the specific variation. For the type “Genotype” it is mostly of type Genotype while single variants can also be detected and for the type of “Haplotype” it is both haplotypes and diplotypes; LOE, that is, the level of evidence of the annotation. (See); the score assigned to the annotation by PharmGKB; the category assigned to the annotation by PharmGKB; the phenotype assigned to the annotation by PharmGKB; the population applicability assigned to the annotation by PharmGKB; the recommendation, that is, the pharmacogenomic annotation text that is assigned by PharmGKB; simple annotation, that is, the text warning for dosage adjustments that might be needed; the risk, that is the text warning for potential ADR risks that might come up, and the publications, that is, the URL to the source of the scientific support.

This table contains the gene-drug-variant-pharmacogenomic annotation relationships as indicated by PharmGKB.

This table was created by text mining the tables of the clinicalAnnotations.zip file provided by PharmGKB. The simple_annotation and risk fields can be generated by going through the summary_text. These fields are filled only for the case of annotations with loe “1A”, “1B”, “2A” or “2B” in some embodiments.

Pharmacogenomics platform 100 is configured to receive manual corrections as data input at data processor 110 such as to correct data arising from inconsistencies in data received from external sources such as PharmGKB. For example, the genotype given for heterozygous variants may not be in the format (REF) (ALT). This can be corrected so that these variations can be called. While many annotations refer to diplotypes variant identified is just a haplotype. For the cases of annotations with LOE “1A”, “1B”, “2A” or “2B”, the texts from PharmGKB can be used to turn the haplotypes into diplotypes, as well as to identify the simple_annotation and risk fields.

The table containing data relating to predictions generated by machine learning engine 130 include hgvsc, that is, the id of the variant represented in the format (CHROMOSOME): g. (POSITION) (REF)> (ALT); the gene symbol of the gene that the variant belongs; the chromosome that the variant is found; the position that the variant is found; the reference allele of the variant; the rs id of the variant if it exists; the prediction class predicted by machine learning engine 130. Classes are “Normal,” “Decreased” and “No” meaning function; and the probability of the variant assigned to the predicted class being correct.

This table contains variants whose effect on the functionality of the produced protein is predicted using machine learning engine 130.

To create this table the data from https://sites.google.com/site/jpopgen/dbNSFP is retrieved. Then the columns matching those of interest and those that are important to make predictions with the machine learning model are selected. The machine learning model is used to predict the functionality status of the variants of interest. The class with the highest probability is then selected per variant.

The table containing data relating to drug groups include an auto increment id of type integer; the drug name; class1, that is, the sub-category of the drug; class2, that is, the category of the drug; class3, that is, the general category of the drug; the summary of the drugs usage; is_group, that is, an indicator about whether drug name relates to a group of drugs or not.

This table is important to group drugs by their therapeutic area in the final report.

This table is generated using the drugs table provided by PharmGKB. The ATC_identifier was taken for all drugs of interest and the https://www.whocc.no/webpage was used to transform this identifier in the classes of interest. For cases that did not have an ATC_identifier, the classes were identified and provided to data processor 110.

The table containing data relating to haplotype calls includes an auto increment id of type integer; the gene symbol that the haplotype refers to. In one example embodiment, the haplotypes of interest relate to the genes ABCG2, DPYD, F5, NUDT15, SLCO1B1, TPMT, TYMS, UGT1A1 and VKORC1; diplotype, that is, the haplotype of the gene; and the total variants that consist of the diplotype. In other embodiments, other genes are used. Specifically, reference cases are indicated as empty strings and variant cases contain all variants from both haplotypes separated by comma and sorted as texts. The variant representation used for each variation is the (CHROMOSOME). (POSITION) (ALLELE). As used herein, delimiters or methods other than those specified can be used to mark the data or text.

Most calls of the diplotypes are made using the modified StellarPGx workflow (for CYP family genes), the graphtyper (for HLA family genes) and UGT2B17 depth (for UGT2B17 diplotype) during the workflow. These tools though do not provide the haplotypes for all the genes of interest. The other diplotypes are called using this table and the combinatorics approach originally suggested by the StellarPGx workflow.

The table was generated using the “(GENE SYMBOL) Allele definition table” from https://www.pharmgkb.org/page/pgxGeneRef for genes of interest. In these tables the rs code is transformed in the format (CHROMOSOME). (POSITION) (ALLELE). The variations per haplotype are identified and all possible diplotypes are created by generating all the possible variations. The vector of total variants is then sorted and pasted in a comma separated format.

The table containing data relating to activity score includes an auto increment id of type integer; allele, that is, the haplotype of the gene; the gene symbol that the haplotype activity refers to. In an example embodiment, the activities of interest relate to the gene DPYD; and the activity assigned to the haplotype. The activities of interest can relate to other genes in other examples.

This table is used to call the diplotype of DPYD for pathogenic cases that arise due to fact that the gene is highly polymorphic.

This table was generated using the “(GENE SYMBOL) Allele Functionality Table” from https://www.pharmgkb.org/page/pgxGeneRef for genes of interest.

The table containing data relating to phenotypes table an auto increment id of type integer; the diplotype of the gene. The diplotype is the combination of the two haplotypes that are identified for a gene; the activity assigned to the diplotype. In some pharmacogenomic annotations, from the called diplotype and phenotype it is important to know what the specific activity of the diplotype is; the phenotype (mainly metabolizing statuses) that is assigned to the diplotype. Indeterminate cases are possible given that they are valid results for diplotypes whose clinical importance has not been assigned yet; priority status, that is, an indicator of the severity of the called phenotype. It indicates whether the called phenotype relates to a normal case or a case that special treatment is needed when drugs are prescribed; and the gene symbol that the diplotype refers to. Currently the diplotypes of interest in an example embodiment relate to the genes ABCG2, CYP2B6, CYP2C19, CYP2C9, CYP2D6, CYP3A4, CYP3A5, CYP4F2, DPYD, F5, NUDT15, SLCO1B1, TPMT, UGT1A1 and VKORC1. In other examples, other diplotypes are used.

In some embodiments, database generator 120 is configured to determine the diplotypes for genes of interest. For example, some functions of the workflow are used to determine the diplotypes for genes of interest. Using these diplotypes the metabolizing status or phenotype of genes of interest can be identified using this table. This identification is important since many annotations found on the dosing_recommendations and label_recommendations tables are of type “Metabolizing_status.” Identifying the metabolizing status using these tables allows the user to detect the pharmacogenomic annotations associated with a specific sample. It is also important to identify the general consultation of the VIP pharmacogenes that were selected by pharmacogenomic platform 100. Most rows are important to call the metabolizing statuses and use them to mine pharmacogenomic annotations from the appropriate tables. The entries for the genes ABCG2, F5 and VKORC1 exist to appropriately show the result of the general consultation table and not to call annotations since they are called variants.

This table was created using the “(GENE SYMBOL) Diplotype-Phenotype Table” from https://www.pharmgkb.org/page/pgxGeneRef for genes of interest; and the CYP3A4 diplotypes-phenotypes are not provided by the tables but can be detected using this webpage https://www.pharmgkb.org/guidelineAnnotation/PA166265421.

The CDS table includes the following fields, namely, an auto increment id of type integer; the phenotype (metabolizing statuses). Indeterminate cases are possible given that they are valid results for diplotypes whose clinical importance has not been assigned yet; the activity assigned to the phenotype. In some general consultation texts, expect from the called phenotype it is important to know what the specific activity of the phenotype is; priority status, that is, an indicator of the severity of the called phenotype. It indicates whether the called phenotype relates to a normal case or a case that special treatment is needed when drugs are prescribed; the text of the general consultation per case; and the gene symbol of the VIP genes. In an example embodiment, the genes identified considered as VIP by pharmacogenomic platform 100 are CYP2B6, CYP2C19, CYP2C9, CYP2D6, CYP3A5, CYP4F2, DPYD, F5, HLA-A, HLA-B, NUDT15, SLCO1B1, TPMT, UGT1A1 and VKORC1. In some embodiments other genes are selected as VIP.

This table is important to provide general consultation for the phenotypic results of the genes identified as VIP from pharmacogenomic platform 100.

This table is generated using the “(GENE SYMBOL) Example CDS Table” from https://www.pharmgkb.org/page/pgxGeneRef for genes of interest; and for genes that no such table exists a custom text is generated, such as from manual data input.

The haplotype function table includes the following fields, namely, an auto increment id of type integer; the gene symbol that the haplotype refers to. Currently the haplotypes of interest relate to the genes CYP2B6, CYP2C19, CYP2C9, CYP2D6, CYP3A5, DPYD, NUDT15, SLCO1B1, TPMT and UGT1A1; the haplotype of the gene; and the functionality of the gene.

Some pharmacogenomic annotations provided by PharmGKB for the type of “Haplotype,” only refer to a haplotype instead of a diplotype and refer to the cases in the following context “Patients with the X haplotype in combination with a (Decrease/No/Normal Function) allele . . . ” These cases for elevated level of evidence “1A”, “1B”, “2A” and “2B” are curated using a script and received as data input at data processor 110 to refer to diplotype cases. For level of evidence “3”, for example, if no data is available, when a haplotype is referred to in an annotation, instead of the haplotype, the diplotype and the functionalities of the two haplotypes are returned using the current table.

This table is generated using the “(GENE SYMBOL) Allele Functionality Table” from https://www.pharmgkb.org/page/pgxGeneRef for genes of interest, according to some embodiments.

Many of FDA's pharmacogenomic annotations are not related to pharmacogenes but inform about a pharmacogenomic effect related to a disease with genomic predisposition. These annotations have their allele annotated as “Deficient” on the label recommendations table. This table contains variations that were detected as important for these cases. The list of variants can be further validated, the rs codes should be used when possible and it should be identified whether the variant is important for Dominant or Recessive type of variation.

This table can be generated by database generator 130 by going through the FDA list of annotations with “Deficient” tag, identifying the disease that they are related and identifying the associated variations using Clin Var data taken from the FTP site of.

### The Two-Part Report Generator

Report generator 130 is configured to include a-nextflow repository, which stores the method. In some embodiments, report generator 130 is configured to transform FASTQ files to a fully personalized pharmacogenomic report in a suitable format such as HTML or JSON. In an example implementation, in some embodiments, the FASTQ files are paired-end and are generated using short-reads technology; the host (local computer or a virtual machine) that will be used to run the workflow has Nextflow installed, along with the prerequisite packages. Nextflow version must support DSL1 in some embodiments. To install Nextflow the steps listed at the following link can be used: https://www.nextflow.io/docs/latest/getstarted.html #requirements. Nextflow may use Bash 3.2 (or later) and Java 11 (or later, up to 18). The host can meet these conditions. A user can skip the installation part and just use the nextflow executable which is located in the present folder; the host (local computer or a VM) that will be used to run the workflow has the msgen Microsoft Genomics cli installed as described in the following repository which is hereby incorporated by reference in its entirety; and the host (local computer or a VM) that will be used to run the workflow has the jq tool which can parse arguments from json files. This tool can be installed using the steps described at the following link; the workflow is developed to run on the cloud in an example embodiment. The appropriate repository images and the mentioned tools are required to run the processes of our workflow using FASTQ files are input.

In some embodiments, where the aforementioned conditions are met for the host machine that will be used to run the workflow as well as for the associated cloud provider subscription. In some embodiments the data processor 110 is configured to execute the workflow as shown in FIG. 1. To successfully run the workflow, it requires FASTQ files as input and a label (a name) given to the output file. The results are stored on the cloud in a container and the workspace of the run can be found in a different container. An example of executing the workflow for the sample ERR1955323 is shown in FIG. 2.

In some embodiments, the method or workflow executed by report generator 130 is separated into two steps, namely, a step making a call through the msgen API to generate the bam, bai, VCF and logs files from the original FASTQ files. The outputs of this step are stored on cloud storage; and a workflow uses the outputs of the previous step to generate the personalized pharmacogenomic report. In some embodiments, these files can be stored in one or more other storage units, for example. Display generator 150 is configured to receive data from report generator 130 and generate interface units representing the report at a physical display, such as a local computer display.

In some embodiments,—mentioned steps are executed using Azure resources (or another external server) instead of local resources. The main difference of the two steps is the fact that the first step is not implemented as part of the nextflow workflow for two reasons, namely, due to the fact that the .bam, .bai and .files are not pulled locally and there is no channel defined for merging the two processes; and the msgen call is time consuming and running it on a node of an Azure Batch Pool can increase costs accordingly per report).

The decision to address this problem was to execute the msgen part from the host environment and make the workflow wait for its execution.

### First Part: From FASTQ to Raw Variants of Pharmacogenomic Interest

The system Data processor 110 transforms the FASTQ files to bam, bai and raw VCF files is one of the first steps. The msgen API call is executed as shown in FIG. 3, according to some embodiments. This generates a BAM file in the cloud, which includes the respective BAM index file after aligning the FASTQ files against the reference genome along with a VCF file of all the variants found.

The In some embodiments, report generator 130 is configured to implement the first part of the method as follows, which is the first part of the total workflow (method). Report generator 130 is configured to take as input the FASTQ file(s), such as received from data processor 110, and it generates the bam, bai and raw VCF of a sample. The default human genome version is defined by the hg38 ml attribute. During the execution, this step can be traced by using the command shown in FIG. 4 in a separate terminal. The tools used for this purpose are called genomic tools.

### The Second Part: From Raw Variants to a Pharmacogenomic Report

In some embodiments, the next step of the method implemented by report generator 130 is to generate a personalized pharmacogenomic report from the files generated from thegenomic tools. This workflow is the second and final step of the total pharmacogenomic platform 100. To generate this report for an individual, bam, bai, VCF as well as reference files are used by report generator 130. The bam, bai and VCF files are created using the genomic tools.

In some embodiments, report generator 130 is configured to run the method (workflow) on the cloud using a node and a repository container storage platform.

In some embodiments, the workflow is executed automatically from the proprietary script's execution. The workflow is not run separately. However, in some embodiments described herein, the workflow is described as running separately.

To run the workflow, a nextflow script can be executed given the appropriate cloud credentials, and after the results have been generated for the sample of interest, the workflow can be executed by running the command shown in FIG. 5.

For example, the workflow for sample ERR1955326 can be executed with the command shown in FIG. 6A, using the nextflow executable at the parent directory of this repository. It is expected that the msgen workflow was executed on the ERR1955326_R1.fq.gz and ERR1955326_R2.fq.gz FASTQ files, and that the credentials are provided to the file located at the same folder of the nextflow executable.

These files contain information about the variations of an individual and are detected on the “output” blob storage through name pattern matching using the sample_name parameter. There are several other reference files used for report generator 140 to run the workflow, according to some embodiments. Given that these files are the same for every case and do not change (unless desired) they are provided as parameters. In some embodiments, for example, these files are all stored in the humanreference blob container, and the parameters are the paths to these files. The configuration file contains the code that indicates the workflow to run on the cloud using docker images and specifies these images. It is also the part of the workflow where the credentials are parsed. In some embodiments, a more powerful Virtual Machine (VM) can be utilized as depth of sequencing increases.

The outputs of the method can be found on the “output” container in the out-directory subfolder as identified when running the command. In this folder, a personalized pharmacogenomic HTML or JSON report for the sample of interest can be found. These files are stored on the “output” blob container in the subfolder out-directory defined when running the method, according to some embodiments.

This method is the second and final part of the total pharmacogenomic platform 100 method. It takes as input the bam, bai and raw VCF file generated using msgen and creates the personalized pharmacogenomic report for the individual of interest.

Files are copied to the ongoing container and as it grows, the storage container can be deleted and recreated periodically to reduce storage costs. An alternative option is to automatically purge this folder.

In some embodiments, pharmacogenomic platform 100 is configured to implement a method for generation of a pharmacogenomic report. FIG. 7 shows an example method according to some embodiments. The main workflow comprises of three processes and several channels that bind them together. A visual representation of the workflow is shown in FIG. 7, according to some embodiments.

Initally, the reference fasta and fai are parsed into two input channels. The one pair of channels is merged with the input channels of the reference dictionary, hapmap, hapmap index, golden indels, golden indels index and create the reference_ch channel. The other pair creates the ref_ch channel. The other input channels that are created are that of the raw VCF file, the full bed file channel (bed_full_ch), the bed for the UGT2B17 gene (bed_ugt2b17_ch) and the product merge the bam and bai files into the bambai_ch. These final channels are the inputs for two processes that are executed in parallel.

The Raw2Filtered process involves the use of input files, that is, the reference files from the reference_ch, the raw VCF by msgen from the raw_vcf_chd the full bed containing the regions of interest from the full_bed_ch; and produces the output files, that is, the filtered VCF named ${params.sample_name}_filtered. vcf which is directed to the filtered_ch channel and also copied to the params.outdir on the cloud.

The Raw2Filtered process includes the following steps, namely, initially the raw VCF file that is bgzipped and indexed using the command shown in FIG. 8; then the full bed file is used to narrow the VCF to regions of interest using the command shown in FIG. 9, according to some embodiments; this down sampling of the VCF speeds up the commands shown in FIG. 10, according to some embodiments. The downsampled VCF is supplied to the GATK CNNScore Variants (https://gatk.broadinstitute.org/hc/en-us/articles/360037226672-CNNScore Variants) 1D Convolutional neural network applies scoring to the variants; As shown in FIG. 11, the output of this step is the supplied to GATK Filter VariantTranches (https://gatk.broadinstitute.org/hc/en-us/articles/360040098912-FilterVariantTranches) with the recommended parameters for WGS which generates the final filtered VCF.

The docker image container used to run this process is the public broadinstitute/gatk: 4.2.6.1 provided on Docker Hub, according to some embodiments. Other containers can be used, in some embodiments.

The Caller process involves the use of input files, that is, the reference fasta and its index from the ref_ch, the bam file and its index from the bambai_ch and the bed file for the UGT2B17 gene from the bed_ugt2b17_ch; and produces three output files, namely, a TSV file containing the called diplotypes for the CYP family of genes using an adjusted version of the StellarPGX workflow (https://github.com/SBIMB/StellarPGx); a depth file gathering coverage information for the UGT2B17 gene and two other reference genes used for comparison as indicated by the StellarPGx workflow; and a CSV file containing the called diplotypes for the HLA family of genes based on the graphtyper tool (https://github.com/DecodeGenetics/graphtyper), according to some embodiments. External services other than Azure Cloud can be used in some embodiments.

The Caller process includes the following steps, namely, the first step of the processes corrects the download order of the fasta/bam and fai/bai files. Given that the fai/bai file is derived by the fasta/bam file it is expected that its creation date is later than that of the fasta/bam file. Due to its small size this is typically not the case when copied on the node vm because it gets copied first. To correct this problem the code as shown in FIG. 12 is used according to some embodiments; the next step uses the generallcall.sh script to generate the CYP diplotypes tsv. A novel script transforms StellarPGx to a format useful for analysis. The idea is exactly the same as StellarPGx though it is made to make diplotype calls for all CYP genes and to output the results in a TSV format. The output is the file cyp_diplotypes_${params.sample_name}.tsv. The code to run this step is shown in FIG. 13 according to some embodiments; next, the samtools bed cov command is used to identify the depth of the ugt2b17 gene and two other genes for comparison (as indicated by the StellarPGx workflow). The output is the file ${params.sample_name}_ugt2b17_ctrl.depth. This step is executed by the command shown in FIG. 14; Finally, the custom script hlacalls.sh to call the HLA family diplotypes is executed to generate the ${params.sample_name}_hla_results.csv file. This script is based on the graphtyper tool that allows identification of these diplotypes and can be executed by the command shown in FIG. 15.

In an example embodiment, the docker image container image used to run this process is a private Container Registry myengeneimages.azurecr.io/images/caller: v1 as shown in FIG. 6B. The generation of this image is automated and can be regenerated using the provided data in the docker-builds repository.

In an example embodiment, the method of generating the pharmacogenomic report by report generator 140 involves the use of input files, that is, the CYP gene family diplotype calls, the HLA gene family diplotype calls and the UGT2B17 depth coverage file as generated by the Caller process and the filtered file generate by the Raw2FilteredVCF process; and produces a fully personalized pharmacogenomic report. In some embodiments, calls pertaining to other gene families are used.

According to some embodiments, the report generator 140 is configured to generate the pharmacogenomic report (PGxReport) by executing the R workflow package described in steps 1-22. The report is generated by merging the VCF with the database configuration engine 120. The results are then stored in a table to be displayed by the report generator 140.

The docker repository image container image used to run this process is the private Container Registry myengeneimages.azurecr.io/images/pgxreport: v1 as shown in FIG. 6B, according to some embodiments. This image contains both the pgxreport layout as well as the rworkflow to generate the report. The generation of this image is automated and can be regenerated using the provided data in the docker-builds repository.in the docker-builds repository.

In some embodiments, new pharmacogenomic annotations are inputted in the database(s) stored by database configuration engine 120. Report generator 140 is configured to use the bed file containing regions of interest. For example, if the regions of interest change over time, so will the output, accordingly. The report following a determination as to whether the data is configured to match the database schema or patterns of the database.

If the target genome region is changed, then the bed file needs to be updated accordingly. If more genomic regions are tested, or if the existing bed file regions are increased, then more variants may be identified at the filtered VCF, provided that these regions match annotations in our database. To check for gene deletions, the method used for UGT2B17 gene deletions can be applied. We determine the depth coverage of the bed file for the region of interest using samtools, and then calculate the Copy Number Variation (CNV) for this individual. If their copy number is lower than a given threshold, then we can say that a deletion has occurred for that gene for that individual. So to be able to provide more pharmacogenomic annotations concerning the deletion of genes other than UGT2B17, the workflow and the bed file needs to be updated appropriately, according to some embodiments.

If calls for other genes of the HLA or CYP family is of interest the Caller image must be updated, for example as shown in FIG. 6B. The Caller image contains multiple scripts that pull specific regions of genes in the HLA and CYP family, runs the graphtyper software to determine the most likely haplotype. As new information regarding haplotypes is generated by the research community, files used in this image need to be updated.

To update the report layout and solve any potential bugs the rworkflow package pgxreport must be updated for the report generation by report generator 140 as is shown in FIG. 6B.

The method for report generation will now be described, according to some embodiments. Report generator 140 performs translation of the raw genetic variation which consists of the individual's genomic profile and converts it into a useful personalized pharmacogenomic report. The general structure of this method will be provided, according to some embodiments.

In some embodiments the package can be installed locally with a command like the one shown in in FIG. 16; after the installation, the package can be available with a command like the one shown in FIG. 17.

In case the package needs to be imported as a docker image, so that the final report is generated using cloud resources, follow the instructions indicated at the docker-builds repository.

This workflow is part of the final process, and expects that files from previous processes have been generated as inputs to the workflow. These files are expected to include the CYP gene family diplotype calls as generated by the Caller process. The name of the files can follow a pattern or naming convention. In some embodiments, the specific gene family(ies) for the calls differ.

The workflow is configured to receive the pharmacogenomic database credentials so that the rworkflow package of the Report generator 140, can pull the tables from the database. Finally, a threshold for the minimum prediction probability of the machine learning model should be provided (Recommended value >=0.5). In some embodiments, the method is simply configured to receive data (e.g., tables) from a database from database configuration engine 120.

Determination of the Data Generated by the Workflow; from a Raw Set of Genetic Variants to a Personalized Pharmacogenomic Report

The output of the workflow is a list of tables that are used by the report generator 140 to generate the final personalized pharmacogenomic report in HTML, JSON or other format, according to some embodiments.

In some embodiments, the workflow is called to test updates on the final report pushed to a repository and downloaded as a package. The workflow contains several functions implemented by report generator 140, where the several functions are for analyzing the input data and generating the appropriate tables. Apart from the several functions that analyze the data, the package contains two functions that pipe all the functions together to get the results. The first function detects all types of variations (variants, genotypes, haplotypes, diplotypes, metabolizing statuses and machine learning engine 130 predictions) from the input files, uses them to determine the pharmacogenomic annotations from the database that are of relevance for the individual of interest and returns a list of tables of raw format containing this data. The second function generates the tables needed to produce the final personalized report.

To run the workflow and generate the tables of the pharmacogenomic report the code shown in FIG. 18 is used, according to some embodiments.

Embodiments of the workflow will now be described.

This function pipes together several functions of the package to run the code as a workflow. The workflow that is executed by this function is shown in FIG. 19, according to some embodiments. Inputs are colored green, function with red and outputs with blue.

This step generates the downsampled and filter tagged VCF file as input. The variants are filtered to include only those that passed the filtration process after passing them through the HapMap and insertion/deletion files downloaded from the HapMap Project and the 1000G project, respectively. The sm field is used to detect the called genotype and using this genotype the two alleles of each variation are identified. The function then generates several id representations for these variations that are important for downstream analysis. The ids that are generated are the following, namely, id, that is, rs_id if it exists or (CHR):g. (POS)(REF)>(ALT); id_full, that is, uses id from step 1 and generates the id_full of the format: (id): (ALLELE_1)(ALLELE_2); id_less, that is, uses id from step 1 and generates the id_full of the format: (id): (ALLELE); pos_allele_1, that is, id of the format (CHR). (POS)(ALLELE_1); and pos_allele_2, that is, id of the format (CHR). (POS) (ALLELE_2).

Given that using the get_variation_ids( ) function, the variations are represented in a format that is compatible with that of the database, the next step is to pull the tables of the database. The tables can be provided by database configuration engine 120, according to some embodiments. The following steps are implemented by database configuration engine 120 and the described tables are configured by database configuration engine 120, according to some embodiments.

This function is now used to pull the tables from the database(s) It also separates the tables given the type of variation, since different types of variation need different downstream analysis to detect the associated pharmacogenomic annotations. After establishing a connection to the database the function pulls the table containing the dosing PGx annotations from clinical consortiums; the table containing the label PGx annotations from regulatory bodies; the table containing the clinical PGx annotations from PharmGKB; the table containing the predictions made by the machine learning method used by Machine Learning Engine 130; the table containing the general consultation information; the table containing the haplotypes functionality information; the table containing the haplotypes activity score information; the table containing the phenotypes definition information; the table containing the haplotype calls information; the table containing variations related to deficiencies; a table containing drug therapeutic area information; and a table containing the total drugs that are currently tested. It is generated by an SQL query that filters out drugs whose annotations cannot be detected yet. Specifically, it removes, the following, namely, drugs with annotations groups only related with a deficiency caused by a disease. Since disease detection is not yet implemented these drugs must be removed. Exception is the G6PD gene which can be potentially detected by a machine learning model generated by machine learning engine 130; drugs from PharmGKB that are of level 1A given that these are already included in dosing and label recommendations; drugs from PharmGKB of type genotype that are related to variants in Intergenic regions (IGR) and have not been added yet; drugs (‘Acetylcholine’, ‘Bromperidol’, ‘Cavosonstat’, ‘Cysteamine’, ‘Folfox’, ‘Raltitrexed’) from PharmGKB given that they are only related to some INDELS that are not yet called; and drugs from PharmGKB of type haplotype that are related to genes ‘CYP3A7’, ‘GSTMI’, ‘GSTTI’, ‘HLA-DOBI’, ‘HLA-DRB3’, ‘NAT2’, ‘SLC6A4’, ‘UGTIA3’, ‘UGTIA4’, ‘UGTIA6’, ‘UGTIA7’ and ‘UGT2B15’ given that their haplotypes are not called yet; and then the function splits the dosing and label recommendations per type of variation, with possible cases being: variant, genotype, haplotype, diplotype and metabolizing status. Clinical PharmGKB annotations are also split in a similar way to genotypes and haplotypes which is the main categorization used by PharmGKB. Finally, predictions are split to novel and known variations given the existence or not of an rs id.

The function returns all these tables in the format of a list as shown in FIG. 20, according to some embodiments.

Drugs removed from our table are with regards to the uncalled clinical PharmGKB cases and diseases related pharmacogenomic annotations (from FDA). If the data from the external pharmacogenomic (eg. PharmGbK) database or drugs of interest is updated so that excluded drugs are called, the query is updated accordingly. Updating the query without changing the ability to call the variants could lead to a report identifying the associated drugs as safe to use, while there is no testing taking place.

The goal of the following steps is to create a downstream analysis that allows us to merge these tables and detect the personalized pharmacogenomic annotations.

The following step is used to detect variant and genotype level pharmacogenomic annotations from dosing and label recommendations as well as genotype level annotations from clinical PharmGKB. To achieve this the database's variant tables are merged to detect variant level annotations, the database's genotype tables are merged to detect genotype level annotations and the genotypes table from PharmGKB is merged. The outputs are all pharmacogenomic annotations of variant or genotype type from the dosing, label or PharmGKB sources, returned as a list of structure shown in FIG. 21, according to some embodiments.

### Processing of the Highly Polymorphic UGT2B17 Gene and the CYP and HLA Family of Genes

At step 1 of the report generator 140, the haplotypes and diplotypes are identified as genes of interest. It uses the called diplotypes for the highly polymorphic HLA and CYP family of genes, and the depth of the also polymorphic UGT2B17 gene from the process of the workflow, as well as the variants table to call new diplotypes that the process was unable to call. This step contains many steps that are related to bioinformatics and/or pharmacogenomics knowledge. In these cases, the related parts of the code will be provided, and the methodology will be further explained. In some embodiments, diplotypes for different family(ies) are used.

The parsing of data from the haplotypes-diplotypes for CYP calls function of the report generator 140, involves eight steps, namely, the first step of this function is to turn the CYP calls made from the Caller process into a format compatible with the database from database configuration engine 120. The CYP calls is a Tab-Seperated-Value (TSV) file with three columns. The first column is the gene symbol, the second is the called diplotype and the third one is general info that might be generated when the diplotypes are called. For example, the first step in the function is to parse the data and remove unnecessary symbols from the diplotypes columns if they exist. The second step is to separate in new rows, diplotypes that the Caller was unable to match to a single diplotype. These cases are separated by the “or” string in the original call. Given that the CYP caller is based on StellarPGx and the identification of diplotypes is made using combinatorics, there might be some cases that the diplotypes are indiscriminate. These cases are treated as separate rows so that the pharmacogenomic annotations for each case can be called, but later on they are merged at the final report to inform that there are two possible results; the third step is to identify the diplotype of an uncalled gene (not containing *) as “Unidentified/Unidentified”; in the fourth step, the entries of the diplotypes column are not sorted according to the usual nomenclature, e.g. while *1/*2 should not be written as *2/*1. This leads to problems when merging with the database so to avoid this problem the two haplotypes are mined from the diplotype and both possible diplotypes are created as new rows; in the fifth step, the gene symbol is transformed to uppercase. In the sixth step the following corrections are applied: when copy number variations >3 is detected in that gene, then the CYP caller will identify the exact number of copies, e.g., *1/*2x5. In pharmacogenomics, as it can be seen in the database, it is usual for these copy numbers to be identified as x>3, e.g., *1/*2x5 and *1/*2×4 are both *1/*2x>3. This correction is implemented and updates both the haplotypes and the diplotype if needed; In the seventh step, there are some corrections that must be made for the CYP3A5 calls. The CYP caller, can identify the haplotypes *2, *4 and *5, but as can be seen in Pharm Var, these are just subcases of the *3 allele. In pharmacogenomics it is usual for annotations to be provided as *3 so all other calls must be changed to *3. This correction is implemented and updates both the haplotypes and the diplotype if needed; In the eighth step, there are some corrections that must be made for the CYP3A4 calls. The CYP caller identifies the haplotype *36 but as *1G. *36 is more usual in pharmacogenomics, so this correction is implemented and updates both the haplotypes and the diplotype if needed.

To get haplotypes-diplotypes for UGT2B17 from depth involves the UGT2B17 diplotype has three states; *1/*1 if the gene has no deletion, *1/*2 if the gene has one deletion and *2/*2 if the gene has two deletions. To identify the deletion status, the methodology also used by StellarPGx is followed. The methodology is to calculate the depth of UGT2B17 as well as for two reference genome positions which are taken to be the same as the StellarPGx implementation. To calculate the copy number of the UGT2B17 the following steps are taken: calculate av_ugt2b17: the depth over the length of UGT2B17; calculate av_test1 and av_test2: the depth over the length of the reference regions; calculate av_test: The average of av_test1 and av_test2; calculate the copy number: 2*round (av_ugt2b17/av_test); and identify diplotype: If the copy number turns out to be 2 the *1/*1, if 1 then *1/*2 and finally if 0 then *2/*2.

The parsing data haplotypes-diplotypes for HLA calls involves a Comma-Separated-Values (CSV) file of four columns gene_symbol, representation, hap_1 and hap_2 containing the gene symbol, the representation number of digits of the HLA called haplotypes and the first and the second haplotype. The haplotypes are used to generate the diplotypes field and representation is set to the info field.

While StellarPGx and graphtyper are excellent on calling diplotypes for the CYP and HLA families accordingly, they don't generate the diplotypes for other genes that are of interest for pharmacogenomics. These genes can include ABCG2, DPYD, F5, NUDT15, SLCO1B1, TPMT, TYMS, UGTIA1 and VKORC1. To call the diplotypes for these genes the combinatorics methodology proposed by StellarPGx has been used. The possible combination of variations and the associated diplotype are stored in a table of the database. The variants column of the table has a combination of all the variations that form a diplotype. These are provided in a comma separated and text sorted format, with the representation (CHR). (POS) (ALLELE) for each variant.

The steps to achieve these calls include the following, namely, from the variants identified by the pos_allele_1 and pos_allele_2 representations are selected which follow the pattern (CHR). (POS) (ALLELE). Then the variants of this table are split into several rows, containing one variant per row to have all the possible target variants. These variants are further split into a list by the gene that they are related to. A function is then applied to each gene to test whether any detected variants in the sample exist in the total possible variants. If there is no such variant found a reference tag is added to the gene else the variants are pasted and sorted in a comma separated manner as in the original format in the database table containing the haplotype calls. This process creates a list containing the sorted and comma separated variants of a gene if such variants exist. The combinations of variants are stored into a vector and the genes containing variants are identified. The vector of variants is used to detect the associated diplotypes. Pathogenic cases might arise when a gene contains variants that the code did not manage to identify the diplotype. In these cases, the gene's diplotype is marked as “Unidentified/Unidentified.” As the DPYD gene is highly polymorphic, a combination of variants might arise. This is solved in the later steps of the workflow. If such a case comes up the diplotype of the DPYD is updated to a format containing the haplotype names detected separate by the “+” sign.

The total results for the CYP and HLA families, UGT2B17 and the other calls are then merged into a list in the report generator 140.

Process Haplotype and Diplotype Level Pharmacogenomic Annotations, Novel Variation from Machine Learning and Metabolizing Status

The following step is used to detect haplotype and diplotype level pharmacogenomic annotations from dosing and label recommendations as well as haplotype level annotations from clinical PharmGKB. To achieve this the first step is to create a vector containing three ids of the format (GENE-SYMBOL) (HAPLOTYPE-1), (GENE-SYMBOL) (HAPLOTYPE-2) and (GENE-SYMBOL) (DIPLOTYPE). Then the database's haplotype and diplotype tables are filtered using this vector. The outputs are all pharmacogenomic annotations of haplotype or diplotype type from the dosing, label or PharmGKB sources, returned as a list of the structure as shown in FIG. 22, according to some embodiments.

Using machine learning engine 130, the genes: ABCG2, CYP2B6, CYP2C19, CYP2C9, CYP2D6, CYP3A4, CYP3A5, DPYD, G6PD, NAT2, NUDT15, SLCO1B1, TPMT and UGT1A1 are tested for novel variations and their predicted effect on the protein functionality is returned. These tables are filtered to contain only the variants detected in the sample of interest. Genes with no novel variations are tagged with the “No novel variation detected” prediction. In some embodiments, machine learning engine 130 is configured to output the following predictions: “No”, “Decreased”, “Normal” (meaning functionality). For genes with novel variation the following steps are applied per gene, namely, ABCG2: If a “No” variation is detected the functionality is assigned as “Poor Function”, if a “Decreased” variation is detected the functionality is assigned as “Poor Function” else the functionality assigned is “Normal Function”; CYP2B6: If a “No” variation is detected the functionality is assigned as “Poor Metabolizer”, if a “Decreased” variation is detected the functionality is assigned as “Poor Metabolizer” else the functionality assigned is “Normal Metabolizer”; CYP2C19: If a “No” variation is detected the functionality is assigned as “Poor Metabolizer”, if a “Decreased” variation is detected the functionality is assigned as “Intermediate Metabolizer” else the functionality assigned is “Normal Metabolizer”; CYP2C9: If a “No” variation is detected the functionality is assigned as “Poor Metabolizer”, if a “Decreased” variation is detected the functionality is assigned as “Intermediate Metabolizer” else the functionality assigned is “Normal Metabolizer”; CYP2D6: If a “No” variation is detected the functionality is assigned as “Poor Metabolizer”, if a “Decreased” variation is detected the functionality is assigned as “Intermediate Metabolizer” else the functionality assigned is “Normal Metabolizer”; CYP3A4: If a “No” variation is detected the functionality is assigned as “Poor Metabolizer”, if a “Decreased” variation is detected the functionality is assigned as “Intermediate Metabolizer” else the functionality assigned is “Normal Metabolizer”; CYP3A5: If a “No” variation is detected the functionality is assigned as “Poor Metabolizer”, if a “Decreased” variation is detected the functionality is assigned as “Intermediate Metabolizer” else the functionality assigned is “Normal Metabolizer”; DPYD: If a “No” variation is detected the functionality is assigned as “Poor Metabolizer”, if a “Decreased” variation is detected the functionality is assigned as “Intermediate Metabolizer” else the functionality assigned is “Normal Metabolizer”; NAT2: If a “No” variation is detected the functionality is assigned as “Poor Function”, if a “Decreased” variation is detected the functionality is assigned as “Decreased Function” el se the functionality assigned is “Normal Function”; and NUDT15: If a “No” variation is detected the functionality is assigned as “Poor Metabolizer”, if a “Decreased” variation is detected the functionality is assigned as “Intermediate Metabolizer” else the functionality assigned is “Normal Metabolizer”; SLCO1B1: If a “No” variation is detected the functionality is assigned as “Poor Function”, if a “Decreased” variation is detected the functionality is assigned as “Decreased Function” else the functionality assigned is “Normal Function”; TPMT: If a “No” variation is detected the functionality is assigned as “Poor Metabolizer”, if a “Decreased” variation is detected the functionality is assigned as “Intermediate Metabolizer” else the functionality assigned is “Normal Metabolizer”; UGT1A1: If a “No” variation is detected the functionality is assigned as “Poor Metabolizer”, if a “Decreased” variation is detected the functionality is assigned as “Intermediate Metabolizer” else the functionality assigned is “Normal Metabolizer”; and G6PD: If a “No” variation is detected the functionality is assigned as “Deficient”, if a “Decreased” variation is detected the functionality is assigned as “Deficient” else the functionality assigned is “Non Deficient”. In some embodiments, genes other than those listed in the foregoing can be tested and machine learning engine 130 is configured to generate at least one prediction in respect of each of same.

The metabolizing statuses from the diplotypes are identified using a script. The steps taken by this function involve several steps, including the phenotypes table taken by the database. This table contains all the appropriate information to identify the metabolizing statuses from the called diplotypes. There are some steps that must be taken to represent the metabolizing statuses of the HLA-A and HLA-B genes, since they must also be included in the final VIP genes list. Also, some steps must be taken in order to identify the DPYD metabolizing status for cases that have several variants (see step 6 in FIG. 19) Then the HLA-B results are analyzed so that the final “metabolizing status” is assigned one of the following cases: *15:02 positive, *15:02 positive/*57:01 positive, *15:02 positive/*58:01 positive, *57:01 positive/*58:01 positive, *57:01 positive, *58:01 positive or *15:02, *57:01, *58:01 negative. For HLA-A the same action is taken with possible result being: *31:01 positive and *31: 01 negative; the previous results are merged with the phenotypes table and the metabolizing statuses are detected; the generated table is expanded by adding the predictions for genes that had novel variations and which variations led to a non “Normal Function”, “Normal Metabolizer” status; then the UGT2B17 metabolizing status is identified. If the haplotype identified by the depth of the gene is *1/*2 or *2/*2, then the metabolizing status is identified as “Poor Metabolizer” else as “Normal Metabolizer”; If during the merging process the metabolizing status of the DPYD gene has not been identified then it means that the gene has several variations. Finally, activities for all the variants are sorted from lowest to highest value and the sum of the two lowest activities is calculated. If this sum is equal to 0 or 0.5 then the metabolizing status that is assigned a “Poor Metabolizer”, if it is 1 or 1.5 the assigned metabolizing status is “Intermediate Metabolizer” and if it is >1.5 then the assigned status is “Normal Metabolizer”.

In some embodiments, a step includes identifying “Deficient” due to disease cases.

The get_met_annotations( ) step in FIG. 19 is used to detect the metabolizing status level pharmacogenomic annotations from dosing and label recommendations. The database's _met_status tables are used to identify these annotations. In some embodiments, the metabolizing status level pharmacogenomic annotations are obtained from a dosing and label recommendation data source. A table containing the metabolizing statuses is then edited to trim the text from metabolizing statuses starting with “Likely” or “Possible.” The outputs are all pharmacogenomic annotations regarding metabolizing status that are derived from the dosing and label sources, are returned as a list shown in FIG. 23, according to some embodiments.

### Generate Consultation for Very Important Pharmacogenes (VIP)

The get_cds( ) step is used to get the general consultation for Very Important Pharmacogenes. The general consultation for Very Important Pharmacogenes is obtained from the data source. Given that the activity field is also of interest for some general consultation cases the merging of various tables is done using an id of the format (GENE SYMBOL)-(PHENOTYPE)-(ACTIVITY). The same id is created for the metabolizing statuses table created by the get_met_status( ) step. Then uncalled genes the fields of the CDS are updated to use the texts “Unidentified,” “n/a” and “Indeterminate” accordingly. Then genes that do not have a single possible diplotype/metabolizing status associated with them are identified and the rows for these genes are merged using the “or” symbol.

### Collecting all Information and Generating the Report

The report tables generate the final tables of the personalized output report. The workflow that is executed by this function is shown in FIG. 24, according to some embodiments. Inputs are colored green, function with red and outputs with blue.

The get_reactable_cds( ) step generates the general consultation table for the personalized pharmacogenomic report checks if the Priority field and if it is of type Normal/Routine/Low Risk, a green label is attached to the results, or a yellow warning symbol is used. Then the HLA symbol is stripped from the associated diplotypes and the final reactable tables are generated.

The get_reactable_myengene( ) step uses the table containing the predictions made by machine learning engine 130 as well as the metabolizing status table to generate the final reactable table. Initially, these two tables are merged. Some corrections are made for the G6PD and NAT2 genes. If no novel variation, from the list of the novel variations whose effect is predicted by the ML model, was detected then the status is assigned as “Non-Deficient.” Similarly, for NAT2 if no such variation was detected the status is assigned as “Fast” (Accetylator). Unidentified cases are corrected. The texts for unidentified cases are then adjusted. Finally, the metabolizing statuses are edited so that they contain the info about whether they derive from machine learning model 130 predictions for novel variations or not. The final report is formed where it contains indications about identified warnings along with their source.

The get_reactable_label( ) step is used to merge the label pharmacogenomic annotations for all types (variant, genotype, haplotype, metabolizing status). Initially the different tables are imported by this step. Then, using the fields simple_annotation and risk which contain strings about whether the identified annotation poses a problem, a red label is applied, or a green label applied, if they are safe and for annotations with a warning a yellow label is applied. In some embodiments, other colours to denote the same is used. The allele column is then edited to contain the source of evidence as a URL. Specific cases for the DPYD, TPMT, CYP2D6, CYP2C19, that contain the deficiency statement in the annotation text are edited to identify whether the identified case is Partially or Completely Deficient. Then columns are combined together as needed and the final table is produced, sorted by the drug name.

The get_reactable_label_source( ) step is the exact same process with get_reactable_label( ) with the only difference being that the results are sorted by the source of annotation.

The get_reactable_dosing( ) step is the exact same process get_reactable_label( ) with the only difference being that the pharmacogenomic annotations are the dosing related ones.

The get_reactable_dosing_source( ) step is the exact same process get_reactable_dosing( ) with the only difference being that the results are sorted by the source of annotation.

The get_reactable_pharmgkb( ) step is used to create the table for the clinical pharmacogenomic annotations of PharmGKB (or other external data source) with LOE 1A, 1B, 2A, 2B, both the genotype and the haplotype annotations are used. For the case of the haplotype annotations some adjustments must be made. Given that the pharmacogenomic texts from PharmGKB contain things like “A combination of normal and a decreased function allele” while only the one case being provided, the diplotypes for each gene are parsed and the functionality of each haplotype is identified using the haplotype functionality table of the database and added to the called case. Finally, the publication links list is added as columns, according to some embodiments.

The get_reactable_experimental( ) step is the same step as the get_reactable_pharmgkb( ) with the only difference being that experimental annotations of loe 3 are now tested.

To get a list of total drugs results from dosing, label, clinical PharmGKB and experimental PharmGKB are merged into a single table. If a drug has no associated pharmacogenomic annotations, it is indicated as safe by the appropriate texts. If a drug has only experimental annotations the drug is assigned as potentially safe to use by the associated annotations. Otherwise, if other pharmacogenomic annotations of high LOE are identified, they are returned. The final table is then generated. It is grouped by the drug of interest and contains extra info per row, using a custom script, to identify whether the drug is safe, potentially safe, or non-safe to use.

The get_reactable_grouped( ) step is where the whole result from dosing, label, clinical PharmGKB and experimental PharmGKB are merged into a single table, as in get_reactable_total_drugs( ) the previous step, but the difference is that the total drugs table is also used to group the drugs by therapeutic and sub-therapeutic area. The first table contains all the results, the second only the result for drugs with high LOE annotations detected, the third only the drugs with experimental annotations and the fourth only the safe drugs that had no pharmacogenomic annotations detected.

These tables are used by report generator 140 to generate the final report for a sample of interest.

The report generator 140 and the display generator 150 can process and display variants from a collection of different genome profiling technologies. In some embodiments these technologies can be Next Generation Sequencing, third-generation sequencing and SNP arrays. In case a SNP array is used then the report generator 140 takes as input a file containing a subset of pharmacogenomic variants of interest. For example, this file might contain only variants with level of evidence 1A or variants been approved from an external provider, consortium or regulatory body. In that case the display generator 150 shows in the report the variants that belong in this file.

the Machine Learning Engine for the Identification of Variants that Lead to Altered Protein Function.

Machine learning engine 130 implements variant detection platform 2600, according to some embodiments. Embodiments of machine learning engine 130 and variant detection platform 2600 will now be described in detail. The report described in the following can be generated by report generator 140, and variant detection platform 2600 can be integrated with pharmacogenomic platform 100, according to some embodiments.

In some embodiments, variant detection platform 2600 includes one or more processing devices and one or more storage devices. The processing device is configured to execute instructions in memory (or equivalent storage medium) to configure feature generator 2610, variant validator 2620, machine learning model 2630, and loss-of-function detector 2640. A variant includes a genomic variant in a particular gene or group of genes, including single base substitutions, small insertions/deletions, and copy number variants. A genomic variant includes a single nucleotide polymorphism (SNP). In some embodiments, variant detection platform 2600 is configured to generate predictions using a classifier not only on a gene-by-gene basis, but also predict a genomic variation in a group of genes (e.g., so FDA data can map to the variant that is a variation in a group of genes). In some embodiments, a variant includes a gross rearrangement. A gross rearrangement can be a large genomic alteration involving deletion, multiplications, or inversions of large DNA fragments, for example. In some embodiments, copy number variants are not used. Variant detection platform 2600 can connect to data sources (e.g., databases, servers), entities (e.g., servers, local computing devices), displays (e.g., at a computing device), and the like over a network, such as implemented over a wired or wireless connection. Users can engage with variant detection platform 2600 at a computing device, which can transmit data between the computing device and variant detection platform 2600. Data can be transmitted over the network between the various components connected. Input can be received by variant detection platform 2600 and output from variant detection platform 2600 can be transmitted via an I/O unit.

Feature generator 2610 is configured to receive and store, in the memory, one or more features from an annotated variant dataset of at least one variant. The annotated variant dataset can be derived from whole genome sequencing (WGS) or whole exome sequencing (WES) data, not simply specific variants. Feature generator 2610 can receive one or more features from its own processes or output, for example. In some embodiments, the annotated variant dataset is a dataset in a text format, where for each variant to be processed using variant detection platform 2600, the following data is included: LoFtool, DEOGEN2 score, MPC score, BayesDel addAF score, integrated fitCons score, FATHMM score, LIST.S2 score, and Interpro domain. Allele frequency data is also included in some embodiments.

In some embodiments, feature generator 2610 is configured to extract one or more features for variants in an annotated variant dataset and to store the one or more features in the memory. One or more features received and stored in the memory by feature generator 2610 can include feature(s) extracted by feature generator 2610, according to some embodiments. The memory is non-transitory computer memory or other electronic storage medium. Various aspects of variant detection platform 2600 can be stored on another type of storage device such as SSD or HDD or a server. In some embodiments, the annotated variant dataset is generated using a Variant Effect Predictor (VEP). Annotations generated using VEP can be the features extracted by feature generator 2610. In some embodiments, feature generator 2610 is configured to extract such annotations and/or generate new feature(s) such as based on such annotations or outputs from a VEP. For example, the VEP can be the VEP provided by Ensembl and its plugins, such as dbNSFP, features are extracted using VEP. In an example implementation, LoFtool, Blosum62, and Condel plugins are also used to provide additional informative variables for the model. In an example embodiment, feature generator 2610 is configured to use all the possible variables (e.g., features) for variant annotation, such as all variants received in the annotated variant dataset. In some embodiments, feature generator 2610 is instead configured to use a subset of these variables as features. For example, feature generator 2610 can extract a subset of variables as features from the annotated variant dataset and store the same in the memory or transmit the same to another component of variant detection platform 2600. The features can be extracted (e.g., selected) based on those that an optimized (or optimal) model indicates are most important. For example, feature generator 2610 can be configured to extract features based on their impact on a metric of the machine learning model 2630, such as model accuracy, speed, or another metric. The features that increase model accuracy, for example, can be those that are extracted by feature generator 2610.

In some embodiments, the one or more features (stored by feature generator 2610 and/or extracted by feature generator 2610) are extracted from an annotated variant dataset based on impact on model accuracy. The one or more features can be extracted by optimizing model accuracy for a particular annotated variant dataset, for example.

FIG. 27 shows an example process of feature generator 2610 for generating one or more features from an annotated variant dataset, according to some embodiments. The feature(s) can be generated by determining one or more features in the annotated variant dataset. The annotated variant dataset can include any genes. For example, the annotated variant dataset can comprise an entire genome, including variants (known and otherwise). In some embodiments, the annotated variant dataset can comprise an entire genome, including variants, as well as genes that do not contain variants. Example genes are listed in Appendix I. In some embodiments, variant detection platform 2600 is configured to pre-process, using pre-processor, the annotated variant dataset (e.g., annotated variants) received as input to variant detection platform 2600 before the processed annotated variant dataset is used by variant detection platform 2600 for the identification of variants that lead to altered protein function. For example, in some embodiments, variants found within transcripts of interest are selected from the annotated variant dataset. Firstly, variant deduplication is performed by selecting only those located within the variants of interest (examples of which are shown in Table 1). Secondly, the features of interest are selected and, in an example embodiment, these include the following: in-silico scores (LoFtool, DEOGEN2, MPC, BayesDel_addAF, FATHMM, integrated_fitCons, and LIST.S2); interpro domain information; sequence ontology consequence; and identifiers of the variant and the sequence it is identified in (location, allele, symbol, existing variations, HGVS ids). As a next step, features extracted, such as from dbNSFP, that contain multiple predictions for the same variant are unnested (e.g., these variants may be separated by comma (each corresponding to a different transcript)) and only the records corresponding to the transcript of interest (examples of which are shown in Table 1) are maintained. Finally, additional features are generated from the data. For example, in some embodiments, LoF indicator is created and interpro feature is modified as described herein. In some embodiments, the in silico scores are scores other than LoFtool, DEOGEN2, MPC, BayesDel_addAF, FATHMM, integrated_fitCons, and LIST.S2.

In some embodiments, variant detection platform 2600 is configured as follows. Features of interest are selected or extracted from the annotated variant dataset, which is a text file produced by VEP. These features are then used as the only input for the classifier. These features are then used by the loss-of-function detector 2640 as its only input used by it to identify the sequence ontology variants. In particular, the features used by the loss-of-function detector 2640 is the lof indicator.

TABLE 1
Example Transcripts of Interest.

| | Ensemble transcript |
|---|---|
| Gene Symbol | identifier |
| CYP2B6 | ENST00000324071 |
| CYP2C19 | ENST00000371321 |
| CYP2C9 | ENST00000260682 |
| CYP2D6 | ENST00000645361 |
| DPYD | ENST00000370192 |
| NUDT15 | ENST00000258662 |
| SLCO1B1 | ENST00000256958 |
| TPMT | ENST00000309983 |
| UGT1A1 | ENST00000305208 |

Variant validator 2620 is configured to determine one or more validated variants of the annotated variant dataset, each validated variant matching one or more known variants of a known variant dataset, each known variant leading to altered protein function. The one or more validated variants can be extracted by variant validator 2620.

FIG. 28 shows an example process of variant validator 2620, according to some embodiments. In some embodiments, the first variant classification tier of variant detection platform 2600 is comprised of variants that overlap with a set of known variants, such as a manually curated superset of 262 variants from which a final training set was derived. Of the overlapping variants, those that lead to altered protein function (e.g., Increased, Decreased, or No function at all) are characterized as function-altering variants and receive a corresponding tag (e.g., an association with which is stored). The tag can represent a level of evidence, such as “Functionally validated” or “Functionally validated (drug protein interactions/PharmGKB)”. Those that are characterized as having normal function are not included in further processing by variant detection platform 2600. Finally, those that either overlap with the 262 variants and are attributed uncertain/unknown function or, alternatively, do not overlap with the 262 variants are transmitted (e.g., provided to) machine learning model 2630.

Machine learning model 2630 is configured to assign a classification to one or more predicted variants of variants of the annotated variant dataset not selected as validated variants, each predicted variant leading to altered protein function, the assigning by the machine learning model based on at least one of the features stored in the memory. As examples, the classification can represent altered protein function corresponding to predicted variants in CYP2B6, CYP2C19, CYP2C9, CYP2D6, DPYD, NUDT15, RYR1, SLCO1B1, TPMT, UGT1A1, BRCA1, BRCA2, or combination thereof. For example, machine learning model 2630 can be trained on CYP2B6, CYP2C19, CYP2C9, CYP2D6, DPYD, NUDT15, RYR1, SLCO1B1, TPMT, UGTIA1 genes. In some embodiments, other genes are used for training. Predicted variants in other genes are also possible, according to some embodiments. For example, Appendix I lists other such example genes.

The classification can denote increased protein function, decreased protein function, normal protein function (unchanged functionality compared to wild type protein), or no protein function (e.g., variant leads to product(s) with demolished function). In some embodiments, the classifications can be associated with scores representing a likelihood of each classification, such as a score for each possible classification. In some embodiments, the classification assigned can be the classification having the greatest score, for example. In some embodiments, a classification assigned to a variant is a classification that has a score above a threshold amount or range. For example, in some embodiments, for a particular variant, if no classification is assigned because no classification is above a threshold amount or a variant has missing information for all features or scores of interests, machine learning model 2630 does not assign a classification to the particular variant. Such a variant can constitute a variant that is not classified as a predicted variant, in some embodiments.

In some embodiments, variant detection platform 2600 at machine learning model 2630 is configured for use in the field of cancer genomics and treatment individualization. For example, genetic variants in the BRCA1 and BRCA2 genes can be held accountable for familial breast cancer in female patients. As such, variant detection platform 2600 at machine learning model 2630 can be used to predict the pathogenicity of novel genetic variants identified in the BRCA1 and BRCA2 genes and subsequently interpret them according to the pharmacogenomic data, such as existing genotype-phenotype guidelines from the American College of Medical Genetics (ACMG).

FIG. 26 is a schematic diagram of a variant detection platform, according to some embodiments; In some embodiments, variant detection platform 2600 can also be configured for use in personalized treatment for cancer in both solid tumors and hematological malignancies. In particular, in some embodiments, variant detection platform 2600 at interface generator 2650 is configured to provide genome-guided treatment recommendation according to guidelines such as from various regulatory bodies, namely FDA, EMA and PMDA, and research consortia, namely CPIC, the DPWG, and PharmGKB, for the following drug/gene pairs to the corresponding cancer types: Colon cancer: Irinotecan/UGT1A1, 5-fluorouracil/DPYD; Breast cancer: Tamoxifen/CYP2D6; and Acute Lymphoblastic Leukemia: 6-Mercapropurine and Azathioprine/TPMT and NUDT-15.

Early genetic testing can save lives. For example, women with certain BRCA1 or BRCA2 gene variations can have up to an 85 percent lifetime chance of developing breast cancer, compared to a 13 percent chance among the general female population. Women with harmful BRCA1 and BRCA2 variants can also have up to a 39 and 17 percent chance, respectively, of developing ovarian cancer, compared with a 1.3 percent chance among the general female population. The BRCA1 and BRCA2 genetic tests can guide preventive measures, such as increased disease monitoring, chemoprevention, or risk-reducing surgery.

In some embodiments, variant detection platform 2600 is configured for use in specific treatment, including personalized treatment, such as for disease, including mental disease. For example, variant detection platform 2600 can be used to detect variant(s) associated with conditions, such as outlined in gene banks (e.g., DisGeNET). As an example, variant detection platform 2600, such as using machine learning model 2630, is configured for detection of variants in CYP2D6 and in CYP2C19 and generation, such as using interface generator 2650 of user interfaces at a display of pharmacogenomic data in association with the variant(s) detected. This can provide drug-gene association data at a user interface in a tailored and specific configuration. As another example, variant detection platform 2600 is configured for use in combinatorial pharmacogenomic panels. As another example, variant detection platform 2600 is configured for use in the treatment of psychiatric diseases. Variant detection platform 2600 can help identify which patients are more likely to respond to psychotropic and which are likely to experience side effects. For example, the majority of antidepressant and antipsychotic compounds may be metabolized by CYP2D6, CYP2C19, and CYP3A4 enzymes that are mostly expressed in the liver. Variant detection platform 2600 is configured for generating an association (and/or generating a user interface displaying same) between genomic variants and enzymatic activity of CYP2D6 and CYP2C19, as well as pharmacogenomic data (e.g., clinically actionable guidelines) for psychiatric conditions. In particular, the following five genes CYP2C9, CYP2C19, CYP2D6, HLAB*15:02, and HLA-A*31:01 can be identified using variant detection platform 2600 in a provided genome.

FIG. 29 shows an example process of machine learning model 2630, according to some embodiments. In some embodiments, the non-overlapping variants (variants in the annotated variant dataset that are not determined by variant validator 2620 as a matching one or more known variants) that have no missing values in the features (e.g., LoFtool, DEOGEN2, MPC, BayesDel_addAF, FATHMM, integrated_fitCons, LIST.S2, Interpro, and LoF indicator), as well as variants matching one or more known variants of the known variant dataset (e.g., those variants overlapping with the 262 variants) and characterized as having “Unknown/Uncertain function” and also have no missing values in the features (e.g., the foregoing nine features), are provided as input to the machine learning model 2630 and predictions are extracted. The one or more predicted variants are those that lead to altered protein function. In an example, variants that are predicted by machine learning model 2630 to alter protein function are characterized as function-altering variants and receive the “variant detection platform classifier” level of evidence (an association with which can be stored such as in memory or a database). Variants characterized by machine learning model 2630 as normal function are provided to loss-of-function detector 2640. Loss-of-function detector 2640 can be configured to determine whether these variants will be discarded.

Loss-of-function detector 2640 is configured to determine one or more sequence ontology variants of the variants of the annotated variant dataset not selected as validated variants and not classified as predicted variants, each sequence ontology variant being a loss-of-function variant, the determining by the loss-of-function detector based on at least one of the features stored in the memory. In some embodiments, such feature(s) are based on Ensembl's VEP plugin LoFtool. For example, LoFtool can extract and provide variable(s) from which an LoF_indicator is derived, and such feature is provided to loss-of-function detector 2640, and loss-of-function detector 2640 can be configured to select those variants that have (e.g., match) the features indicated to denote a loss-of-function variant. In some embodiments, other feature(s) can be used by loss-of-function detector 2640. The one or more sequence ontology variants can be extracted by loss-of-function detector 2640. In some embodiments, each sequence ontology variant is determined by filtering based on sequence ontology data. In some embodiments, the loss-of-function variant is a splice acceptor variant, a splice donor variant, a stop gained variant, a frameshift variant, a stop lost variant, or a start lost variant. Different sequence ontology variants can be one of any of these loss-of-function variants. A variant leading to altered protein function includes a loss-of-function variant.

FIG. 30 shows an example process of loss-of-function detector 2640, according to some embodiments. In some embodiments, non-overlapping variants with missing values on the features from feature generator 2610 (e.g., nine features: LoFtool, DEOGEN2, MPC, BayesDel_addAF, FATHMM, integrated_fitCons, LIST.S2, Interpro, and LoF indicator), along with variants assessed by the machine learning model 2630 and predicted as normal function are evaluated by loss-of-function detector 2640 to identify potential loss-of-function variants based on the presence of SO consequences indicative of loss-of-function effects, such loss-of-function effects being splice acceptor variant, splice donor variant, stop gained, frameshift variant, stop lost, start lost. Those that have one of these consequences are characterized as function-altering variants and receive a “Loss-of-Function” level of evidence. An association with such level of evidence tag can be stored.

In some embodiments, variant detection platform 2600 is configured to return each validated variant, predicted variant, and sequence ontology variant. As used herein, these may be denoted as returned variants or variants leading to altered protein function. Interface generator 2650 can be configured to generate, on a display, each returned variant, together with data representing drug-gene association.

As an example, variant detection platform 2600 can be used on BRCA1 and BRCA2 genes by receiving an annotated variant dataset that contains such genes. According to some embodiments, interface generator 2650 is configured to receive and map guidelines from a database or computer (e.g., from the ACMG) to the BRCA1 and BRCA2 genes and generate a user interface showing same in association. This can help facilitate personalized treatment for familial breast cancer, both for solid tumors and hematological malignancies.

In an example embodiment of variant detection platform 2600, feature extractor 2610 is configured to extract features from the annotated variant dataset. The annotated variant dataset is a text file produced from a VCF file produced by VEP (from Ensembl) and its plugins. An improved or a best set of features can be identified and only those features can be used (e.g., stored in memory by feature extractor 2600). These features can include features generated based on (e.g., derived from) one or more features produced by VEP and its plugins. These features can include a customized selection of features produced by VEP and/or its plugins. A feature can be a characteristic (variable), and the output variable of VEP and/or its plugins can be optimized. Other features can be used as input variables received by machine learning model 2630.

In this example embodiment, the annotated variant dataset (txt file produced from the VCF file) contains known variants, which can then be identified by variant validator 2620, as well as unknown variants, which can then be identified by the machine learning model 2630. The machine learning model 2630 is configured to assign a classification when identifying that an unknown variant is present in the annotated variant dataset. The accuracy of the assigned classification can be based on the amount of similar data in the dataset, for example. The prediction that this machine learning model 2630 makes is followed by a prediction score denoting how accurate the prediction is. The result of the prediction is considered acceptable if it has a high enough prediction score.

This example machine learning model 2630 is configured to do so for variants that are sufficiently annotated. For example, a variant may have missing information for all the scores of interests and this variant is then not classified. In the case of an intermediate to low number of missing values, in this example embodiment, an imputation method is used, and the corresponding variants are classified. Finally, loss-of-function detector 2640 is configured to validate loss-of-function variants based on the LoF_indicator generated by feature extractor 2610 using an output produced by LoFTool, according to some embodiments.

In an example embodiment, the machine learning model is trained using a training dataset of annotated variants, the training dataset of annotated variants generated based on protein functional domain data, sequence ontology data, at least one prediction score, a LoF indicator feature representing a loss-of-function variant and generated using the sequence ontology data, and an Interpro indicator feature representing an effect on an Interpro domain and generated using the Interpro domain data; wherein each prediction score is generated using LoFtool, DEOGEN2, MPC, BayesDel_addAF, FATHMM, integrated_fitCons, or LIST.S2; wherein the protein functional domain data is Interpro domain data; wherein the sequence ontology data represents a splice acceptor variant, a splice donor variant, a stop gained variant, a frameshift variant, a stop lost variant, a start lost variant, or a combination thereof; and wherein the machine learning classification model comprises decision trees implementing random forest; and wherein the assigning of a classification comprises bootstrap aggregation using the decision trees. As one example, the training dataset of annotated variants is generated by extracting features from a dataset comprising the genes CYP2B6, CYP2C19, CYP2C9, CYP2D6, NUDT15, RYR1, SLCO1B1, TPMT, and UGTIA1. For example, Interpro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool. The variable that was used in an example embodiment of the model was an indicator 0/1 of whether a variant had InterPro domain annotation or not.

In some embodiments, the machine learning model is trained using data determined (e.g., extracted and annotated) by feature generator 2610. Such data comprises features (variables) that can be used by machine learning model 2630 and loss-of-function detector 2640 to predict a variant or determine a loss-of-function variant, respectively.

In some embodiments, other annotations are used other than those provided by VEP and its plugins. In some embodiments, the machine learning model is trained using the data annotated by the feature extractor and which indicates the most important variables from the set of all possible variables provided by the feature extractor.

In an example embodiment, the machine learning model is trained using a training dataset of annotated variants, the training dataset of annotated variants generated based on protein functional domain data, sequence ontology data, at least one prediction score, and features provided by feature extractor, wherein those features achieve a desired threshold of model accuracy.

In some embodiments, the machine learning classification model comprises a machine learning model other than random forest. A variety of algorithm methods can be selected from for particular datasets. As the dataset expands, the algorithm that is most suitable may change. For example, decision trees may be used for dataset A, but for an expanded dataset (e.g., A+x) linear regressor may be used. Example methods include: 1) Random Forests; 2) Support Vector Machines (SVMs); 3) Logistic Regression; 4) Boosting Algorithm; 5) Bayes; 6) Genetic Algorithms; 7) kNN; 8) Lars; 9) MLP; 10) others. The method may be combined with a feature selection method such as RFE, Lasso, Ridge, etc., according to some embodiments.

In some embodiments, the processing device executes instructions in memory to configure an interface generator 2650.

In an example embodiment, interface generator 2650 is configured to generate one or more user interface objects on a graphical interface of a display, the one or more user interface objects representing: variant data, the variant data generated based on each validated variant, each predicted variant, and each sequence ontology variant; wherein the one or more user interface objects is generated based on gene location, functional effect, evidence tag, novelty, or pharmacogenomic data; and wherein each evidence tag is assigned to each validated variant by the variant validator, each predicted variant by the machine learning model, or each sequence ontology variant by the loss-of-function detector. In some embodiments, the one or more user interface objects is not generated based on data representing drug-gene association.

Pharmacogenomic data can include data representing drug-gene association, drug dosing data, pharmacogenetic association data, or other pharmacogenomic data. Data representing drug-gene association can include data representing associations between one or more drugs and a single gene (e.g., drug-drug-gene) or one or more drugs and one or more genes, for example. Pharmacogenomic data can include clinically relevant data, such as dosing scheme(s) for drug(s) (such as in relation to a particular gene or variant), pharmacokinetic data (e.g., based on metabolizer status as denotable by one or more variants returned by variant detection platform 2600), and/or pharmacodynamic data. In some embodiments, interface generator 2650 is configured to automatically retrieve data representing drug-gene association from one or more external sources (e.g., remote server, external database, etc.). The data can be derived or received from sources such as the Food and Drug Administration (FDA), European Medicines Agency (EMA), Pharmaceuticals and Medical Devices Agency (PhMDA), Swissmedic, and/or Health Care Service Corporation, and/or research consortia, such as Clinical Pharmacogenetics Implementation Consortium (CPIC), the Dutch Pharmacogenetics Working Group (DPWG), and the Pharmacogenomics Knowledge Base (PharmGKB). Example pharmacogenomic data is described in Appendix II. In some embodiments, interface generator 2650 is configured to match the data representing drug-gene association with corresponding one or more gene(s) (such as variants) extracted by variant validator 2620, assigned a classification by machine learning model 2630, extracted by loss-of-function detector 2640, and/or a variant generated by variant detection platform 2600. In some embodiments, the external sources can include data relating variants to recommended lifestyle decisions or to disease history or predictions. For example, without limiting any other embodiment, in some embodiments, where there exists a gene that can predict symptom severity due to the infection with SARS-COV-2, then, after suitable training, variant detection platform 2600 can be used to predict how a particular individual, given their genomic data in an annotated variant dataset, would respond to SARS-COV-2 infection.

For example, in some embodiments, after predicting the effect of a variant on protein function using variant detection platform 2600, the effect of the variant on the gene level can be determined. For known variants, pharmacogenomic annotations, associations, recommendations, or guidelines can be found using external data sources. Variant detection platform 2600 can also predict the effect of novel variants. For such variants, especially for the case of variants that lead in loss-of-function at the protein and gene level, pharmacogenomic annotations, associations, recommendations, or guidance can be determined by comparison with existing or known variants and/or pharmacogenomic data. The data used to provide the final gene level pharmacogenomic associations can be mined mainly from PharmGKB as well as from the data that they provide, according to some embodiments. PharmGKB provides pharmacogenomic annotations, associations, recommendations, or guidance from the FDA, EMA, CPIC, DPWG, Swissmedic, and Health Canada Sante Canada.

In some embodiments, interface generator 2650 is configured to: receive pharmacogenomic data; determine an association, if any, between the pharmacogenomic data and each validated variant, each predicted variant, and each sequence ontology variant; and generate the one or more user interface objects to represent the additional data, if any, associated with each validated variant, each predicted variant, and each sequence ontology variant.

FIG. 32 shows an example process of interface generator 2650, according to some embodiments. As shown, variants leading to altered protein function are summarized in a single table. For each gene, the number of variants that have a specific effect on protein function is presented, subdivided by novelty and level of evidence. In addition, these results are supplemented by pharmacogenomic data, such as extracted by FDA and PharmGKB, referring to a specific drug-gene association.

In some embodiments, interface generator 2650 is configured to enable variant detection platform 2600 to be used to specifically enable a clinical user to view, on a single display, a direct mapping or association between one or more variants returned by variant detection platform 2600 (e.g., validated variants, predicted variants, sequence ontology variants) and clinically actionable data, such as pharmacogenomic data. Variant(s) that are newly uncovered by variant detection platform 2600 are presented on the display and, using interface generator 2650, can be presented alongside pharmacogenomic data, allowing the user to be presented with new data and insight for pharmacological intervention. Interface generator 2650 can present one or more drugs that would be predicted to have a less favorable or more favorable effect, in view of the variant(s) determined by variant detection platform 2600. In some embodiments, interface generator 2650 generates and presents interface objects representing such information in a particular arrangement, arranged by variant, novelty, evidence, and pharmacogenomic data (e.g., table of drugs interacting with each variant, table of drugs interacting with each gene grouping that each variant belongs to, etc.). For example, in some embodiments, where one or more variants are returned by variant detection platform 2600 as individually or collectively determined to lead to altered protein function, pharmacogenomic data, where available, is retrieved and presented by interface generator 2650 at a display in association with the one or more variants. Where certain drugs are metabolized by enzymes encoded by the one or more variants determined by variant detection platform 2600, the relevant pharmacogenomic data is retrieved and presented with an interface object representing the one or more variants, according to some embodiments.

An example embodiment will now be described. FIG. 31 shows an example process, for example variant detection platform 2600, according to some embodiments. The variant detection platform 2600 is configured for use to evaluate variants in annotated genomic variants identified either on a single patient or on a single file containing the annotated genomic variants from a cohort study. Example variant detection platform 2600 is configured to identify variants (e.g., Short Nucleotide Variants (SNVs)) that are located within the following genes: CYP2B6, CYP2C19, CYP2C9, CYP2D6, NUDT15, RYR1, SLCO1B1, TPMT, and UGT1A1. The selection of these genes was performed based on data regarding interactions between those genes and drug response, as well as based on the availability of functionally annotated variants representing those genes in PharmGKB gene-specific information tables and PharmVar. In other embodiments, variant detection platform 2600 can be configured to identify variants located within other genes. The coordinates of the variants provided comply with the GRCh38 human genome assembly and the variants to be annotated a priori by the Variant Effect Predictor (VEP) tool, as offered by Ensembl. In other embodiments, other VEP tools can be used.

The example variant detection platform 2600 includes an embodiment of machine learning model 2630 configured to implement the ensembl machine learning algorithm (in this example, Random Forest with 1,000 trees) that was trained using a curated dataset of 190 VEP annotated variants in the above-mentioned genes, which was extracted from PharmGKB and Pharm Var. The Random Forest (RF) algorithm is an example of ensembl machine learning models aimed to improve accuracy by combining distinct classifiers. The RF used combines a large number of decision trees (Ntrees). For classification tasks, bootstrap (resampling with replacement) is performed to create a new training set, derived from the original training data (in this case, the 190 variants), which is used to train a decision tree. This process is performed in parallel Ntrees-times and the predictions of the distinct classifiers are aggregated to provide the final decision, based on majority vote. This process is known as bootstrap aggregation, or bagging.

When a file with annotated variants (an annotated variant dataset) is provided to the example of an embodiment of variant detection platform 2600, the following process is performed: 1) the provided variants are processed in order to select only the required features from VEP and construct features required for the classification task; 2) any overlaps with the superset of 262 variants, from which the 190 training variants were derived, are identified and those that lead to altered protein function (increased, decreased, no function) are extracted; 3) the provided variants that do not overlap with the 262 variants are assessed by the classifier and those that are predicted to lead to altered protein function (increased, decreased, no function) are extracted; 4) finally, the remaining variants (that do not overlap with the 262 variants but did not had adequate information to be processed by the machine learning classifier such as an RF classifier) are filtered based on their attributed Sequence Ontology consequences to identify variants enriched for consequences indicative of Loss-of-Function variants (splice acceptor variant, splice donor variant, stop gained, frameshift variant, stop lost, start lost).

An example embodiment of interface generator 2650 is configured to generate interface object(s) presenting the variants that are selected during steps 2 to 4 in a table and sub-divided according to (i) the gene in which they are located, (ii) the level of evidence (“Functionally validated Pharm Var/PharmGKB)” for function altering variants overlapping with the 262 variants, “variant detection platform classifier” for those predicted by the random forest classifier and “Loss-of-Function” for those suggested by the Sequence Ontology consequence), (iii) their novelty (known or novel variants, respectively). In other embodiments, different tags representing the level of evidence are used. In other embodiments, a machine learning model other than a random forest classifier is used. In other embodiments, a different random forest classifier implementation can be used, such as with a different number of decision trees. This data is presented by interface generator 2650 on a gene-level basis and for each of the pharmacogenes of interest (in this example, nine) a table is provided, which presents the number of function-altering variants by functional effect (increased, decreased, no function), novelty, and level of evidence. In other embodiments, a different data structure other than a table is used. In addition, this data is supplemented by data extracted from the FDA's table of pharmacogenetic associations, which presents observed interactions between gene-drug pairs and provides further information on the affected populations (expressed according to their metabolizer or transporter status). In some embodiments, the data is received from external sources (e.g., database, remote computer, local computer) and the data is pharmacogenomic data. The external sources can be other regulatory bodies or consortiums as described herein. Moreover, additional data on drug-gene association is extracted by PharmGKB, with this information indicating whether this association is characterized according to PharmGKB classifications as “Testing required”, “Testing Recommended”, “Actionable PGx”, or “Informative PGx”.

In an example embodiment of interface generator 2650, in particular, the function altering variants from steps 3 to 5 are summarized in a single table. For each gene, the number of variants that have a specific effect on protein function is presented, subdivided by novelty and level of evidence. In addition, these results are supplemented by information extracted by FDA and PharmGKB, referring to a specific drug-gene association.

### Machine Learning Model Training

In some embodiments, variant detection platform 2600 is configured to train the machine learning model 2650. Variant detection platform 2600 includes: at least one processor; and at least one memory storing computer-executable instructions which, when executed, cause the at least one processor to perform a method, the method comprising: generating at least one annotated variant training dataset, the generating comprising: receiving at least one annotated variant dataset, annotated based on protein functional domain data, sequence ontology data, and at least one prediction score; and applying k-nearest neighbor (kNN) imputation to the at least one annotated variant dataset to generate one or more values for missing data. The method further comprises training the machine learning model using at least one annotated variant training dataset. In some embodiments, the annotations represent features, and the features are based on additional data, and such features are selected based on impact on one or more metrics of machine learning model 2650, such as model accuracy.

In some embodiments, the method comprises: generating at least one annotated variant training dataset, the generating comprising: receiving at least one annotated variant dataset, having features based on protein functional domain data, sequence ontology data, and at least one prediction score; and applying k-nearest neighbor (kNN) imputation to the at least one annotated variant dataset to generate one or more values for variant(s) having missing data for features.

A new dataset having the missing values can be generated.

In some embodiments, the missing values are generated using a method other than kNN imputation. For example, such methods can include: 1) imputation using (mean/median) values, 2) imputation using (most frequent) or (zero/constant) values, 3) imputation using k-NN, 4) imputation using multivariate imputation by chained equation (MICE), 5) imputation using deep learning.

In some embodiments, the features are those that allow machine learning model 2650 to have a model accuracy within a desired threshold.

In some embodiments, at least one annotated variant dataset is annotated using a Variant Effect Predictor (VEP), such as provided by Ensembl and its plugins.

In some embodiments, each prediction score is generated using LoFtool, DEOGEN2, MPC, BayesDel_addAF, FATHMM, integrated_fitCons, or LIST.S2. In some embodiments, each prediction score is generated using other features.

In some embodiments, the protein functional domain data is Interpro domain data.

In some embodiments, the sequence ontology data represents a splice acceptor variant, a splice donor variant, a stop gained variant, a frameshift variant, a stop lost variant, a start lost variant, or a combination thereof.

In some embodiments, generating at least one annotated variant training dataset further comprises: generating a LoF indicator feature using the sequence ontology data, the LoF indicator feature representing a loss-of-function variant.

In some embodiments, generating at least one annotated variant training dataset further comprises: generating an Interpro indicator feature using the Interpro domain data, the Interpro indicator feature representing an effect on an Interpro domain.

In some embodiments, the machine learning model is a random forest classifier having decision trees, the machine learning model configured to assign a classification based on bootstrap aggregation using the decision trees.

In some embodiments, the kNN imputation is kNN imputation with weighted mean.

In some embodiments, generating at least one annotated variant training dataset further comprises: removing data from the at least one annotated variant dataset, wherein the data corresponds to a variant having a percentage greater than or equal to 40%, collectively, of missing values for the annotations, the removing performed before kNN imputation is applied to the at least one annotated variant dataset; and removing data from the at least one annotated variant dataset, wherein the data corresponds to a feature having a percentage greater than or equal to 40%, collectively, of missing values for variants represented in the at least one annotated variant dataset, the removing performed before kNN imputation is applied to the at least one annotated variant dataset. For example, if a variable has more than 40% of missing values, it is removed, according to some embodiments. For example, the feature can be a LoF feature or an Interpro indicator feature or another feature defined by the annotations, where there is no such feature data for >=40% of the variants.

In some embodiments, generating at least one annotated variant training dataset further comprises: performing variant deduplication on the at least one annotated variant dataset to generate at least one new annotated variant dataset; extracting features from the at least one new annotated variant dataset, the features comprising protein functional domain data, sequence ontology data, at least one prediction score, at least one variant identifier, and at least one sequence identifier; generating a LoF indicator feature using the sequence ontology data, the LoF indicator feature representing a loss-of-function variant; and generating an Interpro indicator feature using the Interpro domain data, the Interpro indicator feature representing an effect on an Interpro domain. The extracted features, selected data, and the generated features can then be included in the generated annotated variant training dataset. In some embodiments, for example, there can be two prediction scores as well as data for each of the other features.

In some embodiments, the generating at least one annotated variant training dataset further comprises: for each variant, for each nested feature, if any, (e.g., features extracted from a VEP plugin such as dbNSFP), selecting data representing each prediction corresponding to a transcript belonging to a pre-defined set of transcripts of interest. When one or more features are extracted from at least one annotated variant dataset, this can be from Ensembl's VEP and its plugins, such as dbNSFP.

FIG. 33 shows an example process for training an example machine learning model for an example variant detection platform 2600, according to some embodiments. In some embodiments, variant detection platform 2600 includes a pre-processor. The pre-processor is configured to process the annotated variant dataset before machine learning classification model 2630 receives data derived from same (e.g., features). In some embodiments, such processing by pre-processor proceeds as follows.

In some embodiments shown in FIG. 33, at the pre-processor, at the deduplication and integration of classes stages, at original input file(s) received by variant detection platform 2600, the file can contain information for all the DNA of a patient even if a variant is not present. This file is reduced by selecting only the positions on the DNA where a variant was detected. Concerning the HGVS annotation of a variant, this is returned by using VEP and its plugin dbNSFP, according to some embodiments. HGVSc and HGVSp annotations are returned and HGVSc can be selected.

In some embodiments shown in FIG. 33, at the pre-processor, at the deduplication and handling of inconsistencies stages, the process proceeds as follows. First, a variant might get no or too few annotations by the feature extractor 2610 and these variants are disregarded; and second, a variant might be annotated for most fields in which case missing data is imputed (e.g., using kNN imputation) and are continued to be processed by variant detection platform 2600. In some embodiments, recursive feature elimination (RFE) is used to select the most important features. Other methods can be used. Given a model, RFE trains the model recursively, and on its iteration, removes the least important variable. Applying RFE leads to a set of the most important variables by selecting the set that led to the highest metric of interest, for example, precision metric of interest, such as accuracy of the model.

In some embodiments shown in FIG. 33, at classification model training, at the hyper-parameter tuning stage, the process proceeds as follows. Hyper-parameters in a machine learning model can tune the way the model is trained. Hyper-parameter tuning can proceed by creating a grid of possible values for these parameters, training the model on each point of the grid, and finally selecting the specific values of the hyper-parameter that maximized the metric of interest, for example, accuracy of the model. This can be performed by variant detection platform 2600 when training a machine learning classification model 2630. This can increase the accuracy of the model.

In some embodiments shown in FIG. 33, at the k-fold cross validation stage, the process proceeds as follows. In an example, k is equal to five. At k-fold cross validation, machine learning model 2630 is trained k times by splitting the data in k-folds and using k-1 folds for training and the remaining for testing. Machine learning model 2630 is trained and validated k times on all the possible folds configurations, according to some embodiments. Then, an average accuracy is calculated giving a metric of how accurate the model is. By applying this method to several models, the models are comparable and the one with the highest metric of interest, for example, model accuracy, can be selected as the final model. Using this process, a suitable machine learning model can be selected by variant detection platform 2600 as machine learning model 2630, according to some embodiments.

For example, in some embodiments, 5-fold cross validation of the trained model is performed. Different methods can be tested to find the best one for a given dataset. When testing the methods, different hyper-parameters are used to determine which method under which hyper-parameters fits best. Five-cross validation can be used to split the dataset into five smaller ones to train each differently. This can help train the model in an improved way, as the model can make predictions on unseen data.

In some embodiments, next, allele frequency data (AF) where not used when training the initial machine learning model can now be used. Interface generator 2650 can be configured to incorporate such data in an interface object, such as in a report.

In an example embodiment, to train a multiclass classifier that would distinguish between increased, normal, decreased and no function variants, a set of 190 functionally validated variants were collected from data sources, which are accompanied by VEP annotations. These VEP annotations included Interpro domains, Sequence Ontology defined consequences, as well as the following in silico prediction scores: LoFtool, DEOGEN2, MPC, BayesDel_addAF, FATHMM, integrated_fitCons, and LIST.S2. The sequence ontology consequence was utilized to create a new feature named “LoF indicator”, containing 1 if a consequence indicative of LoF variants was identified and 0 elsewhere. Similarly, Interpro was transformed to indicate whether a variant was affecting an Interpro identified protein domain, by converting missing values to 0 and replacing all other cases with 1. Any missing values in the in-silico scores were filled using kNN imputation method with weighted mean, and the resulting data were used to train a random forest classifier with 1,000 trees. The trained model was saved as an R data file (.rds) and used in the variant detection platform 2600 (training was performed once and the created machine learning model 2630 is used for predictions).

Experiments on embodiments of variant detection platform 2600 will now be described.

As used herein, the following terms have been abbreviated as follows: single nucleotide Variations (SNVs), pharmacogenomics (PGx), Next-generation sequencing (NGS), Whole Exome sequencing (WES), Whole Genome sequencing (WGS), European Medicines Agency (EMA), Food and Drug Administration (FDA), Copy Number Variants (CNVs), minor allele frequency (MAF), Random Forest (RF), Area Under the Curve (AUC), Area Under the Precision-Recall Curve (prAUC).

The field of pharmacogenomics focuses on the way a person's genome affects their response to a certain dose of a specified medication. The main aim is to utilize this information to guide and personalize the treatment in a way that maximizes the clinical benefits and minimizes the risks for the patients, thus fulfilling the promises of personalized medicine. Technological advances in genome sequencing, combined with the development of improved computational methods for the efficient analysis of the huge amount of generated data, have allowed the fast and inexpensive sequencing of a patient's genome, hence rendering its incorporation into clinical routine practice a realistic possibility.

Various patient-specific factors (i.e., ethnicity, age, co-existing conditions, co-administered medications) have been associated with deviations between the expected and the observed effects owing to a specific medication. In addition, a significant percentage of these differential drug responses has been attributed to genetic variants located in genes involved in the processes of pharmacokinetics, pharmacodynamics or even in genes coding for enzymes of the immune system (i.e., HLA genes), commonly described as pharmacogenes. This genetically determined diversity of drug effects, as well as its exploitation towards tailoring the medication scheme is a focus of pharmacogenomics (PGx), and an integral component of personalized medicine. To this end, genotyping platforms, such as DMET™ plus by Affymetrix, can be used to detect well-characterized, common genetic variants. Alternatively, next-generation sequencing (NGS), either Whole Exome Sequencing (WES), Whole Genome Sequencing (WGS) 30X or even targeted resequencing, can be also used for this purpose, thus providing a more comprehensive idea of an individual's genomic composition.

To date, 15% of the approved drugs by the EMA (European Medicines Agency) in the period 1995-2014, and 7% of the drugs approved by the American Food and Drug Administration (FDA), are accompanied by pharmacogenomic recommendations. Interestingly, relevant PGx biomarkers can be either germline variants in pharmacogenes, mostly Single Nucleotide Variations (SNVs) or Copy Number Variants (CNVs), or somatic variants in cancer cells that affect tumor's response to antineoplastic drugs, as well as epigenetic modifications of histones and DNA, which could potentially affect the drug response. The effects of these PGx variants might range from altered drug exposure and hence modified efficacy or side effects, to idiosyncratic reactions.

The results of large-scale NGS analyses unravel several challenges, thus complicating the interpretation of the effects of PGx variants on protein function. For example, a large volume of novel, rare (minor allele frequency: MAF <0.5%), population specific SNVs, which could affect protein function has been detected within protein coding genes. These genes appear to be enriched in potentially damaging variants, owing to the combination of rapid population growth and weak action of purifying selection. Similar observations were applied when focusing on 202 genes, the products of which are molecular targets for drug action). Regarding the genes coding for Phase I metabolic enzymes (CYPs (Cytochrome P450)) and drug transporters (UGT, ABC genes), the majority of the identified SNVs within these genes is ultra-rare (MAF <0.1%) and non-synonymous, while variants that affect splicing sites or lead to loss of the termination codons, as well as nonsense changes are less common. Furthermore, the evaluation of organo anion transporter (OATP) transporter sequences provided by the Genome Aggregation Database (gnomAD) has underlined once again the importance of including novel, rare mutations (MAF <1%) in the pharmacogenomic assays.

Taken together, NGS analyses have the potential to identify a very large number of PGx variants, most of which are novel, rare and with no biochemical or clinical evidence for their impact on protein function. Performing functional expression assays for such large numbers of variants is not always feasible, hence why the evaluation of predictions derived from in silico tools is an alternative approach to this end. Most computational methods used to assess the functional effect of variants in protein level are intended to distinguish neutral from deleterious variants, based on either a hypothesis or the evaluation of a set of properties, including secondary structure, functional sites, protein stability and sequence conservation. A number of methods using unsupervised learning, as well as gene-level scores and ensembl approaches that integrate the predictions and training features of other tools are possible.

Example methods include SIFT, PROVEAN, PolyPhen-2, MutPred, GERP++, Eigen, Eigen-PC, LoFtool, DANN, Revel, and MetaLR/MetaSVM.

However, pharmacogenes and the respective PGx variants tend to differ from genes and variants implicated in disease. The suitability of features considered by available methods is questionable, since genes coding for phase I and II metabolizing enzymes appear to be less conserved evolutionary, possibly due to their limited role in endogenous processes and the fact that only a mild modification of the pharmacokinetics and pharmacodynamics can lead to significant results. Nevertheless, the development of an improved framework for the evaluation of pharmacogenomic variants, by combining different classifiers and appropriately adjusting their prediction thresholds has led to promising results.

Described herein is variant detection platform 2600. In some embodiments, variant detection platform 2600 can be used for the assessment of PGx variants by evaluating in silico protein prediction scores with the use of machine learning (ML), and thus highlighting the PGx variants that are most likely to alter the protein function and consequently have a PGx impact. In some embodiments, variant detection platform 2600 provides a machine learning-based system and method for the computational functional assessment of pharmacogenomic variants.

In an example embodiment of variant detection platform 2600, thoroughly characterized functional level SNVs within genes involved in drug metabolism and transport were used to train a classifier that would categorize novel variants according to their expected effect on protein functionality. This categorization is based on the available in silico prediction and/or conservation scores, which are selected with the use of recursive feature elimination process. Towards this end, information regarding 190 pharmacovariants was leveraged, alongside with four machine learning models, namely AdaBoost, XGBoost, Multinomial logistic regression and Random Forest, of which the performance was assessed through 5-fold cross validation.

All models achieved similar performance towards making informed conclusions, with RF model achieving the highest accuracy (85%, 95% CI: 0.79, 0.90), as well as improved overall performance (Precision 85%, Sensitivity 84%, Specificity 94%) and being used for subsequent analyses. When applied on real world WGS data, the selected RF model identified 2 missense variants, expected to lead to decreased function proteins and 1 to increased. A greater number of variants were highlighted when the approach was used on NGS data derived from targeted resequencing of coding regions. Specifically, 71 variants (out of 156 with sufficient annotation information) were classified as to “Decreased function”, 41 variants as “No” function proteins, and 1 variant in “Increased function”.

In using the example embodiment of variant detection platform 2600, publicly available and human variation data with well-defined protein-level functional consequences was used to train a predictive model for the targeted classification of coding SNVs with regards to their protein function effects. The assigned protein function effect scores were based on the integration and assessment of in vitro biochemical assays, in vivo evidence, and clinical data. Four different models (AdaBoost, XgBoost, RF, Multinomial logistic regression) were trained with a training set consisting of 190 variants, located across 11 pharmacogenes and were assessed with 5-fold cross validation. The applicability of the optimal model was also assessed using NGS data, either whole genome or targeted sequencing data.

For example, the RF-based model can be used for variant prioritization and acting as a scoring tool with interesting clinical applications in the fields of pharmacogenomics and personalised medicine.

### Performance Metrics for the Machine Learning Models Toward the Functional Assessment of PGx Variants

In the example embodiment of variant detection platform 2600, the performance of the classifiers, which were constructed with variables recommended by the recursive feature elimination (RFE) method, was advantageous regardless of the limited sample size of the training set (N=190 variants in 11 genes). More precisely, the metrics computed for the assessed machine learning models were as follows: Random Forest (RF)—Accuracy: 0.85 (95% CI: 0.79, 0.90), Area Under the Curve (AUC)=0.92, Area Under the Precision-Recall Curve (prAUC)=0.73; AdaBoost—Accuracy: 0.82 (95% CI: 0.76, 0.87), AUC: 0.91, prAUC: 0.72; XGBoost—Accuracy: 0.80 (95% CI: 0.73, 0.85), AUC: 0.91, prAUC: 0.73; Multinomial logistic regression —Accuracy: 0.78 (95% CI: 0.72, 0.84), AUC: 0.93, prAUC: 0.74. Multinomial logistic regression led to higher AUC and prAUC values compared to the tree-based approaches, whilst the achieved accuracy was the lowest amongst the assessed models.

In the example embodiment of variant detection platform 2600, RFs were selected to be used for machine learning model 2650 for the described classification task, since the model presented overall improved performance (i.e., accuracy, sensitivity, specificity, and precision) across all four functional classes in the example embodiment used. Regarding the ‘Decreased function’ variants, RFs were more sensitive and precise than the other assessed models, although AdaBoost achieved equal specificity values (FIG. 34). All models performed impressively well towards the ‘Increased function’ category and led to very similar outcomes, while RFs appeared superior for the detection of ‘No function’ variants and AdaBoost and Multinomial Logistic Regression models were more sensitive for the ‘Normal function’ class.

FIG. 34 shows example metrics showing the performance of the different classifiers, namely AdaBoost, Multinomial Logistic Regression, Random Forest, XGBoost. More specifically, the sensitivity, specificity, positive predictive value (pos.pred.value), precision, F1 metric (harmonic mean of precision and recall) and balanced accuracy of the classifiers are provided for each protein function effect class.

The selected machine learning model proved to be highly specific (≥92%) for all 4 functional classes of variants, with lower, but still favorable values of sensitivity (80%-98%), precision (80%-98%) and balanced accuracy (86%-99%). With regards to identifying variants that could lead to proteins with unchanged (normal), reduced or no function, the lowest values of the metrics were observed.

The model was characterized by a better performance for ‘Normal function’ variants (Sensitivity=0.8, Specificity=0.92, Precision=0.84, Balanced Accuracy=0.86), followed closely by ‘No’ function variants (Sensitivity—0.81, Specificity=0.93, Precision=0.81, Balanced Accuracy=0.87) and finally ‘Decreased function’ variants (Sensitivity=0.81, Specificity=0.95, Precision=0.80, Balanced Accuracy=0.88). The classifier performs extremely well for the category of ‘Increased Function’ variants, in which case all computed metrics were above 98% (FIG. 34). To better explain the performance of the RF classifier with respect to four variant classes, the distribution of the training variants for the scores suggested by RFE and included in the classifier is provided in FIG. 35. The improved performance towards ‘Increased function’ can be explained by the better definition of these variants compared to the rest classes (′No′, ‘Decreased’ and ‘Normal function’), which are characterized by a substantial extend of overlapping values, that could complicate their accurate classification.

FIG. 35 shows violin plots depicting the distribution of each functionality class for certain in silico predictions tools, as suggested by the recursive feature elimination (RFE) procedure, according to some embodiments. The graphs show the distribution of values per functionality class (‘decreased’, ‘increased’, ‘no’, ‘normal’) for each one of the 7 RFE-suggested scores, which were used as the training features in the final classifier. These RFE-suggested scores are derived from: LoFtool, DEOGEN2_score, MPC_score, BayesDel_addAF_score, integrated_fitCons_score, FATHMM_score and LIST.S2_score.

Variables that could significantly affect the presented machine learning model were also assessed. More specifically, when it comes to the variable importance, the highest-ranking positions were occupied by these features that RFE suggested as the most informative ones for the classification task. In the present instance, LoFtool emerged as the prominent for the categorization of a variant according to its effect on protein function (see FIG. 54).

FIG. 54 shows example annotation features examined as training variables in an example machine learning model 2630 for the functional assessment of pharmacogenomics variants. These features are ranked according to their suggested interpretational significance from least (bottom) to most important (top).

### Comparing the RF Model Against Other Broadly Used in Silico Tools

As a further step, an assessment of how different, commonly used functionality prediction algorithms would classify the 190 variants that were included in the final training set was performed. Towards this end, ClinPred, Condel, FATHMM, Fathmm-XF, LRT, MetaLR, PolyPhen-2, PROVEAN, and SIFT were selected and the corresponding predictions, as provided by VEP, are presented in FIG. 36. Of these scores, only FATHMM-XF can be also applied to non-coding variants, while the rest are intended for use in coding, non-synonymous SNVs. In addition, ClinPred, Fathmm, and MetaLR classify variants as either ‘Tolerated’ or ‘Damaging’, Condel as ‘Neutral’ or ‘Deleterious’, FATHMM-XF and PROVEAN as ‘Neutral’ or ‘Damaging’, LRT as ‘Deleterious’, ‘Neutral’ or ‘Unknown’, PolyPhen-2 as ‘Benign’, ‘Possibly Damaging’ or ‘Probably Damaging’, and, finally, SIFT as ‘Tolerated’, ‘Tolerated with low confidence’, ‘Deleterious with low confidence’ and ‘Deleterious’. As a first observation, none of these tools covers variants that could lead to gain-of-function. Although this functionality may be provided by B-SIFT, it is not available through VEP, and thus, it was not included in the analysis. Regarding increased function variants, all algorithms, except LRT, categorize these variants as either ‘Damaging’ or ‘Deleterious’. In addition, there is apparent discordance among the tools' classification of ‘decreased’ and ‘normal’ function variants, while most algorithms may be able to identify variants leading to non-functional proteins.

FIG. 36 shows classification of the variants included in the training set as based on broadly used in-silico prediction tools, according to some embodiments. The columns show each functionality class (‘decreased’, ‘increased’, ‘no’ and ‘normal’) and each row shows the distribution of variants within each class according to the predicted variant effect, as assigned per each in silico prediction tool. Predefined cutoff values for classification (as used in VEP and dbNSFP) are implemented. The scores are derived from: ClinPred, Condel, FATHMM, fathmm.XF, LRT, MetaLR, Polyphen-2, PROVEAN and SIFT.

### Application of the Machine Learning Model in NGS Data

### First Case Study (WGS Data)

To further demonstrate the prediction performance of the final RF model, its applicability in “unseen” NGS data was tested, namely those data that have not been previously used to train the machine learning algorithm. We first tested its applicability in WGS data from a patient diagnosed with coeliac disease. From this process, 1,808 variants, including 3 novel, within the 10 pharmacogenes of interest (DPYD, CYP2C19, CYP2C9, SLCO1B1, NUDT15, RYR1, CYP2B6, UGT1A1, CYP2D6, TPMT) were identified. Of these, only six missense variants had adequate information, i.e., no missing values in the incorporated functional prediction scores, to be further processed by the example embodiment of the RF model. With regards to the observed allele frequency, four were found to be common (rs1801159, rs2306283, rs4149056, rs35364374), one had intermediate frequency (rs3745274) and one was ultra-rare (rs762454967)—with MAFs based on GnomAD genomes. Of these 1,808 analyzed variants, no variants categorized as loss-of-function variants (LoF) were determined.

Table 2 presents these variants, alongside their predicted functional impact, as defined by the majority vote of the individual decision trees. For example, a random forest containing 26000 distinct decision trees was built. If most of those votes recommend that the variant belongs to ‘No function’ variants, then this is the class that is attributed to the variant in some embodiments. In addition, the probability of being classified in each class, as based on the votes of all trees of the random forest built, is also provided (Table 2).

TABLE 2
Classification outcomes (prediction and probabilities) for WGS data using the final RF model. The predicted class
is determined based on a majority vote from the individual decision trees of the random forest classifier, while
the presented probabilities depict the corresponding percentage of decision trees voting towards a functional class.

| | | | | | | | Probability |
|---|---|---|---|---|---|---|---|
| Location | | Existing | | | GnomAD | Predicted | of attributed |
| (GRCh38) | Allele | variation | SYMBOL | HGVSc | AF (%) | class | class |
| 1:97515839- | C | rs1801159, | DPYD | ENST00000370192.8:c.1627A > G | 18.49% | Normal | 0.96 |
| 97515839 | | CM033371, | | | | | |
| | | COSV64593269 | | | | | |
| 12:21176804- | G | rs2306283, | SLCO1B1 | ENST00000256958.3:c.388A > G | 53.33% | Normal | 0.66 |
| 21176804 | | CM043776, | | | | | |
| | | COSV57012766 | | | | | |
| 12:21178615- | C | rs4149056, | SLCO1B1 | ENST00000256958.3:c.521T > C | 11.95% | Decreased | 0.88 |
| 21178615 | | CM043777, | | | | | |
| | | COSV57010105 | | | | | |
| 19:38492540- | T | rs35364374 | RYR1 | ENST00000359596.8:c.6178G > T | 4.95% | Increased | 0.38 |
| 38492540 | | | | | | | |
| 19:38499641- | A | rs762454967, | RYR1 | ENST00000359596.8:c.7034G > A | 0.00% | Increased | 0.80 |
| 38499641 | | CM2640865 | | | | | |
| 19:426006936- | T | rs3745274, | CYP2B6 | ENST00000324071.10:c.516G > T | 28.44% | Decreased | 0.72 |
| 426006936 | | CM2630453, | | | | | |
| | | CS080663, | | | | | |
| | | COSV57843253 | | | | | |

This computational process led to the confirmation of 2 missense variants (located within the SLCO1B1 and CYP2B6 genes, respectively) that could potentially lead to proteins with decreased functionality and 1 missense variant classified as ‘increased function’ (located in RYR1). The remaining two variants were predicted to lead to no changes in the protein function (i.e., normal). The rest of the PGx variants had a high rate (over 85%) of missing values in the features of interest and were mostly (N=1,765 out of 1,803; 97.89%) located within intronic regions (FIG. 55). The latter were followed by variants in 3′ prime UTRs (N=20; 1.11%), missense (N=6; 0.33%) and synonymous (N=11; 0.61%) variants. Interestingly, DPYD which encodes for a drug metabolizing enzyme, accumulated more than 1,000 intronic variants.

Regarding the potential clinical actionability of these 6 variants (rs1801159, rs2306283, rs4149056, rs35364374, rs3745274 and rs762454967), an example embodiment of interface generator 2650 was used to retrieve additional information from the PharmGKB database. rs1801159 and rs2306823 were not associated with any predicted changes in the protein function or changes in the dosing guidelines (i.e., normal, or low-level changes respectively). However, changes in treatment were recommended for individuals with the rs4149056 variant genotype, whilst also stating that any additional risk factors should be considered for statin-induced myopathy. Moreover, rs3745274 carried multiple levels of CPIC evidence, for a variety of drugs such: as efavirenz, nevirapine, propofol, imatinib, cyclophosphamide, doxorubicin, mitotane, methadone and 3,4-methylenedioxymethamphetamine. No PGx clinical information was available for rs35364374 and rs762454967 within RYR1, which were both predicted as ‘increased function’ variants.

FIG. 55 shows a distribution of PGx variants identified in the WGS data (first case study), according to some embodiments, that were not processed owing to many missing values. The graph presents the number of PGx variants, by gene, that were not processed any further by the example machine learning model 2630, according to the VEP consequence (i.e., 3′ UTR variant, intronic variant, missense variant, splice region variant and synonymous variant). The pharmacogenes are shaded according to the corresponding PGx group: genes encoding drug metabolizing enzymes or genes encoding drug transporters or other non-metabolizing enzymes.

### Second Case Study (Targeted PGx Sequencing Data)

The second case study consisted of targeted PGx sequencing data from 304 individuals of Greek origin and diagnosed with psychiatric disorders. 343 variants were identified, covering 10 pharmacogenes (DPYD, CYP2C19, CYP2C9, CYP2C8, SLCO1B1, NUDT15, CYP2B6, UGT1A1, CYP2D6, TPMT), 18 of which were attributed as a SO consequence indicative of LoF variants. More specifically, 11 ‘frameshifts’, 6 ‘stop gained’ and 1 ‘start lost’ variants were determined. None of these variants was assessed by the RF model owing to the high levels of missing values (mean: 77% missing values in the scores of interests). The remaining variants were mostly missense (N=205) or synonymous (N=107) (FIG. 56). According to GnomAD genome frequencies in the general population (AF), which were available for 88 of these variants, the dataset was enriched for ‘ultra-rare’ variants (MAF≤0.1%) (N=42), followed by ‘rare’ (0.1%≤MAF<1%) (N=18), ‘low frequency’ (1%≤MAF<5%) (N=14), ‘common’ (MAF≥10%) (N=8) and ‘intermediate’ (5%≤MAF<10%) (N=6) variants.

FIG. 56 shows sequence ontology consequences according to some embodiments for the identified PGx variants, as derived from a Greek cohort of 304 individuals with psychiatric disorders (second case study). 343 PGx variants within the pharmacogenes of interest were identified in this cohort. Amongst the consequences are ‘frameshift’, ‘missense’, ‘missense or splice region’, ‘splice region’, ‘start lost’, ‘stop gained’ and ‘synonymous’ variants.

The dataset of 343 variants included 195 known and 148 novel variants, of which 86 novel and 70 known PGx variants (156 in total) were evaluated by the final RF model (data available upon request). The evaluated variants were mostly missense (i.e., 149 ‘missense’, 7 ‘missense/splice region’). Of these, 71 variants led to ‘Decreased’ function proteins, 41 variants to ‘No’ function proteins, 1 variant in ‘Increased’ function protein and 43 variants have no effect on protein functionality (i.e., ‘normal’ function) (FIG. 37).

FIG. 37 shows protein function predictions based on the final RF model after assessment of the targeted PGx sequencing data from a Greek cohort of 304 individuals, according to some embodiments. The functionality class for the PGx variants, as processed by the RF model, is depicted per each pharmacogene of interest. The numbers denote the total number of variants within each pharmacogene per function class.

To further estimate the potential clinical actionability of the 156 PGx variants, as evaluated by the RF model, additional clinical and variant information was retrieved from PharmGKB. rs1801159, rs1801158, rs2297595 and rs1801160 were not associated with any predicted changes in the protein function, according to the variant annotation by PharmGKB, which constitutes an observation in concordance with the assigned prediction classes by the RF model (i.e., ‘normal’ function class). Moreover, rs67376798 was associated with decreased catalytic activity based on evidence from PharmGKB, thus further confirming the prediction class of the RF model (i.e., ‘decreased’ function class). Similar observations were applied for the variants, namely rs4149056, rs116855232 and rs3745274, for which the following prediction classes were assigned by the RF model: ‘decreased’, ‘no’, ‘decreased’ respectively. PharmGKB provides multiple levels of clinical evidence for these variants, the majority of which were associated with decreased protein activity, therefore confirming the presented model results.

Conventional genetic testing and clinical guidelines focus solely on a small number of well-studied variants or star alleles in pharmacogenes, while the application of NGS techniques provides the possibility to detect a much wider range of (PGx) variants. Recent studies have demonstrated that coding variants are rare, population-specific, and with a significant proportion of them potential affecting the protein product (based on in silico assays and metrics). At the same time, the role of copy number variants (CNVs) within pharmacogenes, as well as variants in non-coding regions, are gaining more attention, with more than 90% of the polymorphisms detected in GWAS pharmacogenomic studies being non-coding. Owing to the limited number of thoroughly documented PGx variants and the incredibly large number of identified genetic mutations that should be experimentally validated, the initial evaluation of these variants found must be performed via the use of in silico tools.

The assessment of the utility of in-silico derived scores, commonly used for variant annotation, toward the characterization of the potential protein function effects of SNVs identified within pharmacogenes was performed on the example embodiment of variant detection platform 2600. Amongst the assessed models (AdaBoost, XGBoost, RF, multinomial logistic regression), RF presented superior performance and was selected as the final classifier. RFs have been also shown to be robust in the presence of outliers or noise, effective, even without configuration, and useful in cases where the number of available ‘-omics’ data is limited, when compared to the number of available variables.

The final classifier required minimum hyperparameter tuning and integrated 7 scores, stand-alone or ensembl ones, and 2 custom created variables. The overall accuracy was equal to 0.85 (95% CI: 0.79, 0.90), with an Area Under the Curve of 0.92 and an Area Under the Precision-Recall Curve (PR AUC) of 0.73. The by-class performance for variants of Normal, Decreased and No Function classes is efficient enough. In some embodiments, these metrics can be improved, such as in terms of sensitivity (0.80, 0.81, and 0.81 respectively). The model appears to be efficient, given the fact that most of the incorporated features are used to distinguish between damaging and benign variants, specifically when it comes to identifying Increased function SNVs. Furthermore, LoFtool, an approach that evaluates the tolerance of a gene to loss-of-function mutations emerged as the most significant determinant of the classification task. The superior performance of the model in identifying ‘Increased’ Function PGx variants, combined with the observation that this specific class in the training dataset represents only two pharmacogenes, might partially justify the importance of the variable.

The possibility of using PGx variants so as to develop classification tools can be explored, though there are limitations and difficulties that accompany this field. Firstly, the most frequently examined properties in such classification tools are the degree of evolutionary conservation, which is observed in lower levels in pharmacogenes and therefore its usefulness is debated by a series of studies, as well as parameters regarding the structure of the respective proteins, which have been observed to lead to small increases in the efficiency of the classifiers produced. Overall, such factors could influence the quality of the output results in classification models.

In addition, the training sets used to train computational models are usually comprised of common polymorphisms against variants (mostly SNVs) related to disease-causality, while in terms of drug response, the modifying effect of common genomic variants cannot be ruled out. Moreover, the resulting scores evaluate the pathogenic potential of the examined variants and classify them into two usually categories according to certain applied thresholds. In contrast, PGx researchers usually focus on the induced change in protein function, which can be distinguished at several levels (e.g., increase, decrease, no change, complete loss of activity), while the differential drug response is not a disease, but a phenotype that occurs under specific conditions (i.e., administration of a specific drug).

For example, the adaptation of proposed classification thresholds and the subsequent integration of selected algorithms, which could provide optimal results for the creation of a comprehensive score, led to a tool with sensitivity and specificity. However, this focused exclusively on the distinction between loss-of-function and neutral variants, hence ignoring PGx variants that would result in a protein product of increased activity, and which are of interest in PGx field.

In some embodiments, variant detection platform 2600 uniquely configures a classifier. Specifically, starting from a VEP annotated VCF file as the input, the classifier quickly leads to a list of PGx variants that could harbor a protein function effect and hence a potential clinical PGx impact. Unlike disease-related variants, there is no state-of-the-art procedure so far which can be used to interpret variants implicated in drug response. Taken together, in some embodiments, the variant detection platform 2600 is configured for variant analysis process automatization and the incorporation of available in silico scores for the evidence-based assessment of pharmacovariants.

Discrepancies have been observed not only amongst different algorithms, or between in silico predictions and in vitro activity, but also when comparing in vitro and in vivo observations. A characteristic example is that of CYP2D6*35, which has not been associated with reduced activity, despite the experimental evidence of reduced hydroxylation capacity of tamoxifen. Moreover, the same variant may affect the response to different drugs in different ways. For example, although the CYP2C8*10 and CYP2C8*13 alleles have been found to affect the N-deethylation of amodiacin, the hydroxylation of paclitaxel-which is also metabolized by CYP2C8—remains unaffected.

In some embodiments, other machine learning (ML) approaches, supervised or not, can be used. Furthermore, significant advantages are expected to emerge from the collection and curation of larger training sets, consisting of larger numbers of variants and covering an additional number of pharmacogenes. Furthermore, more suitable features for the characterization of these PGx variants can also be used.

In some embodiments, there can be the integration of CNVs, as well as non-coding variants related information, using tools for CNV calling and non-coding variants functional assessment. Some embodiments can use well-characterized sets of PGx variants at the level of protein effects, both laboratory and clinical, as well as improved databases to facilitate the export of the requested information. An individual does not carry just one variant in one pharmacogene; therefore the combination of PGx variants is often what results in the overall difference in drug response. In some embodiments, since the contribution of various factors to the response to a given drug is possible, variant detection platform 2600 can implement a comprehensive method through systemic genomics, incorporating a variety of different-omics data.

In some embodiments, variant detection platform 2600 uses a ML approach to classify PGx variants, particularly novel and rare variants, by consequently assigning a protein activity prediction. Overall, the presented model prioritizes annotated PGx variants in different variant effect classes and then assigns a protein function classification after stringent computational assessment and ML processes. Its utility was further showcased by using two real-life datasets to further support the applicability of this model as a clinical support decision tool. Indeed, in some embodiments, a validated, methodical prioritization of the multitude of genomic variants stemming from NGS analyses, as the one presented herein, has the potential to positively contribute towards the large-scale clinical application of pharmacogenomics and facilitate the translation of a patient's genomic profile into actionable clinical information.

### Methods

### Collecting the Training Data

Using some embodiments, an appropriate training set of variants was manually curated using the PGx Gene-specific information tables, created under the collaboration between PharmGKB and CPIC and was subsequently supplemented by additional variants from Pharm Var. This training set consists of 262 variants located across 12 pharmacogenes, with well-defined protein-level functional consequences, based on the integration and assessment of in vitro biochemical assays, in vivo evidence, and clinical observations. After careful data examination and owing to high percentages of missing values, 190 variants within 11 pharmacogenes (Table 4) remained and were used as our training set. The observed functionality is classified into 5 levels (excluding Unknown/Uncertain function): Increased, Normal, Possibly Decreased, Decreased and No function. However, owing to the limited number of observations harboring the levels of ‘Possibly Decreased’ and ‘Decreased’ functions and after careful examination of the available information for those categories, these two levels were combined in one class (Decreased function) (Table 3).

Table 3. Description of the protein function effect classes of PGx variants, which are used as the training data for the final RF model in an example embodiment. The functionality class is split into the following classes (‘decreased’, ‘increased’, ‘no’, ‘normal’), the number of the respective PGx variants per class is also provided, as well as which pharmacogenes are incorporated per each class.

| Functionality Class | Number of variants | Representation of genes |
|---|---|---|
| Decreased | 36 | DPYD, CYP2C19, CYP2C9, |
| | | SLCO1B1, RYR1, CYP2B6, |
| | | UGT1A1, CYP2D6 |
| Increased | 46 | RYR1, CYP2B6 |
| No | 48 | CYP2C19, CYP2C8, DPYD, |
| | | CYP2C9, NUDT15, CYP2B6, |
| | | CYP2D6, TPMT |
| Normal | 60 | DPYD, CYP2C9, SLCO1B1, |
| | | CYP2B6, CYP2D6 |

Table 4. Example list of represented pharmacogenes, which were included in the example training dataset of assessed machine learning models (AdaBoost, Multinomial logistic regression, Random Forest, XGBoost), according to an example embodiment.

| Pharmacogene | Description (HGNC) | Category |
|---|---|---|
| CYP2B6 | Cytochrome P450 family 2 | drug-metabolizing |
| | subfamily B member 6 | enzymes |
| CYP2C9 | Cytochrome P450 family 2 | drug-metabolizing |
| | subfamily C member 9 | enzymes |
| CYP2C19 | Cytochrome P450 family 2 | drug-metabolizing |
| | subfamily C member 19 | enzymes |
| CYP2D6 | Cytochrome P450 family 2 | drug-metabolizing |
| | subfamily D member 6 | enzymes |
| CYP2C8 | Cytochrome P450 family 2 | drug-metabolizing |
| | subfamily F member 8 | enzymes |
| DPYD | Dihydropyrimidine | drug-metabolizing |
| | dehydrogenase | enzymes |
| UGT1A1 | UDP glucuronosyltransferase | drug-metabolizing |
| | family 1 member A1 | enzymes |
| NUDT15 | Nudix hydrolase 15 | drug transporters and |
| | | non-drug metabolizing |
| | | enzymes |
| RYR1 | Ryanodine receptor 1 | drug transporters and |
| | | non-drug metabolizing |
| | | enzymes |
| SLCO1B1 | Solute carrier organic anion | drug transporters and |
| | transporter Family member | non-drug metabolizing |
| | 1B1 | enzymes |
| TPMT | Thiopurine S-methyltransferase | drug-metabolizing |
| | | enzymes |

### Variant Annotation

Using some embodiments, the curated set of pharmacogenomic variants was annotated using the web interface of Ensembl's Variant Effect Predictor (VEP) tool, for the GRCh38 human assembly, as well as the 4.1.a version of the dbSNFP database, which is also provided through VEP. The majority of the retrieved information is available for variants located within protein coding regions and includes: a detailed characterization at a protein level (i.e., database identifiers, codons, amino acids, coordinates, protein domains, computational scores, etc.), overlapping known variants, observed frequencies in different populations (i.e., via the 26000 Genomes Project, the genome Aggregation Database, the Exome Aggregation Consortium data and the Exome Sequencing Project), any related phenotypes (e.g., OMIM, Orphanet, GWAS catalog) or clinical significance (ClinVar), as well as literature references. Furthermore, the attributed consequence, described by using terms as developed in collaboration with Sequence Ontology (SO), and the corresponding impact of a variation are also provided.

Regarding the retrieved frequency data, variants were classified as ‘common’ if the minor allele frequency (MAF) was equal or above 10% (MAF≥10%) and as ‘intermediate’ if the MAF ranged between 5% and 10% (5%≤MAF<10%). Variants were classified as ‘low frequency’ if the MAF ranged between 1% and 5% (1%≤MAF<5%), whilst ‘rare’ variants included these variants of which the MAF was between 0.1% and 1% (0.1%≤MAF<1%). Finally, variants were classified as ultra-rare if the MAF was equal or below 0.1% (MAF≤0.1%).

Features and variants with a high percentage of missing values (≥40%) were excluded, while the remaining values were imputed by using k-Nearest Neighbors algorithm (kNN) with default values for k-neighbors (equal to 5) and inverse weighted mean Gower distances. In addition, a step of backwards variable selection through RFE using Bagged Trees was performed, which recommended the use of 7 out of the 45 variables (LoFtool, DEOGEN2_score, MPC_score, BayesDel_addAF_score, integrated_fitCons_score, FATHMM_score, LIST.S2_score). Furthermore, two binary variables were constructed and included in the analysis: one indicating whether the variant was located within a protein functional domain (according to InterPro annotation) and one representing high impact SO consequences (splice acceptor or donor variants, stop gained, frameshift variants, stop or start lost), enriched for Loss-of-Function (LoF) changes.

### Training of the Machine Learning Model

Preprocessing and ML-related analyses were performed using the R language for statistical programming (version 4.0.2). To exploit the abilities of the abovementioned features toward explaining potential protein function effects of variants derived from NGS analyses, a variety of tree-based methodologies was assessed, alongside with a special case of a neural network acting in a multinomial logistic regression manner. More specifically, Random Forests, Multi-class AdaBoost, XGBoost, and a neural network striped from its hidden layers and activation functions (multinomial logistic regression) were used via the caret package. For the selected tree-based models, hyperparameters were tuned based on the optimization of the accuracy metric, while in multinomial logistic regression, the default parameters were used (Table 5).

Table 5. Summary of example parameters and metric values for tree-based models (AdaBoost, Random Forest, XGBoost), as tested in some embodiments. Parameters denoted with an asterisk (*) were tuned according to the achieved accuracy.

| | | Selected |
|---|---|---|
| Approach | Parameters | values |
| Random | Number of trees (ntree) | 1,000 |
| Forest | *Number of variables randomly sampled as | 2 |
| | candidates at each split (mtry) | |
| AdaBoost | Maximum tree depth (maxdepth) | 5 |
| | *Number of trees (mfinal) | 1,000 |
| | *Coefficient type (coeflearn) | Zhu |
| XGBoost | *Boosting Iterations (nrounds) | 2650 |
| | Maximum Tree Depth (max_depth) | 5 |
| | *Shrinkage (eta) | 0.1 |
| | Minimum Loss Reduction (gamma) | 0 |
| | Subsample Ratio of Columns (colsample_bytree) | 0.8 |
| | Minimum sum of Instance Weight | 1 |
| | (min_child_weight) | |
| | Subsample percentage (subsample) | 1 |

### Evaluation of the Machine Learning Models

The predictive performance of the created models according to some embodiments was assessed via the 5-fold cross validation (CV) method. During n-fold CV, the data are divided to create n equal-sized subsets; n-1 of these are used to train a model and the remaining 1 is used to test its' performance. This process is repeated n times, until all subsets have been used to test the model, while the computed metrics in each iteration are averaged. More specifically, the metrics of interest include the Accuracy, Precision, Sensitivity (True Positive rate), Specificity (True Negative rate), Balanced accuracy (average of precision and recall) and the F-measure (harmonic mean of precision and recall). Since this was a multi-class task, all metrics were computed for each class separately (according to the one-vs-all method), and the performance of the model was calculated using the corresponding weighted average values for each metric. Furthermore, a random forest classifier was trained with the total of 47 features and used to evaluate their predictive importance.

### Testing the Applicability of the Final Machine Learning Model

To further demonstrate the applicability of the machine learning model as implemented in some embodiments, we applied the classifier in data derived from NGS analyses. To this end, variant call format (VCF) files comprised of the results from: (i) a WGS analysis of a single individual of Greek origin diagnosed with coeliac disease, and (ii) a targeted pharmacogene sequencing analysis of 304 individuals of Greek origin diagnosed with psychiatric diseases (71). Firstly, the provided variants were annotated, using the web interface of ensembl VEP tool, while the resulting data were preprocessed to select only these identified in the transcripts of interest. Then, these annotation data were used as an input to our final RF model and the corresponding prediction functionality classes and prediction probabilities were provided.

Last, clinical and variant annotations found in PharmGKB were also curated to extract clinically relevant information for the PGx variants either assessed or missed by the presented RF model.

An example embodiment of variant detection platform 2600 will now be described in relation to an example embodiment of interface generator 2650.

In some embodiments, interface generator 2650 is configured to generate a report displaying outputs from variant detection platform 2600, including externally retrieved data. For example, variant detection platform 2600 can be configured to accept as input the annotated variants identified in a patient's genome (provided as FASTQ, BAM or VCF format) and return a report, such as in an HTML or json format to viewed in a web application and as a pdf. An example report generated by interface generator 2650 is shown in FIG. 38A, FIG. 38B and FIG. 38C. Variant detection platform 2600 can be configured as a web-based platform, according to some embodiments. For example, one or more local computing devices can be used to access variant detection platform 2600 over a network, where variant detection platform 2600 is hosted on a remote server. Variant detection platform 2600 can be configured as a desktop platform, according to some embodiments. For example, variant detection platform 2600 can be run locally or on a local network.

In an example implementation, variant detection platform 2600 is configured to receive genomic data and perform the following functions based on the genomic data. Firstly, variant detection platform 2600 is configured to select known effect variants, which are part of the functionally annotated training set extracted from Pharmacogenomics Knowledge base and the Pharmacogenomics Variation Database and which was used to train the classifier. Secondly, variant detection platform 2600 is configured to select variants that do not overlap with the training set of the classifier and for which the model predicts that they could lead to altered protein function (Increased, Decreased, No function).

In an example implementation, the variants that are extracted from these two levels are supplemented by FDA, EMA as well as PharmGKB information. The additional FDA and EMA information includes details about the dosing scheme regarding certain drugs, for which the guidelines are dependent upon the metabolizer status of the patient. Therefore, in cases where certain drugs are metabolized by enzymes encoded by pharmacogenes, as assessed by the variant detection platform 2600, a summary of FDA and EMA guidelines and extra information is provided. It's worth noting that normal or extensive metabolizers are usually the result of two functional alleles, while ultra-rapid metabolizers refer to individuals in which the respective enzymes present higher activity and come from different combinations of increased function alleles. Finally, intermediate, and poor metabolizers result from the combination of decreased, no-function and/or normal function variants and the respective enzymes present lower activity.

This report presents the results on a (pharmaco) gene-level basis in two separate sections as follows, according to some embodiments.

In the first section, an overview is presented, describing how many variants are potentially affecting drug response. More specifically, a table with the number of variants per functional class (increased, decreased, no function) is presented, while these variants are also further subdivided according to their novelty and the level of evidence, as explained earlier.

In the second section, a summary table of drugs interacting with each assessed pharmacogene is provided, as extracted by FDA.

In this example report, the data provided to variant detection platform 2600 come from a single individual of Greek origin diagnosed with coeliac disease. Overall, 2 function-modifying variants were identified, one in the drug metabolizing enzyme CYP2B6 and one in drug transporter SLCO1B1. In CYP2B6, a known, decreased function variant is found, while FDA provides further information for poor metabolizers administered efavirenz. For example, the patient can be a homozygote for this decreased function allele, and the FDA notes could apply for the patient.

Finally, another known and decreased function variant was found in SLCO1B1. Here, FDA provides information for the associations between intermediate or poor transporters and 4 drugs: three statins used to lower cholesterol-atorvastatin, rosuvastatin and simvastatin, and elagolix, a gonadotropin-releasing hormone antagonist (GnRH antagonist), used for the management of moderate to severe pain associated with endometriosis.

All this information can be presented by an example embodiment of interface generator 2650 and be useful towards facilitating the pharmacogenomic variant interpretation procedure, as well as identifying key variants that could affect drug response through altering the protein function.

FIG. 40, FIG. 41, FIG. 42, FIG. 43, and FIG. 44 show example user interface objects generated by interface generator 2650, according to some embodiments. An annotated variant dataset can be received by variant detection platform 2600 at a user interface similar to that shown in FIG. 40, for example.

### Generating an Annotated Variant Dataset

An example method for generating an annotated variant dataset will now be described, according to some embodiments. The annotated variant dataset can be received by variant detection platform 2600, such as by variant validator 2620, machine learning model 2630, and/or loss-of-function detector 2640. FIG. 39 shows an example process for generation of an annotated variant dataset, according to some embodiments.

In some embodiments, a raw FASTQ, CRAM, or BAM file of a person provided by a genome sequencing center is transformed into a variant calling format (VCF) file (or genomic variant calling format containing information about the variants that the person has in their DNA (deoxyribonucleic acid). The method that the VCF is then transformed into a text file with annotated variants which is the input for the variant detection platform 2600, according to some embodiments.

According to some embodiments, the transformation of the initial FASTQ, CRAM or BAM file to VCF is implemented using the Genomic Analysis Tool Kit (GATK). In other embodiments, other methods for variant calling can be used. GATK provides several ready-to-run workflows for the analysis of Whole Genome Sequencing (WGS) and Whole Exome Sequencing (WES) data, including WGS germline short variant calling (SNPs and indels) and WES germline short variant calling (SNPs and indels). Cloud computing (e.g., Google Cloud) can be used to effectively run a workflow. The first step is the creation of a cloud computing service account, virtual machines (VMs), cloud storage, and enabling of cloud genomics APIs. Given a bucket storage that contains the initial FASTQ, CRAM, or BAM file and a computer (e.g., Linux machine) with access to the project, the tool wdl-runner provided can be used to run any of the workflows of interest (e.g., from a source code repository such as GitHub). Workflows can be created with WDL and Cromwell, for example, allowing for ease of use and changes. Other infrastructure can be used, such as by running the workflows on a single computer or using HPC infrastructure, Google Cloud, Amazon Web Services, Microsoft Azure Cloud, Alibaba Cloud, or other infrastructure. The appropriate JSON files of the workflow are updated to match the cloud computing service storage paths when a workflow is run. After updating the files, the workflow can run with wdl-runner which can automatically create and shut down VMs uploading only the output files and logs on the cloud computing service storage. Since several workflows are needed to transform the initial file to VCF the workflows can be organized in a script which can automate the whole procedure.

According to some embodiments, two methods were developed for the analysis of WGS data. Workflows that are used by GATK are listed as follows.

In method one, the following workflows are used (with the workflows described herein as optional being optional): seq-format-conversion, seq-format-validation, gatk4-data-processing, gatk4-germline-snps-indels, bcftools, and broad-prod-wgs-germline-snps-indels: JointGenotypingWf.

The seq-format-conversion workflow is used in order to transform the initial FASTQ, CRAM, or BAM file in unmapped BAM (uBAM) format which is the format used as input for GATK tools to run. The tool provides several options according to the type of input file.

The seq-format-validation (optional) workflow tests if the uBAM file produced by the seq-format-conversion tool is of a valid format. The workflow can report any errors.

The gatk4-data-processing workflow accepts as input the uBAM file produced by the seq-format-conversion tool and 1) aligns the reads to human genome hg38 using the BWA-MEM aligner, 2) marks duplicated reads in order to not be taken into account more than once, and 3) recalibrates the base quality scores of the reads using a tool called BQSR. The output of the workflow is an analysis ready BAM and its index.

The gatk4-germline-snps-indels workflow accepts as input the BAM produced by the gatk4-data-processing workflow and identifies the variants of the person using a tool called HaplotypeCaller. The output of the workflow can be either a VCF or a gVCF in raw unfiltered format.

If the final output is in VCF format, bcftools (and/or GATK SNPs and INDELS filtering recommendations) can be used to hard filter the data according to the recommendation provided by GATK.

If the final output is in gVCF format, the tool broad-prod-wgs-germline-snps-indels: JointGenotypingWf can be used to filter the variants according to a pre-trained deep learning model called VQSR.

In method two, the following workflows are used (with the workflows described herein as optional being optional): seq-format-conversion, seq-format-validation, broad-prod-wgs-germline-snps-indels, and broad-prod-wgs-germline-snps-indels.

The seq-format-conversion workflow is used in order to transform the initial FASTQ, CRAM or BAM file in unmapped BAM (uBAM) format which is the demanded format for GATK tools to run. The tool provides several options according to the type of input file.

The seq-format-validation (optional) workflow tests if the uBAM file produced by the seq-format-conversion tool is of valid format.

In the broad-prod-wgs-germline-snps-indels workflow, the PairedEndSingleSample Wf workflow accepts as input the uBAM file produced by the seq-format-conversion tool and 1) aligns the reads to human genome hg38 using the BWA-MEM aligner, 2) marks duplicated reads in order to not be taken into account more than once, and 3) recalibrates the base quality scores of the reads using a tool called BQSR. The produced BAM is then used to identify the variants of the user (e.g., person) using a tool called HaplotypeCaller. The output of the workflow is a raw unfiltered g VCF.

In the broad-prod-wgs-germline-snps-indels workflow, the JointGenotypingWf workflow can be used to filter the variants according to a pre-trained deep learning model called VQSR.

In either method, after running the workflow(s), all intermediate files can be removed and only the initial file and the final filtered VCF kept.

The VCF file produced by the described methods contains the variants that the person has. According to some embodiments, the next step of the method is the annotation of those variants using Ensembl Variant Effect Predictor-VEP as well as its plugin database dbNSFP. This annotation step is important since the new variables that are provided for the variants are needed to be able to run a predictive model in variant detection platform 2600. To annotate the VCF file, the VEP command line tool as well as the dbNSFP database can be installed in a Linux machine. Using this tool, the variants are annotated using the following settings: transcript database to use: Ensembl/GENCODE and RefSeq transcripts; Identifiers: Gene symbol, Transcript version, CCDS, Protein, Uniprot, HGVS; Find co-located known variant options: TRUE; Protein domains; SIFT: Prediction and score; PolyPhen: Prediction and score; Extra database: dbNSFP; DbNSFP required fields: All; All other parameters in VEP set to default values.

The output is an annotated text file that can be used as input to variant detection platform 2600. For example, the annotated txt file can be the annotated variant dataset.

FIG. 45A and FIG. 45B show an example interface for a VEP, according to some embodiments. VEP can be accessed using an account provided by an email address, for example. FIG. 46A and FIG. 46B show VEP parameters on an example interface, according to some embodiments. In some embodiments, VEP parameters include: transcript database to use (e.g., Ensembl/GENCODE and RefSeq transcripts); Identifiers: Gene symbol, Transcript version, CCDS, Protein, Uniprot, HGVS; Find co-located known variant options: TRUE; Protein domains; SIFT: Prediction and score; PolyPhen: Prediction and score; Extra database: dbNSFP; DbNSFP required fields: All; and all other parameters in VEP set to default values.

Data used by variant detection platform 2600 can include: classifier, variants used for training, list of transcripts to select, curated FDA recommendations and PharmGKB information, and VEP annotated input data. Rmarkdown can be used to create a report, according to some embodiments. Rshiny can be used for the application, according to some embodiments.

FIG. 47, FIG. 48A and FIG. 48B show example interfaces generated by interface generator 2650, according to some embodiments.

In some embodiments, variant detection platform 2600 is configured to protect patient anonymity. In some embodiments, variant detection platform 2600 is configured for ongoing use, such as real-time new predictions.

In some embodiments, variant detection platform 2600 is configured for full genomic and pharmacogenomic scanning. In some embodiments, variant detection platform 2600 is configured for multiomics, integrative omics, or panomics purposes. Multiomics includes genomics, but also pharmacometabolomics, transcriptomics, proteomics, metabolomics, epigenomics, microbiomics, and biomarkers. These can provide valid diagnostic tests for the qualitative (type of disease) and the quantitative (new or progressing disease or response to treatment) diagnosis of various diseases.

In some embodiments, variant detection platform 2600 is configured for predicting the presence of disease before any symptoms appear and continuous monitoring can confirm if treatment is beneficial or not to the patient. Certain pathological proteins and metabolites can be data input.

In some embodiments, variant detection platform 2600 is configured for assessment of complicated data obtained from the multiomics family in a holistic manner to define geno-pheno-envirotype relationships involved in various diseases.

In some embodiments, variant detection platform 2600 is configured for 1) prediction of disease, 2) initiation of disease, and 3) treatment and follow-up of the disease over the years to come.

In some embodiments, variant detection platform 2600 can be used for detecting genomic variants leading to altered protein function. In some embodiments, variant detection platform 2600 is a machine learning-based clinical decision support tool for medical doctors to use to select the appropriate drug and the drug dose for each patient. In some embodiments, variant detection platform 2600 provides a precision and personalized medicine tool. In some embodiments, variant detection platform 2600 is a translational tool based on machine learning and artificial intelligence, correlating the occurrence of genomic variants (as established by whole genome sequencing) as a means to select the best potential drug therapy that can be used in personalized medicine to maximize drug efficacy and minimize drug toxicity. In some embodiments, variant detection platform 2600 is configured for use with blockchain technology.

In some embodiments, variant detection platform 2600 can use previously published data from the literature as training datasets of the machine learning model to be able to predict the effect of novel variants that are identified using whole genome sequencing. This can help allow personalized medicine to be employed in clinical settings. In some embodiments, variant detection platform 2600 can provide dosing guidelines and can be extendable to correlating variants with disease and intervention. In some embodiments, variant detection platform 2600 uses data from published results of researchers that report mutations and corresponding responses to various drugs.

In some embodiments, variant detection platform 2600 is a bioinformatics platform that utilizes machine learning and artificial intelligence to translate a patient's genomic profile into easy-to-understand information presented in a unique way to clinicians. In some embodiments, variant detection platform 2600 can be used to help rationalize pharmacotherapy and improve the effectiveness of prescribed medication, while reducing the risk of the occurrence of adverse drug reactions.

In some embodiments, variant detection platform 2600 is configured to identify and assess known as well as novel variants, while also providing drug dosing guidelines, that are automatically retrieved from regulatory bodies such as the FDA and the EMA, for each gene in which variants that potentially affect protein function were found.

As a result, variant detection platform 2600 can appeal to both clinicians and diagnostic facilities and can be used to support the selection of the appropriate medication and/or dose. In some embodiments, variant detection platform 2600 can help facilitate personalized medicine, such as to address cancer, without using trial and error in treatment strategies. According to the FDA, adverse drug reactions are the fourth leading cause of death in the United States. In some embodiments, variant detection platform 2600 can help reduce medication-associated errors and associated monetary costs, as well as psychological and physical pain and suffering. In some embodiments, variant detection platform 2600 can help reduce the incidence of patients not responding well to drugs prescribed to them, as well as hospitalizations and wrong prescriptions.

In some embodiments, variant detection platform 2600 uniquely can be configured to determines novel and rare variants of pharmacogenes, without simply relying on compiled reporting of such data, as compiled reports may not be up-to-date or accurately described.

In some embodiments, variant detection platform 2600 is configured to identify DNA variations which will lead to a dysfunctional or non-functional enzymes that will not metabolize a drug of interest.

In some embodiments, variant detection platform 2600 is configured to identify drug alterations, such those that may be suitable or unsuitable for use in view of variants identified by variant detection platform 2600.

In some embodiments, variant detection platform 2600 is configured to translate the genetic profile of a patient into a format that a treating physician can understand within minutes to identify the best medication. For example, variant detection platform 2600 can be configured to detect adverse drug reactions, according to some embodiments. Variant detection platform 2600 (e.g., at its machine learning model) can be modified or optimized for particular purposes or genomes or variants.

In some embodiments, variant detection platform 2600 is configured to translate a patient's genes into personalized drug recommendations, such as to maximize drug effectiveness and minimize drug toxicity.

In some embodiments, variant detection platform 2600 is configured to calculate the likelihood of a novel and/or rare pharmacogene variant being pathogenic or not and as such to be actionable biomarkers. Based on the calculated score, variant detection platform 2600 is configured to fetch corresponding guidelines from data sources, such as database(s) storing data from regulatory bodies (FDA, EMA, etc) and research consortia (CPIC, DPWG).

In some embodiments, variant detection platform 2600 is configured to use machine learning to calculate the pathogenicity scores of novel and/or rare variants in pharmacogenes. Well-described and known clinically actionable pharmacovariants can be used as training datasets.

In some embodiments, variant detection platform 2600 is configured to allow an exhaustive basepair-by-basepair scanning of clinically actionable pharmacogenes hence providing a comprehensive scan of pharmacogenes and as such a more complete reporting of pharmacovariants towards maximizing drug efficacy and minimizing drug toxicity.

In some embodiments, variant detection platform 2600 is configured for machine-learning based calculation of the pathogenicity scores of novel and rare pharmacovariants and the automatic fetching of the corresponding guidelines from the regulatory bodies and research consortia, helping facilitate accuracy and being up-to-date.

In some embodiments, variant detection platform 2600 is configured to allow translation of a genome or sets of genomes into clinically meaningful information. In some embodiments, variant detection platform 2600 is configured to acquire a human genome sequence in digital format. The digital file is uploaded to variant detection platform 2600 and, in a short amount of time of processing, the patient or clinician receives the response/guideline to optimize the required prescription and/or drugs required. In some embodiments, variant detection platform 2600 is configured to be implemented in cloud and web-based technology to analyze either partial or the whole human genome sequence at an affordable cost per case. While others may analyze only specific variants, in some embodiments, variant detection platform 2600 is configured to scan an entire genome sequence and provide reports according to FDA and EMA recommendations. Once a genome is sequenced it never has to be sequenced again, for example.

Variant detection platform 2600 can be configured for use and its outputs applied for purposes in insurance, personalized medicine, large clinics, small clinics, pharmaceutical industry, research and development, national health systems (e.g., OHIP, QHIP, or ABHC in Canada; medicare in the USA), diagnostic laboratories, hospitals, in vitro fertilization, and in medical practices, and/or by corporations' employees, healthcare professionals, or genomic related service providers. Healthcare professionals can include doctors such as cardiologists, oncologists, psychiatrist, neurologists, immunologists, hematologists, pathologists, nephrologists, and can also include internists. Variant detection platform 2600 can receive as input and can be used for detection of variants in any aspect of a genome, human or otherwise. Genetic data can be derived from any people group or country or jurisdiction, such as Canada, the USA, Europe, Japan, Australia, Malaysia, Singapore, Saudi Arabia, and/or China. Variant detection platform 2600 can generate new outputs as well as present the outputs in an improved user interface that facilitates the determination of the correct drug at the correct dose at the correct time for a particular patient. Variant detection platform 2600 can use machine learning to provide new understanding of an individual's genetic profile derived from whole genome sequencing on an ongoing basis, allowing for a predictive genomics service applied to clinical decision making. Variant detection platform 2600 can be used for disease prevention, early detection, diagnosis and treatment, and management of the quality of life of patients and survivors of disease.

As used herein, memory refers to non-transitory memory.

Example alternative embodiments will now be described. The following does not limit the scope of the foregoing embodiments, and the statements are to be understood as describing an alternative embodiment.

Described herein is a method for classifying the effect of an unknown genetic variant of a patient on the patient's response to medication.

The rapid progress of next generation sequencing (NGS) technologies has boosted the deciphering of genomics information on an ever-growing scale. Although this massive progress of the NGS field often led to the discovery of genomics variants and the association of genes with disease phenotypes, the implementation of these findings in clinical trials requires careful consideration. Genes implicated in the processes of drug absorption, distribution, metabolism and excretion (ADME) are highly variable, with this variability reported to contribute to the inter-individual differences in drug response. Interestingly, most of ADME variants have not been experimentally characterized, thus hindering the clinical interpretation of genetic variability and making the translation of genomic data into actionable advice difficult. The potential availability of a vast number of identified genomics variants in a clinical setting emphasizes the importance of developing a method to systematically evaluate and prioritize this information in a format that should be exploited towards guiding medication or dosing choice.

As highlighted in the analysis of NGS derived data, most of the variants located in protein coding genes may be rare, population-specific and enriched in potentially damaging non-synonymous genetic changes. Moreover, the volume of the identified variants makes the experimental evaluation of their effects on protein function difficult or even prohibitive, therefore computational methods have been proposed to predict the functional effect of the identified genomics variants.

Existing in-silico approaches rely heavily on evolutionary conservation and/or structural information, while focusing mainly on disease-associated variations. As a result, when applied to the less constrained pharmacogenes, the outcome is usually conflicting predictions, either amongst the different tools or compared to in vitro or in vivo observations. Nevertheless, the in-silico scores used to predict the functional impact of variants are, especially in the absence of any other experimental or clinical data, a valuable and easily accessible source of information, which, can lead to more reliable predictions, when combined. In addition, the use of in vitro functionally characterized pharmacogenetic variants for the optimization of prediction thresholds and the integration of the best performing combination of algorithms can lead to a highly efficient framework for the evaluation of genetic variants and their categorization as neutral or Loss-of-Function (LoF).

The technological advances of NGS technology over the last decades have led to the identification of a vast number of genomic alterations, most of which are characterized as variants of unknown significance, since their numbers render the experimental or clinical determination of their consequences impossible. Computational tools may aim to prioritize variants that are expected to significantly affect the functionality of the corresponding proteinic products, usually by calculating a score based on certain parameters and by classifying the variants as “Benign” or “Pathogenic/Deleterious”. However, due to the least conserved nature and the characteristics of genes responsible for drug metabolism and transportation (ADMET genes or pharmacogenes), those in silico tools have proven to be inappropriate for the prioritization of variants located in these genes.

Determining the functional consequences of pharmacogenomics variants is of utmost importance for the adjustment of a patient's pharmacotherapy scheme in a way that maximizes clinical benefits, whilst minimizing the risk of Adverse Drug Reactions (ADRs). To achieve that, special caution must be given in the collection of an appropriate training set, as well as in the determination of informative variables that will be used for the training of a machine learning (ML) by algorithm, thus creating a variant classifier.

Existing approaches may only, at most, evaluate variants with regards to their protein damaging potential, without considering variants that lead to products with increased protein function and which are of paramount importance in the field of pharmacogenomics.

Pharmacogenomics studies the way a person's genome affects their response to a given medication, aiming to utilize this information to guide and personalize the treatment in a way that maximizes the clinical outcome while minimizing the dangers for the patients, thus fulfilling the promises of precision medicine. Technological advances in genome sequencing, combined with the development of improved computational methods for the efficient analysis of the huge amount of the obtained data, allow for the fast sequencing of a patient's genome, and with reduced cost, making its incorporation into clinical routine practice a realistic possibility. The potential availability of the vast number of identified genetic variants in a clinical setting highlights the necessity of developing a method for systematic and of effectively evaluating and prioritizing this information to be exploited towards the guidance of medication or dosing choice. A machine learning approach is provided to identify and classify novel or rare pharmacogenomics variants about their possible effects on protein function, which in turn affect drug response. This approach performs data-driven pharmacogenomics variant prioritization, and it is thus recommended as a powerful pharmacogenomics scoring tool with a variety of clinical applications.

Tools or frameworks can be developed where the effect of a variant is considered as being either “neutral” or “Loss-of-Function”, or equivalently, as “Benign” or “Pathogenic/Deleterious”.

However, this classification is not optimal because a variant does not necessarily need to abolish protein function to alter a patient's response in each medication. Effects other than a pathogenic effect or the absence of an effect need to be accounted for.

Indeed, increased metabolism of a substrate, for example, can equally affect the available drug levels and, as a result, lead to toxicity and adverse reactions. A proposed method is to categorize variants as being potentially damaging or not, but also to expand beyond those two commonly evaluated categories, to integrate variants that can lead to proteins with increased function. The method is trained to be able to identify and populate other classes as well, into which unknown variants will be classified according to the method described herein.

According to an embodiment, a machine learning-based method is implemented to perform genomics data processing and interpretation in relation to clinical decision, support and clinical trial design for the determination of drug efficacy and safety for an individual or group of individuals. Techniques of bioinformatics and machine learning algorithms are used to prioritize and predict (or more precisely, to classify) the protein function effect of novel (unknown), known or rare variations in ADMET (absorption, distribution, metabolism, excretion and transport) genes, as derived from next generation sequencing data.

More precisely, according to an embodiment, a machine learning approach is implemented to associate genotype level information, as derived from a standard NGS pipeline (in VCF format), with the potential changes in protein function. Towards this end, a method can be implemented as in the flowchart of FIG. 49.

At step 2410, data is collected. Several computationally determined “pathogenicity” scores are acquired.

At step 2420, variant annotation is performed with the use of common platforms (such as Ensembl's Variant Effect Predictor, VEP), are used. A training set of well-established and thoroughly studied variants located in pharmacogenes is extracted, and preferably curated, for example from the Gene-Specific Tables, resulting from the collaboration between PharmGKB and CPIC (Clinical Pharmacogenetics Implementation Consortium).

At step 2430, extensive data pre-processing and imputation take place for any missing values on the in-silico protein prediction scores (which can use, for example and without limitation, the missForest R package). The resulting data are used (step 2440) to train a random forest model, which is an ensembl classifier, with the purpose of categorizing unseen (i.e., previously unknown and uncharacterized) pharmacovariants. This categorization corresponds to their expected effect on the proteinic product of the gene they affect (i.e., the gene in which the variant is located). Model training also involves hyperparameter selection (step 2450) to obtain the most efficient machine learning method.

The classification task performed in the method classifies the variant into one of four possible functionality classes (Increased, Decreased, Normal, and No Function) which reflect the expected change on protein activity, compared to the wild type product, where ‘Increased’ means augmented protein function, ‘Decreased’ equals to reduced functionality, ‘Normal’ means unchanged functionality, while ‘No’ corresponds to proteinic products with demolished function.

The functional result is a trained classifier which can classify a “new” variant (i.e., a priori unknown variant) depending on the expected effect on protein function and which can be applied on said new variant (step 2460) to perform the classification task.

According to an embodiment, a machine learning method is trained by using a curated set of pharmacogenomics variants, of which the functional implications have been determined through clinical observations, biochemical experiments, or ideally both. The training variables corresponding to these variants are 23 in silico determined scores (SIFT, Mutation Assessor, LRT, PolyPhen-2, M-CAP, FATHMM-MKL, Eigen, Eigen-PC, GenoCanyon, CADD, DANN, MetaLR, MetaSVM, REVEL, Condel, BLOSUM62, phastCons, PhyloP, SiPhy, GERP++, fitCons, LoFtool, MCP), collected through variant annotation performed via Ensembl's Variant Effect Predictor (VEP) tool. The variables correspond to conservation characteristics, in-silico protein damaging scores and gene level scores, in some embodiments.

According to an embodiment, these variants, comprising the training set, are classified (as mentioned above) into 4 categories with regards to their expected consequences on protein function (Increased, Normal, Decreased and No Function), as determined by the trained machine learning algorithm. When running the method, a Random Forest algorithm was used, and the derived model has shown very satisfying results (Balanced Accuracy=0.86, Sensitivity=0.8, Specificity=0.92, F1-score=0.82). Overall, available information is exploited to train a classifier that could efficiently predict the potential protein function effects of newly identified genomic variants.

Referring, for example, to step 2450 mentioned above with respect to FIG. 49, the approach contemplated herein requires minimal hyperparameter tuning, due to the nature of the algorithm used. Moreover, it makes use of easily accessible information for a newly identified variant, while it also considers a range of functional consequences, rather than two ends of a limited spectrum. More precisely, the method as described herein involves multiclass classification (i.e., more than two classes) rather than having two commonly used levels (i.e., benign or damaging) to account for other effects which were previously unaccounted for. The outcome is preferably discrete instead of continuous (i.e., the method is preferably a classifier instead of an estimator).

It should be noted that experimental evaluation of each variant can be extremely time-consuming as well as being notoriously impractical due to the high number of variants and rarity of many of them.

The method makes possible a systematic evaluation of variants located in pharmacogenes and their prioritization in terms of their potential of predicting drug response and toxicity. Pharmacogenes are the genes involved either on the pharmacokinetics or pharmacodynamics of a certain drug, or which are implicated on the development of Adverse Drug Reactions (ADRs) or lack of efficacy in certain drugs. Variants located in pharmacogenes have been shown to modify a patient's drug response, thus affecting the therapeutic outcome. The technological advances of Next Generation Sequencing (NGS) techniques over the last decades led to the identification of many genomic alterations, most of which are characterized as variants of unknown significance, since their numbers render the experimental or clinical determination of their consequences impossible. As a result, computational tools and scores have been developed, thus aiming to prioritize variants that are expected to significantly affect the functionality of the corresponding proteinic products. Although such in silico tools are considered important evidence when evaluating disease-related variants, their application in pharmacogenomics-related variants is complicated by the least conserved nature and the characteristics of genes responsible for drug metabolism and transportation (DMET genes or pharmacogenes).

The knowledge, or at least the tentative classification performed in a predictive manner by a method herein described in some embodiments, of the expected effects of a newly identified pharmacovariant can assist in the determination of an appropriate therapeutic scheme for patients carrying this specific variation, and since the majority of variants identified through NGS techniques has not been experimentally characterized, in silico scores used to predict the functional impact of variants can be a valuable and easily accessible source of information, especially in the absence of any other experimental or clinical data, which can lead to more reliable predictions, when combined together. Furthermore, a variant does not necessarily need to abolish protein function to alter a patient's response in each medication-increased metabolism of a substrate for example can equally affect the available drug levels and as a result lead to toxicity and adverse drug reactions, hence the multiclass classification contemplated herein which accounts for the other effects.

In some embodiments, the instant method categorizes variants as being potentially damaging or not, as well as expanding beyond those two categories. The multiclass classification categorizes unseen pharmacovariants depending on their expected effect on the protein product of the gene they affect. As mentioned above, the four possible functionality classes (Increased, Decreased, Normal, and No) reflect the expected change on protein activity, compared to the wild-type product, where ‘Increased’ means augmented protein function, ‘Decreased’ equals to reduced functionality, ‘Normal’ means unchanged functionality, while ‘No’ corresponds to proteinic products with demolished function.

According to an exemplary embodiment, and without limitation, the entire tool can be developed using the R programming language and accepts as input the annotated data (as text files) resulting from a standard NGS pipeline (for example, in VCF format). Using this method, there is a potential to promote the clinical interpretation of genetic variability and facilitate the translation of genomic data into clinically actionable advice.

Now referring more specifically to the training (step 2640 mentioned above with respect to FIG. 49), the training variables are briefly summarized in the table below, categorized according to their underlying principles. Except for SIFT, PolyPhen, Condel, LoFtool and MCP, which were directly extracted from VEP's plugin function, the remaining scores were collected from dbNSFP 3.5 (also accessed through VEP). Presentation of the 23 in silico scores that were used as training variables are as follows.

| | |
|---|---|
| Based on a theoretical model | SIFT, Mutation Assessor, LRT |
| Supervised ML-approaches | PolyPhen-2, M-CAP, FATHMM-MKL |
| Unsupervised ML-approaches | Eigen, Eigen-PC, GenoCanyon |
| Ensembl systems | CADD, DANN, MetaLR, MetaSVM, |
| | REVEL, Condel |
| Conservation scores | BLOSUM62, phastCons, PhyloP, |
| | SiPhy, GERP++, fitCons |
| Gene-level evaluation methods | LoFtool, MCP |

In some embodiments, a step during the training of the machine-learning algorithm is parameter tuning (step 2650 mentioned above with respect to FIG. 1). Those steps comprise the step of selecting the optimal value of number of decision trees that comprise the random forest (ntrees)—a factor that should ideally be set as high as possible, to achieve stable predictive performance without significantly increasing the computational cost—and then tuning appropriately the number of selected variables (mtry). The training of the model was conducted based on forest's random implementation in the randomForest R package, accessed through its wrapper in the caret R package, which was also used for the parameter tuning steps.

This involved the development of a framework for the systematic evaluation of variants located in pharmacogenes and variant prioritization in terms of their potential of affecting/modifying the protein function. A total of 199 SNVs covering 12 genes have been collected and processed to this end. Initially, the variants were characterized via Ensembl's Variant Effect Predictor web tool, and after re-processing to manage mismatches, inconsistencies and missing values, a final set of 156 SNVs spread over 10 pharmacogenes was used to train a multiclass classifier.

In this step, two sorting models were finally created, one that distinguishes between 5 possible classes (Balanced Accuracy=0.85, Sensitivity=0.77, Specificity=0.92, Precision=0.78, F1-score=0.77), and one between 4 (Balanced Accuracy=0.86, Sensitivity=0.8, Specificity=0.92, F1-score=0.82). Therefore, the empirical results show that a multiclass classification into four classes is the promising in terms of balanced accuracy. Both models are proven to be more effective in detecting variants that lead to increased activity, and less effective for the other categories examined. In addition, both models indicate that the gene-level scores LoFtool and MPC are the most important parameters with a significant difference from the rest, as shown in FIG. 50. LoFtool evaluates the tolerance of a gene in LoF mutations.

More specifically, FIG. 50 illustrates the variable importance plot extracted from the model trained with the original 156 SNVs, categorized into 5 classes. LoFtool is proposed as the most valuable predictive feature. The same observations are repeated when the same 156 variants are categorized into 4 classes (after merging Possibly Decreased and Decreased into one class).

The improved predictive potential towards variants conferring increased function as well the importance of LoFtool can be explained when considering that all “Increased function” training variants belong to the same gene, and as a result, having the same value of LoFtool is expected. To correct such bias, LoFtool was removed from the training variables and the analysis was re-run. The results for the Variable Importance plot for the model trained with the original 156 SNVs (in 5 classes), after removing LoFtool from the training variables, are shown in FIG. 51. The models created were still characterized by promising classification metrics, while they still had an improved performance towards the “Increased function” class.

FIG. 52 illustrates performance metrics computed for the 5-class containing training set, with (all variables) and without (no gene-level variables) LoFtool as a training feature, while FIG. 30 illustrates performance metrics computed for the 4-class containing training set, with (all variables) and without (no gene-level variables) LoFtool as a training feature.

As shown in FIGS. 27 and 28, despite the removal of the variable that was thought to be the biased reason for the impressive performance of the trained models towards “Increased” function variants, the observed metrics remain highly promising. Furthermore, it can be noted that the models trained with the original data, split into 5 unbalanced classes perform poorly with regards to “Decreased” function variants (FIG. 4), with the results being equal to random guess. However, the models' behavior is significantly improved when Possibly Decreased and Decreased classes are unified, creating a new class (FIG. 53). The choice to perform this merging is justified when considering that biologically those classes are substantially identical-their only difference is the level of existing evidence that supports the functional characterization of the corresponding gene products.

This method could be useful in the prioritization of novel or existing variants of unknown significance-located within genes implicated in drug response—with regards to their potential to alter the protein function (whilst also evaluating the direction of this alteration, e.g. upwards or downwards). As a result, emphasis could be given to specific and potentially actionable variants (in a clinical setting), thus saving valuable resources. Also, the present method could be potentially part of an integrated PGx-variant interpretation and pharmacotherapy guiding toolkit.

The method may further include treating, for a condition, the subject in which the unknown genomic variant was analyzed. The predicted effect of the unknown genomic variant may be categorized as having a given effect on protein metabolism or function which would modify the effect of a treatment, e.g., increasing, decreasing, destroying or having no effect on a given treatment. It follows that for the subject, a treatment may be associated with a better prognosis, and with a better treatment outcome than other treatments in view of how one or more of the genomic variants of that subject is or are classified.

Therefore, the choice of an active compound to be given to the subject, or more generally the choice of the treatment, may be adapted in view of the classification, and the method finally comprises the step of treating the subject accordingly. In other words, the subject's condition's response to a given treatment can be predicted depending on how the subject's genomic variant(s) is or are classified, and the treatment determined as being the best treatment in this context is administered to the subject afterwards. For example, if the condition is cancer, the treatment may include surgery, radiation therapy, chemotherapy, immunotherapy, targeted therapy, hormone therapy, stem cell transplant, or precision medicine, as chosen to optimize the outcome of the treatment in view of the predicted effect of the unknown genomic variant.

The method may further include a confidence level or probability for the level of the condition or the effect of each treatment on the condition in view of the classified effect of the genomic variant, or the likelihood of the classification itself. Based on such determined levels of confidence, a treatment plan can be developed to decrease the risk of harm to the subject or optimize the outcome. Methods may further include treating the subject according to the treatment plan.

Finally, the method may further include diagnosing the subject by detecting that the genomic variant is classified as having an effect which is determined to be a medical condition to be treated, and eventually the method may further include treating the patent for this condition.

In some embodiments, there is provided a method for classifying the predicted effect of unknown genomic variants on protein function affecting drug response, the method comprising the steps of: collecting, as an input, genotype level information; for each variant in the genotype level information, associating a plurality of scores, the plurality of scores comprising a gene-level evaluation score; training a model of classification based on the plurality of scores for each of the variants of the inputted genotype level information, into four or more classes of a predicted change on protein activity; and applying the model for an unknown variant to classify said unknown variant into one of the four or more distinct classes.

In some embodiments, the four or more distinct classes comprise exactly four classes which comprise: an ‘Increased’ class reflecting augmented protein function; a ‘Decreased’ class reflecting reduced functionality; a ‘Normal’ class reflecting unchanged functionality; and a ‘No’ class reflecting proteinic products with demolished function.

In some embodiments, the four or more distinct classes comprise exactly five classes which comprise: an ‘Increased’ class reflecting augmented protein function; a ‘Decreased’ class reflecting reduced functionality; a ‘Possibly Decreased’ class reflecting a possibly reduced functionality; a ‘Normal’ class reflecting unchanged functionality; and a ‘No’ class reflecting proteinic products with demolished function.

In some embodiments, the plurality of scores for training comprise a score from an Ensembl system, a conservation score and the gene-level evaluation score.

In some embodiments, the gene-level evaluation score comprises a LoFtool score.

In some embodiments, the gene-level evaluation score comprises at least one of LoFtool and MCP.

In some embodiments, the score from an Ensembl system comprises at least one of CADD, DANN, MetaLR, MetaSVM, REVEL, and Condel.

In some embodiments, the conservation score comprises at least one of BLOSUM62, phastCons, PhyloP, SiPhy, GERP++, and fitCons.

In some embodiments, the plurality of scores for training comprise a score from a score from a theoretical model comprising at least one of SIFT, Mutation Assessor, and LRT.

In some embodiments, the plurality of scores for training comprise a score for a supervised machine learning approach comprising at least one of PolyPhen-2, M-CAP, and FATHMM-MKL.

In some embodiments, the plurality of scores for training comprise a score for a supervised machine learning approach comprising at least one of PolyPhen-2, M-CAP, FATHMM-MKL.

Various embodiments of the invention have been described in detail. Changes in and or additions may be made.

APPENDIX I
Example Genes.

| Chromosome | Genes |
|---|---|
| 10 | GSTO1, CYP2C18, ABCC2, CYP17A1, CHST3, CYP2C8, CYP26C1, CYP2C19, |
| | CYP2C9, MAT1A, CYP2E1 |
| 11 | SLC22A11, SLCO2B1, NNMT, GSTP1, SLC22A8, CHST1, ABCC8, SLC22A6, |
| | SLC29A2 |
| 12 | SLCO1B3, ALDH2, CYP27B1, METTL1, SLCO1B1, ABCC9, SLCO1A2, CHST11 |
| 13 | NUDT-15, ABCC4, SLC15A1, SLC10A2, ATP7B |
| 14 | SLC10A1, SLC7A8, CYP46A1, GSTZ1, SLC7A7 |
| 15 | CYP1A1, CYP1A2, SLC28A2, CYP19A1, SLCO3A1, SLC28A1 |
| 16 | CHST4, QPRT, TPSG1, CES2, ABCC1, SULT1A2, CHST6, NQO1, RPL13, SPG7, |
| | SLC7A5, PRSS53, VKORC1, ABCC6, SULT1A3, SULT1A1, CHST5, SPN, CA5P |
| 17 | PGAP3, ALDH3A2, PNMT, ABCC3 |
| 18 | RALBP1, TYMS, CHST9 |
| 19 | CYP4F12, CYP4F11, CYP2B6, CYP2F1, CYP2B7P1, CYP2A13, CYP2A6, SULT2A1, |
| | CYP2S1, CHST8, CYP4F8, SULT2B1, CYP4F2, CYP4F3, CYP2A7 |
| 1 | DPYD, ARNT, FMO6, EPHX1, CDA, GSTM2, FMO1, FMO2, FMO5, FAAH, |
| | GSTM3, CYP2J2, FMO3, GSTM4, CYP4B1, GSTM1, FMO4, CYP4Z1, NR1I3, |
| | CYP4A11, GSTM5, APOA2, SLC16A1 |
| 20 | SLCO4A1, CYP24A1, PTGIS |
| 21 | CBR1, CBR3, ABCG1, SLC19A1 |
| 22 | SULT4A1, GSTT2, CYP2D6, ARSA, GSTT1, COMT |
| 2 | AOX1, SULT1C4, CHST10, UGT1A4, UGT1A8, HNMT, SLC5A6, SULT1C2, |
| | UGT1A10, UGT1A1, XDH, UGT1A9, UGT1A6, UGT1A3, CYP1B1, ABCB11, |
| | CYP20A1, CYP27A1, UGT1A7, UGT1A5 |
| 3 | SLC22A14, SLC22A13, SLC6A6, CHST2, ABCC5, SLC15A2, CHST13, NR1I2, |
| | CYP8B1, PPARG |
| 4 | UGT2A1, SULT1E1, UGT2B11, ABCG2, ADH1B, ADH7, UGT2B7, UGT2B4, ALB, |
| | ADH1C, UGT2B28, UGT8, ADH1A, UGT2B15, SULT1B1, ADH4, DCK, ADH5, |
| | ADH6, UGT2B17 |
| 5 | HMGCR, NR3C1, SLC22A5, SLC22A4 |
| 6 | CYP39A1, GSTA5, PPARD, SLC25A27, SLC22A1, SLC22A3, GSTM2, GSTA3, |
| | SLC22A2, GSTA1, GSTA4, CYP21A2, GSTA2, SLC22A7, TPMT, SLC29A1 |
| 7 | AHR, PON3, CYP3A43, SLC13A1, PON2, CYP3A7, ABCB4, CYP3A5, CROT, |
| | PON1, CYP3A4, AKAP9, ABP1, TBXAS1, ABCB1, PPP1R9A, CYP51A1, POR |
| 8 | CYP7B1, CYP11B2, EPHX2, CYP7A1, NAT1, SLCO5A1, NAT2, CYP11B1 |
| 9 | SLC28A3, ORM1, ALDH1A1, ORM2, RXRA |
| X | ABCB7, SERPINA7, CHST7, MAOA, G6PD, MAOB, ATP7A |

APPENDIX II
Example Pharmacogenomic Data

| | | Therapeutic | | |
|---|---|---|---|---|
| | Drug | Area | Biomarker | Data Available |
| 0 | Abacavir | Infectious | HLA-B | Boxed Warning, Dosage and |
| | | Diseases | | Administration, Contraindications, |
| | | | | Warnings and Precautions |
| 1 | Abemaciclib | Oncology | ESR | Indications and Usage, Adverse |
| | | | | Reactions, Clinical Studies |
| 2 | Abemaciclib | Oncology | ERBB2 | Indications and Usage, Adverse |
| | | | | Reactions, Clinical Studies |
| 3 | Abemaciclib | Oncology | MKI67 | Clinical Studies |
| 4 | Abrocitinib | Dermatology | CYP2C19 | Dosage and Administration, Use in |
| | | | | Specific Populations, Clinical |
| | | | | Pharmacology |
| 5 | Adagrasib | Oncology | KRAS | Indications and Usage, Dosage and |
| | | | | Administration, Adverse Reactions, |
| | | | | Clinical Pharmacology, Clinical |
| | | | | Studies |
| 6 | Ado- | Oncology | ERBB2 | Indications and Usage, Dosage and |
| | Trastuzumab | | | Administration, Adverse Reactions, |
| | Emtansine | | | Clinical Pharmacology, Clinical |
| | | | | Studies |
| 7 | Aducanumab- | Neurology | APOE | Warnings and Precautions, Clinical |
| | avwa | | | Studies |
| 8 | Afatinib | Oncology | EGFR | Indications and Usage, Dosage and |
| | | | | Administration, Adverse Reactions, |
| | | | | Clinical Studies |
| 9 | Alectinib | Oncology | ALK | Indications and Usage, Dosage and |
| | | | | Administration, Adverse Reactions, |
| | | | | Clinical Pharmacology, Clinical |
| | | | | Studies |
| 10 | Alglucosidase | Inborn Errors | GAA | Warnings and Precautions |
| | Alfa | of Metabolism | | |
| 11 | Allopurinol | Oncology | HLA-B | Warnings |
| 12 | Alpelisib | Oncology | ERBB2 | Indication and Usage, Dosage and |
| | | | | Administration, Adverse Reactions, |
| | | | | Clinical Studies |
| 13 | Alpelisib | Oncology | ESR | Indication and Usage, Dosage and |
| | | | | Administration, Adverse Reactions, |
| | | | | Clinical Studies |
| 14 | Alpelisib | Oncology | PIK3CA | Indication and Usage, Dosage and |
| | | | | Administration, Adverse Reactions, |
| | | | | Clinical Studies |
| 15 | Amifampridine | Neurology | NAT2 | Dosage and Administration, Adverse |
| | | | | Reactions, Use in Specific |
| | | | | Populations, Clinical Pharmacology |
| 16 | Amifampridine | Neurology | NAT2 | Dosage and Administration, Use in |
| | Phosphate | | | Specific Populations, Clinical |
| | | | | Pharmacology |
| 17 | Amikacin | Infectious | MT-RNR1 | Warnings and Precautions |
| | | Diseases | | |
| 18 | Amitriptyline | Psychiatry | CYP2D6 | Precautions |
| 19 | Amivantamab- | Oncology | EGFR | Indications and Usage, Dosage and |
| | vmjw | | | Administration, Adverse Reactions, |
| | | | | Clinical Studies |
| 20 | Amoxapine | Psychiatry | CYP2D6 | Precautions |
| 21 | Amphetamine | Psychiatry | CYP2D6 | Clinical Pharmacology |
| 22 | Anakinra | Rheumatology | NLRP3 | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies |
| 23 | Anastrozole | Oncology | ESR, PGR | Indications and Usage, Adverse |
| | | | | Reactions, Drug Interactions, Clinical |
| | | | | Studies |
| 25 | Arformoterol | Pulmonary | UGT1A1 | Clinical Pharmacology |
| 26 | Arformoterol | Pulmonary | CYP2D6 | Clinical Pharmacology |
| 27 | Aripiprazole | Psychiatry | CYP2D6 | Dosage and Administration, Use in |
| | | | | Specific Populations, Clinical |
| | | | | Pharmacology |
| 28 | Aripiprazole | Psychiatry | CYP2D6 | Dosage and Administration, Use in |
| | Lauroxil | | | Specific Populations, Clinical |
| | | | | Pharmacology |
| 29 | Arsenic | Oncology | PML- | Indications and Usage, Clinical |
| | Trioxide | | RARA | Studies |
| 30 | Articaine and | Anesthesiology | G6PD | Warnings and Precautions |
| | Epinephrine | | | |
| 32 | Asciminib | Oncology | BCR-ABL1 | Indications and Usage, Dosage and |
| | | | | Administration, Adverse Reactions, |
| | | | | Use in Specific Populations, Clinical |
| | | | | Studies |
| 33 | Ascorbic Acid | Endocrinology | G6PD | Dosage and Administration, |
| | | | | Warnings and Precautions, Adverse |
| | | | | Reactions, Patient Counseling |
| | | | | Information |
| 34 | Atezolizumab | Oncology | CD274 | Indications and Usage, Dosage and |
| | | | | Administration, Adverse Reactions, |
| | | | | Clinical Pharmacology, Clinical |
| | | | | Studies |
| 36 | Atezolizumab | Oncology | EGFR | Indications and Usage, Adverse |
| | | | | Reactions, Clinical Studies |
| 37 | Atezolizumab | Oncology | ALK | Indications and Usage, Adverse |
| | | | | Reactions, Clinical Studies |
| 38 | Atezolizumab | Oncology | BRAF | Indications and Usage, Dosage and |
| | | | | Administration, Adverse Reactions, |
| | | | | Clinical Studies |
| 39 | Atomoxetine | Psychiatry | CYP2D6 | Dosage and Administration, |
| | | | | Warnings and Precautions, Adverse |
| | | | | Reactions, Drug Interactions, Use in |
| | | | | Specific Populations, Clinical |
| | | | | Pharmacology |
| 40 | Ascorbic Acid, | Gastroenterology | G6PD | Warnings and Precautions, Adverse |
| | PEG-3350, | | | Reactions |
| | Potassium | | | |
| | Chloride, | | | |
| | Sodium | | | |
| | Ascorbate, | | | |
| | Sodium | | | |
| | Chloride, and | | | |
| | Sodium Sulfate | | | |
| 41 | Avapritinib | Oncology | PDGFRA | Indications and Usage, Dosage and |
| | | | | Administration, Clinical Studies |
| 42 | Avapritinib | Oncology | KIT | Clinical Studies |
| 43 | Avatrombopag | Hematology | F2 | Warnings and Precautions |
| 44 | Avatrombopag | Hematology | F5 | Warnings and Precautions |
| 45 | Avatrombopag | Hematology | PROC | Warnings and Precautions |
| 46 | Avatrombopag | Hematology | PROS1 | Warnings and Precautions |
| 47 | Avatrombopag | Hematology | SERPINC1 | Warnings and Precautions |
| 48 | Avatrombopag | Hematology | CYP2C9 | Clinical Pharmacology |
| 49 | Avelumab | Oncology | CD274 | Clinical Studies |
| 50 | Azacitidine | Oncology | CBL | Clinical Studies |
| 51 | Azacitidine | Oncology | PTPN11 | Clinical Studies |
| 52 | Azacitidine | Oncology | RAS | Clinical Studies |
| 53 | Azathioprine | Rheumatology | TPMT | Dosage and Administration, |
| | | | | Warnings, Precautions, Drug |
| | | | | Interactions, Adverse Reactions, |
| | | | | Clinical Pharmacology |
| 54 | Azathioprine | Rheumatology | NUDT15 | Dosage and Administration, |
| | | | | Warnings, Precautions, Adverse |
| | | | | Reactions, Clinical Pharmacology |
| 55 | Belinostat | Oncology | UGT1A1 | Dosage and Administration, Clinical |
| | | | | Pharmacology |
| 56 | Belzutifan | Oncology | CYP2C19 | Warnings and Precautions, Drug |
| | | | | Interactions, Use in Specific |
| | | | | Populations, Clinical Pharmacology |
| 57 | Belzutifan | Oncology | UGT2B17 | Warnings and Precautions, Drug |
| | | | | Interactions, Use in Specific |
| | | | | Populations, Clinical Pharmacology |
| 58 | Belzutifan | Oncology | VHL | Clinical Studies |
| 59 | Betaine | Inborn Errors | CBS, | Indications and Usage, Warnings and |
| | | of Metabolism | MMADHC, | Precautions, Clinical Pharmacology, |
| | | | MTHFR | Clinical Studies |
| 60 | Binimetinib | Oncology | BRAF | Indications and Usage, Dosage and |
| | | | | Administration, Warnings and |
| | | | | Precautions, Adverse Reactions, Use |
| | | | | in Specific Populations, Clinical |
| | | | | Studies |
| 61 | Binimetinib | Oncology | UGT1A1 | Clinical Pharmacology |
| 62 | Blinatumomab | Oncology | BCR-ABL1 | Adverse Reactions, Clinical Studies |
| 63 | Blinatumomab | Oncology | CD19 | Indications and Usage |
| 64 | Boceprevir | Infectious | IFNL3 | Clinical Pharmacology |
| | | Diseases | | |
| 65 | Bosutinib | Oncology | BCR-ABL1 | Indications and Usage, Dosage and |
| | | | | Administration, Warnings and |
| | | | | Precautions, Adverse Reactions, Use |
| | | | | in Specific Populations, Clinical |
| | | | | Studies |
| 66 | Brentuximab | Oncology | ALK | Use in Specific Populations, Clinical |
| | Vedotin | | | Studies |
| 67 | Brentuximab | Oncology | TNFRSF8 | Indications and Usage, Dosage and |
| | Vedotin | | | Administration, Adverse Reactions, |
| | | | | Use in Specific Populations, Clinical |
| | | | | Studies |
| 68 | Brexpiprazole | Psychiatry | CYP2D6 | Dosage and Administration, Use in |
| | | | | Specific Populations, Clinical |
| | | | | Pharmacology |
| 69 | Brigatinib | Oncology | ALK | Indications and Usage, Dosage and |
| | | | | Administration, Adverse Reactions, |
| | | | | Clinical Studies |
| 70 | Brivaracetam | Neurology | CYP2C19 | Clinical Pharmacology |
| 71 | Bupivacaine | Anesthesiology | G6PD | Warnings |
| 73 | Bupropion | Psychiatry | CYP2D6 | Clinical Pharmacology |
| 74 | Busulfan | Oncology | BCR-ABL1 | Clinical Studies |
| 75 | Cabotegravir | Infectious | HLA-B | Clinical Studies |
| | and Rilpivirine | Diseases | | |
| 76 | Cabotegravir | Infectious | UGT1A1 | Clinical Pharmacology |
| | and Rilpivirine | Diseases | | |
| 77 | Cabozantinib | Oncology | RET | Clinical Studies |
| 78 | Capmatinib | Oncology | MET | Indications and Usage, Dosage and |
| | | | | Administration, Clinical Studies |
| 79 | Capecitabine | Oncology | DPYD | Warnings and Precautions, Clinical |
| | | | | Pharmacology, Patient Counseling |
| | | | | Information |
| 80 | Capecitabine | Oncology | ERBB2 | Indications and Usage, Dosage and |
| | | | | Administration, Adverse Reactions, |
| | | | | Clinical Studies |
| 81 | Capivasertib | Oncology | AKT1 | Indications and Usage, Dosage and |
| | | | | Administration, Adverse Reactions, |
| | | | | Clinical Studies |
| 82 | Capivasertib | Oncology | ERBB2 | Indications and Usage, Dosage and |
| | | | | Administration, Clinical Studies |
| 83 | Capivasertib | Oncology | ESR | Indications and Usage, Dosage and |
| | | | | Administration, Clinical Studies |
| 84 | Capivasertib | Oncology | PIK3CA | Indications and Usage, Dosage and |
| | | | | Administration, Adverse Reactions, |
| | | | | Clinical Studies |
| 85 | Capivasertib | Oncology | PTEN | Indications and Usage, Dosage and |
| | | | | Administration, Adverse Reactions, |
| | | | | Clinical Studies |
| 86 | Carbamazepine | Neurology | HLA-B | Boxed Warning, Warnings, |
| | | | | Precautions |
| 87 | Carbamazepine | Neurology | HLA-A | Warnings |
| 88 | Carglumic Acid | Inborn Errors | NAGS | Indications and Usage, Dosage and |
| | | of Metabolism | | Administration, Warnings and |
| | | | | Precautions, Use in Specific |
| | | | | Populations, Clinical Pharmacology, |
| | | | | Clinical Studies |
| 89 | Cariprazine | Psychiatry | CYP2D6 | Clinical Pharmacology |
| 90 | Carisoprodol | Rheumatology | CYP2C19 | Use in Specific Populations, Clinical |
| | | | | Pharmacology |
| 91 | Carvedilol | Cardiology | CYP2D6 | Drug Interactions, Clinical |
| | | | | Pharmacology |
| 92 | Casimersen | Neurology | DMD | Indications and Usage, Adverse |
| | | | | Reactions, Use in Specific |
| | | | | Populations, Clinical Pharmacology, |
| | | | | Clinical Studies |
| 93 | Ceftriaxone | Infectious | G6PD | Warnings |
| | | Diseases | | |
| 95 | Celecoxib | Rheumatology | CYP2C9 | Dosage and Administration, Use in |
| | | | | Specific Populations, Clinical |
| | | | | Pharmacology |
| 96 | Cemiplimab- | Oncology | ALK | Indications and Usage, Clinical |
| | rwlc | | | Studies |
| 97 | Cemiplimab- | Oncology | CD274 | Indications and Usage, Dosage and |
| | rwlc | | | Administration, Clinical Studies |
| 98 | Cemiplimab- | Oncology | EGFR | Indications and Usage, Clinical |
| | rwlc | | | Studies |
| 99 | Cemiplimab- | Oncology | ROS1 | Indications and Usage, Clinical |
| | rwlc | | | Studies |
| 100 | Ceritinib | Oncology | ALK | Indications and Usage, Dosage and |
| | | | | Administration, Warning and |
| | | | | Precautions, Adverse Reactions, |
| | | | | Clinical Studies |
| 101 | Cerliponase | Inborn Errors | TPP1 | Indications and Usage, Use in |
| | Alfa | of Metabolism | | Specific Populations, Clinical Studies |
| 102 | Cetuximab | Oncology | EGFR | Indications and Usage, Dosage and |
| | | | | Administration, Adverse Reactions, |
| | | | | Clinical Studies |
| 103 | Cetuximab | Oncology | RAS | Indications and Usage, Dosage and |
| | | | | Administration, Warnings and |
| | | | | Precautions, Adverse Reactions, |
| | | | | Clinical Studies |
| 104 | Cetuximab | Oncology | BRAF | Indications and Usage, Dosage and |
| | | | | Administration, Adverse Reactions, |
| | | | | Use in Specific Populations, Clinical |
| | | | | Studies |
| 105 | Cevimeline | Dental | CYP2D6 | Precautions |
| 106 | Chloroprocaine | Anesthesiology | G6PD | Warnings |
| 108 | Chloroquine | Infectious | G6PD | Precautions, Adverse Reactions |
| | | Diseases | | |
| 109 | Chlorpropamide | Endocrinology | G6PD | Precautions |
| 110 | Cholic Acid | Inborn Errors | AMACR, | Indications and Usage, Dosage and |
| | | of Metabolism | AKR1D1, | Administration, Warnings and |
| | | | CYP7A1, | Precautions, Adverse Reactions, Use |
| | | | CYP27A1, | in Specific Populations, Clinical |
| | | | DHCR7, | Studies |
| | | | HSD3B2 | |
| 111 | Cisplatin | Oncology | TPMT | Adverse Reactions |
| 112 | Citalopram | Psychiatry | CYP2C19 | Dosage and Administration, |
| | | | | Warnings, Clinical Pharmacology |
| 113 | Citalopram | Psychiatry | CYP2D6 | Clinical Pharmacology |
| 114 | Clobazam | Neurology | CYP2C19 | Dosage and Administration, Use in |
| | | | | Specific Populations, Clinical |
| | | | | Pharmacology |
| 115 | Clomipramine | Psychiatry | CYP2D6 | Precautions |
| 116 | Clopidogrel | Cardiology | CYP2C19 | Boxed Warning, Warnings and |
| | | | | Precautions, Clinical Pharmacology |
| 117 | Clozapine | Psychiatry | CYP2D6 | Dosage and Administration, Use in |
| | | | | Specific Populations, Clinical |
| | | | | Pharmacology |
| 118 | Cobimetinib | Oncology | BRAF | Indications and Usage, Dosage and |
| | | | | Administration, Adverse Reactions, |
| | | | | Clinical Studies |
| 119 | Codeine | Anesthesiology | CYP2D6 | Boxed Warning, Warnings and |
| | | | | Precautions, Use in Specific |
| | | | | Populations, Patient Counseling |
| | | | | Information |
| 120 | Crizanlizumab- | Hematology | HBB | Adverse Reactions, Clinical Studies |
| | tmca | | | |
| 121 | Crizotinib | Oncology | ALK | Indications and Usage, Dosage and |
| | | | | Administration, Adverse Reactions, |
| | | | | Use in Specific Populations, Clinical |
| | | | | Pharmacology, Clinical Studies |
| 122 | Crizotinib | Oncology | ROS1 | Indications and Usage, Dosage and |
| | | | | Administration, Adverse Reactions, |
| | | | | Use in Specific Populations, Clinical |
| | | | | Studies |
| 123 | Dabrafenib | Oncology | BRAF | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies |
| 124 | Dabrafenib | Oncology | G6PD | Warnings and Precautions, Adverse |
| | | | | Reactions, Patient Counseling |
| | | | | Information |
| 125 | Dabrafenib | Oncology | RAS | Dosage and Administration, |
| | | | | Warnings and Precautions |
| 126 | Daclatasvir | Infectious | IFNL3 | Clinical Studies |
| | | Diseases | | |
| 127 | Dacomitinib | Oncology | EGFR | Indications and Usage, Dosage and |
| | | | | Administration, Adverse Reactions, |
| | | | | Use in Specific Populations, Clinical |
| | | | | Studies |
| 128 | Dapsone | Dermatology | G6PD | Warnings and Precautions, Use in |
| | | | | Specific Populations, Patient |
| | | | | Counseling Information |
| 130 | Dapsone | Infectious | G6PD | Precautions, Adverse Reactions, |
| | | Diseases | | Overdosage |
| 131 | Darifenacin | Urology | CYP2D6 | Clinical Pharmacology |
| 132 | Dasabuvir, | Infectious | IFNL3 | Clinical Studies |
| | Ombitasvir, | Diseases | | |
| | Paritaprevir, | | | |
| | and Ritonavir | | | |
| 133 | Dasatinib | Oncology | BCR-ABL1 | Indications and Usage, Dosage and |
| | | | | Administration, Warnings and |
| | | | | Precautions, Adverse Reactions, Use |
| | | | | in Specific Populations, Clinical |
| | | | | Studies |
| 134 | Denileukin | Oncology | IL2RA | Indications and Usage, Clinical |
| | Diftitox | | | Studies |
| 135 | Desipramine | Psychiatry | CYP2D6 | Precautions |
| 136 | Desflurane | Anesthesiology | CACNA1S, | Contraindications, Warnings and |
| | | | RYR1 | Precautions, Clinical Pharmacology |
| 137 | Desmopressin | Hematology | F8 | Indications and Usage, Dosage and |
| | | | | Administration, Clinical |
| | | | | Pharmacology |
| 138 | Desvenlafaxine | Psychiatry | CYP2D6 | Clinical Pharmacology |
| 139 | Deutetrabenazine | Neurology | CYP2D6 | Dosage and Administration, Use in |
| | | | | Specific Populations, Clinical |
| | | | | Pharmacology |
| 140 | Dexlansoprazole | Gastroenterology | CYP2C19 | Drug Interactions, Clinical |
| | | | | Pharmacology |
| 141 | Dextromethorphan | Neurology | CYP2D6 | Warnings and Precautions, Clinical |
| | and | | | Pharmacology |
| | Quinidine | | | |
| 142 | Diazepam | Neurology | CYP2C19 | Clinical Pharmacology |
| 143 | Dinutuximab | Oncology | MYCN | Clinical Studies |
| 144 | Docetaxel | Oncology | ESR, PGR | Clinical Studies |
| 145 | Dolutegravir | Infectious | UGT1A1 | Clinical Pharmacology |
| | | Diseases | | |
| 146 | Donepezil | Neurology | CYP2D6 | Clinical Pharmacology |
| 148 | Doxepin | Psychiatry | CYP2D6 | Clinical Pharmacology |
| 149 | Doxepin | Psychiatry | CYP2C19 | Clinical Pharmacology |
| 150 | Dronabinol | Gastroenterology | CYP2C9 | Use in Specific Populations, Clinical |
| | | | | Pharmacology |
| 151 | Drospirenone | Gynecology | CYP2C19 | Clinical Pharmacology |
| | and Ethinyl | | | |
| | Estradiol | | | |
| 152 | Duloxetine | Psychiatry | CYP2D6 | Drug Interactions |
| 153 | Durvalumab | Oncology | ALK | Indications and Usage, Clinical |
| | | | | Studies |
| 154 | Durvalumab | Oncology | EGFR | Indications and Usage, Clinical |
| | | | | Studies |
| 155 | Durvalumab | Oncology | CD274 | Clinical Pharmacology, Clinical |
| | | | | Studies |
| 157 | Eculizumab | Neurology | ACHR | Indications and Usage, Clinical |
| | | | | Studies |
| 158 | Eculizumab | Neurology | AQP4 | Indications and Usage, Clinical |
| | | | | Studies |
| 159 | Efavirenz | Infectious | CYP2B6 | Clinical Pharmacology |
| | | Diseases | | |
| 160 | Efgartigimod | Neurology | ACHR | Indications and Usage, Clinical |
| | Alfa-fcab | | | Pharmacology, Clinical Studies |
| 161 | Eflornithine | Oncology | MYCN | Adverse Reactions, Clinical Studies |
| 162 | Elacestrant | Oncology | ESR | Indications and Usage, Dosage and |
| | | | | Administration, Adverse Reactions, |
| | | | | Clinical Studies |
| 163 | Elacestrant | Oncology | ERBB2 | Indications and Usage, Dosage and |
| | | | | Administration, Adverse Reactions, |
| | | | | Clinical Studies |
| 164 | Elagolix | Gynecology | SLCO1B1 | Clinical Pharmacology |
| 165 | Elbasvir and | Infectious | IFNL3 | Clinical Studies |
| | Grazoprevir | Diseases | | |
| 166 | Elexacaftor, | Pulmonary | CFTR | Indications and Usage, Use in |
| | Ivacaftor, and | | | Specific Populations, Clinical |
| | Tezacaftor | | | Pharmacology, Clinical Studies |
| 167 | Eliglustat | Inborn Errors | CYP2D6 | Indications and Usage, Dosage and |
| | | of Metabolism | | Administration, Contraindications, |
| | | | | Warnings and Precautions, Drug |
| | | | | Interactions, Use in Specific |
| | | | | Populations, Clinical Pharmacology, |
| | | | | Clinical Studies |
| 171 | Elosulfase | Inborn Errors | GALNS | Indications and Usage, Warnings and |
| | | of Metabolism | | Precautions, Use in Specific |
| | | | | Populations, Clinical Pharmacology, |
| | | | | Clinical Studies |
| 172 | Eltrombopag | Hematology | F5 | Warnings and Precautions |
| 173 | Eltrombopag | Hematology | SERPINC1 | Warnings and Precautions |
| 176 | Emapalumab- | Hematology | PRF1, | Clinical Studies |
| | lzsg | | RAB27A, | |
| | | | SH2D1A, | |
| | | | STXBP2, | |
| | | | STX11, | |
| | | | UNC13D, | |
| | | | XIAP | |
| 177 | Enasidenib | Oncology | IDH2 | Indications and Usage, Dosage and |
| | | | | Administration, Clinical |
| | | | | Pharmacology, Clinical Studies |
| 178 | Encorafenib | Oncology | BRAF | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies, |
| | | | | Patient Counseling Information |
| 179 | Encorafenib | Oncology | RAS | Dosage and Administration, |
| | | | | Warnings and Precautions, Clinical |
| | | | | Studies |
| 180 | Entrectinib | Oncology | ROS1 | Indications and Usage, Dosage and |
| | | | | Administration, Adverse Reactions, |
| | | | | Use in Specific Populations, Clinical |
| | | | | Pharmacology, Clinical Studies |
| 181 | Entrectinib | Oncology | NTRK | Indications and Usage, Dosage and |
| | | | | Administration, Adverse Reactions, |
| | | | | Use in Specific Populations, Clinical |
| | | | | Pharmacology, Clinical Studies |
| 182 | Eplontersen | Neurology | TTR | Adverse Reactions, Clinical |
| | | | | Pharmacology, Clinical Studies |
| 183 | Erdafitinib | Oncology | FGFR | Indications and Usage, Dosage and |
| | | | | Administration, Adverse Reactions, |
| | | | | Clinical Studies, Patient Counseling |
| | | | | Information |
| 184 | Erdafitinib | Oncology | CYP2C9 | Use in Specific Populations, Clinical |
| | | | | Pharmacology |
| 185 | Eribulin | Oncology | ERBB2 | Clinical Studies |
| 186 | Eribulin | Oncology | ESR, PGR | Clinical Studies |
| 187 | Erlotinib | Oncology | EGFR | Indications and Usage, Dosage and |
| | | | | Administration, Adverse Reactions, |
| | | | | Clinical Studies |
| 188 | Erythromycin | Infectious | G6PD | Precautions |
| | and | Diseases | | |
| | Sulfisoxazole | | | |
| 189 | Escitalopram | Psychiatry | CYP2D6 | Drug Interactions |
| 190 | Escitalopram | Psychiatry | CYP2C19 | Adverse Reactions |
| 191 | Esomeprazole | Gastroenterology | CYP2C19 | Drug Interactions, Clinical |
| | | | | Pharmacology |
| 192 | Estradiol and | Gynecology | PROC | Contraindications |
| | Progesterone | | | |
| 193 | Estradiol and | Gynecology | PROS1 | Contraindications |
| | Progesterone | | | |
| 194 | Estradiol and | Gynecology | SERPINC1 | Contraindications |
| | Progesterone | | | |
| 195 | Estradiol | Gynecology | ESR, PGR | Warnings |
| | Valerate | | | |
| 196 | Eteplirsen | Neurology | DMD | Indications and Usage, Adverse |
| | | | | Reactions, Use in Specific |
| | | | | Populations, Clinical Studies |
| 197 | Etrasimod | Gastroenterology | CYP2C9 | Drug Interactions, Use in Specific |
| | | | | Populations, Clinical Pharmacology |
| 198 | Everolimus | Oncology | ERBB2 | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies |
| 199 | Everolimus | Oncology | ESR | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies |
| 200 | Evinacumab- | Endocrinology | LDLR | Clinical Studies |
| | dgnb | | | |
| 204 | Exemestane | Oncology | ESR, PGR | Indications and Usage, Dosage and |
| | | | | Administration, Clinical Studies |
| 205 | Fam- | Oncology | ERBB2 | Indications and Usage, Dosage and |
| | Trastuzumab | | | Administration, Warnings and |
| | Deruxtecan- | | | Precautions, Adverse Reactions, Use |
| | nxki | | | in Specific Populations, Clinical |
| | | | | Pharmacology, Clinical Studies |
| 206 | Fam- | Oncology | ESR | Clinical Studies |
| | Trastuzumab | | | |
| | Deruxtecan- | | | |
| | nxki | | | |
| 207 | Fesoterodine | Urology | CYP2D6 | Drug Interactions, Clinical |
| | | | | Pharmacology |
| 208 | Fosphenytoin | Neurology | CYP2C9 | Warnings and Precautions, Use in |
| | | | | Specific Populations, Clinical |
| | | | | Pharmacology |
| 209 | Fosphenytoin | Neurology | HLA-B | Warnings and Precautions |
| 210 | Flibanserin | Gynecology | CYP2C9 | Clinical Pharmacology |
| 211 | Flibanserin | Gynecology | CYP2C19 | Adverse Reactions, Use in Specific |
| | | | | Populations, Clinical Pharmacology |
| 212 | Flibanserin | Gynecology | CYP2D6 | Clinical Pharmacology |
| 213 | Fluorouracil | Dermatology | DPYD | Contraindications, Warnings |
| 214 | Fluorouracil | Oncology | DPYD | Warnings and Precautions, Patient |
| | | | | Counseling Information |
| 215 | Fluoxetine | Psychiatry | CYP2D6 | Warnings and Precautions, Drug |
| | | | | Interactions, Clinical Pharmacology |
| 216 | Flurbiprofen | Rheumatology | CYP2C9 | Clinical Pharmacology |
| 217 | Flutamide | Oncology | G6PD | Warnings |
| 218 | Fluvoxamine | Psychiatry | CYP2D6 | Drug Interactions |
| 219 | Formoterol | Pulmonary | CYP2D6 | Clinical Pharmacology |
| 220 | Formoterol | Pulmonary | CYP2C19 | Clinical Pharmacology |
| 221 | Fosdenopterin | Neurology | MOCS1 | Indications and Usage, Dosage and |
| | | | | Administration, Adverse Reactions, |
| | | | | Use in Specific Populations, Clinical |
| | | | | Pharmacology, Clinical Studies |
| 222 | Fruquintinib | Oncology | RAS | Indications and Usage, Clinical |
| | | | | Studies |
| 223 | Fulvestrant | Oncology | ERBB2 | Indications and Usage, Adverse |
| | | | | Reactions, Clinical Studies |
| 224 | Fulvestrant | Oncology | ESR, PGR | Indications and Usage, Adverse |
| | | | | Reactions, Clinical Pharmacology, |
| | | | | Clinical Studies |
| 225 | Futibatinib | Oncology | FGFR2 | Indications and Usage, Dosage and |
| | | | | Administration, Adverse Reactions, |
| | | | | Clinical Studies |
| 226 | Galantamine | Neurology | CYP2D6 | Clinical Pharmacology |
| 227 | Ganaxolone | Neurology | CDKL5 | Indications and Usage, Clinical |
| | | | | Studies |
| 228 | Gefitinib | Oncology | EGFR | Indications and Usage, Dosage and |
| | | | | Administration, Clinical Studies |
| 229 | Gefitinib | Oncology | CYP2D6 | Clinical Pharmacology |
| 230 | Gemtuzumab | Onoclogy | CD33 | Indications and Usage, Dosage and |
| | Ozogamicin | | | Administration, Adverse Reactions, |
| | | | | Clinical Studies |
| 231 | Gentamicin | Infectious | MT-RNR1 | Warnings |
| | | Diseases | | |
| 232 | Gilteritinib | Oncology | FLT3 | Indications and Usage, Dosage and |
| | | | | Administration, Clinical Studies |
| 233 | Givosiran | Gastroenterology | CPOX, | Clinical Studies |
| | | | HMBS, | |
| | | | PPOX | |
| 234 | Glimepiride | Endocrinology | G6PD | Warnings and Precautions, Adverse |
| | | | | Reactions |
| 235 | Glipizide | Endocrinology | G6PD | Precautions |
| 236 | Glyburide | Endocrinology | G6PD | Precautions |
| 237 | Glycerol | Inborn Errors | ASS1, | Indications and Usage, Adverse |
| | Phenylbutyrate | of Metabolism | CPS1, OTC | Reactions, Clinical Studies |
| 238 | Glycerol | Inborn Errors | NAGS | Indications and Usage |
| | Phenylbutyrate | of Metabolism | | |
| 239 | Golodirsen | Neurology | DMD | Indications and Usage, Use in |
| | | | | Specific Populations, Clinical |
| | | | | Pharmacology, Clinical Studies |
| 240 | Goserelin | Oncology | ESR, PGR | Indications and Usage, Clinical |
| | | | | Studies |
| 242 | Hydroxychloro | Infectious | G6PD | Warnings and Precautions, Adverse |
| | quine | Diseases | | Reactions |
| 245 | Ibrutinib | Oncology | MYD88 | Clinical Studies |
| 246 | Iloperidone | Psychiatry | CYP2D6 | Dosage and Administration, |
| | | | | Warnings and Precautions, Drug |
| | | | | Interactions, Clinical Pharmacology |
| 247 | Imatinib | Oncology | KIT | Indications and Usage, Dosage and |
| | | | | Administration, Clinical Studies |
| 248 | Imatinib | Oncology | BCR-ABL1 | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies |
| 249 | Imatinib | Oncology | PDGFRB | Indications and Usage, Dosage and |
| | | | | Administration, Clinical Studies |
| 250 | Imatinib | Oncology | FIP1L1- | Indications and Usage, Dosage and |
| | | | PDGFRA | Administration, Clinical Studies |
| 251 | Imipramine | Psychiatry | CYP2D6 | Precautions |
| 253 | Indacaterol | Pulmonary | UGT1A1 | Clinical Pharmacology |
| 254 | Inebilizumab- | Neurology | AQP4 | Indications and Usage, Clinical |
| | cdon | | | Studies |
| 255 | Infigratinib | Oncology | FGFR2 | Indications and Usage, Dosage and |
| | | | | Administration, Clinical Studies |
| 256 | Inotersen | Neurology | TTR | Adverse Reactions, Clinical |
| | | | | Pharmacology |
| 257 | Inotuzumab | Oncology | BCR-ABL1 | Clinical Studies |
| | Ozogamicin | | | |
| 258 | Ipilimumab | Oncology | HLA-A | Clinical Studies |
| 260 | Ipilimumab | Oncology | CD274 | Indications and Usage, Dosage and |
| | | | | Administration, Use in Specific |
| | | | | Populations, Clinical Studies |
| 261 | Ipilimumab | Oncology | ALK | Indications and Usage, Adverse |
| | | | | Reactions, Clinical Studies |
| 262 | Ipilimumab | Oncology | EGFR | Indications and Usage, Adverse |
| | | | | Reactions, Clinical Studies |
| 263 | Irinotecan | Oncology | UGT1A1 | Dosage and Administration, |
| | | | | Warnings and Precautions, Clinical |
| | | | | Pharmacology |
| 267 | Isoflurane | Anesthesiology | CACNA1S, | Contraindications, Warnings, Clinical |
| | | | RYR1 | Pharmacology |
| 269 | Isosorbide | Cardiology | CYB5R | Overdosage |
| | Dinitrate | | | |
| 270 | Isosorbide | Cardiology | CYB5R | Overdosage |
| | Mononitrate | | | |
| 271 | Ivacaftor | Pulmonary | CFTR | Indications and Usage, Adverse |
| | | | | Reactions, Use in Specific |
| | | | | Populations, Clinical Pharmacology, |
| | | | | Clinical Studies |
| 272 | Ivacaftor and | Pulmonary | CFTR | Indications and Usage, Adverse |
| | Lumacaftor | | | Reactions, Use in Specific |
| | | | | Populations, Clinical Studies |
| 273 | Ivacaftor and | Pulmonary | CFTR | Indications and Usage, Adverse |
| | Tezacaftor | | | Reactions, Use in Specific |
| | | | | Populations, Clinical Pharmacology, |
| | | | | Clinical Studies |
| 274 | Ivosidenib | Oncology | IDH1 | Indications and Usage, Dosage and |
| | | | | Administration, Clinical |
| | | | | Pharmacology, Clinical Studies |
| 275 | Ixabepilone | Oncology | ERBB2 | Clinical Studies |
| 276 | Ixabepilone | Oncology | ESR, PGR | Clinical Studies |
| 277 | Lacosamide | Neurology | CYP2C19 | Clinical Pharmacology |
| 278 | Lansoprazole | Gastroenterology | CYP2C19 | Drug Interactions, Clinical |
| | | | | Pharmacology |
| 279 | Lapatinib | Oncology | ERBB2 | Indications and Usage, Dosage and |
| | | | | Administration, Adverse Reactions, |
| | | | | Use in Specific Populations, Clinical |
| | | | | Studies |
| 280 | Lapatinib | Oncology | ESR, PGR | Indications and Usage, Dosage and |
| | | | | Administration, Adverse Reactions, |
| | | | | Use in Specific Populations, Clinical |
| | | | | Studies |
| 281 | Lapatinib | Oncology | HLA- | Clinical Pharmacology |
| | | | DQA1 | |
| 282 | Lapatinib | Oncology | HLA-DRB1 | Clinical Pharmacology |
| 283 | Larotrectinib | Oncology | NTRK | Indications and Usage, Dosage and |
| | | | | Administration, Adverse Reactions, |
| | | | | Clinical Studies |
| 284 | Lecanemab- | Neurology | APOE | Boxed Warning, Warnings and |
| | irmb | | | Precautions, Clinical Studies, Patient |
| | | | | Counseling Information |
| 285 | Ledipasvir and | Infectious | IFNL3 | Clinical Studies |
| | Sofosbuvir | Diseases | | |
| 287 | Leniolisib | Pulmonary | PIK3CD | Clinical Studies |
| 288 | Leniolisib | Pulmonary | PIK3R1 | Clinical Studies |
| 290 | Lesinurad | Rheumatology | CYP2C9 | Drug Interactions, Clinical |
| | | | | Pharmacology |
| 291 | Letrozole | Oncology | ESR, PGR | Indications and Usage, Adverse |
| | | | | Reactions, Clinical Studies |
| 294 | Lidocaine and | Anesthesiology | G6PD | Warnings and Precautions, Clinical |
| | Prilocaine | | | Pharmacology |
| 295 | Lidocaine and | Anesthesiology | G6PD | Warnings and Precautions |
| | Tetracaine | | | |
| 297 | Lofexidine | Anesthesiology | CYP2D6 | Use in Specific Populations |
| 299 | Lonafarnib | Inborn Errors | LMNA | Indications and Usage, Adverse |
| | | of Metabolism | | Reactions, Use in Specific |
| | | | | Populations, Clinical Studies |
| 300 | Lonafarnib | Inborn Errors | ZMPSTE24 | Indications and Usage, Use in |
| | | of Metabolism | | Specific Populations |
| 301 | Lorlatinib | Oncology | ALK | Indications and Usage, Dosage and |
| | | | | Administration, Adverse Reactions, |
| | | | | Clinical Studies |
| 302 | Lorlatinib | Oncology | ROS1 | Adverse Reactions |
| 303 | Lumasiran | Urology | AGXT | Indications and Usage, Adverse |
| | | | | Reactions, Use in Specific |
| | | | | Populations, Clinical Pharmacology, |
| | | | | Clinical Studies |
| 304 | Luspatercept- | Hematology | HBB | Clinical Studies |
| | aamt | | | |
| 305 | Lusutrombopag | Hematology | F2 | Warnings and Precautions |
| 306 | Lusutrombopag | Hematology | F5 | Warnings and Precautions |
| 307 | Lusutrombopag | Hematology | PROC | Warnings and Precautions |
| 308 | Lusutrombopag | Hematology | PROS1 | Warnings and Precautions |
| 309 | Lusutrombopag | Hematology | SERPINC1 | Warnings and Precautions |
| 310 | Lutetium Lu | Oncology | SSTR | Indications and Usage, Adverse |
| | 177 Dotatate | | | Reactions, Clinical Pharmacology, |
| | | | | Clinical Studies |
| 311 | Lutetium Lu | Oncology | FOLH1 | Indications and Usage, Dosage and |
| | 177 Vipivotide | | | Administration, Adverse Reactions, |
| | Tetraxetan | | | Clinical Studies |
| 312 | Mafenide | Infectious | G6PD | Warnings, Adverse Reactions |
| | | Diseases | | |
| 313 | Maralixibat | Gastroenterology | JAG1 | Clinical Studies |
| 314 | Margetuximab- | Oncology | ERBB2 | Indications and Usage, Adverse |
| | cmkb | | | Reactions, Clinical Pharmacology, |
| | | | | Clinical Studies |
| 315 | Margetuximab- | Oncology | FCGR2A | Clinical Pharmacology |
| | cmkb | | | |
| 316 | Margetuximab- | Oncology | FCGR2B | Clinical Pharmacology |
| | cmkb | | | |
| 317 | Margetuximab- | Oncology | FCGR3A | Clinical Pharmacology |
| | cmkb | | | |
| 318 | Mavacamten | Cardiology | CYP2C19 | Dosage and Administration, Clinical |
| | | | | Pharmacology |
| 319 | Meclizine | Neurology | CYP2D6 | Warnings and Precautions |
| 320 | Meloxicam | Anesthesiology | CYP2C9 | Use in Specific Populations, Clinical |
| | | | | Pharmacology |
| 321 | Mepivacaine | Anesthesiology | G6PD | Warnings |
| 323 | Mepolizumab | Oncology | FIP1L1- | Adverse Reactions, Clinical Studies |
| | | | PDGFRA | |
| 324 | Mercaptopurine | Oncology | TPMT | Dosage and Administration, |
| | | | | Warnings and Precautions, Adverse |
| | | | | Reactions, Clinical Pharmacology |
| 325 | Mercaptopurine | Oncology | NUDT15 | Dosage and Administration, |
| | | | | Warnings and Precautions, Clinical |
| | | | | Pharmacology |
| 326 | Methylene Blue | Hematology | G6PD | Contraindications, Warnings and |
| | | | | Precautions |
| 327 | Metoclopramide | Gastroenterology | CYB5R | Use in Specific Populations |
| 328 | Metoclopramide | Gastroenterology | G6PD | Use in Specific Populations, |
| | | | | Overdosage |
| 329 | Metoclopramide | Gastroenterology | CYP2D6 | Dosage and Administration, Use in |
| | | | | Specific Populations, Clinical |
| | | | | Pharmacology |
| 330 | Metoprolol | Cardiology | CYP2D6 | Clinical Pharmacology |
| 331 | Metreleptin | Endocrinology | LEP | Contraindications |
| 332 | Midostaurin | Oncology | FLT3 | Indications and Usage, Dosage and |
| | | | | Administration, Adverse Reactions, |
| | | | | Clinical Studies |
| 333 | Midostaurin | Oncology | NPM1 | Clinical Studies |
| 334 | Midostaurin | Oncology | KIT | Clinical Studies |
| 335 | Migalastat | Inborn Errors | GLA | Indications and Usage, Dosage and |
| | | of Metabolism | | Administration, Clinical |
| | | | | Pharmacology, Clinical Studies |
| 336 | Mirabegron | Urology | CYP2D6 | Clinical Pharmacology |
| 337 | Mirvetuximab | Oncology | FOLR1 | Indications and Usage, Dosage and |
| | Soravtansine- | | | Administration, Clinical Studies |
| | gynx | | | |
| 338 | Mitapivat | Hematology | PKLR | Clinical Studies |
| 339 | Mivacurium | Anesthesiology | BCHE | Warnings, Precautions, Clinical |
| | | | | Pharmacology |
| 340 | Mobocertinib | Oncology | EGFR | Indications and Usage, Dosage and |
| | | | | Administration, Adverse Reactions, |
| | | | | Clinical Studies |
| 341 | Modafinil | Psychiatry | CYP2D6 | Clinical Pharmacology |
| 342 | Mycophenolic | Transplantation | HPRT1 | Warnings and Precautions |
| | Acid | | | |
| 343 | Nalidixic Acid | Infectious | G6PD | Precautions, Adverse Reactions |
| | | Diseases | | |
| 344 | Nateglinide | Endocrinology | CYP2C9 | Drug Interactions |
| 345 | Nebivolol | Cardiology | CYP2D6 | Dosage and Administration, Clinical |
| | | | | Pharmacology |
| 346 | Nedosiran | Nephrology | AGXT | Indications and Usage, Adverse |
| | | | | Reactions, Use in Specific |
| | | | | Populations, Clinical Pharmacology, |
| | | | | Clinical Studies |
| 347 | Nefazodone | Psychiatry | CYP2D6 | Precautions |
| 348 | Neomycin | Infectious | MT-RNR1 | Warnings |
| | | Diseases | | |
| 349 | Neratinib | Oncology | ERBB2 | Indications and Usage, Adverse |
| | | | | Reactions, Clinical Studies |
| 350 | Neratinib | Oncology | ESR, PGR | Clinical Studies |
| 351 | Nilotinib | Oncology | BCR-ABL1 | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies |
| 352 | Nilotinib | Oncology | UGT1A1 | Clinical Pharmacology |
| 353 | Niraparib | Oncology | BRCA | Indications and Usage, Dosage and |
| | | | | Administration, Clinical Studies |
| 354 | Nitrofurantoin | Infectious | G6PD | Warnings, Adverse Reactions |
| | | Diseases | | |
| 355 | Nirogacestat | Oncology | APC | Clinical Studies |
| 356 | Nirogacestat | Oncology | CTNNB1 | Clinical Studies |
| 357 | Nivolumab | Oncology | BRAF | Adverse Reactions, Clinical Studies |
| 358 | Nivolumab | Oncology | CD274 | Indications and Usage, Dosage and |
| | | | | Administration, Use in Specific |
| | | | | Populations, Clinical Pharmacology, |
| | | | | Clinical Studies |
| 360 | Nivolumab | Oncology | EGFR | Indications and Usage, Adverse |
| | | | | Reactions, Clinical Studies |
| 361 | Nivolumab | Oncology | ALK | Indications and Usage, Adverse |
| | | | | Reactions, Clinical Studies |
| 362 | Nivolumab | Oncology | ERBB2 | Adverse Reactions, Clinical Studies |
| 363 | Nivolumab and | Oncology | BRAF | Clinical Studies |
| | Relatlimab- | | | |
| | rmbw | | | |
| 364 | Nivolumab and | Oncology | CD274 | Clinical Studies |
| | Relatlimab- | | | |
| | rmbw | | | |
| 365 | Nivolumab and | Oncology | LAG3 | Clinical Studies |
| | Relatlimab- | | | |
| | rmbw | | | |
| 366 | Nortriptyline | Psychiatry | CYP2D6 | Precautions |
| 367 | Nusinersen | Neurology | SMN2 | Clinical Pharmacology, Clinical |
| | | | | Studies |
| 368 | Obinutuzumab | Oncology | MS4A1 | Clinical Studies |
| 369 | Odevixibat | Gastroenterology | ABCB11 | Indications and Usage, Clinical |
| | | | | Pharmacology, Clinical Studies |
| 370 | Odevixibat | Gastroenterology | ATP8B1 | Indications and Usage, Clinical |
| | | | | Pharmacology, Clinical Studies |
| 371 | Odevixibat | Gastroenterology | JAG1 | Clinical Studies |
| 372 | Odevixibat | Gastroenterology | NOTCH2 | Clinical Studies |
| 373 | Olaparib | Oncology | BRCA | Indications and Usage, Dosage and |
| | | | | Administration, Warnings and |
| | | | | Precautions, Adverse Reactions, |
| | | | | Clinical Studies |
| 374 | Olaparib | Oncology | ERBB2 | Indications and Usage, Dosage and |
| | | | | Administration, Adverse Reactions, |
| | | | | Clinical Studies |
| 375 | Olaparib | Oncology | ESR, PGR | Indications and Usage, Clinical |
| | | | | Studies |
| 378 | Olaparib | Oncology | PPP2R2A | Clinical Studies |
| 379 | Olaratumab | Oncology | PDGFRA | Clinical Studies |
| 380 | Oliceridine | Anesthesiology | CYP2D6 | Warnings and Precautions, Drug |
| | | | | Interactions, Use in Specific |
| | | | | Populations, Clinical Pharmacology |
| 381 | Olutasidenib | Oncology | IDH1 | Indications and Usage, Dosage and |
| | | | | Administration, Adverse Reactions, |
| | | | | Use in Specific Populations, Clinical |
| | | | | Pharmacology, Clinical Studies |
| 382 | Omacetaxine | Oncology | BCR-ABL1 | Clinical Studies |
| 383 | Ombitasvir, | Infectious | IFNL3 | Clinical Studies |
| | Paritaprevir, | Diseases | | |
| | and Ritonavir | | | |
| 384 | Omeprazole | Gastroenterology | CYP2C19 | Drug Interactions, Clinical |
| | | | | Pharmacology |
| 385 | Oxymetazoline | Anesthesiology | G6PD | Warnings and Precautions |
| | and Tetracaine | | | |
| 387 | Ondansetron | Gastroenterology | CYP2D6 | Clinical Pharmacology |
| 388 | Osimertinib | Oncology | EGFR | Indications and Usage, Dosage and |
| | | | | Administration, Adverse Reactions, |
| | | | | Clinical Studies |
| 389 | Ospemifene | Gynecology | CYP2C9 | Clinical Pharmacology |
| 390 | Ospemifene | Gynecology | CYP2B6 | Clinical Pharmacology |
| 391 | Oxcarbazepine | Neurology | HLA-B | Warnings and Precautions |
| 392 | Palbociclib | Oncology | ESR | Indications and Usage, Adverse |
| | | | | Reactions, Clinical Studies |
| 393 | Palbociclib | Oncology | ERBB2 | Indications and Usage, Adverse |
| | | | | Reactions, Clinical Studies |
| 394 | Paliperidone | Psychiatry | CYP2D6 | Clinical Pharmacology |
| 395 | Palonosetron | Gastroenterology | CYP2D6 | Clinical Pharmacology |
| 396 | Panitumumab | Oncology | EGFR | Adverse Reactions, Clinical |
| | | | | Pharmacology, Clinical Studies |
| 397 | Panitumumab | Oncology | RAS | Indications and Usage, Dosage and |
| | | | | Administration, Warnings and |
| | | | | Precautions, Adverse Reactions, |
| | | | | Clinical Studies |
| 398 | Pantoprazole | Gastroenterology | CYP2C19 | Clinical Pharmacology |
| 399 | Parathyroid | Inborn Errors | CASR | Indications and Usage, Clinical |
| | Hormone | of Metabolism | | Studies |
| 400 | Paroxetine | Psychiatry | CYP2D6 | Drug Interactions, Clinical |
| | | | | Pharmacology |
| 401 | Patisiran | Neurology | TTR | Adverse Reactions, Clinical |
| | | | | Pharmacology, Clinical Studies |
| 402 | Pazopanib | Oncology | UGT1A1 | Clinical Pharmacology |
| 403 | Pazopanib | Oncology | HLA-B | Clinical Pharmacology |
| 404 | Peginterferon | Infectious | IFNL3 | Clinical Pharmacology |
| | Alfa-2b | Diseases | | |
| 405 | Pegloticase | Rheumatology | G6PD | Boxed Warning, Contraindications, |
| | | | | Warnings and Precautions, Adverse |
| | | | | Reactions, Patient Counseling |
| | | | | Information |
| 406 | Pembrolizumab | Oncology | BRAF | Adverse Reactions, Clinical Studies |
| 407 | Pembrolizumab | Oncology | CD274 | Indications and Usage, Dosage and |
| | | | | Administration, Adverse Reactions, |
| | | | | Use in Specific Populations, Clinical |
| | | | | Studies |
| 409 | Pembrolizumab | Oncology | EGFR | Indications and Usage, Adverse |
| | | | | Reactions, Clinical Studies |
| 410 | Pembrolizumab | Oncology | ALK | Indications and Usage, Adverse |
| | | | | Reactions, Clinical Studies |
| 412 | Pembrolizumab | Oncology | ERBB2 | Indications and Usage, Adverse |
| | | | | Reactions, Clinical Studies |
| 413 | Pemetrexed | Oncology | ALK | Indications and Usage, Adverse |
| | | | | Reactions, Clinical Studies |
| 414 | Pemetrexed | Oncology | EGFR | Indications and Usage, Adverse |
| | | | | Reactions, Clinical Studies |
| 415 | Pemetrexed | Oncology | CD274 | Clinical Studies |
| 416 | Pemigatinib | Oncology | FGFR1 | Indications and Usage, Dosage and |
| | | | | Administration, Adverse Reactions, |
| | | | | Use in Specific Populations, Clinical |
| | | | | Studies |
| 417 | Pemigatinib | Oncology | FGFR2 | Indications and Usage, Dosage and |
| | | | | Administration, Clinical Studies |
| 418 | Perphenazine | Psychiatry | CYP2D6 | Precautions, Clinical Pharmacology |
| 419 | Pertuzumab | Oncology | ERBB2 | Indications and Usage, Dosage and |
| | | | | Administration, Warnings and |
| | | | | Precautions, Adverse Reactions, |
| | | | | Clinical Pharmacology, Clinical |
| | | | | Studies |
| 420 | Pertuzumab | Oncology | ESR, PGR | Clinical Studies |
| 421 | Phenytoin | Neurology | CYP2C9 | Warnings and Precautions, Use in |
| | | | | Specific Populations, Clinical |
| | | | | Pharmacology |
| 422 | Phenytoin | Neurology | CYP2C19 | Clinical Pharmacology |
| 423 | Phenytoin | Neurology | HLA-B | Warnings and Precautions |
| 424 | Pimozide | Psychiatry | CYP2D6 | Dosage and Administration, |
| | | | | Precautions |
| 425 | Piroxicam | Rheumatology | CYP2C9 | Clinical Pharmacology |
| 426 | Pirtobrutinib | Oncology | BTK | Clinical Studies |
| 429 | Pirtobrutinib | Oncology | IGHV | Clinical Studies |
| 430 | Pirtobrutinib | Oncology | TP53 | Clinical Studies |
| 431 | Pitolisant | Psychiatry | CYP2D6 | Dosage and Administration, Use in |
| | | | | Specific Populations, Clinical |
| | | | | Pharmacology |
| 432 | Plazomicin | Infectious | MT-RNR1 | Warnings and Precautions |
| | | Diseases | | |
| 433 | Polatuzumab | Oncology | BCL2 | Clinical Studies |
| | Vedotin-piiq | | | |
| 434 | Polatuzumab | Oncology | BCL6 | Clinical Studies |
| | Vedotin-piiq | | | |
| 435 | Polatuzumab | Oncology | MYC | Clinical Studies |
| | Vedotin-piiq | | | |
| 436 | Ponatinib | Oncology | BCR-ABL1 | Indications and Usage, Warnings and |
| | | | | Precautions, Adverse Reactions, Use |
| | | | | in Specific Populations, Clinical |
| | | | | Studies |
| 437 | Pralsetinib | Oncology | CCDC6- | Indications and Usage, Dosage and |
| | | | RET, | Administration, Adverse Reactions, |
| | | | KIF5B- | Use in Specific Populations, Clinical |
| | | | RET, RET | Pharmacology, Clinical Studies |
| 438 | Prasugrel | Cardiology | CYP2C19 | Use in Specific Populations, Clinical |
| | | | | Pharmacology, Clinical Studies |
| 439 | Prasugrel | Cardiology | CYP2C9 | Use in Specific Populations, Clinical |
| | | | | Pharmacology, Clinical Studies |
| 440 | Prasugrel | Cardiology | CYP3A5 | Use in Specific Populations, Clinical |
| | | | | Pharmacology, Clinical Studies |
| 441 | Prasugrel | Cardiology | CYP2B6 | Use in Specific Populations, Clinical |
| | | | | Pharmacology, Clinical Studies |
| 442 | Primaquine | Infectious | G6PD | Contraindications, Warnings, |
| | | Diseases | | Precautions, Adverse Reactions, |
| | | | | Overdosage |
| 443 | Primaquine | Infectious | CYB5R | Precautions, Adverse Reactions |
| | | Diseases | | |
| 444 | Probenecid | Rheumatology | G6PD | Adverse Reactions |
| 446 | Propafenone | Cardiology | CYP2D6 | Dosage and Administration, |
| | | | | Warnings and Precautions, Drug |
| | | | | Interactions, Clinical Pharmacology |
| 447 | Propranolol | Cardiology | CYP2D6 | Clinical Pharmacology |
| 448 | Protriptyline | Psychiatry | CYP2D6 | Precautions |
| 449 | Quinidine | Cardiology | CYP2D6 | Precautions |
| 450 | Quinine Sulfate | Infectious | G6PD | Warnings and Precautions |
| | | Diseases | | |
| 451 | Quinine Sulfate | Infectious | CYP2D6 | Drug Interactions |
| | | Diseases | | |
| 452 | Quizartinib | Oncology | FLT3 | Indications and Usage, Dosage and |
| | | | | Administration, Warnings and |
| | | | | Precautions, Adverse Reactions, |
| | | | | Clinical Studies |
| 453 | Rabeprazole | Gastroenterology | CYP2C19 | Drug Interactions, Clinical |
| | | | | Pharmacology |
| 454 | Raloxifene | Oncology | ESR | Clinical Studies |
| 455 | Raltegravir | Infectious | UGT1A1 | Clinical Pharmacology |
| | | Diseases | | |
| 456 | Ramucirumab | Oncology | EGFR | Indications and Usage, Dosage and |
| | | | | Administration, Adverse Reactions, |
| | | | | Clinical Studies |
| 457 | Ramucirumab | Oncology | RAS | Clinical Studies |
| 458 | Rasburicase | Oncology | G6PD | Boxed Warning, Contraindications, |
| | | | | Warnings and Precautions |
| 459 | Rasburicase | Oncology | CYB5R | Boxed Warning, Contraindications, |
| | | | | Warnings and Precautions |
| 460 | Ravulizumab- | Neurology | ACHR | Indications and Usage, Clinical |
| | CWVZ | | | Studies |
| 461 | Regorafenib | Oncology | RAS | Indications and Usage, Clinical |
| | | | | Studies |
| 462 | Repotrectinib | Oncology | ROS1 | Indications and Usage, Dosage and |
| | | | | Administration, Adverse Reactions, |
| | | | | Use in Specific Populations, Clinical |
| | | | | Studies |
| 463 | Ribociclib | Oncology | ESR, PGR | Indications and Usage, Adverse |
| | | | | Reactions, Clinical Studies |
| 464 | Ribociclib | Oncology | ERBB2 | Indications and Usage, Adverse |
| | | | | Reactions, Clinical Studies |
| 465 | Rimegepant | Neurology | CYP2C9 | Clinical Pharmacology |
| 466 | Risdiplam | Neurology | SMN1, | Clinical Studies |
| | | | SMN2 | |
| 467 | Risperidone | Psychiatry | CYP2D6 | Clinical Pharmacology |
| 468 | Rituximab | Oncology | MS4A1 | Indications and Usage, Dosage and |
| | | | | Administration, Adverse Reactions, |
| | | | | Use in Specific Populations, Clinical |
| | | | | Studies |
| 469 | Rivaroxaban | Cardiology | F5 | Clinical Studies |
| 470 | Ropeginterferon | Hematology | JAK2 | Clinical Pharmacology, Clinical |
| | Alfa-2b-njft | | | Studies |
| 471 | Ropivacaine | Anesthesiology | G6PD | Warnings |
| 473 | Rosuvastatin | Endocrinology | SLCO1B1 | Clinical Pharmacology |
| 474 | Rozanolixizum | Neurology | ACHR | Indications and Usage, Clinical |
| | ab-noli | | | Pharmacology, Clinical Studies |
| 475 | Rozanolixizum | Neurology | MUSK | Indications and Usage, Clinical |
| | ab-noli | | | Pharmacology, Clinical Studies |
| 476 | Rucaparib | Oncology | BRCA | Indications and Usage, Dosage and |
| | | | | Administration, Adverse Reactions, |
| | | | | Clinical Studies |
| 477 | Rucaparib | Oncology | CYP2D6 | Clinical Pharmacology |
| 478 | Rucaparib | Oncology | CYP1A2 | Clinical Pharmacology |
| 480 | Sacituzumab | Oncology | UGT1A1 | Warnings and Precautions, Clinical |
| | Govitecan-hziy | | | Pharmacology |
| 481 | Sacituzumab | Oncology | BRCA | Clinical Studies |
| | Govitecan-hziy | | | |
| 482 | Sacituzumab | Oncology | ESR | Indications and Usage, Adverse |
| | Govitecan-hziy | | | Reactions, Use in Specific |
| | | | | Populations, Clinical Studies |
| 483 | Sacituzumab | Oncology | ERBB2 | Indications and Usage, Adverse |
| | Govitecan-hziy | | | Reactions, Use in Specific |
| | | | | Populations, Clinical Studies |
| 485 | Satralizumab- | Neurology | AQP4 | Indications and Usage, Adverse |
| | mwge | | | Reactions, Clinical Studies |
| 486 | Selpercatinib | Oncology | RET | Indications and Usage, Dosage and |
| | | | | Administration, Adverse Reactions, |
| | | | | Use in Specific Populations, Clinical |
| | | | | Studies |
| 487 | Setmelanotide | Endocrinology | LEPR | Indications and Usage, Dosage and |
| | | | | Administration, Adverse Reactions, |
| | | | | Use in Specific Populations, Clinical |
| | | | | Studies |
| 488 | Setmelanotide | Endocrinology | PCSK1 | Indications and Usage, Dosage and |
| | | | | Administration, Adverse Reactions, |
| | | | | Use in Specific Populations, Clinical |
| | | | | Studies |
| 489 | Setmelanotide | Endocrinology | POMC | Indications and Usage, Dosage and |
| | | | | Administration, Adverse Reactions, |
| | | | | Use in Specific Populations, Clinical |
| | | | | Studies |
| 490 | Sevoflurane | Anesthesiology | CACNAIS, | Contraindications, Warnings, Clinical |
| | | | RYR1 | Pharmacology |
| 491 | Simeprevir | Infectious | IFNL3 | Clinical Pharmacology, Clinical |
| | | Diseases | | Studies |
| 492 | Siponimod | Neurology | CYP2C9 | Dosage and Administration, |
| | | | | Contraindications, Drug Interactions, |
| | | | | Use in Specific Populations, Clinical |
| | | | | Pharmacology |
| 493 | Sodium Nitrite | Toxicology | G6PD | Warnings and Precautions |
| 495 | Sodium | Neurology | ALDH5A1 | Contraindications |
| | Oxybate | | | |
| 496 | Sodium | Inborn Errors | ASS1, | Indications and Usage, Dosage and |
| | Phenylbutyrate | of Metabolism | CPS1, OTC | Administration, Adverse Reactions, |
| | | | | Clinical Pharmacology |
| 497 | Sofosbuvir | Infectious | IFNL3 | Clinical Studies |
| | | Diseases | | |
| 498 | Sofosbuvir and | Infectious | IFNL3 | Clinical Studies |
| | Velpatasvir | Diseases | | |
| 499 | Sofosbuvir, | Infectious | IFNL3 | Clinical Studies |
| | Velpatasvir, | Diseases | | |
| | and | | | |
| | Voxilaprevir | | | |
| 500 | Sotorasib | Oncology | KRAS | Indication and Usage, Dosage and |
| | | | | Administration, Adverse Reactions, |
| | | | | Clinical Pharmacology, Clinical |
| | | | | Studies |
| 501 | Streptomycin | Infectious | MT-RNR1 | Warnings |
| | | Diseases | | |
| 502 | Succimer | Hematology | G6PD | Clinical Pharmacology |
| 503 | Succinylcholine | Anesthesiology | BCHE | Warnings, Precautions |
| 504 | Succinylcholine | Anesthesiology | CACNAIS, | Boxed Warning, Contraindications, |
| | | | RYR1 | Warnings, Precautions, Adverse |
| | | | | Reactions, Clinical Pharmacology |
| 505 | Sulfadiazine | Infectious | G6PD | Warnings |
| | | Diseases | | |
| 506 | Sulfamethoxaz | Infectious | G6PD | Precautions |
| | ole and | Diseases | | |
| | Trimethoprim | | | |
| 508 | Sulfasalazine | Gastroenterolo | G6PD | Precautions |
| | | gy | | |
| 510 | Synthetic | Gynecology | PROC | Contraindications |
| | Conjugated | | | |
| | Estrogens, A | | | |
| 511 | Synthetic | Gynecology | PROS1 | Contraindications |
| | Conjugated | | | |
| | Estrogens, A | | | |
| 512 | Synthetic | Gynecology | SERPINC1 | Contraindications |
| | Conjugated | | | |
| | Estrogens, A | | | |
| 513 | Tafamidis | Cardiology | TTR | Clinical Pharmacology, Clinical |
| | | | | Studies |
| 514 | Tafenoquine | Infectious | G6PD | Dosage and Administration, |
| | | Diseases | | Contraindications, Warnings and |
| | | | | Precautions, Use in Specific |
| | | | | Populations, Patient Counseling |
| | | | | Information |
| 515 | Talazoparib | Oncology | BRCA | Indications and Usage, Dosage and |
| | | | | Administration, Adverse Reactions, |
| | | | | Clinical Studies |
| 516 | Talazoparib | Oncology | ERBB2 | Indications and Usage, Adverse |
| | | | | Reactions, Clinical Studies |
| 518 | Tamoxifen | Oncology | ESR, PGR | Indications and Usage, Adverse |
| | | | | Reactions, Clinical Pharmacology, |
| | | | | Clinical Studies |
| 519 | Tamoxifen | Oncology | F5 | Warnings and Precautions |
| 520 | Tamoxifen | Oncology | F2 | Warnings and Precautions |
| 521 | Tamoxifen | Oncology | CYP2D6 | Clinical Pharmacology |
| 522 | Tamsulosin | Urology | CYP2D6 | Warnings and Precautions, Adverse |
| | | | | Interactions, Clinical Pharmacology |
| 523 | Tebentafusp- | Oncology | HLA-A | Indications and Usage, Dosage and |
| | tebn | | | Administration, Clinical Studies |
| 527 | Telaprevir | Infectious | IFNL3 | Clinical Pharmacology, Clinical |
| | | Diseases | | Studies |
| 528 | Tepotinib | Oncology | ALK | Clinical Studies |
| 529 | Tepotinib | Oncology | EGFR | Clinical Studies |
| 530 | Tepotinib | Oncology | MET | Indications and Usage, Dosage and |
| | | | | Administration, Adverse Reactions, |
| | | | | Use in Specific Populations, Clinical |
| | | | | Studies |
| 531 | Tetrabenazine | Neurology | CYP2D6 | Dosage and Administration, |
| | | | | Warnings and Precautions, Use in |
| | | | | Specific Populations, Clinical |
| | | | | Pharmacology |
| 532 | Thioguanine | Oncology | TPMT | Dosage and Administration, |
| | | | | Warnings, Precautions, Clinical |
| | | | | Pharmacology |
| 533 | Thioguanine | Oncology | NUDT15 | Dosage and Administration, |
| | | | | Warnings, Precautions, Clinical |
| | | | | Pharmacology |
| 534 | Thioridazine | Psychiatry | CYP2D6 | Contraindications, Warnings, |
| | | | | Precautions |
| 535 | Ticagrelor | Cardiology | CYP2C19 | Clinical Pharmacology |
| 536 | Tipiracil and | Oncology | ERBB2 | Indications and Usage, Adverse |
| | Trifluridine | | | Reactions, Clinical Studies |
| 537 | Tipiracil and | Oncology | RAS | Indications and Usage, Clinical |
| | Trifluridine | | | Studies |
| 538 | Tobramycin | Infectious | MT-RNR1 | Warnings and Precautions |
| | | Diseases | | |
| 539 | Tofersen | Oncology | SOD1 | Indications and Usage, Use in |
| | | | | Specific Populations, Clinical Studies |
| 540 | Tolazamide | Endocrinology | G6PD | Precautions |
| 541 | Tolbutamide | Endocrinology | G6PD | Precautions |
| 542 | Tolterodine | Urology | CYP2D6 | Warnings and Precautions, Drug |
| | | | | Interactions, Clinical Pharmacology |
| 543 | Toremifene | Oncology | ESR | Indications and Usage, Clinical |
| | | | | Studies |
| 544 | Tramadol | Anesthesiolog | CYP2D6 | Boxed Warning, Warnings and |
| | | y | | Precautions, Use in Specific |
| | | | | Populations, Clinical Pharmacology, |
| | | | | Patient Counseling Information |
| 545 | Trametinib | Oncology | BRAF | Indications and Usage, Dosage and |
| | | | | Administration, Adverse Reactions, |
| | | | | Use in Specific Populations, Clinical |
| | | | | Pharmacology, Clinical Studies |
| 546 | Trametinib | Oncology | G6PD | Adverse Reactions |
| 547 | Trametinib | Oncology | RAS | Warnings and Precautions |
| 548 | Trastuzumab | Oncology | ERBB2 | Indications and Usage, Dosage and |
| | | | | Administration, Clinical |
| | | | | Pharmacology, Clinical Studies |
| 549 | Trastuzumab | Oncology | ESR, PGR | Clinical Studies |
| 550 | Tretinoin | Oncology | PML- | Indications and Usage, Dosage and |
| | | | RARA | Administration, Warnings and |
| | | | | Precautions, Patient Counseling |
| | | | | Information |
| 551 | Triheptanoin | Inborn Errors | ACADVL, | Indications and Usage, Clinical |
| | | of Metabolism | CPT2, | Studies |
| | | | HADHA, | |
| | | | HADHB | |
| 552 | Tremelimumab | Oncology | ALK | Indications and Usage, Clinical |
| | -actl | | | Studies |
| 553 | Tremelimumab | Oncology | EGFR | Indications and Usage, Clinical |
| | -actl | | | Studies |
| 554 | Tremelimumab | Oncology | CD274 | Clinical Studies |
| | -actl | | | |
| 555 | Trimipramine | Psychiatry | CYP2D6 | Precautions |
| 556 | Trofinetide | Neurology | MECP2 | Clinical Studies |
| 557 | Tucatinib | Oncology | ERBB2 | Indications and Usage, Dosage and |
| | | | | Administration, Adverse Reactions, |
| | | | | Clinical Studies |
| 559 | Tucatinib | Oncology | RAS | Clinical Studies |
| 560 | Umeclidinium | Pulmonary | CYP2D6 | Clinical Pharmacology |
| 561 | Upadacitinib | Rheumatology | CYP2D6 | Clinical Pharmacology |
| 562 | Ustekinumab | Dermatology | IL12A, | Warnings and Precautions |
| | | and | IL12B, | |
| | | Gastroenterolo | IL23A | |
| | | gy | | |
| 563 | Valbenazine | Neurology | CYP2D6 | Dosage and Administration, |
| | | | | Warnings and Precautions, Use in |
| | | | | Specific Populations, Clinical |
| | | | | Pharmacology |
| 564 | Valproic Acid | Neurology | POLG | Boxed Warning, Contraindications, |
| | | | | Warnings and Precautions |
| 566 | Vemurafenib | Oncology | BRAF | Indications and Usage, Dosage and |
| | | | | Administration, Warnings and |
| | | | | Precautions, Adverse Reactions, Use |
| | | | | in Specific Populations, Clinical |
| | | | | Pharmacology, Clinical Studies, |
| | | | | Patient Counseling Information |
| 567 | Vemurafenib | Oncology | RAS | Warnings and Precautions, Adverse |
| | | | | Reactions |
| 570 | Venetoclax | Oncology | TP53 | Clinical Studies |
| 571 | Venetoclax | Oncology | IDH1 | Clinical Studies |
| 572 | Venetoclax | Oncology | IDH2 | Clinical Studies |
| 573 | Venetoclax | Oncology | IGHV | Clinical Studies |
| 574 | Venetoclax | Oncology | NPM1 | Clinical Studies |
| 575 | Venetoclax | Oncology | FLT3 | Clinical Studies |
| 576 | Venlafaxine | Psychiatry | CYP2D6 | Drug Interactions, Use in Specific |
| | | | | Populations, Clinical Pharmacology |
| 577 | Viloxazine | Psychiatry | CYP2D6 | Clinical Pharmacology |
| 578 | Viloxazine | Psychiatry | SLCO1B1 | Clinical Pharmacology |
| 579 | Viltolarsen | Neurology | DMD | Indications and Usage, Adverse |
| | | | | Reactions, Use in Specific |
| | | | | Populations, Clinical Pharmacology, |
| | | | | Clinical Studies |
| 580 | Vincristine | Oncology | BCR-ABL1 | Indications and Usage, Adverse |
| | | | | Reactions, Clinical Studies |
| 581 | Voriconazole | Infectious | CYP2C19 | Clinical Pharmacology |
| | | Diseases | | |
| 582 | Vortioxetine | Psychiatry | CYP2D6 | Dosage and Administration, Clinical |
| | | | | Pharmacology |
| 583 | Voxelotor | Hematology | HBB | Clinical Pharmacology, Clinical |
| | | | | Studies |
| 584 | Vutrisiran | Neurology | TTR | Adverse Reactions, Clinical |
| | | | | Pharmacology, Clinical Studies |
| 585 | Warfarin | Hematology | CYP2C9 | Dosage and Administration, Drug |
| | | | | Interactions, Clinical Pharmacology |
| 586 | Warfarin | Hematology | VKORC1 | Dosage and Administration, Clinical |
| | | | | Pharmacology |
| 587 | Warfarin | Hematology | PROS1 | Warnings and Precautions |
| 588 | Warfarin | Hematology | PROC | Warnings and Precautions |
| 589 | Zanubrutinib | Oncology | MYD88 | Adverse Reactions, Clinical Studies |
| 591 | Zanubrutinib | Oncology | TP53 | Clinical Studies |
| 592 | Zilucoplan | Neurology | ACHR | Indications and Usage, Clinical |
| | | | | Studies |

What is claimed is:

1. A computer-implemented system for pharmacogenomic determination, the system comprising:
a data processor configured to receive pharmacogenomic data representing at least one pharmacogenomic annotation in association with at least one gene;
a database configuration engine configured to receive at least one genomic variation of the at least one gene and to search the pharmacogenomic data for at least one association with each genomic variation to return the associated data, the associated data being a haplotype or diplotype and a phenotype;
a report generator configured to generate at least one report comprising the associated data with the genomic variation associated; and
a display generator configured to generate a display based on the at least one report, the display further comprising at least one interface element representing the associated data with the genomic variation associated.

2. The computer-implemented system of claim 1, wherein the phenotype comprises adverse drug reactions, metabolizing status, efficacy indications, dosing data, alternative drug data, pharmacogenomic indication, or prescribing data.

3. The computer-implemented system of claim 1,
wherein the report generator is configured to receive at least one text-based file representing at least one genetic sequence and generate at least one binary file representing at least one genetic sequence, at least one index file for the at least one binary file, and at least one text file for the at least one binary file.

4. The computer-implemented system of claim 1, further comprising a machine learning engine configured to predict at least one genomic variant, wherein at least one of the at least one genomic variation is determined as the at least one genomic variant.

5. The computer-implemented system of claim 4, wherein the machine learning engine is configured to detect genomic variants leading to altered protein function, the machine learning engine comprising:
a non-transitory memory storing one or more features from an annotated variant dataset of at least one variant;
a variant validator configured to determine one or more validated variants of the annotated variant dataset, each validated variant matching one or more known variants of a known variant dataset, each known variant leading to altered protein function;
a machine learning model configured to assign a classification to one or more predicted variants of variants of the annotated variant dataset not selected as validated variants, each predicted variant leading to altered protein function, the assigning by the machine learning model based on at least one of the one or more features stored in the memory; and
a loss-of-function detector configured to determine one or more sequence ontology variants of the variants of the annotated variant dataset not selected as validated variants and not classified as predicted variants, each sequence ontology variant being a loss-of-function variant, the determining by the loss-of-function detector based on at least one of the features stored in the memory,
the annotated variant dataset is generated using a Variant Effect Predictor (VEP),
each sequence ontology variant is determined by filtering based on sequence ontology data,
the loss-of-function variant is a splice acceptor variant, a splice donor variant, a stop gained variant, a frameshift variant, a stop loss variant, or a start loss variant.

6. The computer-implemented system of claim 5,
wherein the machine learning model is trained using a training dataset of annotated variants, the training dataset of annotated variants generated based on protein functional domain data, sequence ontology data, at least one prediction score, a LoF indicator feature representing a loss-of-function variant and generated using the sequence ontology data, and an Interpro indicator feature representing an effect on an Interpro domain and generated using the Interpro domain data;
wherein the protein functional domain data is Interpro domain data; and
wherein the sequence ontology data represents a splice acceptor variant, a splice donor variant, a stop gained variant, a frameshift variant, a stop lost variant, a start lost variant, or a combination thereof.

7. The system of claim 5, further comprising:
an interface generator configured to generate one or more user interface objects on a graphical interface of a display, the one or more user interface objects representing:
variant data, the variant data generated based on each validated variant, each predicted variant, and each sequence ontology variant;
wherein the one or more user interface objects is generated based on gene location, functional effect, evidence tag, novelty, or pharmacogenomic data; and
wherein each evidence tag is assigned to each validated variant by the variant validator, each predicted variant by the machine learning model, or each sequence ontology variant by the loss-of-function detector.

8. The system of claim 7, wherein the interface generator is configured to:
receive additional data;
determine an association, if any, between the additional data and each validated variant, each predicted variant, and each sequence ontology variant; and
generate the one or more user interface objects to represent the additional data, if any, associated with each validated variant, each predicted variant, and each sequence ontology variant.

9. The system of claim 5, wherein the classification represents altered protein function corresponding to predicted variants in CYP2B6, CYP2C19, CYP2C9, CYP2D6, DPYD, NUDT15, RYR1, SLCO1B1, TPMT, UGT1A1, BRCA1, BRCA2, or combination thereof.

10. The system of claim 5, further comprising:
using the one or more validated variants, the one or more predicted variants, and the one or more sequence ontology variants to determine a clinical intervention,
using the one or more validated variants, the one or more predicted variants, and the one or more sequence ontology variants to determine responsiveness for a treatment of psychiatric disease,
using the one or more validated variants, the one or more predicted variants, and the one or more sequence ontology variants for multiomics.

11. The system of claim 4, further comprising:
at least one processor; and at least one non-transitory memory storing computer-executable instructions which, when executed, cause the at least one processor to perform a method, the method comprising:
generating at least one annotated variant training dataset, the generating comprising:
receiving at least one annotated variant dataset, annotated based on protein functional domain data, sequence ontology data, and at least one prediction score; and
applying k-nearest neighbour (kNN) imputation to the at least one annotated variant dataset to generate one or more values for missing data; and
training the machine learning model using the at least one annotated variant training dataset,
wherein the at least one annotated variant dataset is annotated using a Variant Effect Predictor (VEP).

12. The system of claim 11,
wherein each prediction score is generated using LoFtool, DEOGEN2, MPC, BayesDel_addAF, FATHMM, integrated_fitCons, or LIST.S2,
wherein the protein functional domain data is Interpro domain data,
wherein the sequence ontology data represents a splice acceptor variant, a splice donor variant, a stop gained variant, a frameshift variant, a stop lost variant, a start lost variant, or a combination thereof,
wherein generating at least one annotated variant training dataset further comprises:
generating a LoF indicator feature using the sequence ontology data, the LoF indicator feature representing a loss-of-function variant,
wherein generating at least one annotated variant training dataset further comprises:
generating an Interpro indicator feature using the Interpro domain data, the Interpro indicator feature representing an effect on an Interpro domain.

13. The system of claim 11,
wherein the machine learning model is a random forest classifier having decision trees, the machine learning model configured to assign a classification based on bootstrap aggregation using the decision trees,
wherein the kNN imputation is kNN imputation with weighted mean,
wherein generating at least one annotated variant training dataset further comprises:
removing data from the at least one annotated variant dataset, wherein the data corresponds to a variant having a percentage greater than or equal to 40%, collectively, of missing values for the annotations, the removing performed before kNN imputation is applied to the at least one annotated variant dataset; and
removing data from the at least one annotated variant dataset, wherein the data corresponds to a feature having a percentage greater than or equal to 40%, collectively, of missing values for variants represented in the at least one annotated variant dataset, the removing performed before kNN imputation is applied to the at least one annotated variant dataset.

14. The system of claim 11, wherein generating at least one annotated variant training dataset further comprises:
performing variant deduplication on the at least one annotated variant dataset to generate at least one new annotated variant dataset;
extracting features from the at least one annotated variant dataset, the features comprising protein functional domain data, sequence ontology data, at least one prediction score, at least one variant identifier, and at least one sequence identifier;
generating a LoF indicator feature using the sequence ontology data, the LoF indicator feature representing a loss-of-function variant; and
generating an Interpro indicator feature using the Interpro domain data, the Interpro indicator feature representing an effect on an Interpro domain.

15. A computer-implemented method for pharmacogenomic determination, the method comprising:
receiving pharmacogenomic data representing at least one pharmacogenomic annotation in association with at least one gene;
receiving at least one genomic variation of the at least one gene, searching the pharmacogenomic data for at least one association with each genomic variation, and returning the associated data, the associated data being a haplotype or diplotype and a phenotype;
generating at least one report comprising the associated data with the genomic variation associated; and
generating a display based on the at least one report, the display further comprising at least one interface element representing the associated data with the genomic variation associated.

16. The computer-implemented method of claim 5, further comprising predicting at least one genomic variant, wherein at least one of the at least one genomic variation is determined as the at least one genomic variant.

17. The computer-implemented method of claim 5,
wherein the at least one text-based file is a FASTQ file,
wherein the at least one binary file is at least one BAM file, the at least one index file is at least one bai file, and the at least one format file is at least one VCF file.

18. A non-transitory computer readable medium storing a set of machine-interpretable instructions, which, when executed, cause a processor to perform a method for pharmacogenomic determination, the method comprising:
receiving pharmacogenomic data representing at least one pharmacogenomic annotation in association with at least one gene;
receiving at least one genomic variation of the at least one gene and searching the pharmacogenomic data for at least one association with each genomic variation to return the associated data; the associated data being a haplotype or diplotype and a phenotype;
generating at least one report comprising the associated data with the genomic variation associated; and
generate a display based on the at least one report, the display further comprising at least one interface element representing the associated data with the genomic variation associated.
